(19) United States
(12) Patent Application Publication
(10) Pub. No.: US 2020/0113912 A1
(43) Pub. Date: Apr. 16, 2020

(54) Methods and Compositions for the Treatment of Disease with Immune Stimulatory Conjugates

(71) Applicant: Silverback Therapeutics, Inc., Seattle, WA (US)

(72) Inventors: Valerie Odegard, Seattle, WA (US); Peter Baum, Seattle, WA (US); Sean Wesley Smith, Seattle, WA (US); Craig Alan Coburn, Seattle, WA (US); Peter Armstrong Thompson, Seattle, WA (US)

(73) Assignee: Silverback Therapeutics, Inc., Seattle, WA (US)

(21) Appl. No.: 16/567,730

(22) Filed: Sep. 11, 2019

Related U.S. Application Data
(60) Provisional application No. 62/730,499, filed on Sep. 12, 2018.
(60) Provisional application No. 62/810,816, filed on Feb. 26, 2019.
(60) Provisional application No. 62/816,992, filed on Mar. 12, 2019.

(51) Int. Cl. A61K 31/55; C07K 16/30; C07K 16/46; C07K 16/28

(52) U.S. Cl. CPC A61K 31/55; C07K 16/30; A61K 45/06; C07K 16/2803; C07K 16/462

(57) ABSTRACT

Methods and conjugates are disclosed for alleviating toxicity(ies) associated with administration of immune-stimulatory conjugates, and in particular for alleviating toxicity(ies) associated with intravenous administration of such conjugates.

### CROSS-REFERENCE TO RELATED APPLICATIONS

This application claims priority to U.S. Provisional Application No. 62/730,499, filed Sep. 12, 2018, U.S. Provisional Application No. 62/810,816, filed Feb. 26, 2019, and U.S. Provisional Application No. 62/816,992, filed Mar. 12, 2019, each of which is incorporated by reference herein in its entirety for any purpose.

### FIELD

The present application relates to immune-stimulatory conjugates and methods of administering immune-stimulatory conjugates.

### BACKGROUND

One of the leading causes of death in the United States is cancer. Conventional methods of cancer treatment, like chemotherapy, surgery, or radiation therapy, tend to be highly toxic and/or nonspecific to a cancer, resulting in limited efficacy and harmful side effects. The immune system has the potential to be a powerful, specific tool in fighting cancers. This observation has led to the development of immunotherapeutics as drug candidates for clinical trials. Immunotherapeutics can act by boosting a specific immune response and have the potential to be a powerful anti-cancer treatment. Like chemotherapy, administration of immunotherapeutics may cause side effects in patients. These side effects may be different than those associated with conventional methods of cancer treatment and will require different methods or techniques for management in patients.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

The present disclosure provides methods and compositions for managing toxicity associated with administration of immune-stimulatory conjugates.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings of which:

FIGS. 1A-D show that wild-type mice dosed IV with HER2-TLR7 exhibited clinical signs of anaphylaxis (1A, 1D), while T- and B-cell deficient SCID mice (1B, 1D) and B-cell deficient J_{H} −/− mice (1C, 1D) did not.

FIG. 2 shows that pre-treatment of mice with B cell-depleting antibody prior to IV dosing with HER2-TLR7 reduced clinical signs of anaphylaxis.

FIGS. 3A-B show that both wild-type (3A) and mast cell-deficient (3B) mice dosed IV with HER2-TLR7 exhibited clinical signs of anaphylaxis.

FIG. 4 shows the effects of depletion of various effector cells in mice prior to a second, weekly dose of HER2-TLR7 on observed rectal temperatures.

FIGS. 5A-B show the level of anti-drug antibodies (ADAs) (5A) and IgG1 antibodies (5B) following IV or SC administration of naked HER2 mAb and HER2-TLR7.

FIGS. 6A-B show the plasma level results from pharmacokinetic studies of HER2-TLR7 following SC and IV administration of 5 mg/kg in mice (6A) and following SC administration of 50 mg/kg in mice (6B).

FIG. 7 shows that a platelet-activating factor (PAF) inhibitor and an anti-histamine, but not dexamethasone, administered prior to IV dosing of HER2-TLR7, mitigated toxicity.

FIG. 8 shows that epinephrine administered after IV dosing of HER2-TLR7 mitigated toxicity.

FIG. 9 shows improved survival following SC dosing of HER2-TLR7 in mice when compared to HER2 mAb alone.

FIG. 10 shows pharmacodynamic profiles from cynomolgus monkeys administered four doses of 6 mg/kg or 12 mg/kg of HER2-TLR8 by subcutaneous injection.

FIGS. 11A-D show that tumor growth slowed in mice following repeat-dose subcutaneous dosing of HER2-TLR7 compared to mice treated with anti-HER2 mAb and PBS controls (11A, HER2 mAb; 11B, HER2-TLR7; 11C, PBS) and that mice treated with HER2-TLR showed a significant survival advantage over controls (11D).

FIGS. 12A-B show tumor volume results for naïve mice and mice pre-treated with subcutaneous HER2-TLR7 challenged with colon carcinoma cells (12A, naïve mice vs. 5 mg/kg pre-treated mice; 12B, naïve mice vs. 20 mg/kg pre-treated mice), demonstrating that mice re-challenged with colon carcinoma cells were protected.

FIG. 13 shows tumor volume results for mice challenged with HER2-negative CT26 cells (mice pre-treated with SC HER2-TLR7 at 50 mg/kg as compared to naïve mice), demonstrating that re-challenged mice were protected from growth of HER2-negative CT26 tumor cells.

FIGS. 14A-B show HER2-TLR7 and TLR7 payload induced of TNF-α production from mouse bone marrow-derived macrophages in the presence of HER2-positive cells, while TLR7 payload but not HER2-TLR7 stimulated TNF-α production in the presence of HER2-negative cells (14A, BMDM+SK-BR-3; 14B, BMDM+MDA-MG-468).

FIGS. 15A-D show elevated cytokines, chemokines, and infiltration/activation of immune cells in HER2+CT26 tumor bearing mice 48 hours after treatment with a single dose of HER2-TLR7 (15A, IFNγ; 15B, IL-1α; 15C, MCP-1; 15D, MIP1α).

FIGS. 16A-F show elevated cytokines, chemokines, and infiltration/activation of immune cells in HER2+CT26 tumor bearing mice 48 hours after treatment with the third of three doses of HER2-TLR7 (16A, IFNγ; 16B, IL-6; 16C, MCP-1; 16D, IP-10; 16E, CXCL1; 16F, CXCL2).

FIGS. 17A-G show an expanded AH-1+ tumor antigen cell population (17A), an increase in the macrophage M1 to M2 ratio (17B), an expansion of AH-1 responsive CD8+ T cells (17C), elevated tumor cell surface PD-L1 expression (17D, 17E), and elevated neutrophil infiltrate (17F, 17G) 48 hours after a single dose, or 48 hours after the third of three doses, of HER2-TLR7.

### DEFINITIONS

Additional aspects and advantages of the present disclosure will become apparent to those skilled in this art from the following detailed description, wherein illustrative aspects of the present disclosure are shown and described. As will be appreciated, the present disclosure is capable of other and different aspects, and its several details are capable of modifications in various respects, all without departing from the disclosure. Accordingly, the descriptions are to be regarded as illustrative in nature, and not as restrictive.

As used herein, “% identity” or “identical”, in the context of the comparison of a polynucleotide, peptide, polypeptide, or protein sequence to another polynucleotide, peptide, polypeptide, or protein sequence, refers to the identity of those sequences. Identity is expressed in terms of a percentage of sequence identity of a first sequence to a second sequence. Percent (%) sequence identity with respect to a reference polynucleotide sequence is the percentage of nucleotides in a candidate sequence that are identical with the nucleotides in the reference polynucleotide sequence after aligning the sequences. Percent (%) sequence identity with respect to a reference amino acid sequence is the percentage of amino acid residues in a sequence that are identical with the amino acid residues in the reference amino acid sequence after aligning the sequences and introducing gaps, if necessary, to achieve the maximum percent sequence identity, and not considering any conservative substitutions as part of the sequence identity.

As used herein, the abbreviations for the natural L-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Unless otherwise specified, X can indicate any amino acid.

As used herein, an “antigen” refers to an antigenic substance that can elicit an immune response in a host. An antigen can be a peptide, polypeptide, protein, polysaccharide, lipid, or glycolipid, which can be recognized by an antibody or other an antigen binding domain. Exposure of immune cells to one or more of these antigens can elicit a rapid cell division and differentiation response resulting in the formation of clones of the exposed T cells and B cells. B cells can differentiate into plasma cells which in turn can produce antibodies which selectively bind to the antigens.

As used herein, a “tumor antigen” refers to an antigenic substance present on a cancer cell that can be recognized by an antibody or antigen binding domain and is preferentially present on a cancer cell as compared to normal (non-cancerous) cells.

As used herein, a “tumor associated antigen” is an antigenic substance that is preferentially present in the extra-cellular environment of cancer cells as compared to the extra-cellular environment of normal (non-cancerous) ells.

As used herein, a “solid tumor antigen” refers to an antigenic substance present on a cancer cell of a solid tumor that can be recognized by an antibody or antigen binding domain and is preferentially present on a cancer cell as compared to normal (non-cancerous) cells. Solid tumors include brain, breast, lung, liver, kidney, pancreatic, colorectal, ovarian, head and neck, bone, skin, mesothelioma, bladder, stomach, prostate, thyroid, uterine and cervical/endometrial cancers. Solid tumors include sarcomas and carcinoma.

As used herein, the term “antibody” refers to an immunoglobulin molecule that specifically binds to, or is immunologically reactive toward, a specific antigen. The term antibody includes, for example, polyclonal, monoclonal, genetically engineered, and antigen binding fragments thereof. An antibody can be, for example, murine, chimeric, humanized, a heteroconjugate, bispecific, diabody, triabody, or tetrabody. An antigen binding fragment can include, for example, a Fab, Fab′, F(ab′)₂, Fv, rIgG, scFv, hcAbs (heavy chain antibodies), a single domain antibody, V_{HH}, V_{NAR}, sdAbs, or nanobody.

As used herein, an “antibody construct” refers to a construct, such as a protein, that includes at least one antigen binding domain and an Fc domain.

As used herein, an “antigen binding domain” refers to a binding domain from an antibody or from a non-antibody that can specifically bind to an antigen. Antigen binding domains can be numbered when there is more than one antigen binding domain in a given conjugate or antibody construct (e.g., first antigen binding domain, second antigen binding domain, third antigen binding domain, etc.). Different antigen binding domains in the same conjugate or construct can bind to target the same antigen or to different antigens (e.g., a first antigen binding domain can specifically bind to a first tumor antigen and a second antigen binding domain can specifically bind to a second tumor antigen).

As used herein, an “Fc domain” refers to a domain from an Fc portion of an antibody or a domain from a non-antibody molecule that can specifically bind to an Fc receptor, such as a Fcgamma receptor or an FcRn receptor. An Fc domain from an antibody can be, for example, a C_{H}1, C_{H}2, C_{H}3 and/or C_{H}4 domain or an Fc receptor binding portion thereof. An Fc domain can also include an Fc region, comprising multiple antibody Fc domains.

As used herein, “recognize” and “specifically bind” with regard to an antigen binding domain interaction with an antigen refer to the specific association or specific binding between the antigen binding domain and the antigen, as compared with the interaction of the antigen binding domain with a different antigen (i.e., non-specific binding). In some embodiments, an antigen binding domain that recognizes or specifically binds to an antigen has a dissociation constant (KD) of <<100 nM, <10 nM, <1 nM, <0.1 nM, <0.01 nM, or <0.001 nM (e.g. 10⁻⁸ M or less, e.g. from 10⁻⁸ M to 10⁻¹³ M, e.g., from 10⁻⁹ M to 10⁻¹³ M).

As used herein, “substantially similar binding affinity” means a binding affinity that differs by less than 30%, or less than 20%, or less than 10% compared to the binding affinity of a reference molecule, where binding affinity is being compared between two different molecules for the same target.

As used herein, an “Fc null” refers to an Fc domain that exhibits weak to no binding to any of the Fcgamma receptors. In some embodiments, an Fc null domain or region exhibits a reduction in binding affinity (e.g., increase in Kd) to Fc gamma receptors of at least 1000-fold.

As used herein, a “myeloid cell” refers to a dendritic cell, a macrophage, a monocyte, a neutrophil, a myeloid derived suppressor cell (MDSC).

As used herein, an “antigen presenting cell” or “APC” refers to a cell that can present antigen to a T-, or B-cell, in a productive manner leading to activation and/or expansion of T-, or B-cell clones specific for said antigen. Nonlimiting exemplary APCs include dendritic cells, macrophages, monocytes, and B cells. In some embodiments, an antigen presenting cell is a dendritic cell, a macrophage, or a monocyte.

As used herein, an “immune stimulatory compound” is a compound or other molecule that directly or indirectly activates or stimulates an immune cell, such as a myeloid cell or an APC.

As used herein, a “myeloid cell agonist” refers to a compound that activates or stimulates an immune response by a myeloid cell.

As used herein, the term “B-cell depleting agent” refers to an agent that, when administered to a subject, causes a reduction in the number of B cells in the subject. In some embodiments, a B-cell depleting agent binds a B cell surface molecule, such as, for example, CD20, CD22, or CD19. In some embodiments, a B-cell depleting agent inhibits a B cell survival factor, such as, for example, BLyS or APRIL. B-cell depleting agents include, but are not limited to, anti-CD20 antibodies, anti-CD19 antibodies, anti-CD22 antibodies, anti-BLyS antibodies, TACI-Ig, BR3-Fc, and anti-BR3 antibodies. Nonlimiting exemplary B-cell depleting agents include rituximab, ocrelizumab, ofatumumab, epratuzumab, MEDI-51 (anti-CD19 antibody), belimumab, BR3-Fc, AMG-623, and atacicept.

As used herein, the term “conjugate” refers to an antibody construct attached to at least one immune stimulatory compound, optionally via a linker(s).

As used herein, an “immune-stimulatory conjugate” refers to a conjugate that activates or stimulates the immune system or a portion thereof, as determined by an in vitro or in vivo assay.

As used herein, an “immune cell” refers to a T cell, B cell, NK cell, NKT cell, or an antigen presenting cell. In some embodiments, an immune cell is a T cell, B cell, NK cell, or NKT cell. In some embodiments, an immune cell is an antigen presenting cell. In some embodiments, an immune cell is not an antigen presenting cell.

As used herein, the term “maximum tolerated dose” or MTD refers to the highest dose of a drug or treatment that does not cause unacceptable side effects.

The terms “salt” or “pharmaceutically acceptable salt” refer to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term-C_{x-y}alkylene- refers to a substituted or unsubstituted alkylene chain with from x to y carbons in the alkylene chain. For example —C₁₋₆alkylene- may be selected from methylene, ethylene, propylene, butylene, pentylene, and hexylene, any one of which is optionally substituted.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively. The term-C_{x-y}alkenylene- refers to a substituted or unsubstituted alkenylene chain with from x to y carbons in the alkenylene chain. For example, —C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term-C_{x-y}alkynylene- refers to a substituted or unsubstituted alkynylene chain with from x to y carbons in the alkenylene chain. For example, —C₂₋₆alkenylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” refers to a divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). Unless stated otherwise specifically in the specification, an alkylene chain is optionally substituted by one or more substituents such as those substituents described herein.

“Alkenylene” refers to a divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene). Unless stated otherwise specifically in the specification, an alkenylene chain is optionally substituted by one or more substituents such as those substituents described herein.

“Alkynylene” refers to a divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene). Unless stated otherwise specifically in the specification, an alkynylene chain is optionally substituted by one or more substituents such as those substituents described herein.

“Heteroalkylene” refers to a divalent hydrocarbon chain including at least one heteroatom in the chain, containing no unsaturation, and preferably having from one to twelve carbon atoms and from one to 6 heteroatoms, e.g., —O—, —NH—, —S—. The heteroalkylene chain is attached to the rest of the molecule through a single bond and to the radical group through a single bond. The points of attachment of the heteroalkylene chain to the rest of the molecule and to the radical group are through the terminal atoms of the chain. In other embodiments, a heteroalkylene comprises one to five carbon atoms and from one to three heteroatoms. In other embodiments, a heteroalkylene comprises one to four carbon atoms and from one to three heteroatoms. In other embodiments, a heteroalkylene comprises one to three carbon atoms and from one to two heteroatoms. In other embodiments, a heteroalkylene comprises one to two carbon atoms and from one to two heteroatoms. In other embodiments, a heteroalkylene comprises one carbon atom and from one to two heteroatoms. In other embodiments, a heteroalkylene comprises five to eight carbon atoms and from one to four heteroatoms. In other embodiments, a heteroalkylene comprises two to five carbon atoms and from one to three heteroatoms. In other embodiments, a heteroalkylene comprises three to five carbon atoms and from one to three heteroatoms. Unless stated otherwise specifically in the specification, a heteroalkylene chain is optionally substituted by one or more substituents such as those substituents described herein.

The term “carbocycle” as used herein refers to a saturated, unsaturated or aromatic ring in which each atom of the ring is carbon. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, and naphthyl. The term “unsaturated carbocycle” refers to carbocycles with at least one degree of unsaturation and excluding aromatic carbocycles. Examples of unsaturated carbocycles include cyclohexadiene, cyclohexene, and cyclopentene.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. The term “unsaturated heterocycle” refers to heterocycles with at least one degree of unsaturation and excluding aromatic heterocycles. Examples of unsaturated heterocycles include dihydropyrrole, dihydrofuran, oxazoline, pyrazoline, and dihydropyridine.

The term “heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also includes polycyclic ring systems having two or more rings in which two or more carbons are common to two adjoining rings wherein at least one of the rings is heteroaromatic, e.g., the other rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

The term “substituted” refers to moieties having substituents replacing a hydrogen on one or more carbons or substitutable heteroatoms, e.g., —NH—, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

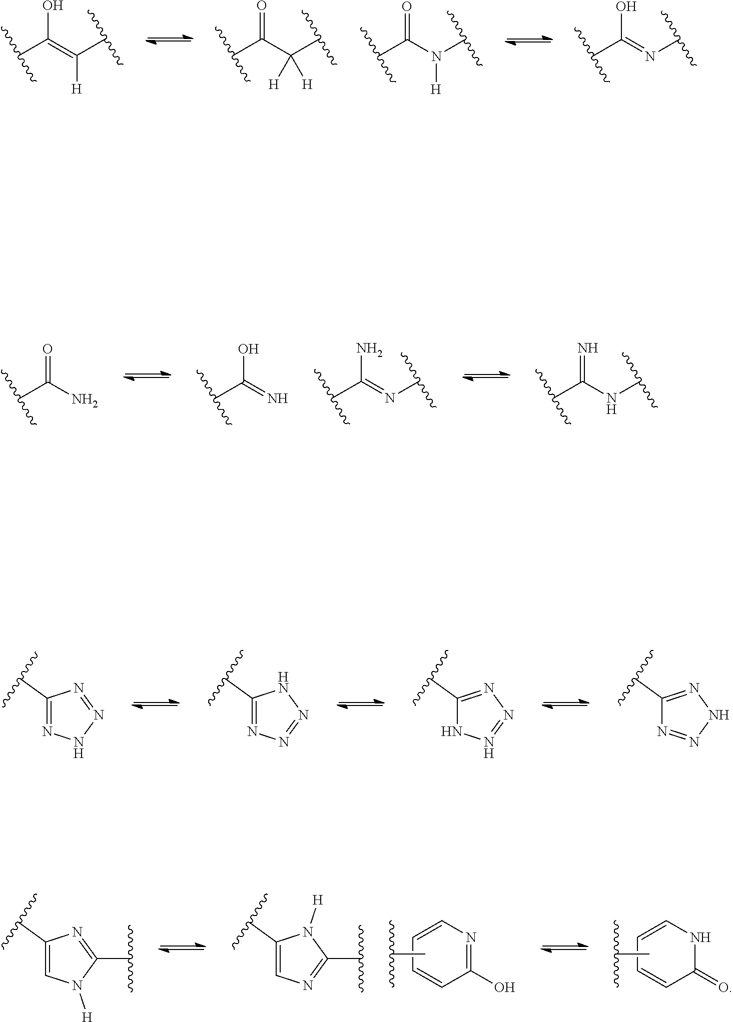

The phrases “intravenous administration” and “administered intravenously” as used herein refer to injection or infusion of a conjugate into a vein of a subject.

The phrases “intravenous slow infusion” and “IV slow infusion” as used here refer to an intravenous infusion that results in a Tmax of about 4 hours or more.

The phrases “subcutaneous administration”, “subcutaneously administering” and the like refer to administration of a conjugate into the subcutis of a subject. For clarity, a subcutaneous administration is distinct from an intratumoral injection into a tumor or cancerous lesion located in the subcuta.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject, according to the route of administration.

The phrase “targeting moiety” refers to a structure that has a selective affinity for a target molecule relative to other non-target molecules. A targeting moiety binds to a target molecule. A targeting moiety may include, for example, an antibody, a peptide, a ligand, a receptor, or a binding portion thereof. The target biological molecule may be a biological receptor or other structure of a cell such as a tumor antigen. A targeting moiety is often specific for a particular cell surface antigen, so as to target an immune-stimulatory compound to a target cell or disease site.

A “small molecule” is an organic compound with a molecular weight of less than 1500, or 100, or 900, or 750, or 600, or 500 Daltons. In some embodiments, a small molecule agonist has an octanol-water partition coefficient (log P) in the range of from 3 to 6, or from 4 to 5, or from 2 to 4. In some embodiments, a small molecule agonist has a polar surface area of less than 200, or less than 150 Å². In some embodiments, the small molecule agonist has not more than five, or not more than three, hydrogen bond donors, and not more than 10, or not more than three hydrogen bond acceptors. A small molecule myeloid cell agonist is not a protein, a polysaccharide, or a nucleic acid.

In addition, it should be understood that individual compounds, or groups of compounds, derived from the various combinations of structures and substituents described herein, are disclosed by the present application to the same extent as if each compound or group of compounds was set forth individually. Thus, selection of particular structures or particular substituents is within the scope of the present disclosure.

The term “about” as used herein in the context of a number refers to a range centered on that number and spanning 10% less than that number and 10% more than that number. The term “about” used in the context of a range refers to an extended range spanning 10% less than that the lowest number listed in the range and 10% more than the greatest number listed in the range. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have,” and “comprise” are used synonymously, which terms and variants thereof are intended to be construed as non-limiting.

The phrase “at least one of” when followed by a list of items or elements refers to an open-ended set of one or more of the elements in the list, which may but does not necessarily include more than one of the elements.

### DETAILED DESCRIPTION

The present inventors have surprisingly discovered that when TLR agonists (e.g., TLR7 and TLR8 agonists) are administered as immune-stimulatory conjugates to a subject, the mode of delivery can be important. Bolus repetitive IV administration can lead to anaphylaxis toxicities. The present inventors have discovered that if the immune-stimulatory conjugate is administered in a manner that results in a Tmax of greater than about 4 hours following each dose, it can be safely administered. Further, the present inventors have discovered that the anaphylaxis toxicities associated with the bolus repetitive IV administration are B-cell mediated and can be diminished with administration with a B-cell depleting agent.

The presently described methods and conjugates provide, inter alia, methods for alleviating or avoiding toxicity(ies) associated with administration of immune-stimulatory conjugates, and in particular for alleviating or avoiding toxicity(ies) associated with intravenous administration (i.e., bolus repetitive intravenous administration) of such conjugates. Generally, anaphylaxis-like toxicity associated with bolus repetitive IV administration is not observed until a subsequent dose is administered at least 7 or 8 days after administration of the first dose. That is, multiple doses may be administered for the first about 7 days without causing anaphylaxis-like toxicity, but a subsequent dose administered after about 7 days can cause anaphylaxis-like toxicity. The methods provide for administration of immune-stimulatory conjugates in a manner that minimizes and/or avoids anaphylaxis-like toxicity regardless of time between doses, for example, by administration of immune-stimulatory conjugates in a manner that results in a Tmax of the immune-stimulatory conjugates of greater than about 4 hours. It some aspects, administration may be by subcutaneous administration. In other aspects, administration may be by intravenous slow infusion. In some aspects, toxicities that can be alleviated, spared, or avoided are anaphylaxis-like toxicities. In some embodiments, the toxicity that is alleviated, spared, or avoided is anaphylaxis-like toxicity. A therapeutically effective regimen comprises at least two or at least three cycles of administration of the conjugate to a subject. Doses of the conjugate within a cycle can be a single dose or as split doses. The doses can be the same or different within a cycle or between cycles.

Immune-stimulatory conjugates useful in the present methods include an antibody construct attached to at least one immune-stimulatory compound typically via a linker(s). The antibody construct has at least one antigen binding domain and an Fc domain. In some embodiments, a conjugate has from 1 to 20 immune-stimulatory compounds per antibody construct, typically from 1 to 8.

Described herein is a method for treating a disease treatable by a TLR agonist, comprising administering to a subject with cancer an effective regimen of an immune-stimulatory conjugate comprising (a) a targeting moiety that specifically binds to an antigen expressed on a disease cell and (b) an immune-stimulatory compound that is a TLR agonist, wherein the effective regimen comprises at least two cycles of administration of the conjugate to the subject, and wherein the effective regimen results in a Tmax of the immune-stimulatory conjugate in the subject of greater than about 4 hours following each administration of the immune-stimulatory conjugate

In some aspects, the disease treatable by the TLR agonist is cancer. Accordingly, described herein is a method for treating cancer, comprising administering to a subject with cancer an effective regimen of an immune-stimulatory conjugate comprising (a) a targeting moiety that specifically binds to a tumor antigen or a tumor associated antigen and (b) an immune-stimulatory compound that is a TLR agonist, wherein the effective regimen comprises at least two cycles of administration of the conjugate to the subject, and wherein the effective regimen results in a Tmax of the immune-stimulatory conjugate in the subject of greater than about 4 hours following each administration of the immune-stimulatory conjugate.

In some aspects, the disease treatable by the TLR agonist is a viral infection. Accordingly, described herein is a method for treating a viral infection, comprising administering to a subject with a viral infection an effective regimen of an immune-stimulatory conjugate comprising (a) a targeting moiety that specifically binds to (i) an antigen present on a cell infected with the virus or (ii) a viral antigen from a virus infecting a cell and (b) an immune-stimulatory compound that is a TLR agonist, wherein the effective regimen comprises at least two cycles of administration of the conjugate to the subject, and wherein the effective regimen results in a Tmax of the immune-stimulatory conjugate in the subject of greater than about 4 hours following each administration of the immune-stimulatory conjugate.

Also described herein is a method of eliciting targeted immune stimulation in a subject, comprising administering an effective regimen of an immune-stimulatory conjugate comprising (a) a targeting moiety that specifically binds to an antigen expressed on a disease cell (e.g., a tumor antigen or a tumor associated antigen) and (b) an immune-stimulatory compound that is a TLR agonist, wherein the effective regimen comprises at least two cycles of administration of the conjugate to the subject, and wherein the effective regimen results in a Tmax of the immune-stimulatory conjugate in the subject of greater than about 4 hours following each administration of the immune-stimulatory conjugate.

The present disclosure further relates to a method for treating a disease treatable with a TLR agonist (e.g., cancer or a viral disease), comprising subcutaneously administering to a subject in need thereof an effective regimen of an immune-stimulatory conjugate comprising (a) a targeting moiety that specifically binds to the relevant antigen (e.g., a tumor antigen or a tumor associated antigen or a viral antigen or another antigen associated with the disease) and (b) an immune-stimulatory compound that is a TLR agonist, wherein the effective regimen comprises at least two cycles of administration of the conjugate to the subject and a total dose of greater than 0.4 mg/kg of the immune-stimulatory conjugate per cycle.

The present disclosure also relates to a method for treating disease (e.g., cancer, a viral disease or another disease treatable with a TLR agonist) comprising administering to a subject in need thereof a B-cell depleting agent and an effective regimen of an immune-stimulatory conjugate comprising (a) a targeting moiety that specifically binds to a tumor antigen or a tumor associated antigen and (b) an immune-stimulatory compound that is a TLR agonist.

Also described herein is a method of eliciting targeted immune stimulation in a subject, comprising administering to a subject in need thereof a B-cell depleting agent and an effective regimen of an immune-stimulatory conjugate comprising (a) a targeting moiety that specifically binds to a tumor antigen or a tumor associated antigen and (b) an immune-stimulatory compound that is a is a TLR agonist.

### Antibody Construct

An immune-stimulatory conjugate as described herein has an antibody construct that includes one or more antigen binding domains and an Fc domain. Each antigen binding domain specifically binds to an antigen. An antibody construct can have, for example, a first antigen binding domain that specifically binds to a first antigen, a second antigen binding domain that specifically binds to a second antigen, and an Fc domain. An antibody construct can be an antibody, wherein the antibody has an antigen binding domain, or pair of antigen binding domains, that specifically bind(s) to an antigen, and an Fc domain. An antibody construct can be a bispecific antibody, wherein the bispecific antibody comprises a first antigen binding domain that specifically binds to a first antigen, a second antigen binding domain that specifically binds to a second antigen, and an Fc domain.

### Antigen Binding Domains

An antigen binding domain can be an antigen-binding portion of an antibody or an antibody fragment that retains the ability to specifically bind to an antigen. An antigen binding domain typically recognizes a single antigen. An antibody construct typically includes, for example, one or two antigen binding domains, although more can be included in an antibody construct. An antibody construct can include two antigen binding domains, in which each antigen binding domain recognizes the same antigen. An antibody construct can include two antigen binding domains, in which each antigen binding domain recognizes the same epitope on the antigen. An antibody construct can include two antigen binding domains in which each antigen binding domain recognizes a different epitope of the same antigen. An antibody construct can include two antigen binding domains in which each antigen binding domain recognizes different antigens. An antibody construct can have three antigen binding domains in which each antigen binding domain recognizes a different antigen. An antibody construct can have three antigen binding domains in which two of the antigen binding domains recognize the same antigen and the third recognizes a different antigen.

An antigen binding domain of an antibody construct can be selected from any portion of an antibody that specifically binds to an antigen. In some embodiments, an antigen binding domain can be a monoclonal antibody, a recombinant antibody, or an antigen binding fragment thereof, for example, a heavy chain variable domain (VH) and a light chain variable domain (VL), a Fab, Fab′, F(ab′)₂, Fv, rIgG, scFv, hcAb (heavy chain antibody), a single domain antibody, V_{HH}, V_{NAR}, sdAbs, or nanobody.

In some embodiments, an antigen binding domain is a non-antibody molecule that specifically binds to an antigen, including, but not limited to, a DARPin, an affimer, an avimer, a knottin, a monobody, lipocalin, an anticalin, ‘T-body’, an affibody, a peptibody, an affinity clamp, an aptamer, or peptide.

In some embodiments, an antigen binding domain is other than an antibody or antigen binding fragment thereof, such as a bicyclic peptide (e.g., a Bicycle®), as described in Published International Application No. WO2014/140342, WO2013/050615, WO2013/050616, and WO2013/050617 (the disclosures of which are incorporated by reference herein).

In certain embodiments, an antigen binding domain specifically binds to an antigen, such as those selected from CD5, CD25, CD37, CD33, CD45, BCMA, CS-1, PD-L1, B7-H3, B7-DC (PD-L2), HLD-DR, carcinoembryonic antigen (CEA), TAG-72, EpCAM, MUC1, folate-binding protein (FOLR1), A33, G250 (carbonic anhydrase IX), prostate-specific membrane antigen (PSMA), GD2, GD3, GM2, Ley, CA-125, CA19-9 (MUC1 sLe(a)), epidermal growth factor, HER2, IL-2 receptor, EGFRvIII (de2-7 EGFR), fibroblast activation protein (FAP), a tenascin, a metalloproteinase, endosialin, avB3, LMP2, EphA2, PAP, AFP, ALK, polysialic acid, TRP-2, fucosyl GM1, mesothelin (MSLN), PSCA, sLe(a), GM3, BORIS, Tn, TF, GloboH, STn, CSPG4, AKAP-4, SSX2, Legumain, Tie 2, Tim 3, VEGFR2, PDGFR-B, ROR2, TRAIL1, MUC16, EGFR, CMET, HER3, MUC1, MUC15, CA6, NAPI2B, TROP2, CLDN18.2, RON, LY6E, FRAlpha, DLL3, PTK7, LIV1, ROR1, CLDN6, GPC3, ADAM12, LRRC15, CDH6, TMEFF2, TMEM238, GPNMB, ALPPL2, UPK1B, UPK2, LAMP-1, LY6K, EphB2, STEAP, ENPP3, CDH3, Nectin4, LYPD3, EFNA4, GPA33, SLITRK6 or HAVCR1.

In certain embodiments, an antigen binding domain specifically binds to a non-proteinaceous or glycoantigen, such as GD2, GD3, GM2, Ley, polysialic acid, fucosyl GM1, GM3, Tn, STn, sLe(animal), or GloboH.

In certain embodiments, an antigen binding domain specifically binds to a solid tumor antigen. In some embodiments, the solid tumor antigen is preferentially present on sarcoma or carcinoma cell(s). In some embodiments, the solid tumor antigen is preferentially present on a sarcoma cell(s). In some embodiments, the solid tumor antigen is preferentially present on a carcinoma cell(s).

In some embodiments, the solid tumor antigen is present on cells of a brain, breast, lung, liver, kidney, pancreatic, colorectal, ovarian, head and neck, bone, skin, mesothelioma, bladder, stomach, prostate, thyroid, uterine or cervical/endometrial cancer.

In some embodiments, the solid tumor antigen is an antigen present on breast cancer, such as HER2, TROP2, LIV-1, CDH3 (p-cadherin), MUC1, Sialo-epitope CA6, PTK7, GPNMB, LAMP-1, LRRC15, ADAM12, EPHA2, TNC, LYPD3, EFNA4 and CLDN6.

In some embodiments, the solid tumor antigen is an antigen present on brain cancer, such as EGFRvIII, TNC and DLL-3.

In some embodiments, the solid tumor antigen is an antigen present on lung cancer, such as mesothelin, HER2, EGFR, PD-L1, MSLN, LY6K, CD56, PTK7, FOLR1, DLL3, SLC34A2, CECAM5, MUC16, LRRC15, ADAM12, EGFRvIII, LYPD3, EFNA4 and MUC1.

In some embodiments, the solid tumor antigen is an antigen present on liver cancer, such as GPC3, EPCAM, CECAM5.

In some embodiments, the solid tumor antigen is an antigen present on kidney cancer, such as HAVCR1, ENPP3, CDH6, CD70, and cMET.

In some embodiments, the solid tumor antigen is an antigen present on pancreatic cancer, such as PTK7, MUC16, MSLN, LRRC15, ADAM12, EFNA4, MUC5A and MUC1.

In some embodiments, the solid tumor antigen is an antigen present on colorectal cancer, such as EPHB2, TMEM238, CECAM5, LRRC15, ADAM12, EFNA4 and GPA33.

In some embodiments, the solid tumor antigen is an antigen present on ovarian cancer, such as MUC16, MUC1, MSLN, FOLR1, sTN, VTCN1, HER2, PTK7, FAP, TMEM238, LRRC15, CLDN6, SLC34A2 and EFNA4.

In some embodiments, the solid tumor antigen is an antigen present on head and neck cancer, such as LY6K, PTK7, LRRC15, ADAM12, LYPD3, EFNA4 and TNC.

In some embodiments, the solid tumor antigen is an antigen present on bone cancer, such as EPHA2, LRRC15, ADAM12, GPNMB, TP-3 and CD248.

In some embodiments, the solid tumor antigen is an antigen present on mesothelioma, such as MSLN.

In some embodiments, the solid tumor antigen is an antigen present on bladder cancer, such as LY6K, PTK7, UPK1B, UPK2, TNC, Nectin4, SLITRK6, LYPD3, EFNA4 and HER2.

In some embodiments, the solid tumor antigen is an antigen present on stomach cancer, such as HER2, EPHB2, TMEM238, CECAM5 and EFNA4.

In some embodiments, the solid tumor antigen is an antigen present on prostate cancer, such as PSMA, FOLH1, PTK7, STEAP, TMEFF2 (TENB2), OR51E2, SLC30A4 and EFNA4.

In some embodiments, the solid tumor antigen is an antigen present on thyroid cancer, such as PTK7.

In some embodiments, the solid tumor antigen is an antigen present on uterine cancer, such as present on uterine cancer such as LY6K, PTK7, EPHB2, FOLR1, ALPPL2, MUC16 and EFNA4.

In some embodiments, the solid tumor antigen is an antigen present on cervical/endometrial cancer, such as LY6K, PTK7, MUC16, LYPD3, EFNA4 and MUC1.

In some embodiments, the solid tumor antigen is an antigen present on a sarcoma, such as LRRC15.

In some aspects, the antigen is a liver cell antigen. In some aspects, the liver cell antigen is expressed on a canalicular cell, Kupffer cell, hepatocyte, or any combination thereof. In some aspects, the liver cell antigen is a hepatocyte antigen. In some aspects, the liver cell antigen is selected from the group consisting of ASGR1 (asialoglycoprotein receptor 1), ASGR2 (asialoglycoprotein receptor 2), TRF2, UGT1A1, SLC22A7, SLC13A5, SLC22A1, and C9. In some aspects, the liver cell antigen is selected from the group consisting of ASGR1, ASGR2, and TRF2. In some aspects, the liver cell antigen is expressed on a liver cell infected with a virus selected from the group consisting of HBV and HCV.

In some aspects, the antigen is a viral antigen from a virus selected from the group consisting of HBV and HCV. In some aspects, the viral antigen is an HBV antigen. In some aspects, the viral antigen is HBsAg, HBcAg, or HBeAg. In some aspects, the viral antigen is HBsAg.

### Fc Domain

An antibody construct includes an Fc domain. An Fc domain is a structure that can bind to one or more Fc receptors (FcRs). An Fc domain can be from an antibody. An Fc domain can be from an IgG antibody. An Fc domain can be from an IgG1, IgG2, or IgG4 antibody. An Fc domain can be a portion of, or all of, an Fc region (e.g, C_{H}1, C_{H}2, C_{H}3, and C_{H}4, according to the type of antibody).

An Fc domain can be part of an antibody that forms an antibody construct. An Fc domain also can be covalently attached to an antigen binding domain(s) to form an antibody construct. An antibody construct can have an antigen binding domain(s) and an Fc domain, wherein the Fc domain is covalently attached to the antigen binding domain(s). An antibody construct can have an antigen binding domain(s) and Fc domain, wherein the Fc domain is covalently attached to an antigen binding domain(s) as an Fc domain-antigen binding domain(s) fusion protein. An antibody construct can have an antigen binding domain(s) and Fc domain, wherein the Fc domain is covalently attached to an antigen binding domain by a linker.

An Fc domain can be a domain of an antibody that can bind to an FcR(s). FcRs are organized into classes (e.g., gamma (γ), alpha (α) and epsilon (ε)) based on the class of antibody that the FcR recognizes. The FcαR class binds to IgA and includes several isoforms, FcαRI (CD89) and FcαμR. The FcγR class binds to IgG and includes several isoforms, FcγRI (CD64), FcγRIIA (CD32a), FcγRIIB (CD32b), FcγRIIIA (CD16a), and FcγRIIIB (CD16b). An FcγRIIIA (CD16a) can be an FcγRIIIA (CD16a) F158 variant or a V158 variant. FcRs also can be FcRn receptors.

Each FcγR isoform can differ in binding affinity to the Fc domain of the IgG antibody. For example, FcγRI can bind to IgG with greater affinity than FcγRII or FcγRIII. The affinity of a particular FcγR isoform to an IgG can be controlled, in part, by a glycan (e.g., oligosaccharide) at position CH2 84.4 of the IgG antibody. For example, fucose containing CH2 84.4 glycans can reduce IgG affinity for FcγRIIIA. In addition, G0 glucans can have increased affinity for FcγRIIIA due to the lack of galactose and terminal GlcNAc moiety.

Binding of an Fc domain to an FcR can enhance an immune response. FcR-mediated signaling that can result from an Fc domain binding to an FcR and can lead to the maturation of immune cells. FcR-mediated signaling that can result from an Fc domain binding to an FcR can lead to the maturation of dendritic cells (DCs). FcR-mediated signaling that can result from an Fc domain binding to an FcR can lead to antibody dependent cellular cytotoxicity. FcR-mediated signaling that can result from an Fc domain binding to an FcR can lead to more efficient immune cell antigen uptake and processing. FcR-mediated signaling that can result from an Fc domain binding to an FcR can promote the expansion and activation of T cells. FcR-mediated signaling that can result from an Fc domain binding to an FcR can promote the expansion and activation of CD8+ T cells. FcR-mediated signaling that can result from an Fc domain binding to an FcR can influence immune cell regulation of T cell responses. FcR-mediated signaling that can result from an Fc domain binding to an FcR can influence immune cell regulation of T cell responses. FcR-mediated signaling that can result from an Fc domain binding to an FcR can influence dendritic cell regulation of T cell responses. FcR-mediated signaling that can result from an Fc domain binding to an FcR can influence functional polarization of T cells (e.g., polarization can be toward a TH1 cell response).

An Fc domain can be modified, such as by a modification of the amino acid sequence, to alter the recognition of an FcR for the Fc domain. Such modification(s) may still allow for FcR-mediated signaling, depending on the modification. A modification can be a substitution of an amino acid at a residue of an Fc domain for a different amino acid at that residue. A modification can be an insertion or deletion of an amino acid at a residue of an Fc domain. A modification can permit binding of an FcR to a site on the Fc domain to which the that the FcR may not otherwise bind. A modification can increase binding affinity of an FcR to the Fc domain. A modification can decrease binding affinity of an FcR to the Fc domain.

An Fc domain can be a variant of a naturally occurring Fc domain (e.g., a wild type Fc domain) and can comprise at least one amino acid change as compared to the sequence of a wild-type Fc domain. An amino acid change in an Fc domain can allow the antibody construct or conjugate to bind to at least one Fc receptor with greater affinity compared to a wild-type Fc domain. An amino acid change in an Fc domain can allow the antibody construct or conjugate to bind to at least one Fc receptor with lessor affinity compared to a wild-type Fc domain.

In some embodiments, an Fc domain exhibits increased binding affinity to one or more Fc receptors. In some embodiments, an Fc domain exhibits increased binding affinity to one or more Fcgamma receptors. In some embodiments, an Fc domain exhibits increased binding affinity to FcRn receptors. In some embodiments, an Fc domain exhibits increased binding affinity to Fcgamma and FcRn receptors. In other embodiments, an Fc domain exhibits the same or substantially similar binding affinity to Fcgamma and/or FcRn receptors as compared to a wild-type Fc domain from an IgG antibody (e.g., IgG1 antibody).

In some embodiments, an Fc domain exhibits decreased binding affinity to one or more Fc receptors. In some embodiments, an Fc domain exhibits decreased binding affinity to one or more Fcgamma receptors. In some embodiments, an Fc domain exhibits decreased binding affinity to FcRn receptors. In some embodiments, an Fc domain exhibits decreased binding affinity to Fcgamma and FcRn receptors. In some embodiments, an Fc domain is an Fc null domain. In some embodiments, an Fc domain exhibits decreased binding affinity to FcRn receptors, but exhibits the same or increased binding affinity to one or more Fcgamma receptors as compared to a wildtype Fc domain. In some embodiments, an Fc domain exhibits increased binding affinity to FcRn receptors, but exhibits the same or decreased binding affinity to one or more Fcgamma receptors.

An Fc domain may have one or more, two or more, three or more, or four or more amino acid substitutions that decrease binding of the Fc domain to an Fc receptor. In certain embodiments, an Fc domain has decreased binding affinity for one or more of FcγRI (CD64), FcγRIIA (CD32), FcγRIIIA (CD16a), FcγRIIIB (CD16b), or any combination thereof. In order to decrease binding affinity of an Fc domain to an Fc receptor, the Fc domain may comprise one or more amino acid substitutions that reduces the binding affinity of the Fc domain to an Fc receptor. In other embodiments, an Fc domain exhibits the same or substantially similar binding affinity to one or more of FcγRI (CD64), FcγRIIA (CD32), FcγRIIIA (CD16a), FcγRIIIB (CD16b), or any combination thereof as compared to a wild-type Fc domain from an IgG antibody (e.g., IgG1 antibody). In some embodiments, an Fc domain can comprise a sequence of an IgG isoform that has been modified from the wild-type IgG sequence. In some embodiments, the Fc domain can comprise a sequence of the IgG1 isoform that has been modified from the wild-type IgG1 sequence. In some embodiments, the modification comprises substitution of one or more amino acids that reduce binding affinity of an IgG Fc domain to all Fcγ receptors.

A modification can be substitution of E233, L234 and L235, such as E233P/L234V/L235A or E233P/L234V/L235A/AG236, according to the EU index of Kabat. A modification can be a substitution of P238, such as P238A, according to the EU index of Kabat. A modification can be a substitution of D265, such as D265A, according to the EU index of Kabat. A modification can be a substitution of N297, such as N297A, according to the EU index of Kabat. A modification can be a substitution of A327, such as A327Q, according to the EU index of Kabat. A modification can be a substitution of P329, such as P239A, according to the EU index of Kabat.

In some embodiments, an IgG Fc domain comprises at least one amino acid substitution that reduces its binding affinity to FcγR1, as compared to a wild-type or reference IgG Fc domain. A modification can comprise a substitution at F241, such as F241A, according to the EU index of Kabat. A modification can comprise a substitution at F243, such as F243A, according to the EU index of Kabat. A modification can comprise a substitution at V264, such as V264A, according to the EU index of Kabat. A modification can comprise a substitution at D265, such as D265A according to the EU index of Kabat.

In some embodiments, an IgG Fc domain comprises at least one amino acid substitution that increases its binding affinity to FcγR1, as compared to a wild-type or reference IgG Fc domain. A modification can comprise a substitution at A327 and P329, such as A327Q/P329A, according to the EU index of Kabat.

In some embodiments, the modification comprises substitution of one or more amino acids that reduce binding affinity of an IgG Fc domain to FcγRII and FcγRIIIA receptors. A modification can be a substitution of D270, such as D270A, according to the EU index of Kabat. A modification can be a substitution of Q295, such as Q295A, according to the EU index of Kabat. A modification can be a substitution of A327, such as A237S, according to the EU index of Kabat.

In some embodiments, the modification comprises substitution of one or more amino acids that increases binding affinity of an IgG Fc domain to FcγRII and FcγRIIIA receptors. A modification can be a substitution of T256, such as T256A, according to the EU index of Kabat. A modification can be a substitution of K290, such as K290A, according to the EU index of Kabat.

In some embodiments, the modification comprises substitution of one or more amino acids that increases binding affinity of an IgG Fc domain to FcγRII receptor. A modification can be a substitution of R255, such as R255A, according to the EU index of Kabat. A modification can be a substitution of E258, such as E258A, according to the EU index of Kabat. A modification can be a substitution of S267, such as S267A, according to the EU index of Kabat. A modification can be a substitution of E272, such as E272A, according to the EU index of Kabat. A modification can be a substitution of N276, such as N276A, according to the EU index of Kabat. A modification can be a substitution of D280, such as D280A, according to the EU index of Kabat. A modification can be a substitution of H285, such as H285A, according to the EU index of Kabat. A modification can be a substitution of N286, such as N286A, according to the EU index of Kabat. A modification can be a substitution of T307, such as T307A, according to the EU index of Kabat. A modification can be a substitution of L309, such as L309A, according to the EU index of Kabat. A modification can be a substitution of N315, such as N315A, according to the EU index of Kabat. A modification can be a substitution of K326, such as K326A, according to the EU index of Kabat. A modification can be a substitution of P331, such as P331A, according to the EU index of Kabat. A modification can be a substitution of S337, such as S337A, according to the EU index of Kabat. A modification can be a substitution of A378, such as A378A, according to the EU index of Kabat. A modification can be a substitution of E430, such as E430, according to the EU index of Kabat.

In some embodiments, the modification comprises substitution of one or more amino acids that increases binding affinity of an IgG Fc domain to FcγRII receptor and reduces the binding affinity to FcγRIIIA receptor. A modification can be a substitution of H268, such as H268A, according to the EU index of Kabat. A modification can be a substitution of R301, such as R301A, according to the EU index of Kabat. A modification can be a substitution of K322, such as K322A, according to the EU index of Kabat.

In some embodiments, the modification comprises substitution of one or more amino acids that decreases binding affinity of an IgG Fc domain to FcγRII receptor but does not affect the binding affinity to FcγRIIIA receptor. A modification can be a substitution of R292, such as R292A, according to the EU index of Kabat. A modification can be a substitution of K414, such as K414A, according to the EU index of Kabat.

In some embodiments, the modification comprises substitution of one or more amino acids that decreases binding affinity of an IgG Fc domain to FcγRII receptor and increases the binding affinity to FcγRIIIA receptor. A modification can be a substitution of S298, such as S298A, according to the EU index of Kabat. A modification can be substitution of S239, 1332 and A330, such as S239D/I332E/A330L. A modification can be substitution of S239 and 1332, such as S239D/I332E.

In some embodiments, the modification comprises substitution of one or more amino acids that decreases binding affinity of an IgG Fc domain to FcγRIIIA receptor. A modification can be substitution of F241 and F243, such as F241S/F243 S or F241I/F243I, according to the EU index of Kabat.

In some embodiments, the modification comprises substitution of one or more amino acids that decreases binding affinity of an IgG Fc domain to FcγRIIIA receptor and does not affect the binding affinity to FcγRII receptor. A modification can be a substitution of S239, such as S239A, according to the EU index of Kabat. A modification can be a substitution of E269, such as E269A, according to the EU index of Kabat. A modification can be a substitution of E293, such as E293A, according to the EU index of Kabat. A modification can be a substitution of Y296, such as Y296F, according to the EU index of Kabat. A modification can be a substitution of V303, such as V303A, according to the EU index of Kabat. A modification can be a substitution of A327, such as A327G, according to the EU index of Kabat. A modification can be a substitution of K338, such as K338A, according to the EU index of Kabat. A modification can be a substitution of D376, such as D376A, according to the EU index of Kabat.

In some embodiments, the modification comprises substitution of one or more amino acids that increases binding affinity of an IgG Fc domain to FcγRIIIA receptor and does not affect the binding affinity to FcγRII receptor. A modification can be a substitution of E333, such as E333A, according to the EU index of Kabat. A modification can be a substitution of K334, such as K334A, according to the EU index of Kabat. A modification can be a substitution of A339, such as A339T, according to the EU index of Kabat. A modification can be substitution of S239 and 1332, such as S239D/I332E.

In some embodiments, the modification comprises substitution of one or more amino acids that increases binding affinity of an IgG Fc domain to FcγRIIIA receptor. A modification can be substitution of L235, F243, R292, Y300 and P396, such as L235V/F243L/R292P/Y300L/P396L (IgG1VLPLL) according to the EU index of Kabat. A modification can be substitution of S298, E333 and K334, such as S298A/E333A/K334A, according to the EU index of Kabat. A modification can be substitution of K246, such as K246F, according to the EU index of Kabat.

Other substitutions in an IgG Fc domain that affect its interaction with one or more Fcγ receptors are disclosed in U.S. Pat. Nos. 7,317,091 and 8,969,526 (the disclosures of which are incorporated by reference herein).

In some embodiments, an IgG Fc domain comprises at least one amino acid substitution that reduces the binding affinity to FcRn, as compared to a wild-type or reference IgG Fc domain. A modification can comprise a substitution at H435, such as H435A according to the EU index of Kabat. A modification can comprise a substitution at 1253, such as I253A according to the EU index of Kabat. A modification can comprise a substitution at H310, such as H310A according to the EU index of Kabat. A modification can comprise substitutions at 1253, H310 and H435, such as I253A/H310A/H435A according to the EU index of Kabat.

A modification can comprise a substitution of one amino acid residue that increases the binding affinity of an IgG Fc domain for FcRn, relative to a wildtype or reference IgG Fc domain. A modification can comprise a substitution at V308, such as V308P according to the EU index of Kabat. A modification can comprise a substitution at M428, such as M428L according to the EU index of Kabat. A modification can comprise a substitution at N434, such as N434A according to the EU index of Kabat or N434H according to the EU index of Kabat. A modification can comprise substitutions at T250 and M428, such as T250Q and M428L according to the EU index of Kabat. A modification can comprise substitutions at M428 and N434, such as M428L and N434S, N434A or N434H according to the EU index of Kabat. A modification can comprise substitutions at M252, S254 and T256, such as M252Y/S254T/T256E according to the EU index of Kabat. A modification can be a substitution of one or more amino acids selected from P257L, P257N, P257I, V279E, V279Q, V279Y, A281S, E283F, V284E, L306Y, T307V, V308F, Q311V, D376V, and N434H. Other substitutions in an IgG Fc domain that affect its interaction with FcRn are disclosed in U.S. Pat. No. 9,803,023 (the disclosure of which is incorporated by reference herein).

In some embodiments, an antibody construct is a human IgG2 antibody, including an IgG2 Fc region. In some embodiments, the heavy chain of the human IgG2 antibody can be mutated at cysteines as positions 127, 232, or 233. In some embodiments, the light chain of a human IgG2 antibody can be mutated at a cysteine at position 214. The mutations in the heavy and light chains of the human IgG2 antibody can be from a cysteine residue to a serine residue.

### Fusion Proteins

In an antibody construct, the first antigen binding domain and additional antigen binding domains (if present) can be attached to the Fc domain as a fusion protein. The first antigen binding domain and a second antigen binding domain can be attached to the Fc domain at an N-terminal end of the Fc domain. The first antigen binding domain can be attached to the Fc domain at an N-terminal end of the Fc domain, and the second antigen binding domain can be attached to the Fc domain at a C-terminal end. The first antigen binding domain can be attached to the Fc domain at an N-terminal end of the Fc domain and the second antigen binding domain can be attached to the Fc domain at a C-terminal end via a polypeptide linker. In some embodiments, the polypeptide linker ranges from about 10 to about 25 amino acids and can, for example, have the sequence [G4S]n where n=2 to about 5.

In some embodiments, the first antigen binding domain can be attached to the Fc domain at a C-terminal end of the Fc domain, and the second antigen binding domain can be attached to the Fc domain at an N-terminal end. The first antigen binding domain and an Fc domain can comprise an antibody and the second binding domain can comprise a single chain variable fragment (scFv) attached to the antibody. The first antigen binding domain, second antigen binding domain and an Fc domain can comprise an antibody and an optional third binding domain can comprise a single chain variable fragment (scFv) attached to the antibody. The second antigen binding domain and an Fc domain can comprise an antibody and a first binding domain can comprise a single chain variable fragment (scFv). A single chain variable fragment can comprise a heavy chain variable domain and a light chain variable domain of an antibody. The first antigen binding domain of the fusion protein can be attached to the second antigen binding domain at a heavy chain variable domain of the single chain variable fragment of the first antigen binding domain (HL orientation). Alternatively, the first antigen binding domain of the fusion protein can be attached to the second antigen binding domain at a light chain variable domain of the single chain variable fragment of the first binding domain (LH orientation). In either orientation, the first antigen binding domain and the second antigen binding domain can be attached via a polypeptide linker. In some embodiments, the polypeptide linker can vary in length from about 15 to about 25 amino acids, and can, for example, have the sequence [G4S]n where n=3 to about 5.

In some embodiments, when a first antigen binding domain and an Fc domain comprise an antibody and the second antigen binding domain comprises a single chain variable fragment (scFv), the second antigen binding domain of the fusion protein can be attached to the first antigen binding domain at a heavy chain variable domain of the single chain variable fragment of the first antigen binding domain (HL orientation). Alternatively, the second antigen binding domain of the fusion protein can be attached to the first antigen binding domain at a light chain variable domain of the single chain variable fragment of the first antigen binding domain (LH orientation).

An antibody construct can comprise a first antigen binding domain and a second antigen binding domain, wherein the second antigen binding domain can be attached to the first antigen binding domain. The antibody construct can comprise an antibody having a light chain and a heavy chain. The first antigen binding domain can comprise a Fab fragment of the light and heavy chains. The second antigen binding domain can be attached to the light chain at a C-terminus or C-terminal end of the light chain as a fusion protein. The second antigen binding domain can comprise a single chain variable fragment (scFv).

An antibody construct can comprise a first antigen binding domain, a second antigen binding domain, and an Fc domain, wherein the first antigen binding domain and the second antigen binding domain are attached to the Fc domain as a fusion protein.

### Antibodies

An antibody construct can comprise an antibody, which can have an antigen binding domain or domains and an Fc domain. An antibody can include of two light chain polypeptides (light chains) and two heavy chain polypeptides (heavy chains), held together covalently by disulfide linkages. The N-terminal regions of the light and heavy chains together form the antigen recognition site of an antibody. The sites that can recognize and can bind to antigen consist of three complementarity determining regions (CDRs), or hypervariable regions, that lie within the framework of the heavy chain variable regions and light chain variable regions at the N-terminal ends of the two heavy and two light chains. The constant domains provide the general framework of the antibody and may not be involved directly in binding the antibody to an antigen, but can be involved in various effector functions, such as participation of the antibody in antibody-dependent cellular cytotoxicity (ADCC).

An antibody of an antibody construct can comprise an antibody of any type, which can be assigned to different classes of immunoglobins, e.g., IgA, IgD, IgE, IgG, and IgM. Several different classes can be further divided into isotypes, e.g., IgG1, IgG2, IgG3, IgG4, IgA1, and IgA2. The heavy-chain constant regions (Fc) that correspond to the different classes of immunoglobulins can be α, δ, ε, γ, and μ, respectively. The light chains can be one of either kappa or κ and lambda or λ, based on the amino acid sequences of the constant domains. An antibody construct can also comprise an antigen-binding fragment or recombinant form of an antibody, including but not limited to a Fab, Fab′, F(ab′)₂, Fv, rIgG, scFv, hcAb (heavy chain antibody), a single domain antibody, V_{HH}, V_{NAR}, sdAbs, or nanobody, that can specifically bind to an antigen.

An antigen binding domain of an antibody typically includes one or more light chain (LC) CDRs (LCDRs) and one or more heavy chain (HC) CDRs (HCDRs), one or more LCDRs or one or more HCDRs. For example, an antigen binding domain of an antibody can comprise one or more of the following: a light chain complementary determining region 1 (LCDR1), a light chain complementary determining region 2 (LCDR2), or a light chain complementary determining region 3 (LCDR3). For another example, an antigen binding domain can comprise one or more of the following: a heavy chain complementary determining region 1 (HCDR1), a heavy chain complementary determining region 2 (HCDR2), or a heavy chain complementary determining region 3 (HCDR3). In some embodiments an antigen binding domain comprises all of the following: a light chain complementary determining region 1 (LCDR1), a light chain complementary determining region 2 (LCDR2), a light chain complementary determining region 3 (LCDR3), a heavy chain complementary determining region 1 (HCDR1), a heavy chain complementary determining region 2 (HCDR2), and a heavy chain complementary determining region 3 (HCDR3). Unless stated otherwise, the CDRs described herein can be defined according to the IMGT (the international ImMunoGeneTics information) system.

An antigen binding domain can comprise only the heavy chain of an antibody (e.g., including the HC CDRs) and does not include any other portion of the antibody). An antigen binding domain can comprise only the variable domain of the heavy chain of an antibody. Alternatively, an antigen binding domain can comprise only the light chain of an antibody (e.g., including the light chain CDRs). An antigen binding domain can comprise only the variable domain of the light chain of an antibody.

An antibody can be chimeric or humanized. Chimeric and humanized forms of non-human (e.g., murine) antibodies can be intact (full length) chimeric immunoglobulins, immunoglobulin chains or antigen binding fragments thereof (such as Fv, Fab, Fab′, F(ab′)₂ or other target-binding subdomains of antibodies), which can contain sequences derived from non-human immunoglobulin. In general, the humanized antibody can comprise substantially all of at least one, and typically two, variable domains, in which all or substantially all of the CDR regions correspond to those of a non-human immunoglobulin and all or substantially all of the framework (FR) regions are those of a human immunoglobulin sequence. A humanized antibody can also comprise at least a portion of an immunoglobulin constant region (Fc), an Fc domain, typically that of a human immunoglobulin consensus sequence.

An antibody described herein can be a human antibody. As used herein, “human antibodies” can include antibodies having, for example, the amino acid sequence of a human immunoglobulin and include antibodies isolated from human immunoglobulin libraries or from animals transgenic for one or more human immunoglobulins and that typically do not express endogenous immunoglobulins. Human antibodies can be produced using transgenic mice which are incapable of expressing functional endogenous immunoglobulins, but which can express human immunoglobulin genes. Completely human antibodies that recognize a selected epitope can be generated using guided selection. In this approach, a selected non-human monoclonal antibody, e.g., a mouse antibody, is used to guide the selection of a completely human antibody recognizing the same epitope

An antibody described herein can be a bispecific antibody or a dual variable domain antibody (DVD). Bispecific and DVD antibodies are monoclonal, often human or humanized, antibodies that have binding specificities for at least two different antigens.

An antibody described herein can be derivatized or otherwise modified. For example, derivatized antibodies can be modified by glycosylation, acetylation, pegylation, phosphorylation, amidation, derivatization by known protecting/blocking groups, proteolytic cleavage, or the like.

An antibody described herein can specifically bind to a cancer antigen. An antibody can specifically bind to a solid tumor antigen.

In some embodiments, the antibody can be trastuzumab, cetuximab, panitumumab, ofatumumab, belimumab, ipilimumab, pertuzumab, tremelimumab, nivolumab, pembrolizumab, atezolizumab, MDX-1105 (WO 2007/005874), dacetuzumab, urelumab, MPDL3280A, lambrolizumab, blinatumomab, nimotuzumab, zalutumumab, onartuzumab, patritumab, clivatuzumab, sofituzumab, edrecolomab, adecatumumab, anetumab, huDS6, lifastuzumab, sacituzumab, PR1A3, humanized PR1A3, humanized Ab2-3, claudiximab, AMG595, ABT806, sibrotuzumab, DS-8895a variant 1, DS-8895a variant 2, MEDI-547, narnatumab, RG7841, farletuzumab, mirvetuximab, J591 variant 1, J591 variant 2, rovalpituzumab, PF-06647020, ladiratuzumab, cirmtuzumab, ladiratuzumab, huLiv1-14 (WO 2012078688), Liv1-1.7A4 (US 2011/0117013), huLiv1-22 (WO 2012078688), 4H11 (US2013/0171152), 4H5 (US2013/0171152), glembatumumab, oportuzumab, enfortumab, depatuxizumab, the antibody of ASG-15ME, huM25 (WO2017/095808A1), or codrituzumab.

In some embodiments, the antibody can be an antigen binding domains of trastuzumab, cetuximab, panitumumab, ofatumumab, belimumab, ipilimumab, pertuzumab, tremelimumab, nivolumab, pembrolizumab, atezolizumab, MDX-1105 (WO 2007/005874), dacetuzumab, urelumab, MPDL3280A, lambrolizumab, blinatumomab, nimotuzumab, zalutumumab, onartuzumab, patritumab, clivatuzumab, sofituzumab, edrecolomab, adecatumumab, anetumab, huDS6, lifastuzumab, sacituzumab, PR1A3, humanized PR1A3, humanized Ab2-3, claudiximab, AMG595, ABT806, sibrotuzumab, DS-8895a variant 1, DS-8895a variant 2, MEDI-547, narnatumab, RG7841, farletuzumab, mirvetuximab, J591 variant 1, J591 variant 2, rovalpituzumab, PF-06647020, ladiratuzumab, cirmtuzumab, ladiratuzumab, huLiv1-14 (WO 2012078688), Liv1-1.7A4 (US 2011/0117013), huLiv1-22 (WO 2012078688), 4H11 (US2013/0171152), 4H5 (US2013/0171152) glembatumumab, oportuzumab, enfortumab, depatuxizumab, the antibody of ASG-15ME, huM25 (WO2017/095808A1), or codrituzumab.

In some embodiments, the antibody comprises LCDR1, LCDR2, LCDR3, HCDR1, HCDR2 and HCDR3, according to the IMGT system, of trastuzumab, cetuximab, panitumumab, ofatumumab, belimumab, ipilimumab, pertuzumab, tremelimumab, nivolumab, pembrolizumab, atezolizumab, MDX-1105 (WO 2007/005874), dacetuzumab, urelumab, MPDL3280A, lambrolizumab, blinatumomab, nimotuzumab, zalutumumab, onartuzumab, patritumab, clivatuzumab, sofituzumab, edrecolomab, adecatumumab, anetumab, huDS6, lifastuzumab, sacituzumab, PR1A3, humanized PR1A3, humanized Ab2-3, claudiximab, AMG595, ABT806, sibrotuzumab, DS-8895a variant 1, DS-8895a variant 2, MEDI-547, narnatumab, RG7841, farletuzumab, mirvetuximab, J591 variant 1, J591 variant 2, rovalpituzumab, PF-06647020, ladiratuzumab, cirmtuzumab, ladiratuzumab, huLiv1-14 (WO 2012078688), Liv1-1.7A4 (US 2011/0117013), huLiv1-22 (WO 2012078688), 4H11 (US2013/0171152), 4H5 (US2013/0171152) glembatumumab, oportuzumab, enfortumab, depatuxizumab, the antibody of ASG-15ME, huM25 (WO2017/095808A1), or codrituzumab.

In some embodiments, an antibody specifically binds to a breast cancer antigen. The antibody can be, for example, trastuzumab, pertuzumab, sacituzumab, ladiratuzumab, huLiv1-14 (WO 2012078688), Liv1-1.7A4 (US 2011/0117013), huLiv1-22 (WO 2012078688), huDS6, glembatumumab, PF-0664720, MEDI-547, DS-8895a variant 1 or DS-08895a variant 2. In some embodiments, an antibody comprises the antigen binding domains of trastuzumab, pertuzumab, sacituzumab, ladiratuzumab, huLiv1-14 (WO 2012078688), Liv1-1.7A4 (US 2011/0117013), huLiv1-22 (WO 2012078688), huDS6, glembatumumab, PF-0664720, MEDI-547, DS-8895a variant 1 or DS-08895a variant 2. In some embodiments, an antibody comprises LCDR1, LCDR2, LCDR3, HCDR1, HCDR2 and HCDR3, according to the IMGT system, of trastuzumab, pertuzumab, sacituzumab, ladiratuzumab, huLiv1-14 (WO 2012078688), Liv1-1.7A4 (US 2011/0117013), huLiv1-22 (WO 2012078688), huDS6, glembatumumab, PF-0664720, MEDI-547, DS-8895a variant 1 or DS-08895a variant 2.

In some embodiments, an antibody specifically binds to an antigen present on brain cancer. The antibody can be, for example, the antibody of AMG595, ABT806, rovalpituzumab or depatuxizumab. In some embodiments, an antibody comprises the antigen binding domains of the antibody of AMG595, ABT806, rovalpituzumab or depatuxizumab. In some embodiments, an antibody comprises LCDR1, LCDR2, LCDR3, HCDR1, HCDR2 and HCDR3, according to the IMGT system, the antibody of AMG595, ABT806, rovalpituzumab or depatuxizumab.

In some embodiments, an antibody specifically binds to an antigen present on lung cancer. The antibody can be, for example, panitumumab, cetuximab, pembrolizumab, nivolumab, atezolizumab, and nimotuzumab, lifastuzumab, anetumab, PF-0664720, farletuzumab, rovalpituzumab, lifastuzumab, sofituzumab, huDS6, ABT806, AMG595 or huM25 (WO2017/095808A1). In some embodiments, an antibody comprises the antigen binding domains of panitumumab, cetuximab, pembrolizumab, nivolumab, atezolizumab, and nimotuzumab, lifastuzumab, anetumab, PF-0664720, farletuzumab, rovalpituzumab, lifastuzumab, sofituzumab, huDS6, ABT806, AMG595 or huM25 (WO2017/095808A1). In some embodiments, an antibody comprises LCDR1, LCDR2, LCDR3, HCDR1, HCDR2 and HCDR3, according to the IMGT system, panitumumab, cetuximab, pembrolizumab, nivolumab, atezolizumab, and nimotuzumab, lifastuzumab, anetumab, PF-0664720, farletuzumab, rovalpituzumab, lifastuzumab, sofituzumab, huDS6, ABT806, AMG595 or huM25 (WO2017/095808A1).

In some embodiments, an antibody specifically binds to an antigen present on liver cancer. The antibody can be, for example, codrituzumab, oportuzumab or humanized PR1A3. In some embodiments, an antibody comprises the antigen binding domains of codrituzumab, oportuzumab or humanized PR1A3. In some embodiments, an antibody comprises LCDR1, LCDR2, LCDR3, HCDR1, HCDR2 and HCDR3, according to the IMGT system, codrituzumab, oportuzumab or humanized PR1A3.

In some embodiments, an antibody specifically binds to an antigen present on kidney cancer. The antibody can be, for example, AGS-16M8F, AGS-16C3, the antibody of CDX-014 or onartuzumab. In some embodiments, an antibody comprises the antigen binding domains of AGS-16M8F, AGS-16C3, the antibody of CDX-014 or onartuzumab. In some embodiments, an antibody comprises LCDR1, LCDR2, LCDR3, HCDR1, HCDR2 and HCDR3, according to the IMGT system, AGS-16M8F, AGS-16C3, the antibody of CDX-014 or onartuzumab.

In some embodiments, an antibody specifically binds to an antigen present on pancreatic cancer. The antibody can be, for example, PF-0664720, clivatuzumab, 4H11(US2013/0171152), 4H5 (US2013/0171152), anetumumab, huDS6, sofituzumab, huM25 (WO2017/095808A1), or RG7841. In some embodiments, an antibody comprises the antigen binding domains of PF-0664720, clivatuzumab, 4H11 (US2013/0171152), 4H5 (US2013/0171152), anetumumab, huDS6, sofituzumab, huM25 (WO2017/095808A1), or RG7841. In some embodiments, an antibody comprises LCDR1, LCDR2, LCDR3, HCDR1, HCDR2 and HCDR3, according to the IMGT system, PF-0664720, clivatuzumab, 4H11 (US2013/0171152), 4H5 (US2013/0171152), anetumumab, huDS6, sofituzumab, huM25 (WO2017/095808A1), or RG7841.

In some embodiments, an antibody specifically binds to an antigen present on colorectal cancer. The antibody can be, for example, huM25 (WO2017/095808A1), PR1A3, humanized PR1A3, pantumumab, cetuximab, nimotuzumab or zalutumumab. In some embodiments, an antibody comprises the antigen binding domains of huM25 (WO2017/095808A1), PR1A3, humanized PR1A3, pantumumab, cetuximab, nimotuzumab or zalutumumab. In some embodiments, an antibody comprises LCDR1, LCDR2, LCDR3, HCDR1, HCDR2 and HCDR3, according to the IMGT system, huM25 (WO2017/095808A1), PR1A3, humanized PR1A3, pantumumab, cetuximab, nimotuzumab or zalutumumab.

In some embodiments, an antibody specifically binds to an antigen present on ovarian cancer. The antibody can be, for example, sofituzumab, 4H11 (US2013/0171152, 4H5 (US2013/0171152), huDS6, farletuzumab, anetumab, trastuzumab, pertuzumab, PF-0664720, sibrotuzumab, huM25 (WO2017/095808A1) or lifastuzumab. In some embodiments, an antibody comprises the antigen binding domains of sofituzumab, 4H11 (US2013/0171152, 4H5 (US2013/0171152), huDS6, farletuzumab, anetumab, trastuzumab, pertuzumab, PF-0664720, sibrotuzumab, huM25 (WO2017/095808A1) or lifastuzumab. In some embodiments, an antibody comprises LCDR1, LCDR2, LCDR3, HCDR1, HCDR2 and HCDR3, according to the IMGT system, sofituzumab, 4H11 (US2013/0171152, 4H5 (US2013/0171152), huDS6, farletuzumab, anetumab, trastuzumab, pertuzumab, PF-0664720, sibrotuzumab, huM25 (WO2017/095808A1) or lifastuzumab.

In some embodiments, an antibody specifically binds to an antigen present on head and neck cancer. The antibody can be, for example, cetuximab, panitumumab, nimtuzumab, PF-0664720, pantumumab, cetuximab, nimotuzumab or zalutumumab. In some embodiments, an antibody comprises the antigen binding domains of cetuximab, panitumumab, nimtuzumab, PF-0664720, pantumumab, cetuximab, nimotuzumab or zalutumumab. In some embodiments, an antibody comprises LCDR1, LCDR2, LCDR3, HCDR1, HCDR2 and HCDR3, according to the IMGT system, cetuximab, panitumumab, nimtuzumab, PF-0664720, pantumumab, cetuximab, nimotuzumab or zalutumumab.

In some embodiments, an antibody specifically binds to an antigen present on bone cancer. The antibody can be, for example, huM25 (WO2017/095808A1), DS-8895a variant 1, DS-8895a variant 2 or glembatumab. In some embodiments, an antibody comprises the antigen binding domains of huM25 (WO2017/095808A1), DS-8895a variant 1, DS-8895a variant 2 or glembatumab. In some embodiments, an antibody comprises LCDR1, LCDR2, LCDR3, HCDR1, HCDR2 and HCDR3, according to the IMGT system, huM25 (WO2017/095808A1), DS-8895a variant 1, DS-8895a variant 2 or glembatumab.

In some embodiments, an antibody specifically binds to an antigen present on skin cancer.

In some embodiments, an antibody specifically binds to an antigen present on mesothelioma.

In some embodiments, an antibody specifically binds to an antigen present on cervical/endometrial cancer. The antibody can be, for example, PF-0664720, anetumumab, 4H11 (US2013/0171152), 4H5 (US2013/0171152), huDS6, or sofituzumab. In some embodiments, an antibody comprises the antigen binding domains of PF-0664720, anetumumab, 4H11 (US2013/0171152), 4H5 (US2013/0171152), huDS6, or sofituzumab. In some embodiments, an antibody comprises LCDR1, LCDR2, LCDR3, HCDR1, HCDR2 and HCDR3, according to the IMGT system, PF-0664720, anetumumab, 4H11 (US2013/0171152), 4H5 (US2013/0171152), huDS6, or sofituzumab.

In some embodiments, an antibody specifically binds to an antigen present on bladder cancer. The antibody can be, for example, enfortumab, trastuzumab, pertuzumab or SLITRK6. In some embodiments, an antibody comprises the antigen binding domains of enfortumab, trastuzumab, pertuzumab or SLITRK6. In some embodiments, an antibody comprises LCDR1, LCDR2, LCDR3, HCDR1, HCDR2 and HCDR3, according to the IMGT system, enfortumab, trastuzumab, pertuzumab or SLITRK6.

In some embodiments, an antibody specifically binds to an antigen present on stomach cancer. The antibody can be, for example, sofituzumab, anetumab, pertuzumab, trastuzumab or humanized PR1A3. In some embodiments, an antibody comprises the antigen binding domains of sofituzumab, anetumab, pertuzumab, trastuzumab or humanized PR1A3. In some embodiments, an antibody comprises LCDR1, LCDR2, LCDR3, HCDR1, HCDR2 and HCDR3, according to the IMGT system, sofituzumab, anetumab, pertuzumab, trastuzumab or humanized PR1A3.

In some embodiments, an antibody specifically binds to an antigen present on prostate cancer. The antibody can be, for example, mirvetuximab, J591 variant 1 or J591 variant 2. In some embodiments, an antibody comprises the antigen binding domains of mirvetuximab, J591 variant 1 or J591 variant 2. In some embodiments, an antibody comprises LCDR1, LCDR2, LCDR3, HCDR1, HCDR2 and HCDR3, according to the IMGT system, mirvetuximab, J591 variant 1 or J591 variant 2.

In some embodiments, an antibody specifically binds to an antigen present on thyroid cancer.

In some embodiments, an antibody specifically binds to an antigen present on uterine cancer. The antibody can be, for example, PF-0664720, farletuzumab, sofituzumab, 4H11 (US2013/0171152 or 4H5 (US2013/0171152). In some embodiments, an antibody comprises the antigen binding domains of PF-0664720, farletuzumab, sofituzumab, 4H11 (US2013/0171152 or 4H5 (US2013/0171152). In some embodiments, an antibody comprises LCDR1, LCDR2, LCDR3, HCDR1, HCDR2 and HCDR3, according to the IMGT system, PF-0664720, farletuzumab, sofituzumab, 4H11 (US2013/0171152 or 4H5 (US2013/0171152).

In some embodiments, an antibody specifically binds to an antigen present on a sarcoma.

In some embodiments, an antibody specifically binds to an antigen present on a liver cell and the subject has a viral infection (e.g., HBV or HCV). The antibody can be, for example, an antibody that binds to ASGR1 or ASGR2.

### Immune-Stimulatory Compounds

The antibody constructs are attached to immune stimulatory compounds, typically via a linker(s) to form immune-stimulatory conjugates. An antibody construct can be attached to one or more immune-stimulatory compounds, typically from about 1 to about 10 compounds per antibody construct.

In some embodiments, an immune stimulatory compound activates human immune cells, including but not limited to dendritic cells, macrophages, monocytes, myeloid-derived suppressor cells, NK cells, B cells, T cells, or tumor cells, or a combination thereof. In some embodiments, an immune-stimulatory compound is a myeloid cell agonist. A myeloid cell agonist is a compound that activates or stimulates an immune response by a myeloid cell. For example, a myeloid cell agonist can stimulate an immune response by causing the release of cytokines by myeloid cells, which results in the activation of immune cells. The stimulation of an immune response by a myeloid cell agonist can be measured in vitro by co-culturing immune cells (e.g., peripheral blood mononuclear cells (PBMCs)) with cells targeted by the conjugate and measuring cytokine release, chemokine release, proliferation of immune cells, upregulation of immune cell activation markers, and/or ADCC. Exemplary assays are described in the Examples. ADCC can be measured by determining the percentage of remaining target cells in the co-culture after administration of the conjugate with the target cells and PBMCs.

In general, an immune stimulatory compound acts on toll like receptors (TLRs), nucleotide-oligomerization domain-like receptors (NOD), RIG-I-Like receptors (RLR), c-type lectin receptors (CLR), or cytosolic DNA Sensors (CDS), or a combination thereof.

In some embodiments, an immune stimulatory compound comprises a ligand of one or more TLRs selected from the group consisting of: TLR2, TLR3, TLR4, TLR5, TLR7, TLR8, TLR7/TLR8, TLR9, and TLR10.

In some embodiments, an immune-stimulatory compound is a myeloid cell agonist. In some embodiments, a myeloid cell agonist is a ligand of TLR2 selected from the group consisting of: (a) a heat killed bacteria product, preferably HKAL, HKEB, HKHP, HKLM, HKLP, HKLR, HKMF, HKPA, HKPG, or HKSA, HKSP, and (b) a cell-wall components product, preferably LAM, LM, LPS, LIA, LIA, PGN, FSL, Pam2CSK4, Pam3CSK4, or Zymosan.

In some embodiments, a myeloid cell agonist is a ligand of TLR3 selected from the group consisting of: rintatolimod, poly-ICLC, RIBOXXON®, Apoxxim, RIBOXXIM®, IPH-33, MCT-465, MCT-475, and ND-1.1.

In some embodiments, a myeloid cell agonist is a ligand of TLR4 selected from the group consisting of LPS, MPLA or a pyrimido[5,4-b]indole such as those described in WO 2014/052828 (U of Cal).

In some embodiments, the myeloid cell agonist is a ligand of TLR5 selected from the group consisting of: FLA and Flagellin.

In some embodiments, the myeloid cell agonist is a ligand of TLR6.

In certain embodiments, a myeloid cell agonist is a TLR7 agonist and/or a TLR8 agonist. In certain embodiments, the myeloid cell agonist is a TLR7 agonist. In certain embodiments, the myeloid cell agonist is a TLR8 agonist. In some embodiments, the myeloid cell agonist selectively agonizes TLR7 and not TLR8. In other embodiments, the myeloid cell agonist selectively agonizes TLR8 and not TLR7.

In certain embodiments, a myeloid cell agonist is a TLR7 agonist. In certain embodiments, the TLR7 agonist is selected from an imidazoquinoline, an imidazoquinoline amine, a thiazoquinoline, an aminoquinoline, an aminoquinazoline, a pyrido[3,2-d]pyrimidine-2,4-diamine, a pyrimidine-2,4-diamine, a 2-aminoimidazole, an 1-alkyl-1H-benzimidazol-2-amine, a tetrahydropyridopyrimidine, a heteroarothiadiazide-2,2-dioxide, a benzonaphthyridine, a thieno[3,2-d]pyrimidine, a 4-amino-imidazoquinoline, an imidazo-pyridinone, an imidazo-pyrimidinone, a purine, a fused pyrimidine-lactam, an imidazo[4,5-c]quinoline-4-amine, an imidazo[4,5-c]quinoline, a pyrimidine, a benzazepine, an imidazo-pyridine, a pyrrolo-pyrimidine, a 2-amino-quinazoline, a guanosine analog, an adenosine analog, a thymidine homopolymer, an ssRNA, CpG-A, PolyG10, and PolyG3. In certain embodiments, the TLR7 agonist is selected from an imidazoquinoline, an imidazoquinoline amine, a thiazoquinoline, an aminoquinoline, an aminoquinazoline, a pyrido[3,2-d]pyrimidine-2,4-diamine, a pyrimidine-2,4-diamine, a 2-aminoimidazole, a 1-alkyl-1H-benzimidazol-2-amine, a tetrahydropyridopyrimidine, a heteroarothiadiazide-2,2-dioxide, a benzonaphthyridine, a thieno[3,2-d]pyrimidine, a 4-amino-imidazoquinoline, an imidazo-pyridinone, an imidazo-pyrimidinone, a purine, a fused pyrimidine-lactam, an imidazo[4,5-c]quinoline-4-amine, an imidazo[4,5-c]quinoline, a pyrimidine, a benzazepine, an imidazo-pyridine, a pyrrolo-pyrimidine, and a 2-amino-quinazoline, but is other than a guanosine analog, an adenosine analog, a thymidine homopolymer, an ssRNA, CpG-A, PolyG10, and PolyG3. In some embodiments, a TLR7 agonist is a non-naturally occurring compound. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the TLR7 modulator compounds disclosed in US20160168164 (Janssen, thieno[3,2-d]pyrimidine derivatives), US 20150299194 (Roche, 4-amino-imidazoquinoline derivatives), US20110098248 (Gilead Sciences, imidazo-pyridinone, imidazo-pyrimidinone, and purine derivatives), US20100143301 (Gilead Sciences, fused pyrimidine-lactam derivatives), and US20090047249 (Gilead Sciences, purine derivatives), and these publications are incorporated by reference herein. Further examples of TLR7 modulators include compounds disclosed in WO2018/009916 (Stanford University/Bolt Biotherapeutics, imidazo[4,5-c]quinolin-4-amine derivatives), WO2018/112108 (Bolt Biotherapeutics, imidazo[4,5-c]quinoline, pyrimidine, benzazepine, imidazo-pyridine, pyrrolo-pyrimidine, and purine derivatives), US2019/0055247 (Bristol-Myers Squibb, purine derivatives), WO2018/198091 (Novartis, pyrrolo-pyrimidine derivatives), US2017/0121421 (Novartis, pyrrolo-pyrimidine derivatives), U.S. Pat. No. 10,253,003 (Janssen, 2-amino-quinazoline derivatives), and U.S. Pat. No. 10,233,184 (Roche, imidazo-pyrimidinone derivatives), and these publications are incorporated by reference herein. In some embodiments, a TLR7 agonist has an EC50 value of 500 nM or less by PBMC assay measuring TNFalpha or IFNalpha production. In some embodiments, a TLR7 agonist has an EC50 value of 100 nM or less by PBMC assay measuring TNFalpha or IFNalpha production. In some embodiments, a TLR7 agonist has an EC50 value of 50 nM or less by PBMC assay measuring TNFalpha or IFNalpha production. In some embodiments, a TLR7 agonist has an EC50 value of 10 nM or less by PBMC assay measuring TNFalpha or IFNalpha production.

In certain embodiments the myeloid cell agonist is a TLR8 agonist. In certain embodiments, the TLR8 agonist is selected from the group consisting of a benzazepine, an imidazoquinoline, a thiazoloquinoline, an aminoquinoline, an aminoquinazoline, a pyrido[3,2-d]pyrimidine-2,4-diamine, a pyrimidine-2,4-diamine, a 2-aminoimidazole, an 1-alkyl-1H-benzimidazol-2-amine, a tetrahydropyridopyrimidine, a pyrido[3,2-d]pyrimidine, a dihydropyrimidinyl benzazepine carboxamide, a benzo[b]azepine, benzazepine dicarboxamide derivatives with a tertiary amide, benzazepine dicarboxamide derivatives with a secondary amide, a quinazoline, a pyrido[3,2-d]pyrimidine, a diamino-pyrimidine, an amino-quinazoline, a heterocyclic-substituted 2-amino-quinazoline, a diamino-pyrimidine, a piperidino-pyrimidine, an alkylamino-pyrimidine, an 8-substituted benzoazepine, an amino-diazepine, an amino-benzo-diazepine, an amido-indole, an amido-benzimidazole, a phenyl sulfonamide, a dihydropteridinone, a fused amino-pyrimidine, a quinazoline, a pyrido-pyrimidine, an amino-substituted benzazepine, a pyrrolo-pyridine, an imidazo-pyridine derivatives, an amino-benzazepine, and a ssRNA. In certain embodiments, a TLR8 agonist is selected from the group consisting of a benzazepine, an imidazoquinoline, a thiazoloquinoline, an aminoquinoline, an aminoquinazoline, a pyrido[3,2-d]pyrimidine-2,4-diamine, a pyrimidine-2,4-diamine, a 2-aminoimidazole, an 1-alkyl-1H-benzimidazol-2-amine, a tetrahydropyridopyrimidine, a pyrido[3,2-d]pyrimidine, a dihydropyrimidinyl benzazepine carboxamide, a benzo[b]azepine, benzazepine dicarboxamide derivatives with a tertiary amide, benzazepine dicarboxamide derivatives with a secondary amide, a quinazoline, a pyrido[3,2-d]pyrimidine, a diamino-pyrimidine, an amino-quinazoline, a heterocyclic-substituted 2-amino-quinazoline, a diamino-pyrimidine, a piperidino-pyrimidine, an alkylamino-pyrimidine, an 8-substituted benzoazepine, an amino-diazepine, an amino-benzo-diazepine, an amido-indole, an amido-benzimidazole, a phenyl sulfonamide, a dihydropteridinone, a fused amino-pyrimidine, a quinazoline, a pyrido-pyrimidine, an amino-substituted benzazepine, a pyrrolo-pyridine, an imidazo-pyridine derivatives, and an amino-benzazepine, and is other than a ssRNA. In some embodiments, a TLR8 agonist is a non-naturally occurring compound. Examples of TLR8 agonists include motolimod, resiquimod, 3M-051, 3M-052, MCT-465, IMO-4200, VTX-763, VTX-1463, and the TLR8 modulator compounds disclosed in US20180086755 (Gilead, pyrido[3,2-d]pyrimidine derivatives), WO2017216054 (Roche, dihydropyrimidinyl benzazepine carboxamide derivatives), WO2017190669 (Shanghai De Novo Pharmatech, benzo[b]azepine derivatives), WO2016142250 (Roche, benzazepine dicarboxamide derivatives), WO2017202704 (Roche, benzazepine dicarboxamide derivatives with a tertiary amide), WO2017202703 (Roche, benzazepine dicarboxamide derivatives with a secondary amide), US20170071944 (Gilead, quinazoline and pyrido[3,2-d]pyrimdine derivatives), US20140045849 (Janssen, diamino-pyrimidine derivatives), US20140073642 (Janssen, amino-quinazoline derivatives), WO2014056953 (Janssen, pyrrolo[3,2-d]pyrimidine derivatives), WO2014076221 (Janssen, heterocyclic substituted 2-amino-quinazoline derivatives), WO2014128189 (Janssen, diamino-pyrimidine derivatives), US20140350031 (Janssen, piperidino-pyrimidine derivatives), WO2014023813 (Janssen, alkyl-aminopyrimidine derivatives), US20080234251 (Array Biopharma, 8-substituted benzoazepine derivatives), US20080306050 (Array Biopharma, amino-diazepine derivatives), US20100029585 (VentiRx Pharma, amino-benzazepine derivatives), US20110092485 (VentiRx Pharma, amino-benzazepine derivatives), US20110118235 (VentiRx Pharma, amino-benzazepine derivatives), US20120082658 (VentiRx Pharma, amino-benzazepine VTX-378), US20120219615 (VentiRx Pharma), US20140066432 (VentiRx Pharma, amino-benzazepine VTX-2337), US20140088085 (VentiRx Pharma, amino-benzazepine and amino-benzo-diazepine derivatives), US20140275167 (Novira Therapeutics, amido-indole and amido-benzimidazole derivatives), and US20130251673 (Novira Therapeutics, phenyl sulfonamide derivatives), and these publications are incorporated by reference herein. Further examples of TLR8 modulators include compounds disclosed in US2016/0108045 (Gilead, dihydropteridinone derivatives), US2018/0065938 (Gilead, fused amino-pyrimidine derivatives), US2018/0263985 (Gilead, quinazoline and pyrido-pyrimidine derivatives), WO2017/046112 (Roche, amino-substituted benzazepine derivatives), WO2016/096778 (Roche, amino-substituted benzazepine derivatives), US2019/0016808 (Birdie Biopharmaceuticals, pyrrolo- or imidazo-pyridine derivatives or amino-benzazepine derivatives), and these publications are incorporated by reference herein. In some embodiments, the TLR8 agonist comprises the structure:

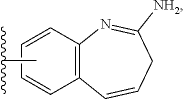

wherein the structure is optionally substituted at any position other than the —NH₂ position. In some embodiments, a TLR8 agonist has an EC50 value of 500 nM or less by PBMC assay measuring TNFalpha production. In some embodiments, a TLR8 agonist has an EC50 value of 100 nM or less by PBMC assay measuring TNFalpha production. In some embodiments, a TLR8 agonist has an EC50 value of 50 nM or less by PBMC assay measuring TNFalpha production. In some embodiments, a TLR8 agonist has an EC50 value of 10 nM or less by PBMC assay measuring TNFalpha production.

In some embodiments, a TLR8 agonist is a benzazepine selected from compounds 1.1-1.2, 1.4-1.20, 1.23-1.27, 1.29-1.46, 1.48, and 1.50-1.67, as shown in the Examples.

In some embodiments, a myeloid cell agonist is a ligand of TLR9 selected from the group consisting of: ODN1585, ODN1668, ODN1826, PF-3512676 (ODN2006), ODN2007, ODN2216, ODN2336, ODN2395, BB-001, BB-006, CYT-003, IMO-2055, IMO-2125, IMO-3100, IMO-8400, IR-103, IMO-9200, agatolimod, DIMS-9054, DV-1079, DV-1179, AZD-1419, leftolimod (MGN-1703), litenimod, and CYT-003-QbG10.

In other embodiments, the myeloid agonist selectively agonizes TLR9, TLR3, TLR4, TLR2, TLR5, RIG-I, STING, cGAS, NOD1, NOD2, NOD1/NOD2, NRLP3, ALPK1, MDA5 AIM2, IRE1 and PERK.

In some embodiments, a myeloid cell agonist is a ligand of TLR10.

In some embodiments, a myeloid cell agonist is a ligand of a ligand of nucleotide-oligomerization domain (NOD)-like selected from the group consisting of: NOD1 agonist (C12-iE-DAP, iE-DAP, Tri-DAP), NOD2 agonist (L18-MDP, MDP, M-TriLYS, M-TriLYS-D-ASN, Murabutide, N-Glycolyl-MDP), and NOD1/NOD2 agonists (M-TriDAP, PGN).

In some embodiments, a myeloid cell agonist is a ligand of one or more RIG-I-Like receptors (RLR) selected from the group consisting of: S′ppp-dsRNA, Poly (dA:dT), Poly(dG:dC), and Poly (I:C).

In some embodiments, a myeloid cell agonist is a ligand of one or more C-type lectin receptors (CLR) selected from the group consisting of: Cnrdlan AL, HKCA, HKSC, WGP, Zymosan, and Trehalose-6,6-dibehenate.

In some embodiments, a myeloid cell agonist is a ligand of one or more Cytosolic DNA Sensors (CDS) selected from the group consisting of: ADU-S100, c-GMP, c-G-AMP, c-G-GMP, c-A-AMP, c-di-AMP, c-di-IMP, c-di-GMP, c-di-UMP, HSV-60, ISD, pCpG, Poly (dA:dT), Poly(dG:dC), Poly (dA), VACV-70 and ca-mangostin and the compounds disclosed in WO2018156625 (U of Texas), WO 2018152453 (Eisai), WO 2018138685 (Janssen), WO2018100558 (Takeda), WO2018098203 (Janssen), WO2018065360 (Biolog Life Sciences), WO2018060323 (Boehringer Ingelheim), WO2018045204 (IFM Therapeutics), WO2018009466 (Aduro), WO 2017161349 (Immune Sensor), WO2017123669, WO2017123657, WO2017027646 (Merck), WO2017027645 (Merck), WO2016120305 (GSK), WO2016096174 (InvivoGen), and US20140341976 (Aduro).

In some embodiments, the myeloid cell agonist is a ligand of an inflammasome inducer selected from the group consisting of: (a) NLRP3 inflammasome protein complex, preferably alum Crystals, ATP, CPPD Crystals, Hennozoin, MSU Crystals, Nano-Si 02, Nigericin, and (b) AIM2 inflammasome protein complex, such as Poly (dA:dT).

In certain aspects, a TLR8 agonist or a TLR7 agonist is selected from Category A or Category B, respectively, as further described herein. Variables and Formula of the Compounds of Category A (TLR8 agonists) are described in the section entitled Compounds of Category A, and variables and Formula of the Compounds of Category B (TLR7 agonists) are described in the subsequent section, entitled Compounds of Category B. Formulas and variables of the Compounds of Category A and the Compounds of Category B may overlap in nomenclature, e.g., Formula IA for both Compounds of Category A and Category B; however variables and Formula descriptions are not intended to be interchangeable between the categories.

In some aspects, the myeloid cell agonist is a benzazepine-4-carboxamide compound. In some aspects, the benzazepine-4-carboxamide compound has the structure of Formula X-1:

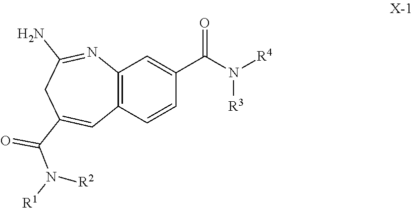

wherein:
R¹ is C₃₋₇alkyl;
R² is C₃₋₇alkyl or C₃₋₇cycloalkyl-C₁₋₇alkyl;
R³ is hydrogen;
R⁴ is selected from the group consisting of
   C₁₋₇alkyl, said C₁₋₇alkyl being unsubstituted or substituted by one or two groups selected from the group consisting of phenyl and heteroaryl, said heteraryl being an aromatic 5- or 6-membered ring which comprises one, two, or three atoms selected from nitrogen, oxygen, and/or sulfur;
   C₃₋₇cycloalkyl, said C₃₋₇cycloalkyl being unsubstituted or substituted by phenyl or phenylamino-C₁₋₄alkyl, and
   heterocyclyl, said heterocyclyl being a saturated 3- to 7-membered ring containing one heteroatom selected from N and O and being unsubstituted or substituted by phenyl.
      Structures of Formula X-1 are described, for example, in PCT Publication No. WO2017/202703.

In some aspects, the the myeloid cell agonist is a benzazepine-dicarboxamide compound.

In some aspects, the benzazepine-dicarboxamide compound has the structure of Formula X-2:

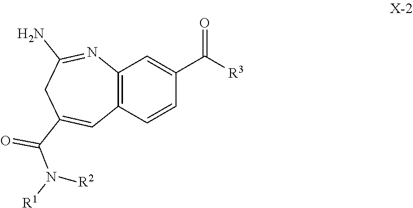

wherein:
R¹ is C₃₋₇alkyl;
R² is C₃₋₇alkyl or C₃₋₇cycloalkyl-C₁₋₇alkyl;
R³ is a heterocycle selected from

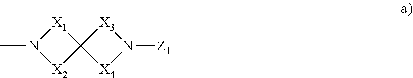

wherein
X₁ is (CH₂)ₘ wherein m is 1 or 2;
X₂ is (CH₂)ₙ wherein n is 1 or 2;
X₃ is (CH₂)ₒ wherein o is 1 or 2;
X₄ is (CH₂)ₚ wherein p is 1 or 2; and
Z₁ is phenyl, wherein phenyl is unsubstituted or substituted by one or two groups selected from the group consisting of C₁₋₇alkyl, halogen, halogen-C₁₋₇alkyl, C₁₋₇alkoxy, hydroxy-C₁₋₇alkyl, amino-C₁₋₇alkyl, C₁₋₇alkyl-amino-C₁₋₇alkyl, and di-C₁₋₇alkyl-amino-C₁₋₇alkyl; or

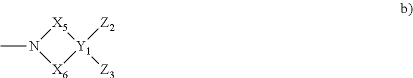

wherein
X₅ is (CH₂)_{q} wherein q is 1 or 2;
X₆ is (CH₂)ᵣ wherein r is 1 or 2;
Y₁ is a carbon or nitrogen atom;
Z₂ is hydrogen; and
Z₃ is selected from the group consisting of hydrogen, C₁₋₇alkoxy, C₂₋₇alkenyloxy, phenyl, phenyl-C₁₋₇alkyl, phenyl-C₁₋₇alkyloxy, phenyl-C₁₋₇alkylamino, phenylamino-C₁₋₇alkyl, phenylamino, wherein phenyl is unsubstituted or substituted by one or two groups selected from the group consisting of C₁₋₇alkyl, halogen, halogen-C₁₋₇alkyl, C₁₋₇alkoxy, hydroxy-C₁₋₇alkyl, amino-C₁₋₇alkyl, C₁₋₇alkyl-amino-C₁₋₇alkyl, and di-C₁₋₇alkyl-amino-C₁₋₇alkyl; or

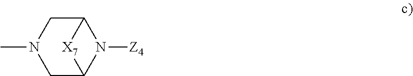

wherein
X₇ is (CH₂)ₛ wherein s is 1 or 2; and
Z₄ is phenyl, wherein phenyl is unsubstituted or substituted by one or two groups selected from the group consisting of C₁₋₇alkyl, halogen, halogen-C₁₋₇alkyl, C₁₋₇alkoxy, hydroxy-C₁₋₇alkyl, amino-C₁₋₇alkyl, C₁₋₇alkyl-amino-C₁₋₇alkyl, and di-C₁₋₇alkyl-amino-C₁₋₇alkyl; or

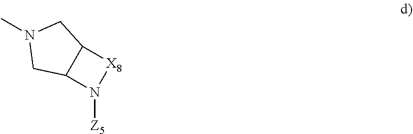

wherein
X₈ is (CH₂)ₜ wherein t is 1 or 2; and
Z₅ is phenyl, wherein phenyl is unsubstituted or substituted by one or two groups selected from the group consisting of C₁₋₇alkyl, halogen, halogen-C₁₋₇alkyl, C₁₋₇alkoxy, hydroxy-C₁₋₇alkyl, amino-C₁₋₇alkyl, C₁₋₇alkyl-amino-C₁₋₇alkyl, and di-C₁₋₇alkyl-amino-C₁₋₇alkyl.
   Compounds of Formula X-2 are described, for example, in PCT Publication No. WO2017/202704.

In some aspects, the the myeloid cell agonist is a benzazepine sulfonamide compound. In some aspects, the benzazepine sulfonamide compound has the structure of Formula X-3:

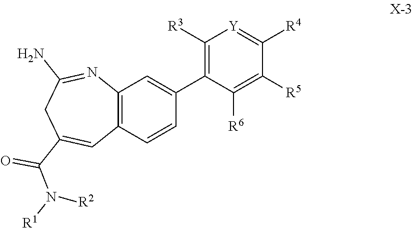

wherein
R¹ and R² are the same or different and are selected from the group consisting of C₁₋₇alkyl, hydroxy-C₂₋₇alkyl, amino-C₂₋₇alkyl, C₂₋₇alkenyl, and C₃₋₇alkynyl;
R³ is hydrogen or C₁₋₇alkyl;
R⁶ is hydrogen or C₁₋₇alkyl;
one of R⁴ and R⁵ is selected from the group consisting of hydrogen, C₁₋₇alkyl, halogen-C₁₋₇alkyl, and C₁₋₇alkoxy,
and the other one of R⁴ and R⁵ is

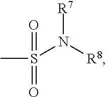

wherein R⁷ and R⁸ are the same or different and are selected from the group consisting of hydrogen, C₁₋₇alkyl, halogen-C₁₋₇alkyl, hydroxy-C₁₋₇alkyl, hydroxy-C₁₋₇alkoxy-C₁₋₇alkyl, amino-C₁₋₇alkyl, C₁₋₇alkyl-amino-C₁₋₇alkyl, amino-C₁₋₇alkoxy-C₁₋₇alkyl, C₁₋₇alkyl-amino-C₁₋₇alkoxy-C₁₋₇alkyl, amino-C₁₋₇alkyl-carbonyl, and C₁₋₇alkyl-xamino-C₁₋₇alkyl-carbonyl; or
R⁷ and R⁸ together with the nitrogen atom they are attached to form a 4- to 6-membered heterocycle which is unsubstituted or substituted with a group selected from the group consisting of amino, C₁₋₇alkyl-amino, hydroxy, and hydroxy-C₁₋₇alkyl, and which may contain an additional N—R¹⁰ group, wherein R¹⁰ is selected from the group consisting of hydrogen, amino-C₁₋₇alkyl, and C₁₋₇alkyl-amino-C₁₋₇alkyl; and
Y is N or CR⁹;
   wherein R⁹ is selected from the group consisting of hydrogen, C₁₋₇alkyl, and halogen-C₁₋₇ alkyl.
      Compounds of Formula X-3 are described, for example, in PCT Publication No. WO2016/096778.

In some aspects, the myeloid cell agonist is a dihydropyrimidinyl benzazepine carboxamide compound. In some aspects, the dihydropyrimidinyl benzazepine carboxamide compound has the structure of Formula X-4:

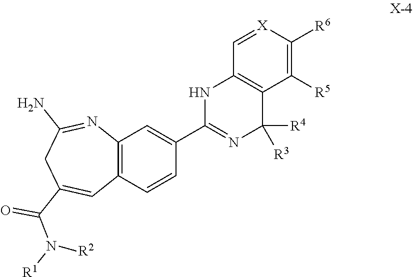

wherein
R¹ is C₃₋₇alkyl;
R² is C₃₋₇alkyl or C₃₋₇cycloalkyl-C₁₋₇alkyl;
R³ is hydrogen or C₁₋₇alkyl;
R⁴ is hydrogen or C₁₋₇alkyl;
R⁵ is selected from the group consisting of hydrogen, halogen, C₁₋₇alkyl, and C₁₋₇alkoxy;
R⁶ is selected from the group consisting of hydrogen, halogen, C₁₋₇alkyl, and C₁₋₇alkoxy; and
X is N or CR⁷, wherein R⁷ is selected from the group consisting of hydrogen, halogen, C₁₋₇alkyl, and C₁₋₇alkoxy.
   Compounds of Formula X-4 are described, for example, in PCT Publication No. WO2017/216054.

In some aspects, the myeloid cell agonist is a sulfinylphenyl or sulfonimidoylphenyl benzazepine compound. In some aspects, the sulfinylphenyl or sulfonimidoylphenyl benzazepine compound has the structure of Formula X-5:

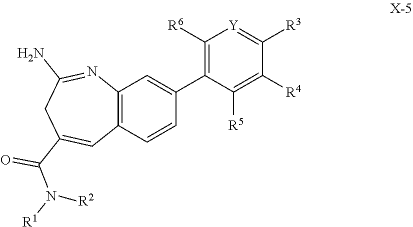

wherein
X is CR⁷ or N;
R¹ is C₃₋₇alkyl or C₃₋₇cycloalkyl;
R² is selected from the group consisting of C₃₋₇alkyl, hydroxy-C₁₋₇alkyl, C₃₋₇-alkynyl, amino-C₁₋₇alkoxy-C₁₋₇alkoxy-C₁₋₇alkyl, halogen-C₁₋₇alkyl, and C₃₋₇cycloalkyl-C₁₋₇alkyl;
one of R³ and R⁴ is

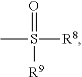

and the other one of R³ and R⁴ is selected from the group consisting of hydrogen, C₁₋₇alkyl, and halogen;
R⁵, R⁶, and R⁷ are independently from each other selected from hydrogen, C₁₋₇alkyl, and halogen;
R⁸ is C₁₋₇alkyl; and
R⁹ is absent or is ═N—R¹⁰, wherein R¹⁰ is selected from the group consisting of hydrogen, C₁₋₇alkyl, halogen-C₁₋₇alkyl, hydroxy-C₁₋₇alkyl, and hydroxy-C₁₋₇alkoxy-C₁₋₇alkyl.
   Compounds of Formula X-5 are described, for example, in PCT Publication No. WO2017/046112.

In some aspects, the myeloid cell agonist is a TLR modulator compound that has the structure of Formula X-6:

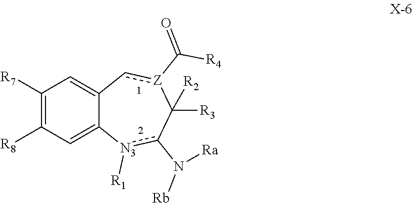

wherein
[symbol] (1) is a double bond or a single bond;
[symbol] (2) is a single bond or is double bond and R₁ is absent;
R₂ and R₃ are independently selected from H and lower alkyl, or R₂ and R₃ are connected to form a saturated carbocycle having from 3 to 7 ring members;
one of R₇ and R₈ is —NR_{f}R_{g},

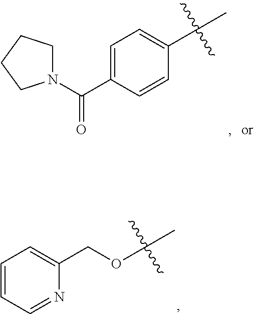 [SMILES: *c1ccc(C(=O)N2CCCC2)cc1 ; *OCc1ccccn1]

and the other is hydrogen;
where R_{f} and R_{g} are lower alkyl or R_{f} and R_{g} together with the nitrogen to which they are attached form a saturated heterocyclic ring having 4 to 6 ring members;
R₄ is —NR_{c}R_{d} or —OR₁₀;
   R_{c} and R_{d} are lower alkyl, where the alkyl is optionally substituted with one or more —OH;
   R₁₀ is alkyl, where the alkyl is optionally substituted with one or more —OH;
Z is C and [symbol] (1) is a double bond, or Z is N and [symbol] (1) is a single bond;
Rₐ and R_{b} are independently selected from H, alkyl, alkenyl, alkynyl, and R^{e}, wherein the alkyl is optionally substituted with one or more —OR¹⁰, or R^{e};
   R^{e} is selected from —NH₂, —NH(alkyl), and —N(alkyl)₂;
R¹ is absent when [symbol] (2) is a double bond, or when [symbol] (2) is a single bond, R¹ and one of R^{a} or R^{b} are taken together with the atoms to which they are attached to form a saturated, partially unsaturated, or unsaturated heterocycle having 5-7 ring members, and the other of R^{a} or R^{b} is hydrogen or is absent as necessary to accommodate ring unsaturation.

In some aspects, the myeloid cell agonist is a TLR modulator compound that has the structure of Formula X-7:

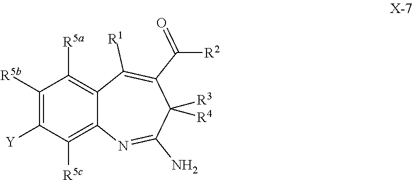

wherein
Y is CF₂CF₃, CF₂CF₇R⁶, or an aryl or heteroaryl ring, wherein said aryl and heteroaryl rings are substituted with one or more groups independently selected from alkenyl, alkynyl, Br, CN, OH, NR⁶R⁷, C(═O)R⁸, NR⁶SO₂R⁷, (C₁-C₆ alkyl)amino, R⁶OC(═O)CH═CH₂—, SR⁶ and SO₂R⁶, and wherein the aryl and heteroaryl rings are optionally further substituted with one or more groups independently selected from F, Cl, CF₃, CF₃O—, HCF₂O—, alkyl, heteroalkyl and ArO—;
R¹, R³ and R⁴ are independently selected from H, alkyl, alkenyl, alkynyl, heteroalkyl, cycloalkyl, cycloalkenyl, heterocycloalkyl, aryl and heteroaryl, wherein the alkyl, alkenyl, alkynyl, heteroalkyl, cycloalkyl, cycloalkenyl, heterocycloalkyl, aryl and heteroaryl are optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(O)CH═CH₂—, NR⁶SO₂R⁷, SR⁶ and SO₂R⁶,
or R³ and R⁴ together with the atom to which they are attached form a saturated or partially unsaturated carbocyclic ring, wherein the carbocyclic ring is optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH₂—, NR⁶SO₂R⁷, SR⁶ and SO₂R⁶;
R² and R⁸ are independently selected from H, OR⁶, NR⁶R⁷, alkyl, alkenyl, alkynyl, heteroalkyl, cycloalkyl, cycloalkenyl, heterocycloalkyl, aryl and heteroaryl, wherein the alkyl, alkenyl, alkynyl, heteroalkyl, cycloalkyl, cycloalkenyl, heterocycloalkyl, aryl and heteroaryl are optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(O)NR⁶R⁷, (C₁-C₆ alkyl)amino, CH₃OCH₂O—, R⁶OC(═O)CH═CH₂—, NR⁶SO₂R⁷, SR⁶ and SO₂R⁶;
R^{5a}, R^{5b}, and R^{5c} are independently H, F, Cl, Br, I, OMe, CH₃, CH₂F, CHF₂ or CF₃; and
R⁶ and R⁷ are independently selected from H, alkyl, alkenyl, alkynyl, heteroalkyl, cycloalkyl, cycloalkenyl, heterocycloalkyl, aryl and heteroaryl, wherein said alkyl, alkenyl, alkynyl, heteroalkyl, cycloalkyl, cycloalkenyl, heterocycloalkyl, aryl and heteroaryl are optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(O)CH═CH₂—, NR⁶SO₂R⁷, SR⁶ and SO₂R⁶,
or R⁶ and R⁷ together with the atom to which they are attached form a saturated or partially unsaturated heterocyclic ring, wherein said heterocyclic ring is optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆alkyl)amino, CH₃OCH₂O—, R⁶OC(═O)CH═CH₂—, NR⁶SO₂R⁷, SR⁶ and SO₂R⁶.

In some aspects, the myeloid cell agonist is a TLR modulator compound that has the structure of Formula X-8:

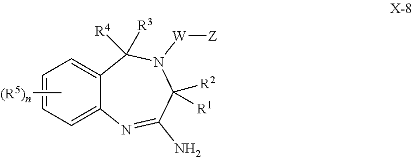

wherein
W is —C(O)—;
Z is H, alkyl, alkenyl, alkynyl, heteroalkyl, cycloalkyl, heterocycloalkyl, aryl, heteroaryl, OR⁶ or NR⁶R⁷, wherein the alkyl, alkenyl, alkynyl, heteroalkyl, cycloalkyl, heterocycloalkyl, aryl and heteroaryl are optionally substituted with one or more groups independently selected from alkyl, alkenyl, alkynyl. F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OCC═O)CH═CH₂—, NR⁶SO₂R⁷, SR⁶ and SO₂R⁶;
R¹, R², R³ and R⁴ are independently selected from H, alkyl, alkenyl, alkynyl, heteroalkyl, cycloalkyl, cycloalkenyl, heterocycloalkyl, aryl and heteroaryl, wherein said alkyl, alkenyl, alkynyl, heteroalkyl, cycloalkyl, cycloalkenyl, heterocycloalkyl, aryl, and heteroaryl are optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(C═O)CH═CH₂—, NR⁶SO₂R⁷, SR₆ and SO₂R⁶,
or R¹ and R² together with the atom to which they are attached form a saturated or partially unsaturated carbocyclic ring, wherein said carbocyclic ring is optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH₂—, NR⁶SO₂R⁷, SR⁶ and SO₂R⁶,
or R³ and R⁴ together are oxo;
R⁵ is H, F, Cl, Br, I, OMe, CH₃, CH₂F, CHF₂, CF₃ or CF₂CF₃;
R⁶ and R⁷ are independently selected from H, alkyl, alkenyl, alkynyl, heteroalkyl, cycloalkyl, cycloalkenyl, heterocycloalkyl, aryl, and heteroaryl, wherein said alkyl, alkenyl, alkynyl, heteroalkyl, cycloalkyl cycloalkenyl, heterocycloalkyl, aryl, and heteroaryl are optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH₂—, NR⁶SO₂R⁷, SR⁶ and SO₂R⁶;
or R⁶ and R⁷ together with the atom to which they are attached form a saturated or partially unsaturated heterocyclic ring, wherein said heterocyclic ring is optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH₂—, NR⁶SO₂R⁷, SR⁶ and SO₂R⁶; and
n is 0, 1, 2, 3 or 4.

Compounds of Formula X-6, X-7, and X-8 are described, for example, in U.S. Publication No. US2019/0016808 and US2014/0088085.

In some aspects, the myeloid cell agonist is a TLR modulator compound that has the structure of Formula X-9:

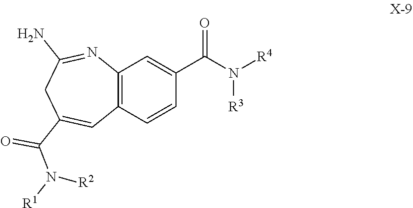

wherein
R¹ is C₃₋₇alkyl or C₃₋₇cycloalkyl;
R² is selected from the group consisting of C₁₋₇alkyl, hydroxy-C₁₋₇alkyl, C₂₋₇alkenyl, C₃₋₇alkynyl, amino-C₁₋₇alkoxy-C₁₋₇alkyl, amino-C₁₋₇alkoxy-C₁₋₇alkoxy-C₁₋₇alkyl, halogen-C₁₋₇alkyl, C₃₋₇cycloalkyl-C₁₋₇alkyl, and phenyl-C₁₋₇alkyl, wherein phenyl is unsubstituted or substituted by amino-C₁₋₇alkyl;
R³ is hydrogen;
R⁴ is selected from the group consisting of
   phenyl, said phenyl being unsubstituted or substituted by one or two groups selected from the group consisting of C₁₋₇alkyl, halogen, halogen-C₁₋₇alkyl, C₁₋₇alkoxy, hydroxy-C₁₋₇alkyl, amino-C₁₋₇alkyl, C₁₋₇alkyl-amino-C₁₋₇alkyl, di-C₁₋₇alkyl-amino-C₁₋₇alkyl, amino-C₂₋₇alkenyl, C₁₋₇alkyl-amino-C₂₋₇alkenyl, di-C₁₋₇alkyl-amino-C₂₋₇alkenyl, amino-C₂₋₇alkynyl, C₁₋₇alkyl-amino-C₂₋₇alkynyl, di-C₁₋₇alkyl-amino-C₂₋₇alkynyl, benzyloxycarbonylamino-C₁₋₇alkyl, amino-C₁₋₇alkoxy, amino-C₁₋₇alkoxy-C₁₋₇alkoxy, amino-C₁₋₇alkoxy-C₁₋₇alkyl, amino-C₁₋₇alkoxy-C₁₋₇alkoxy-C₁₋₇alkyl, C₁₋₇alkylsulfonyl, heterocyclylcarbonyl, and phenyl-C₁₋₇alkyl, wherein phenyl is unsubstituted or substituted by C₁₋₇alkoxy or amino-C₁₋₇alkyl; or
   heteroaryl, said heteroaryl being a 5- or 6-membered aromatic ring containing one, two, or three heteroatoms selected from N, O, or S, and being unsubstituted or substituted by one or two groups selected from the group consisting of C₁₋₇alkyl, halogen, halogen-C₁₋₇alkyl, C₁₋₇alkoxy, hydroxy-C₁₋₇alkyl, amino-C₁₋₇alkyl, C₁₋₇alkyl-amino-C₁₋₇alkyl, di-C₁₋₇alkyl-amino-C₁₋₇alkyl, amino-C₂₋₇alkenyl, C₁₋₇alkyl-amino-C₂₋₇alkenyl, di-C₁₋₇alkyl-amino-C₂₋₇alkenyl, amino-C₂₋₇alkynyl, C₁₋₇alkyl-amino-C₂₋₇alkynyl, di-C₁₋₇alkyl-amino-C₂₋₇alkynyl, benzyloxycarbonylamino-C₁₋₇alkyl, amino-C₁₋₇alkoxy, amino-C₁₋₇alkoxy-C₁₋₇alkoxy, amino-C₁₋₇alkoxy-C₁₋₇alkyl, amino-C₁₋₇alkoxy-C₁₋₇alkoxy-C₁₋₇alkyl, C₁₋₇alkylsulfonyl, heterocyclylcarbonyl, and phenyl-C₁₋₇alkyl, wherein phenyl is unsubstituted or substituted by C₁₋₇alkoxy or amino-C₁₋₇alkyl.
      Compounds of Formula X-9 are described, for example, in PCT Publication No. WO2016/142250.

### Compounds of Category A, TLR8 Agonists

In some aspects, the present disclosure provides a TLR8 agonist represented by the structure of Formula (IIA):

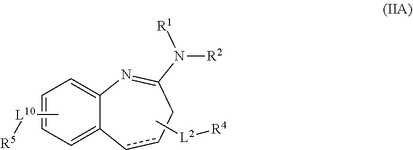

or a pharmaceutically acceptable salt thereof, wherein:
[symbol] represents an optional double bond;
L¹⁰ is —X¹⁰—;
L² is selected from —X²—, —X²—C₁₋₆ alkylene-X²—, —X²—C₂₋₆ alkenylene-X²—, and —X²—C₂₋₆ alkynylene-X²—, each of which is optionally substituted on alkylene, alkenylene or alkynylene with one or more R¹²;
X¹⁰ is selected from —C(O)—, and —C(O)N(R¹⁰)—*, wherein * represents where X¹⁰ is bound to R⁵;
X² at each occurrence is independently selected from a bond, —O—, —S—, —N(R¹⁰)—, —C(O)—, —C(O)O—, —OC(O)—, —OC(O)O—, —C(O)N(R¹⁰)—, —C(O)N(R¹⁰)C(O)—, —C(O)N(R¹⁰)C(O)N(R¹⁰), —N(R¹⁰)C(O)—, —N(R¹⁰)C(O)N(R¹⁰)—, —N(R¹⁰)C(O)O—, —OC(O)N(R¹⁰)—, —C(NR¹⁰)—, —N(R¹⁰)C(NR¹⁰)—, —C(NR¹⁰)N(R¹⁰)—, —N(R¹⁰)C(NR¹⁰)N(R¹⁰)—, —S(O)₂—, —OS(O)—, —S(O)O—, —S(O), —OS(O)₂—, —S(O)₂O, —N(R¹⁰)S(O)₂—, —S(O)₂N(R¹⁰)—, —N(R¹⁰)S(O)—, —S(O)N(R¹⁰)—, —N(R¹⁰)S(O)₂N(R¹⁰)—, and —N(R¹⁰)S(O)N(R¹⁰)—;
R¹ and R² are independently selected from hydrogen; and C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), and —CN;
R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, wherein each C₃₋₁₂ carbocycle, and 3- to 12-membered heterocycle in R⁴ is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R⁵ is selected from unsaturated C₄₋₈ carbocycle; bicyclic carbocycle; and fused 5-5, fused 5-6, and fused 6-6 bicyclic heterocycle, wherein R⁵ is optionally substituted and wherein substituents are independently selected at each occurrence from: halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, wherein each C₃₋₁₂ carbocycle, and 3- to 12-membered heterocycle in R⁵ is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R¹⁰ is independently selected at each occurrence from hydrogen, —NH₂, —C(O)OCH₂C₆H₅; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and haloalkyl; and
R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R¹² is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₁₋₆ alkyl, C₂₋₆ alkenyl, C₂₋₆ alkynyl;
wherein any substitutable carbon on the benzazepine core is optionally substituted by a substituent independently selected from R¹² or two substituents on a single carbon atom combine to form a 3- to 7-membered carbocycle.

In some embodiments, the compound of Formula (IIA) is represented by Formula (IIB):

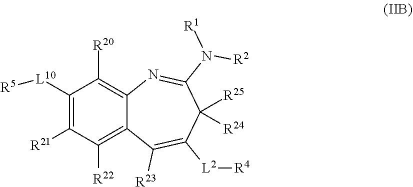

or a pharmaceutically acceptable salt thereof, wherein:
R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; and
R²⁴ and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In some embodiments, R²⁰, R²¹, R²², and R²³ are independently selected from hydrogen, halogen, —OH, —OR¹⁰, —NO₂, —CN, and C₁₋₁₀ alkyl. R²⁰, R²¹, R²², and R²³ may be each hydrogen. In certain embodiments, R²¹ is halogen. In certain embodiments, R²¹ is hydrogen. In certain embodiments, R²¹ is —OR¹⁰. For example, R²¹ may be —OCH₃.

In some embodiments, R²⁴ and R²⁵ are independently selected from hydrogen, halogen, —OH, —NO₂, —CN, and C₁₋₁₀ alkyl, or R²⁴ and R²⁵ taken together form an optionally substituted saturated C₃₋₇ carbocycle. In certain embodiments, R²⁴ and R²⁵ are each hydrogen. In other embodiments, R²⁴ and R²⁵ taken together form an optionally substituted saturated C₃₋₅ carbocycle, wherein substituents are selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; and C₁₋₁₀ alkyl, C₂₋₁₀ alkenyl, C₂₋₁₀ alkynyl, each of which is independently optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle.

In some embodiments, R¹ is hydrogen. In some embodiments, R² is hydrogen. In some embodiments, R² is-C(O)—.

In some embodiments, L¹⁰ is selected from —C(O)N(R¹⁰)—*. In certain embodiments, R¹⁰ of —C(O)N(R¹⁰)—* is selected from hydrogen and C₁₋₆ alkyl. For example, L¹⁰ may be —C(O)NH—*.

In some embodiments, R⁵ is an optionally substituted bicyclic carbocycle. In certain embodiments, R⁵ is an optionally substituted 8- to 12-membered bicyclic carbocycle. R⁵ may be an optionally substituted 8- to 12-membered bicyclic carbocycle substituted with one or more substituents independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆ alkynyl. In certain embodiments, R⁵ is an optionally substituted 8- to 12-membered bicyclic carbocycle substituted with one or more substituents independently selected from —OR¹⁰, —N(R¹⁰)₂, and ═O. In some embodiments, R⁵ is an optionally substituted indane, and optionally substituted tetrahydronaphthalene. R⁵ may be selected from:

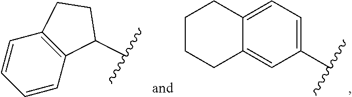 [SMILES: *C1CCc2ccccc21 ; *c1ccc2c(c1)CCCC2]

any one of which is optionally substituted. For example, the R⁵ is selected from:

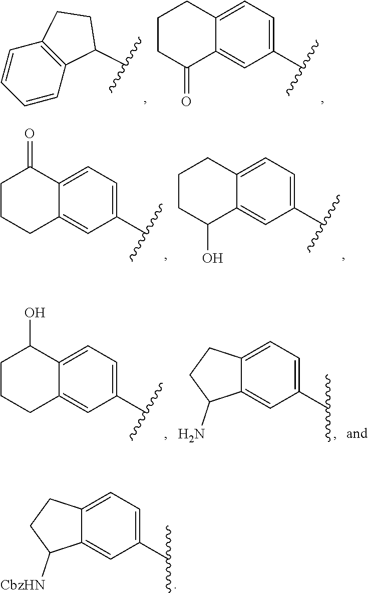

In some embodiments, R⁵ is an optionally substituted unsaturated C₄₋₈ carbocycle. In certain embodiments, R⁵ is an optionally substituted unsaturated C₄₋₆ carbocycle. In certain embodiments, R⁵ is an optionally substituted unsaturated C₄₋₆ carbocycle with one or more substituents independently selected from optionally substituted C₃₋₁₂ carbocycle, and optionally substituted 3- to 12-membered heterocycle. R⁵ may be an optionally substituted unsaturated C₄₋₆ carbocycle with one or more substituents independently selected from optionally substituted phenyl, optionally substituted 3- to 12-heterocycle, optionally substituted C₁₋₁₀ alkyl, optionally substituted C₂₋₁₀ alkenyl, and halogen.

In some embodiments, R⁵ is selected from an optionally substituted fused 5-5, fused 5-6, and fused 6-6 bicyclic heterocycle. In certain embodiments, R⁵ is an optionally substituted fused 5-5, fused 5-6, and fused 6-6 bicyclic heterocycle with one or more substituents independently selected from —C(O)OR¹⁰, —N(R¹⁰)₂, —OR¹⁰, and optionally substituted C₁₋₁₀ alkyl. In certain embodiments, R⁵ is an optionally substituted fused 5-5, fused 5-6, and fused 6-6 bicyclic heterocycle substituted with —C(O)OR¹⁰. In certain embodiments, R⁵ is an optionally substituted fused 6-6 bicyclic heterocycle. For example, the fused 6-6 bicyclic heterocycle may be an optionally substituted pyridine-piperidine. In some embodiments, L¹⁰ is bound to a carbon atom of the pyridine of the fused pyridine-piperidine. In certain embodiments, R⁵ is selected from tetrahydroquinoline, tetrahydroisoquinoline, tetrahydronaphthyridine, cyclopentapyridine, and dihydrobenzoxaborole, any one of which is optionally substituted. R⁵ may be an optionally substituted tetrahydronaphthyridine. In some embodiments, R⁵ is selected from:

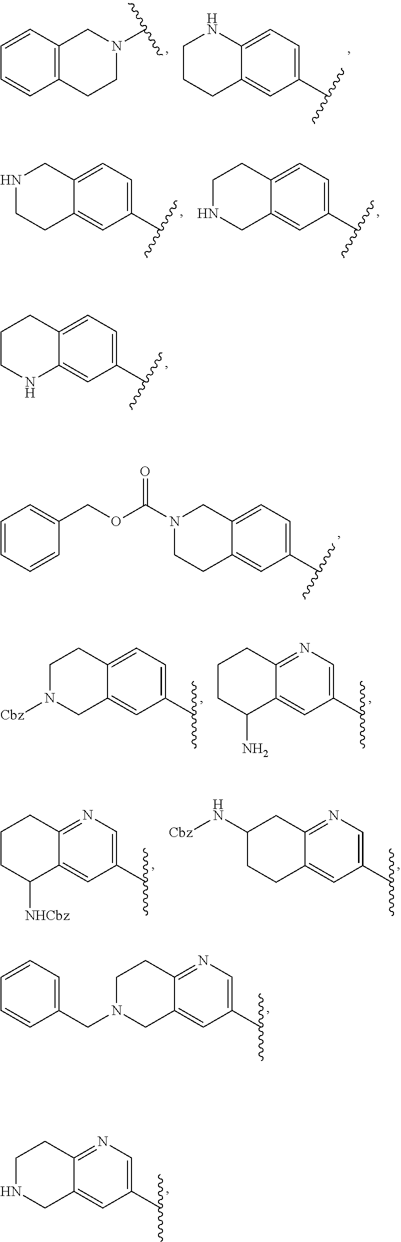

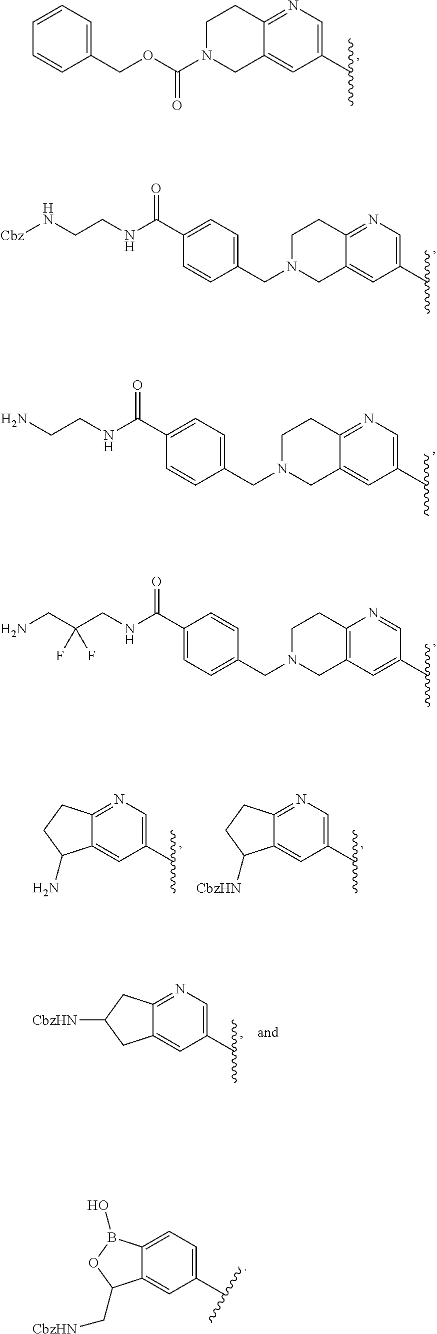

In some embodiments, when R⁵ is substituted, substituents on R⁵ are independently selected at each occurrence from: halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl. In certain embodiments, the substituents on R⁵ are independently selected at each occurrence from: halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle. In certain embodiments, the substituents on R⁵ are independently selected at each occurrence from: halogen, —OR¹⁰, —SR¹⁰, —C(O)N(R¹⁰)₂, —N(R¹⁰)C(O)R¹⁰, —N(R¹⁰)₂, —C(O)R¹⁰, —C(O)OR¹⁰, —OC(O)R¹⁰, —NO₂, ═O, and —CN; and C₁₋₁₀ alkyl optionally substituted with one or more substituents independently selected from halogen, —OR¹⁰, —SR¹⁰, —N(R¹⁰)₂, —C(O)R¹⁰, —C(O)OR¹⁰, —NO₂, ═O, and —CN. In some embodiments, R⁵ is not substituted.

In some embodiments, L² is selected from —C(O)—, and —C(O)NR¹⁰—. In some embodiments, L² is —C(O)—. In some embodiments, L² is selected from —C(O)NR¹⁰—. R¹⁰ of —C(O)NR¹⁰— may be selected from hydrogen and C₁₋₆ alkyl. For example, L² may be —C(O)NH—.

In some embodiments, R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle and 3- to 12-membered, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl. In some embodiments, R⁴ is selected from: —OR¹⁰, and —N(R¹⁰)₂; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl. In certain embodiments, R⁴ is —N(R¹⁰)₂. R¹⁰ of —N(R¹⁰)₂ may be independently selected at each occurrence from optionally substituted C₁₋₆ alkyl. In certain embodiments, R¹⁰ of —N(R¹⁰)₂ is independently selected at each occurrence from methyl, ethyl, propyl, and butyl, any one of which is optionally substituted. For example, R⁴ may be

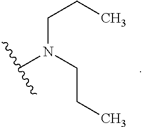 [SMILES: *N(CCC)CCC]

In certain embodiments, -L²-R⁴ is

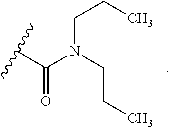 [SMILES: *C(=O)N(CCC)CCC]

In some embodiments, R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle; and C₃₋₁₀ carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₁₋₆ alkyl, C₂₋₆ alkenyl, and C₂₋₆ alkynyl. In certain embodiments, R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; and C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle.

In some embodiments, the compound of Formula (IIB) is a compound of Formula (IIC):

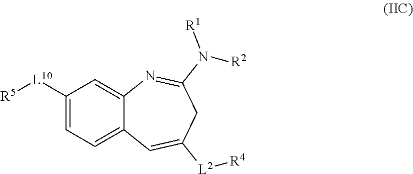

or a pharmaceutically acceptable salt thereof,
wherein:
R¹ and R² are hydrogen;
L² is —C(O)—;
R⁴ is —N(R¹⁰)₂;
R¹⁰ is independently selected at each occurrence from hydrogen, —NH₂, —C(O)OCH₂C₆H₅; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and haloalkyl;
L¹⁰ is —C(O)N(R¹⁰)—*, wherein * represents where L¹⁰ is bound to R⁵; and
R⁵ is a fused 5-5, fused 5-6, or fused 6-6 bicyclic heterocycle, wherein R⁵ is optionally substituted and wherein substituents are independently selected at each occurrence from:
   halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
   C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
   C₃₋₁₂ carbocycle, and 3- to 12-membered heterocycle, each of which is optionally substituted with one or more substituents independently selected from halogen, OR¹⁰, —SR¹⁰,
      —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl.

In certain embodiments, R¹⁰ of —N(R¹⁰)₂ is independently selected at each occurrence from methyl, ethyl, propyl, and butyl, any one of which is optionally substituted; and/or R¹⁰ of —C(O)N(R¹⁰)—* is hydrogen.

In certain embodiments, R⁴ is

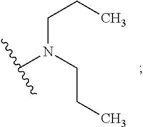 [SMILES: *N(CCC)CCC]

and/or R¹⁰ of —C(O)N(R¹⁰)—* is hydrogen.

In some embodiments, the compound is selected from:

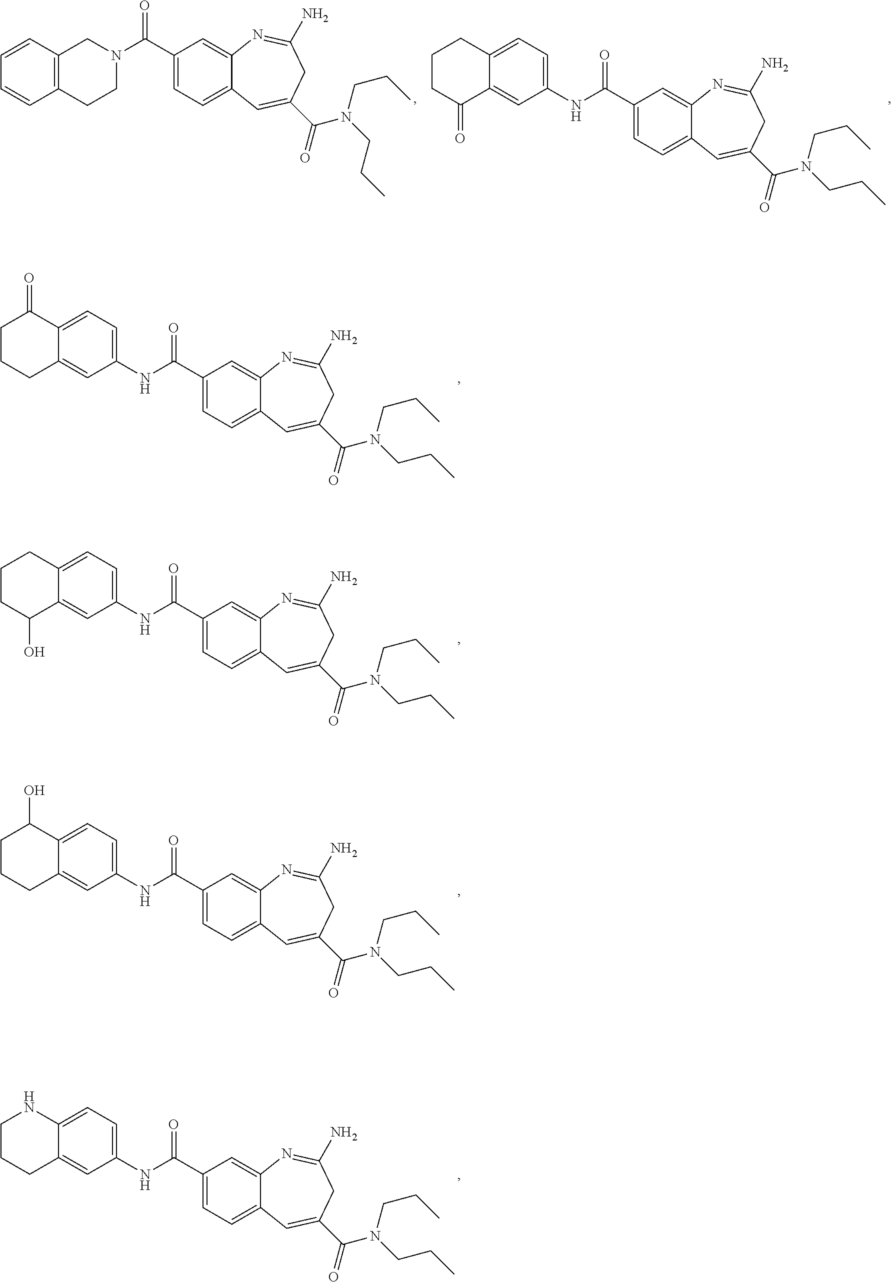

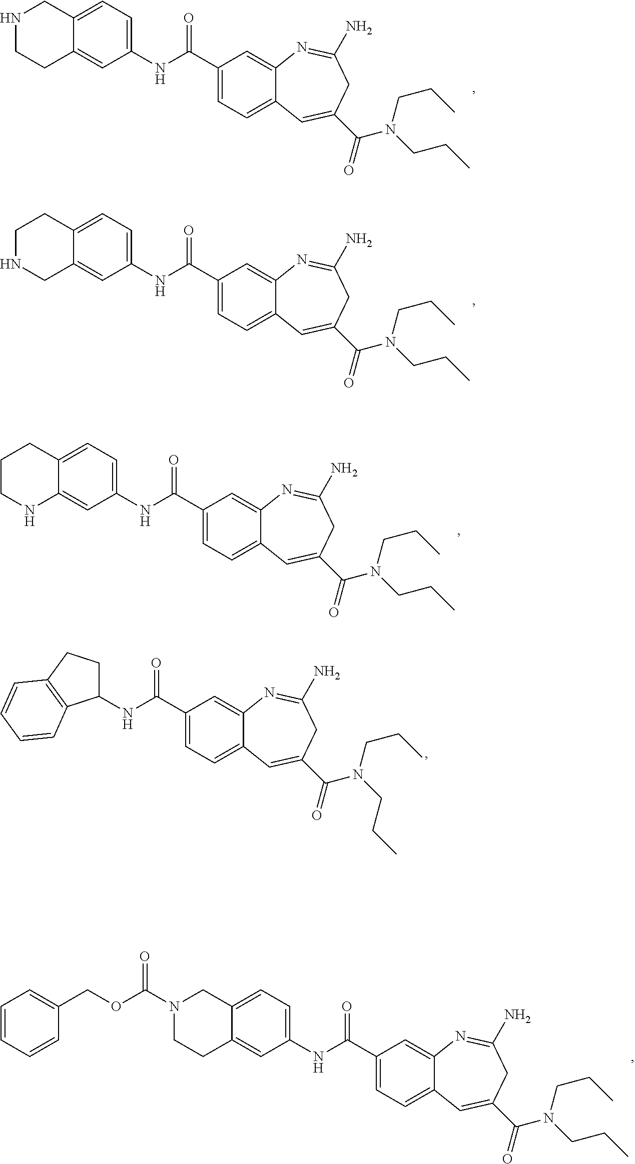 [SMILES: CCCN(CCC)C(=O)C1=Cc2ccc(C(=O)Nc3ccc4c(c3)CCNC4)cc2N=C(N)C1 ; CCCN(CCC)C(=O)C1=Cc2ccc(C(=O)Nc3ccc4c(c3)CNCC4)cc2N=C(N)C1 ; CCCN(CCC)C(=O)C1=Cc2ccc(C(=O)Nc3ccc4c(c3)NCCC4)cc2N=C(N)C1 ; CCCN(CCC)C(=O)C1=Cc2ccc(C(=O)NC3CCc4ccccc43)cc2N=C(N)C1 ; CCCN(CCC)C(=O)C1=Cc2ccc(C(=O)Nc3ccc4c(c3)CCN(C(=O)OCc3ccccc3)C4)cc2N=C(N)C1]

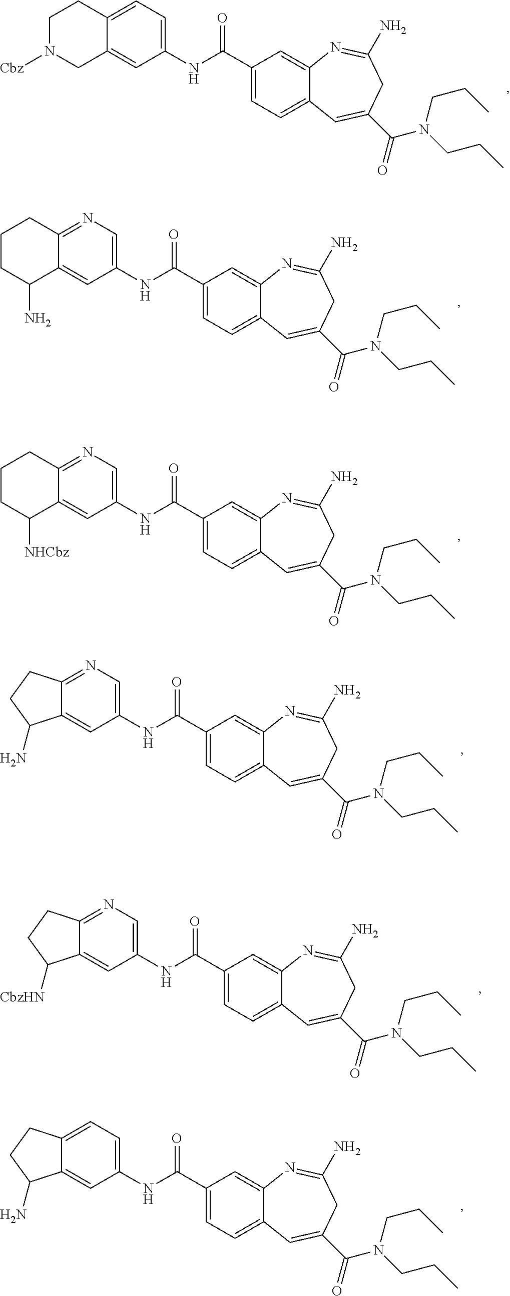 [SMILES: CCCN(CCC)C(=O)C1=Cc2ccc(C(=O)Nc3ccc4c(c3)CN(C(=O)OCc3ccccc3)CC4)cc2N=C(N)C1 ; CCCN(CCC)C(=O)C1=Cc2ccc(C(=O)Nc3cnc4c(c3)C(N)CCC4)cc2N=C(N)C1 ; CCCN(CCC)C(=O)C1=Cc2ccc(C(=O)Nc3cnc4c(c3)C(NC(=O)OCc3ccccc3)CCC4)cc2N=C(N)C1 ; CCCN(CCC)C(=O)C1=Cc2ccc(C(=O)Nc3cnc4c(c3)C(N)CC4)cc2N=C(N)C1 ; CCCN(CCC)C(=O)C1=Cc2ccc(C(=O)Nc3cnc4c(c3)C(NC(=O)OCc3ccccc3)CC4)cc2N=C(N)C1 ; CCCN(CCC)C(=O)C1=Cc2ccc(C(=O)Nc3ccc4c(c3)C(N)CC4)cc2N=C(N)C1]

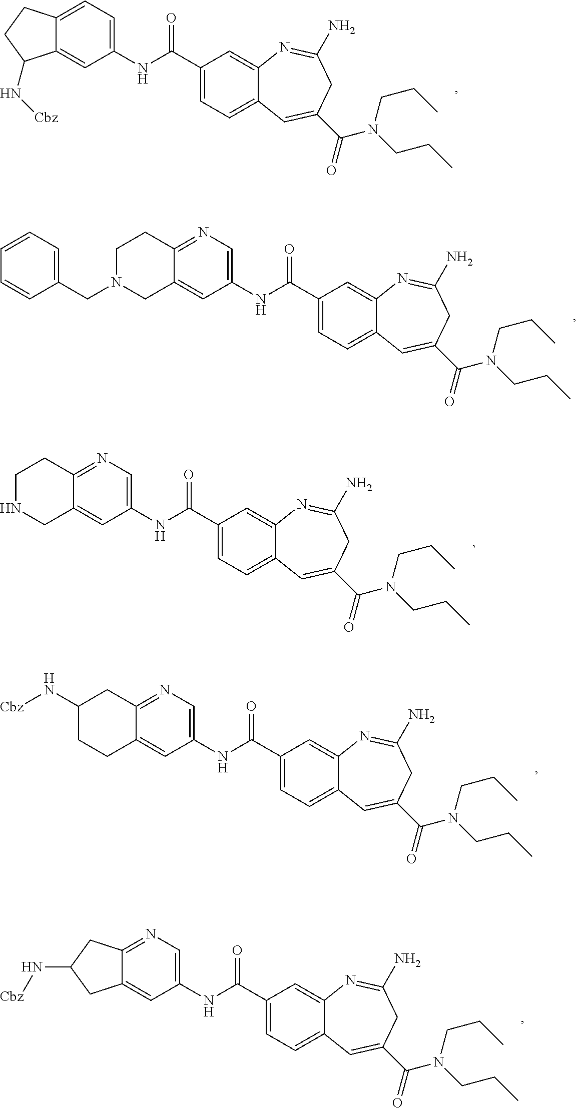 [SMILES: CCCN(CCC)C(=O)C1=Cc2ccc(C(=O)Nc3ccc4c(c3)C(NC(=O)OCc3ccccc3)CC4)cc2N=C(N)C1 ; CCCN(CCC)C(=O)C1=Cc2ccc(C(=O)Nc3cnc4c(c3)CN(Cc3ccccc3)CC4)cc2N=C(N)C1 ; CCCN(CCC)C(=O)C1=Cc2ccc(C(=O)Nc3cnc4c(c3)CNCC4)cc2N=C(N)C1 ; CCCN(CCC)C(=O)C1=Cc2ccc(C(=O)Nc3cnc4c(c3)CCC(NC(=O)OCc3ccccc3)C4)cc2N=C(N)C1 ; CCCN(CCC)C(=O)C1=Cc2ccc(C(=O)Nc3cnc4c(c3)CC(NC(=O)OCc3ccccc3)C4)cc2N=C(N)C1]

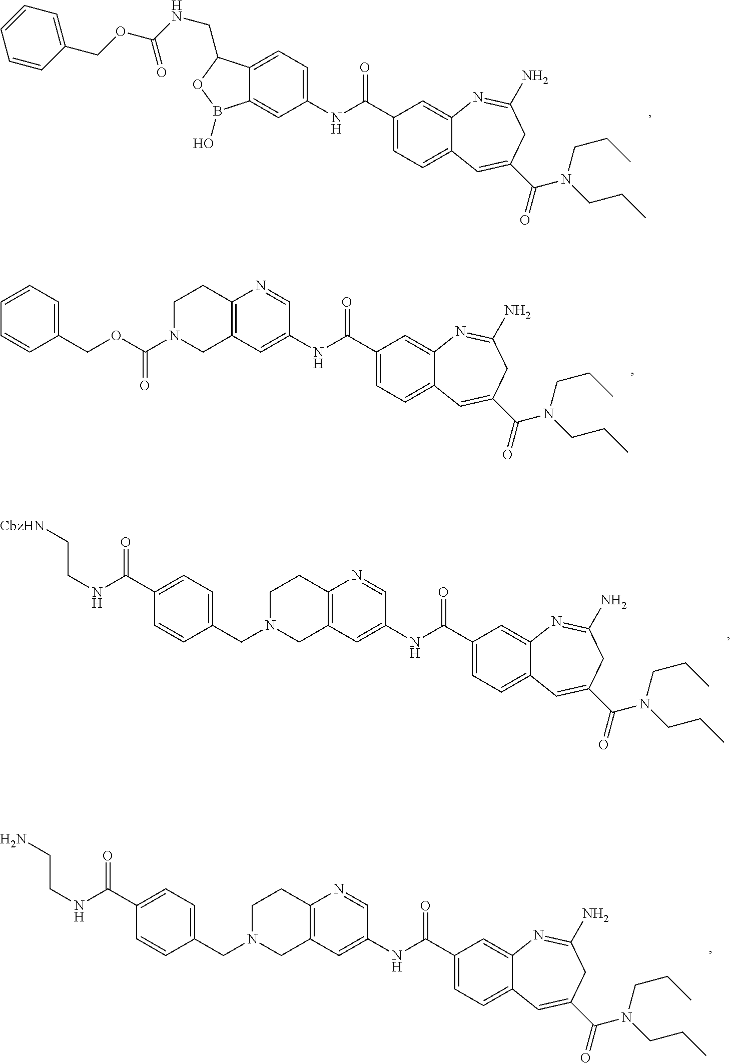 [SMILES: CCCN(CCC)C(=O)C1=Cc2ccc(C(=O)Nc3ccc4c(c3)B(O)OC4CNC(=O)OCc3ccccc3)cc2N=C(N)C1 ; CCCN(CCC)C(=O)C1=Cc2ccc(C(=O)Nc3cnc4c(c3)CN(C(=O)OCc3ccccc3)CC4)cc2N=C(N)C1 ; CCCN(CCC)C(=O)C1=Cc2ccc(C(=O)Nc3cnc4c(c3)CN(Cc3ccc(C(=O)NCCNC(=O)OCc5ccccc5)cc3)CC4)cc2N=C(N)C1 ; CCCN(CCC)C(=O)C1=Cc2ccc(C(=O)Nc3cnc4c(c3)CN(Cc3ccc(C(=O)NCCN)cc3)CC4)cc2N=C(N)C1]

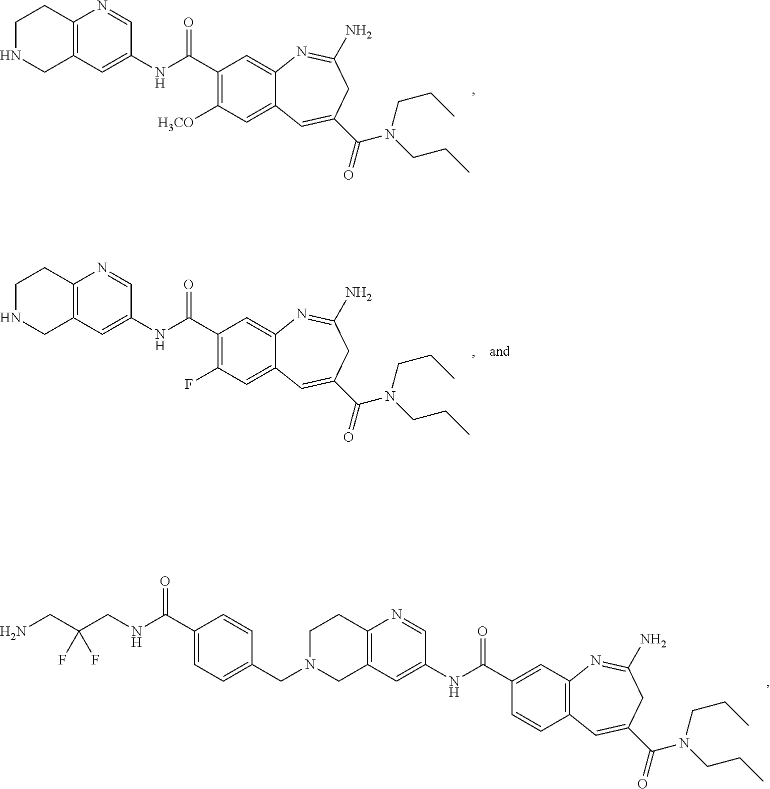 [SMILES: CCCN(CCC)C(=O)C1=Cc2cc(OC)c(C(=O)Nc3cnc4c(c3)CNCC4)cc2N=C(N)C1 ; CCCN(CCC)C(=O)C1=Cc2cc(F)c(C(=O)Nc3cnc4c(c3)CNCC4)cc2N=C(N)C1 ; CCCN(CCC)C(=O)C1=Cc2ccc(C(=O)Nc3cnc4c(c3)CN(Cc3ccc(C(=O)NCC(F)(F)CN)cc3)CC4)cc2N=C(N)C1]

and a salt of any one thereof.

In some aspects, the present disclosure provides a compound represented by the structure of Formula (IIIA):

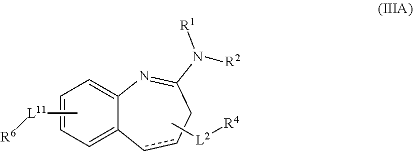

or a pharmaceutically acceptable salt thereof, wherein:
[symbol] represents an optional double bond;
L¹¹ is —X¹¹—;
L² is selected from —X²—, —X²—C₁₋₆ alkylene-X²—, —X²—C₂₋₆ alkenylene-X²—, and —X²—C₂₋₆ alkynylene-X²—, each of which is optionally substituted on alkylene, alkenylene or alkynylene with one or more R¹²;
X¹¹ is selected from —C(O)— and —C(O)N(R¹⁰)—*, wherein * represents where X¹¹ is bound to R⁶;
X² at each occurrence is independently selected from a bond, —O—, —S—, —N(R¹⁰)—, —C(O)—, —C(O)O—, —OC(O)—, —OC(O)O—, —C(O)N(R¹⁰)—, —C(O)N(R¹⁰)C(O)—, —C(O)N(R¹⁰)C(O)N(R¹⁰)—, —N(R¹⁰)C(O)—, —N(R¹⁰)C(O)N(R¹⁰)—, —N(R¹⁰)C(O)O—, —OC(O)N(R¹⁰)—, —C(NR¹⁰)—, —N(R¹⁰)C(NR¹⁰)—, —C(NR¹⁰)N(R¹⁰)—, —N(R¹⁰)C(NR¹⁰)N(R¹⁰)—, —S(O)₂—, —OS(O)—, —S(O)O—, —S(O)—, —OS(O)₂—, —S(O)₂O—, —N(R¹⁰)S(O)₂—, —S(O)₂N(R¹⁰)—, —N(R¹⁰)S(O)—, —S(O)N(R¹⁰)—, —N(R¹⁰)S(O)₂N(R¹⁰)—, and —N(R¹⁰)S(O)N(R¹⁰)—;
R¹ and R² are independently selected from hydrogen; C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), and —CN;
R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, wherein each C₃₋₁₂ carbocycle, and 3- to 12-membered heterocycle in R⁴ is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R⁶ is selected from phenyl and 5- or 6-membered heteroaryl, any one of which is substituted with one or more substituents selected from R⁷ and R⁶ is further optionally substituted by one or more additional substituents independently selected from R¹²;
R⁷ is selected from —C(O)NHNH₂, —C(O)NH—C₁₋₃ alkylene-NH(R¹⁰), —C(O)CH₃, —C₁₋₃ alkylene-NHC(O)OR¹¹, —C₁₋₃alkylene-NHC(O)R¹⁰, —C₁₋₃alkylene-NHC(O)NHR¹⁰, —C₁₋₃alkylene-NHC(O)—C₁₋₃alkylene-R¹⁰, and a 3- to 12-membered heterocycle optionally substituted with one or more substituents independently selected from R¹²;
R¹⁰ is independently selected at each occurrence from hydrogen, —NH₂, —C(O)OCH₂C₆H₅; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, —C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R¹¹ is selected from C₃₋₁₂ carbocycle and 3- to 12-membered heterocycle, each of which is optionally substituted with one or more substituents independently selected from R¹²; and
R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R¹² is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₁₋₆ alkyl, C₂₋₆ alkenyl, and C₂₋₆ alkynyl; and
wherein any substitutable carbon on the benzazepine core is optionally substituted by a substituent independently selected from R¹² or two substituents on a single carbon atom combine to form a 3- to 7-membered carbocycle.

In some embodiments, the compound of Formula (IIIA) is represented by Formula (IIIB):

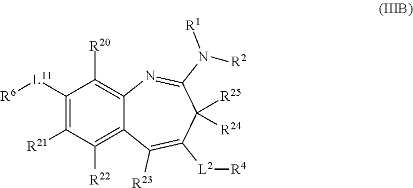

or a pharmaceutically acceptable salt thereof, wherein:
R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; and
R²⁴ and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In some embodiments, R²⁰, R²¹, R²², and R²³ are independently selected from hydrogen, halogen, —OH, —NO₂, —CN, and C₁₋₁₀ alkyl. In certain embodiments, R²⁰, R²¹, R²², and R²³ are each hydrogen. In some embodiments, R²⁴ and R²⁵ are independently selected from hydrogen, halogen, —OH, —NO₂, —CN, and C₁₋₁₀ alkyl, or R²⁴ and R²⁵ taken together form an optionally substituted saturated C₃₋₇ carbocycle. In certain embodiments, R²⁴ and R²⁵ are each hydrogen. In certain embodiments, R²⁴ and R²⁵ taken together form an optionally substituted saturated C₃₋₅ carbocycle.

In some embodiments, R¹ is hydrogen. In some embodiments, R² is hydrogen.

In some embodiments, L¹¹ is selected from —C(O)N(R¹⁰)— *. In some embodiments, R¹⁰ of —C(O)N(R¹⁰)—* is selected from hydrogen and C₁₋₆ alkyl. For example, L¹¹ may be —C(O)NH—*.

In some embodiments, R⁶ is phenyl substituted with R⁷ and R⁶ is further optionally substituted with one or more additional substituents independently selected from R¹². In some embodiments, R⁶ is selected from phenyl substituted with one or more substituents independently selected from —C(O)NHNH₂, —C(O)NH—C₁₋₃alkylene-NH(R¹⁰), —C₁₋₃alkylene-NHC(O)R¹⁰, and —C(O)CH₃; and 3- to 12-membered heterocycle, which is optionally substituted with one or more substituents selected from —OH, —N(R¹⁰)₂, —NHC(O)(R¹⁰), —NHC(O)O(R¹⁰), —NHC(O)N(R¹⁰)₂, —C(O)R¹⁰, —C(O)N(R¹⁰)₂, —C(O)₂R¹⁰, and —C₁₋₃alkylene-(R¹⁰) and R⁶ is further optionally substituted with one or more additional substituents independently selected from R¹². For example, R⁶ may be selected from:

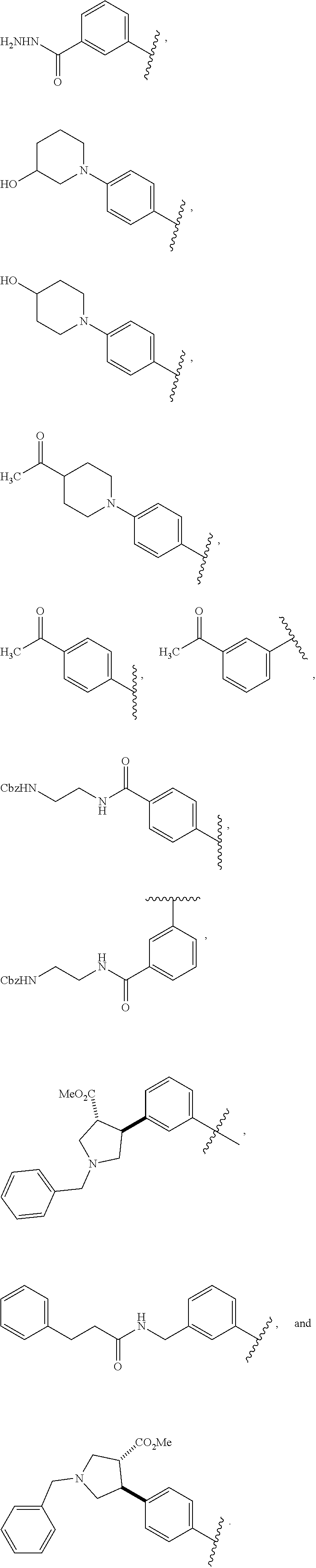

In some embodiments, R⁶ is selected from a 5- and 6-membered heteroaryl substituted with one or more substituents independently selected from R⁷, and R⁶ is further optionally substituted with one or more additional substituents selected from R¹². In certain embodiments, R⁶ is selected from 5- and 6-membered heteroaryl substituted with one or more substituents independently selected from —C(O)CH₃, —C₁₋₃alkylene-NHC(O)OR¹⁰, —C₁₋₃alkylene-NHC(O)R¹⁰, —C₁₋₃alkylene-NHC(O)NHR¹⁰, and —C₁₋₃alkylene-NHC(O) —C₁₋₃alkylene-(R¹⁰); and 3- to 12-membered heterocycle, which is optionally substituted with one or more substituents selected from —OH, —N(R¹⁰)₂, —NHC(O)(R¹⁰), —NHC(O)O(R¹⁰), —NHC(O)N(R¹⁰)₂, —C(O)R¹⁰, —C(O)N(R¹⁰)₂, —C(O)₂R¹⁰, and —C₁₋₃alkylene-(R¹⁰), and R⁶ is optionally further substituted with one or more additional substituents independently selected from R¹². R⁶ may be selected from substituted pyridine, pyrazine, pyrimidine, pyridazine, furan, pyran, oxazole, thiazole, imidazole, pyrazole, oxadiazole, oxathiazole, and triazole, and R⁶ is optionally further substituted with one or more additional substituents independently selected from R¹². In some embodiments, R⁶ is substituted pyridine and R⁶ is optionally further substituted with one or more additional substituents independently selected from R¹². R⁶ may be represented as follows:

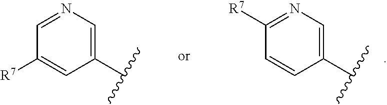

In some embodiments, R⁶ is substituted pyridine, and wherein R⁷ is —C₁₋₃alkylene-NHC(O)—C₁₋₃alkylene-R¹⁰. In certain embodiments, R⁷ is-C₁alkylene-NHC(O)—C₁alkylene-R¹⁰. In certain embodiments, R⁷ is —C₁alkylene-NHC(O)—C₁alkylene-NH₂. In some embodiments, R⁶ is selected from:

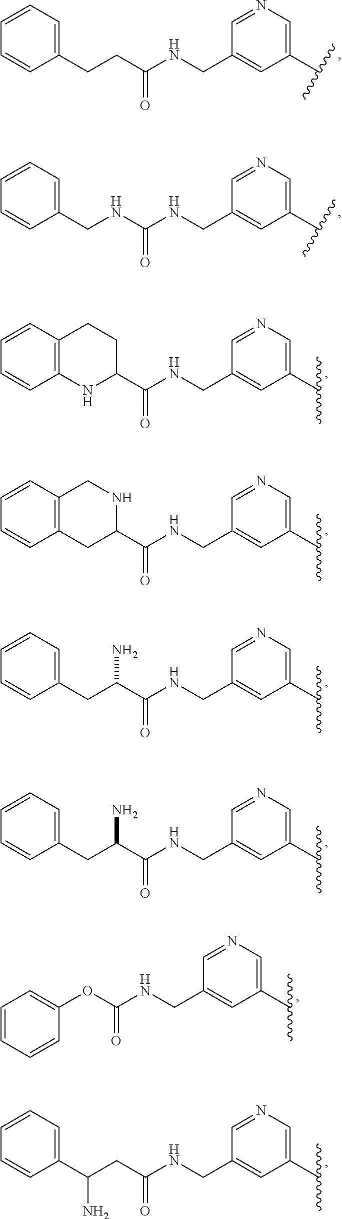

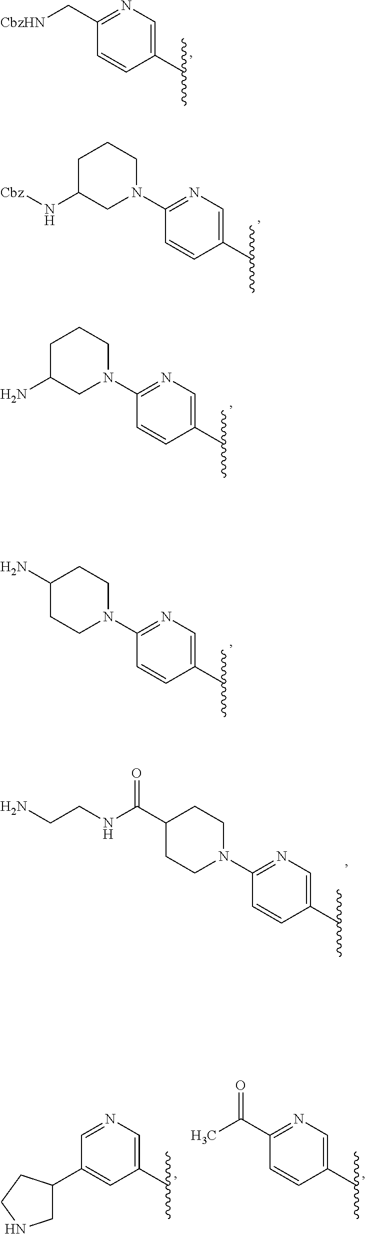

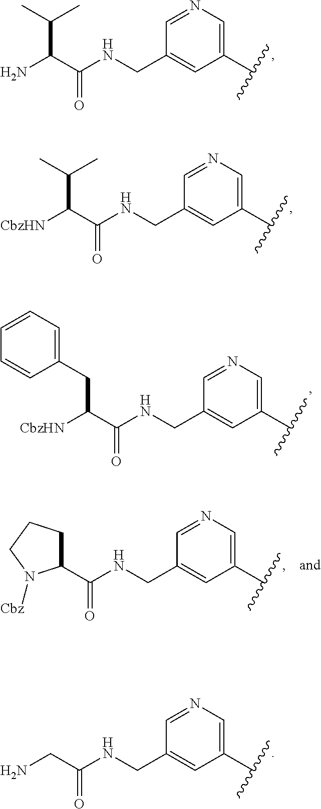 [SMILES: *c1cncc(CNC(=O)[C@@H](N)C(C)C)c1 ; *c1cncc(CNC(=O)[C@@H](NC(=O)OCc2ccccc2)C(C)C)c1 ; *c1cncc(CNC(=O)[C@H](Cc2ccccc2)NC(=O)OCc2ccccc2)c1 ; *c1cncc(CNC(=O)[C@@H]2CCCN2C(=O)OCc2ccccc2)c1 ; *c1cncc(CNC(=O)CN)c1]

In certain embodiments, R⁶ is

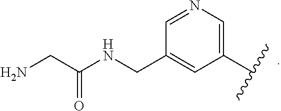 [SMILES: *c1cncc(CNC(=O)CN)c1]

In some embodiments, L² is selected from —C(O)—, and —C(O)NR¹⁰—. In some embodiments, L² is selected from —C(O)NR¹⁰—. R¹⁰ of —C(O)NR¹⁰— may be selected from hydrogen and C₁₋₆ alkyl. For example, L² may be —C(O)NH—. In some embodiments, L² is —C(O)—.

In some embodiments, R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle and 3- to 12-membered, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl. In some embodiments, R⁴ is selected from: —OR¹⁰ and —N(R¹⁰)₂; and C₁₋₁₀ alkyl, C₂₋₁₀ alkenyl, C₂₋₁₀ alkynyl, C₃₋₁₂ carbocycle and 3- to 12-membered heterocycle, each of which is independently optionally substituted at each occurrence with one or more substituents selected from halogen, —OR¹⁰, —SR¹⁰, —N(R¹⁰)₂, —S(O)R¹⁰, —S(O)₂R¹⁰—C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl. In certain embodiments, R⁴ is —N(R¹⁰)₂. R¹⁰ of —N(R¹⁰)₂ may be independently selected at each occurrence from optionally substituted C₁₋₆ alkyl. In some embodiments, R¹⁰ of —N(R¹⁰)₂ is independently selected at each occurrence from methyl, ethyl, propyl, and butyl, any of which are optionally substituted. For example, R⁴ may be

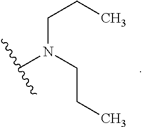 [SMILES: *N(CCC)CCC]

In some embodiments, -L²-R⁴ is

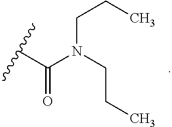 [SMILES: *C(=O)N(CCC)CCC]

In some embodiments, R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is independently optionally substituted at each occurrence with one or more substituents selected from halogen, —OR¹⁰, —SR¹⁰, —N(R¹⁰)₂, —C(O)R¹⁰, —C(O)N(R¹⁰)₂, —N(R¹⁰)C(O)R, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle; and C₃₋₁₀ carbocycle and 3- to 10-membered heterocycle, each of which is independently optionally substituted at each occurrence with one or more substituents selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₁₋₆ alkyl, C₂₋₆ alkenyl, and C₂₋₆ alkynyl. In certain embodiments, R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is independently optionally substituted at each occurrence with one or more substituents selected from halogen, —OR¹⁰, —SR¹⁰, —N(R¹⁰)₂, —C(O)R¹⁰, —C(O)N(R¹⁰)₂, —N(R¹⁰)C(O)R, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle.

In some embodiments, the compound is selected from:

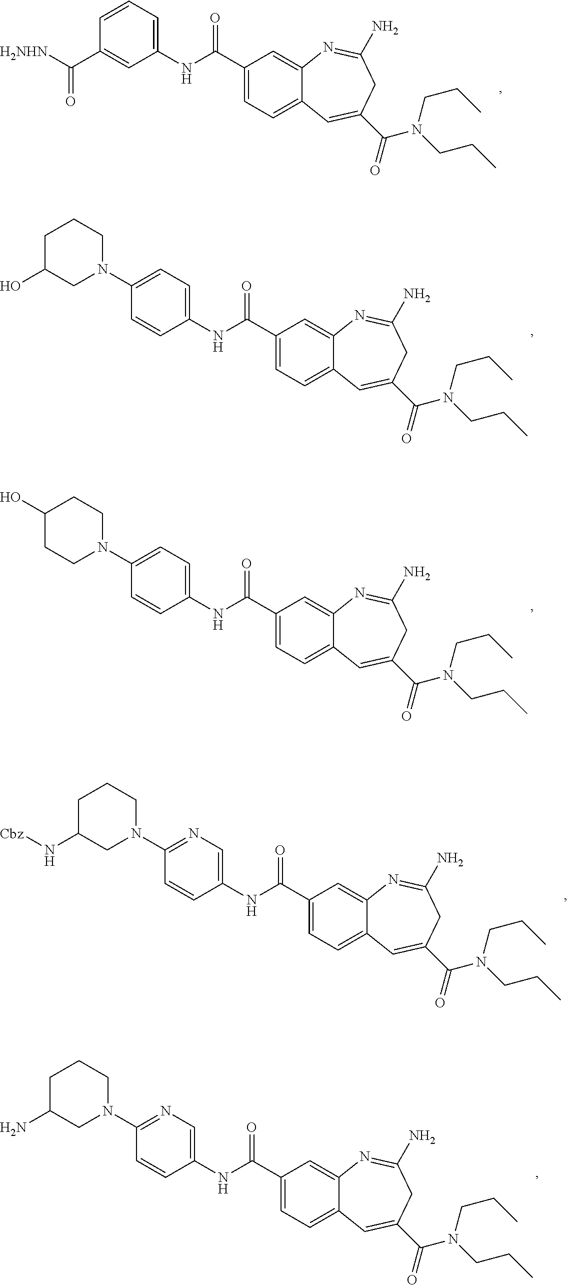

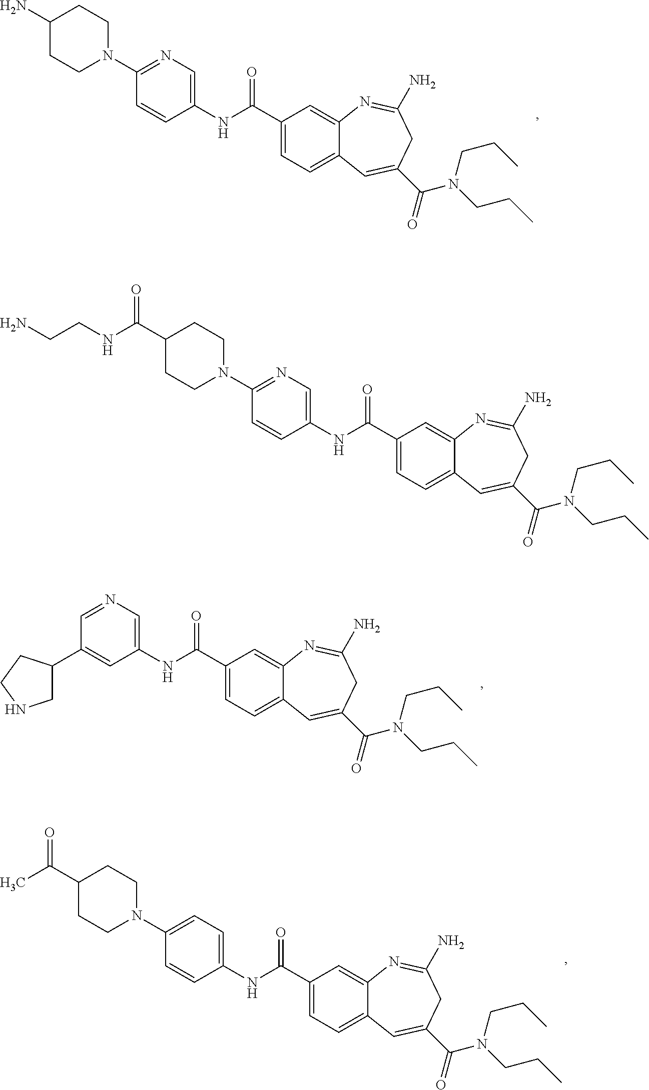

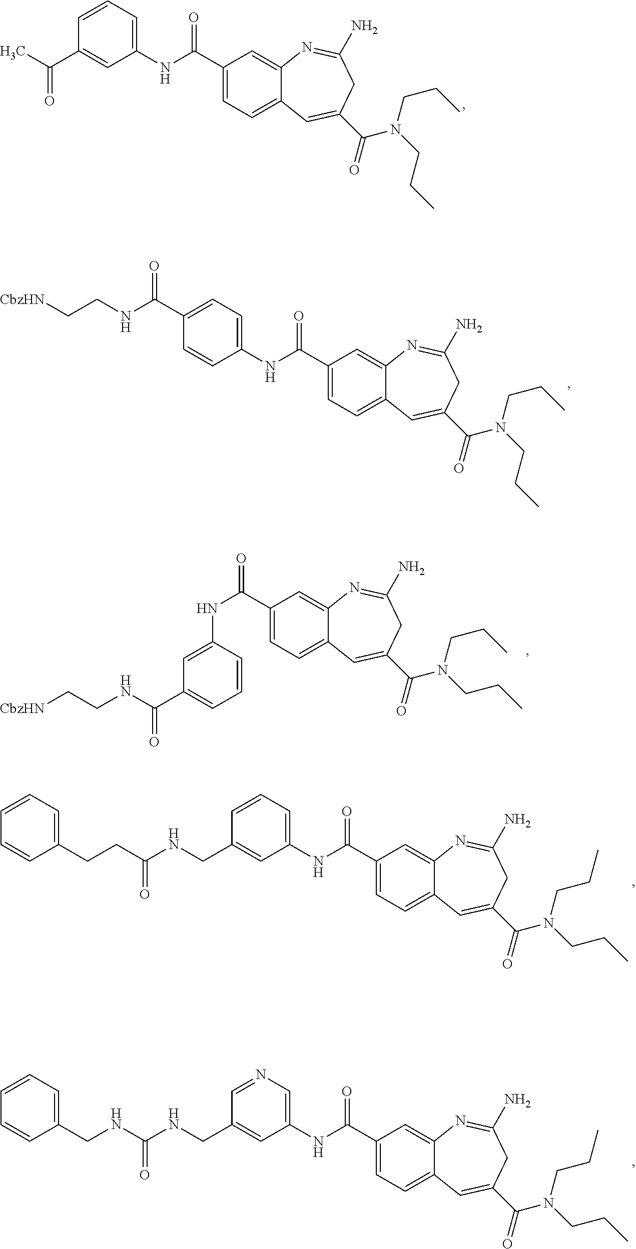 [SMILES: CCCN(CCC)C(=O)C1=Cc2ccc(C(=O)Nc3cccc(C(C)=O)c3)cc2N=C(N)C1 ; CCCN(CCC)C(=O)C1=Cc2ccc(C(=O)Nc3ccc(C(=O)NCCNC(=O)OCc4ccccc4)cc3)cc2N=C(N)C1 ; CCCN(CCC)C(=O)C1=Cc2ccc(C(=O)Nc3cccc(C(=O)NCCNC(=O)OCc4ccccc4)c3)cc2N=C(N)C1 ; CCCN(CCC)C(=O)C1=Cc2ccc(C(=O)Nc3cccc(CNC(=O)CCc4ccccc4)c3)cc2N=C(N)C1 ; CCCN(CCC)C(=O)C1=Cc2ccc(C(=O)Nc3cncc(CNC(=O)NCc4ccccc4)c3)cc2N=C(N)C1]

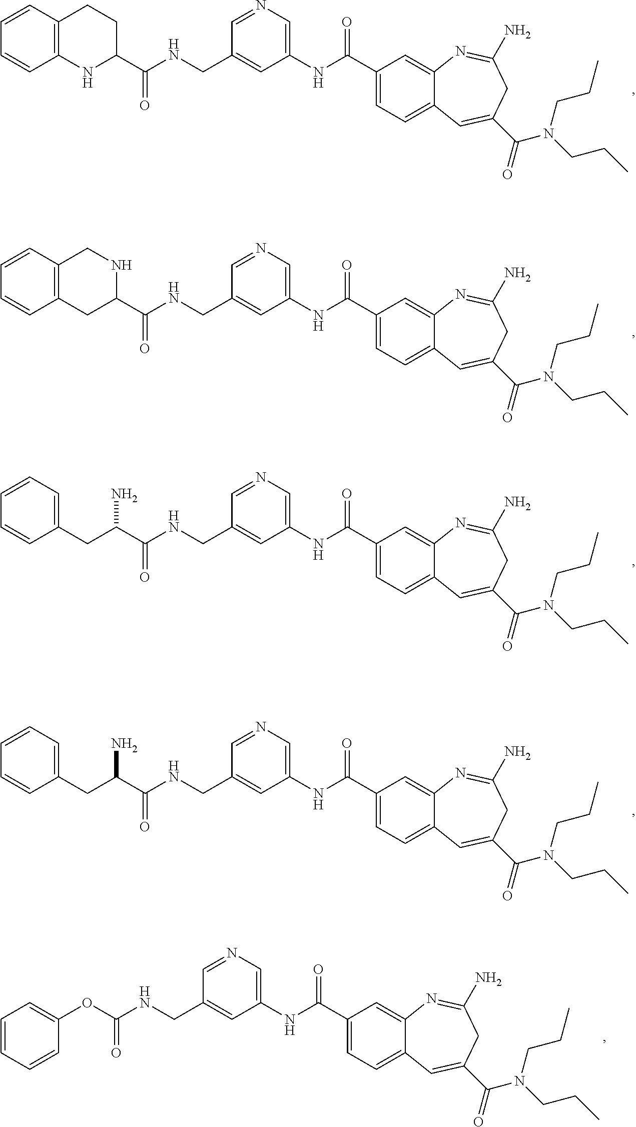 [SMILES: CCCN(CCC)C(=O)C1=Cc2ccc(C(=O)Nc3cncc(CNC(=O)C4CCc5ccccc5N4)c3)cc2N=C(N)C1 ; CCCN(CCC)C(=O)C1=Cc2ccc(C(=O)Nc3cncc(CNC(=O)C4Cc5ccccc5CN4)c3)cc2N=C(N)C1 ; CCCN(CCC)C(=O)C1=Cc2ccc(C(=O)Nc3cncc(CNC(=O)[C@@H](N)Cc4ccccc4)c3)cc2N=C(N)C1 ; CCCN(CCC)C(=O)C1=Cc2ccc(C(=O)Nc3cncc(CNC(=O)[C@H](N)Cc4ccccc4)c3)cc2N=C(N)C1 ; CCCN(CCC)C(=O)C1=Cc2ccc(C(=O)Nc3cncc(CNC(=O)Oc4ccccc4)c3)cc2N=C(N)C1]

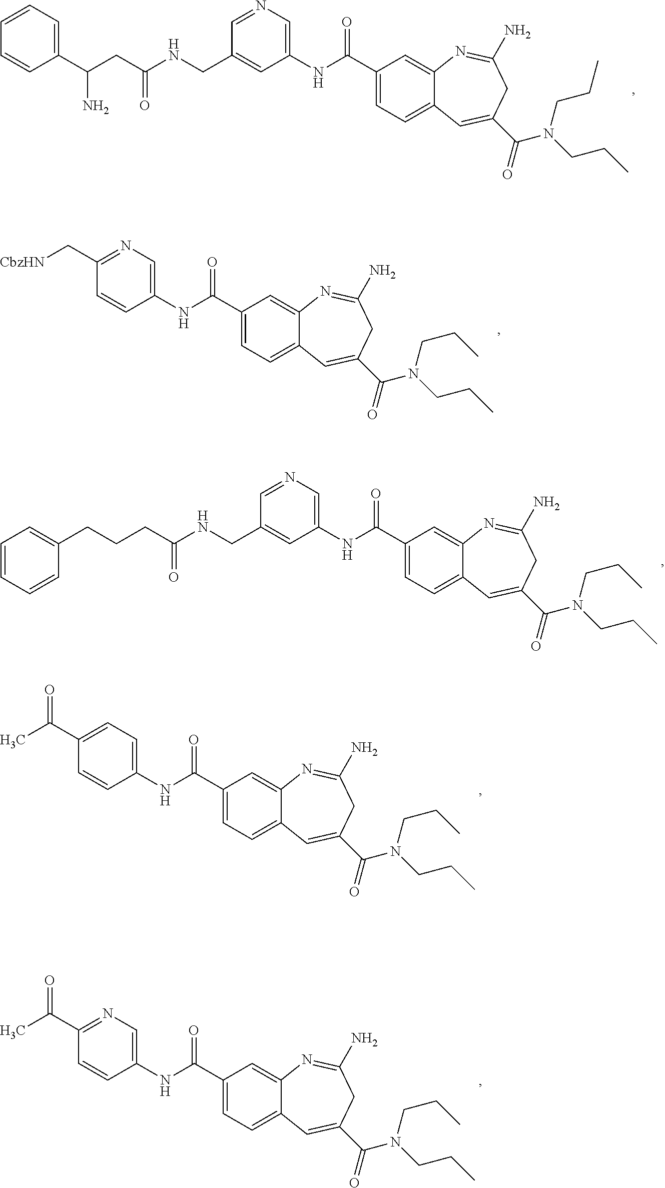

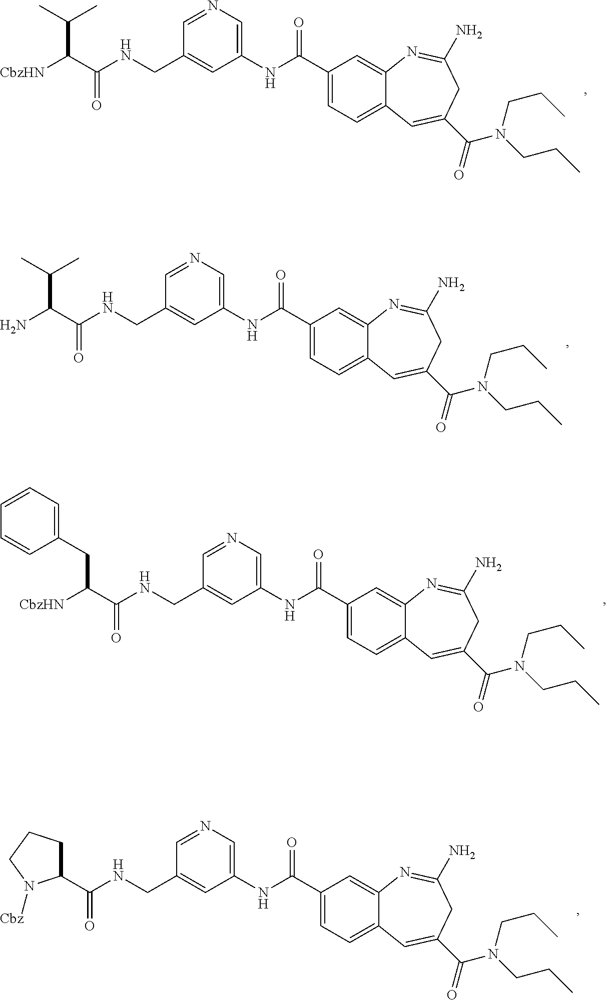 [SMILES: CCCN(CCC)C(=O)C1=Cc2ccc(C(=O)Nc3cncc(CNC(=O)[C@@H](NC(=O)OCc4ccccc4)C(C)C)c3)cc2N=C(N)C1 ; CCCN(CCC)C(=O)C1=Cc2ccc(C(=O)Nc3cncc(CNC(=O)[C@@H](N)C(C)C)c3)cc2N=C(N)C1 ; CCCN(CCC)C(=O)C1=Cc2ccc(C(=O)Nc3cncc(CNC(=O)[C@H](Cc4ccccc4)NC(=O)OCc4ccccc4)c3)cc2N=C(N)C1 ; CCCN(CCC)C(=O)C1=Cc2ccc(C(=O)Nc3cncc(CNC(=O)[C@@H]4CCCN4C(=O)OCc4ccccc4)c3)cc2N=C(N)C1]

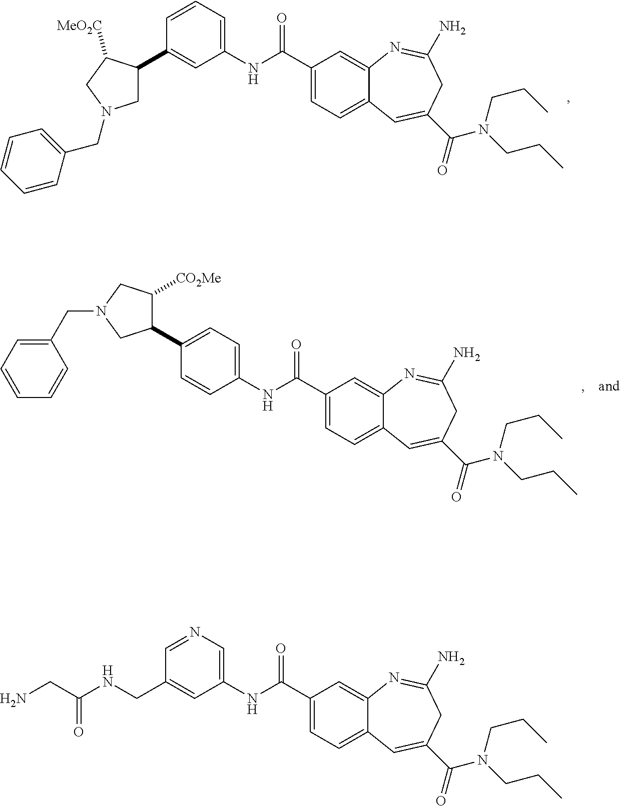

and a salt of any one thereof.

In some aspects, the present disclosure provides a compound represented by the structure of Formula (IA):

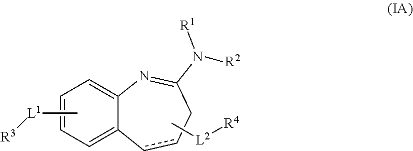

or a pharmaceutically acceptable salt thereof, wherein:
[symbol] represents an optional double bond;
L¹ is selected from —X¹—, —X²—C₁₋₆ alkylene-X²—C₁₋₆ alkylene-, —X²—C₂₋₆ alkenylene-X²—, and —X²—C₂₋₆ alkynylene-X²—, each of which is optionally substituted on alkylene, alkenylene or alkynylene with one or more R¹²;
L² is selected from —X²—, —X²—C₁₋₆ alkylene-X²—, —X²—C₂₋₆ alkenylene-X²—, and
—X²—C₂₋₆ alkynylene-X²—, each of which is optionally substituted on alkylene, alkenylene or alkynylene with one or more R¹²;
X¹ is selected from —S—*, —N(R¹⁰)—*, —C(O)O—*, —OC(O)—*, —OC(O)O—*, —C(O)N(R¹⁰)C(O)— *, —C(O)N(R¹⁰)C(O)N(R¹⁰)*, —N(R¹⁰)C(O)—*, —CR¹⁰₂N(R¹⁰)C(O)—*, —N(R¹⁰)C(O)N(R¹⁰)—*, —N(R¹⁰)C(O)O—*, —OC(O)N(R¹⁰)—*, —C(NR¹⁰)—*, —N(R¹⁰)C(NR¹⁰)—*, —C(NR¹⁰)N(R¹⁰)—*, —N(R¹⁰)C(NR¹⁰)N(R¹⁰)—*, —S(O)₂—*, —OS(O)—*, —S(O)O—*, —S(O), —OS(O)₂—*, —S(O)₂O*, —N(R¹⁰)S(O)₂—*, —S(O)₂N(R¹⁰)—*, —N(R¹⁰)S(O)—*, —S(O)N(R¹⁰)—*, —N(R¹⁰)S(O)₂N(R¹⁰)—*, and —N(R¹⁰)S(O)N(R¹⁰)—*, wherein * represents where X¹ is bound to R³;
X² is independently selected at each occurrence from —O—, —S—, —N(R¹⁰)—, —C(O)—, —C(O)O—, —OC(O)—, —OC(O)O—, —C(O)N(R¹⁰)—, —C(O)N(R¹⁰)C(O)—, —C(O)N(R¹⁰)C(O)N(R¹⁰), —N(R¹⁰)C(O)—, —N(R¹⁰)C(O)N(R¹⁰)—, —N(R¹⁰)C(O)O—, —OC(O)N(R¹⁰)—, —C(NR¹⁰)—, —N(R¹⁰)C(NR¹⁰)—, —C(NR¹⁰)N(R¹⁰)—, —N(R¹⁰)C(NR¹⁰)N(R¹⁰)—, —S(O)₂—, —OS(O)—, —S(O)O—, —S(O), —OS(O)₂—, —S(O)₂O, —N(R¹⁰)S(O)₂—, —S(O)₂N(R¹⁰)—, —N(R¹⁰)S(O)—, —S(O)N(R¹⁰)—, —N(R¹⁰)S(O)₂N(R¹⁰)—, and —N(R¹⁰)S(O)N(R¹⁰)—;
R¹ and R² are independently selected from hydrogen; C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), and —CN;
R³ is selected from optionally substituted C₃₋₁₂ carbocycle, and optionally substituted 3- to 12-membered heterocycle, wherein substituents on R³ are independently selected at each occurrence from: halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, wherein each C₃₋₁₂ carbocycle, and 3- to 12-membered heterocycle in R³ is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, wherein each C₃₋₁₂ carbocycle, and 3- to 12-membered heterocycle in R⁴ is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R¹⁰ is independently selected at each occurrence from: hydrogen, —NH₂, —C(O)OCH₂C₆H₅; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl; and
R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R¹² is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₁₋₆ alkyl, C₂₋₆ alkenyl, and C₂₋₆ alkynyl; and
wherein any substitutable carbon on the benzazepine core is optionally substituted by a substituent independently selected from R¹² or two substituents on a single carbon atom combine to form a 3- to 7-membered carbocycle.

In some embodiments, the compound of Formula (IA) is represented by Formula (IB):

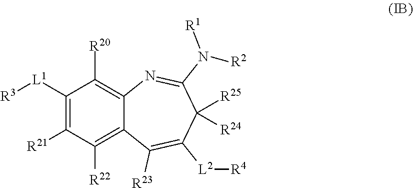

or a pharmaceutically acceptable salt thereof, wherein:
R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; and
R²⁴ and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In some embodiments, R²⁰, R²¹, R²², and R²³ are independently selected from hydrogen, halogen, —OH, —NO₂, —CN, and C₁₋₁₀ alkyl. In certain embodiments, R²⁰, R²¹, R²², and R²³ are each hydrogen.

In some embodiments, R²⁴ and R²⁵ are independently selected from hydrogen, halogen, —OH, —NO₂, —CN, and C₁₋₁₀ alkyl, or R²⁴ and R²⁵ taken together form an optionally substituted saturated C₃₋₇ carbocycle. In some embodiments, R²⁴ and R²⁵ are each hydrogen. In some embodiments, R²⁴ and R²⁵ taken together form an optionally substituted saturated C₃₋₅ carbocycle.

In some embodiments, R¹ is hydrogen. In some embodiments, R² is hydrogen.

In some embodiments, L¹ is selected from —N(R¹⁰)C(O)—*, —S(O)₂N(R¹⁰)—*, —CR¹⁰₂N(R¹⁰)C(O)—*and —X²—C₁₋₆ alkylene-X²—C₁₋₆ alkylene-. In some embodiments, L¹ is selected from —N(R¹⁰)C(O)—*. In certain embodiments, R¹⁰ of —N(R¹⁰)C(O)—* is selected from hydrogen and C₁₋₆ alkyl. For example, L¹ may be —NHC(O)—*. In some embodiments, L¹ is selected from —S(O)₂N(R¹⁰)—*. In certain embodiments, R¹⁰ of —S(O)₂N(R¹⁰)—* is selected from hydrogen and C₁₋₆ alkyl. For example, L¹ is —S(O)₂NH—*. In some embodiments, L¹ is —CR¹⁰₂N(R¹⁰)C(O)—*. In certain embodiments, L¹ is selected from —CH₂N(H)C(O)—* and —CH(CH₃)N(H)C(O)—*.

In some embodiments, R³ is selected from optionally substituted C₃₋₁₂ carbocycle, and optionally substituted 3- to 12-membered heterocycle, wherein substituents on R³ are independently selected at each occurrence from: halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl. In certain embodiments, R³ is selected from optionally substituted C₃₋₁₂ carbocycle, and optionally substituted 3- to 12-membered heterocycle, wherein substituents on R³ are independently selected at each occurrence from: halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle.

In some embodiments, R³ is selected from an optionally substituted aryl and an optionally substituted heteroaryl. In some embodiments, R³ is an optionally substituted heteroaryl. R³ may be an optionally substituted heteroaryl substituted with one or more substituents independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆ alkynyl. In certain embodiments, R³ is selected from an optionally substituted 6-membered heteroaryl. For example, R³ may be an optionally substituted pyridine. In some embodiments, R³ is an optionally substituted aryl. In certain embodiments, R³ is an optionally substituted aryl substituted with one or more substituents independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆ alkynyl. R³ may be an optionally substituted phenyl. In certain embodiments, R³ is selected from pyridine, phenyl, tetrahydronaphthalene, tetrahydroquinoline, tetrahydroisoquinoline, indane, cyclopropylbenzene, cyclopentapyridine, and dihydrobenzoxaborole, any one of which is optionally substituted. R³ may be selected from:

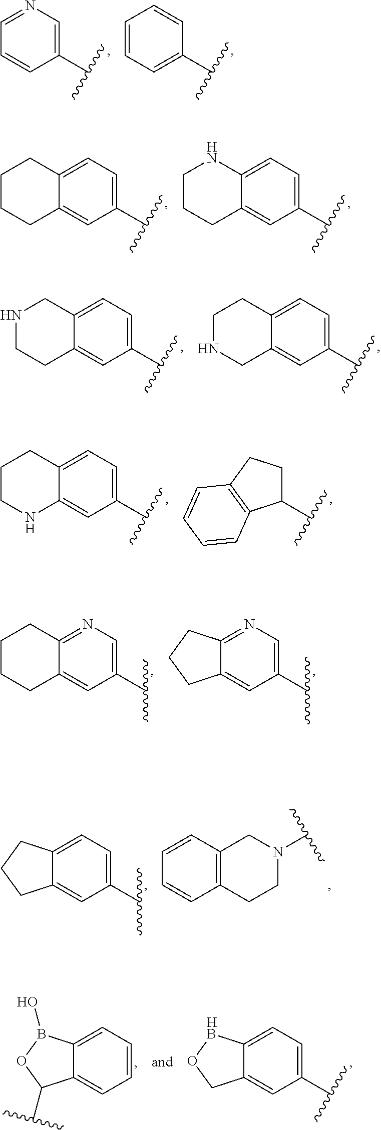

any one of which is optionally substituted. For example, R³ may be selected from:

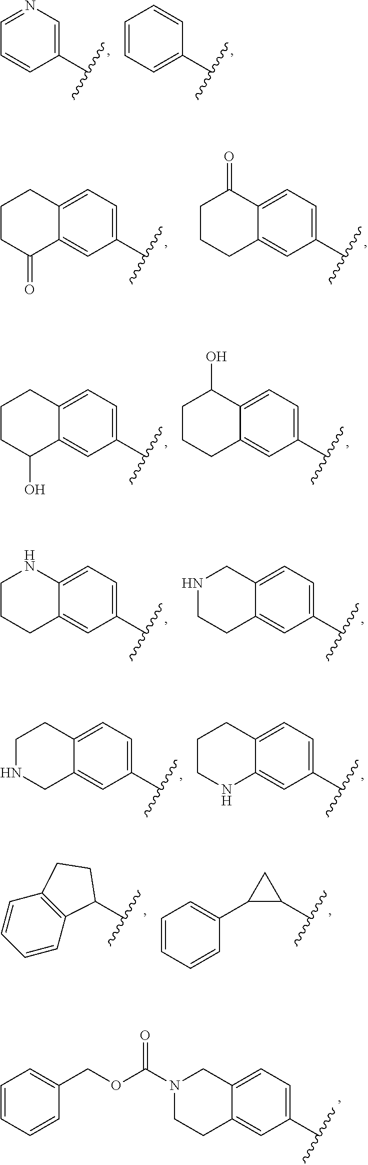

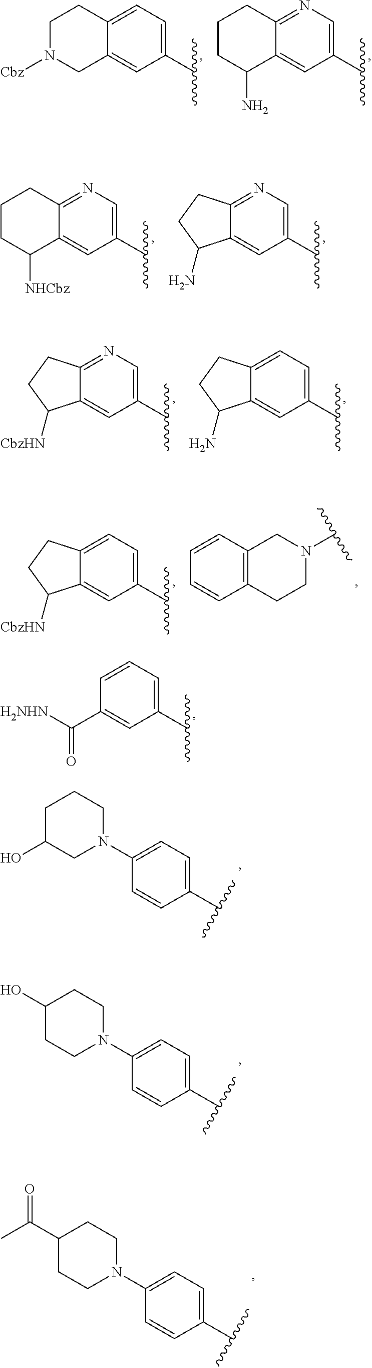

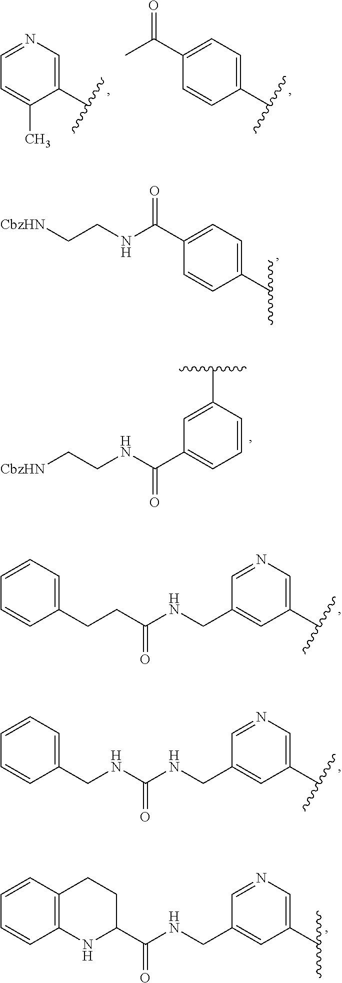

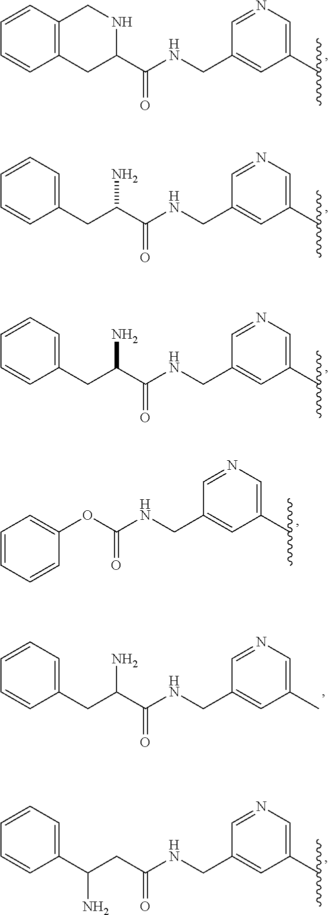 [SMILES: *c1cncc(CNC(=O)C2Cc3ccccc3CN2)c1 ; *c1cncc(CNC(=O)[C@@H](N)Cc2ccccc2)c1 ; *c1cncc(CNC(=O)[C@H](N)Cc2ccccc2)c1 ; *c1cncc(CNC(=O)Oc2ccccc2)c1 ; Cc1cncc(CNC(=O)C(N)Cc2ccccc2)c1 ; *c1cncc(CNC(=O)CC(N)c2ccccc2)c1]

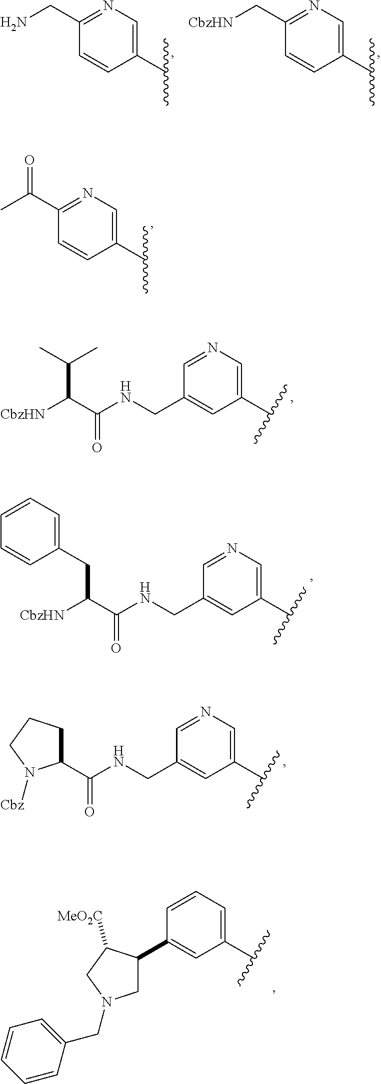

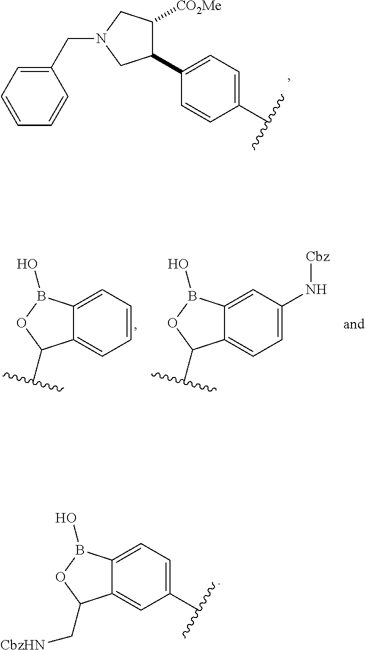

In some embodiments, L² is selected from —C(O)—, and —C(O)NR¹⁰—. In certain embodiments, L² is —C(O)—. In certain embodiments, L² is selected from —C(O)NR¹⁰—. R¹⁰ of —C(O)NR¹⁰— may be selected from hydrogen and C₁₋₆ alkyl. For example, L² may be —C(O)NH—.

In some embodiments, R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl.

In some embodiments, R⁴ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle. In some embodiments, R⁴ is selected from: —OR¹⁰, and —N(R¹⁰)₂; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl. In certain embodiments, R⁴ is —N(R¹⁰)₂. R¹⁰ of —N(R¹⁰)₂ may be independently selected at each occurrence from optionally substituted C₁₋₆ alkyl. In certain embodiments, R¹⁰ of —N(R¹⁰)₂ is independently selected at each occurrence from methyl, ethyl, propyl, and butyl, any one of which is optionally substituted. For example, R⁴ may be

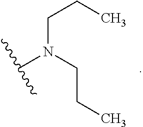 [SMILES: *N(CCC)CCC]

In certain embodiments, L²-R⁴ is

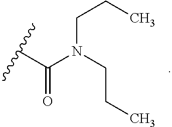 [SMILES: *C(=O)N(CCC)CCC]

In some embodiments, R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle; and C₃-10 carbocycle and 3- to 10-membered heterocycle, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₁₋₆ alkyl, C₂₋₆ alkenyl, C₂₋₆ alkynyl. In some embodiments, R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle.

In some embodiments, the compound is selected from:

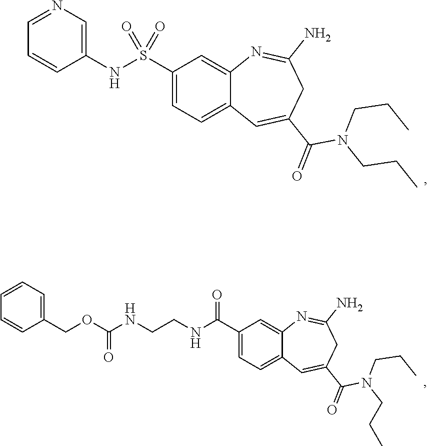 [SMILES: CCCN(CCC)C(=O)C1=Cc2ccc(S(=O)(=O)Nc3cccnc3)cc2N=C(N)C1 ; CCCN(CCC)C(=O)C1=Cc2ccc(C(=O)NCCNC(=O)OCc3ccccc3)cc2N=C(N)C1]

and a salt of any one thereof.

In some aspects, the present disclosure provides a compound represented by the structure of Formula (IVA):

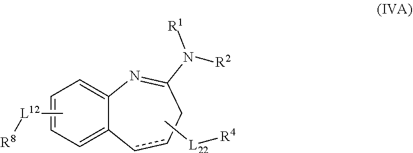

or a pharmaceutically acceptable salt thereof, wherein:
[symbol] represents an optional double bond;
L¹² is selected from —X³—, —X³—C₁₋₆ alkylene-X³—, —X³—C₂₋₆ alkenylene-X³—, and —X³—C₂₋₆ alkynylene-X³—, each of which is optionally substituted on alkylene, alkenylene, or alkynylene with one or more substituents independently selected from R¹²;
L²² is independently selected from —X⁴—, —X⁴—C₁₋₆ alkylene-X⁴—, —X⁴—C₂₋₆ alkenylene-X⁴—, and —X⁴—C₂₋₆ alkynylene-X⁴—, each of which is optionally substituted on alkylene, alkenylene, or alkynylene with one or more substituents independently selected from R¹⁰;
X³ and X⁴ are independently selected at each occurrence from a bond, —O—, —S—, —N(R¹⁰)—, —C(O)—, —C(O)O—, —OC(O)—, —OC(O)O—, —C(O)N(R¹⁰)—, —C(O)N(R¹⁰)C(O)—, —C(O)N(R¹⁰)C(O)N(R¹)—, —N(R¹⁰)C(O)—, —N(R¹⁰)C(O)N(R¹⁰)—, —N(R¹⁰)C(O)O—, —OC(O)N(R¹⁰)—, —C(NR¹⁰)—, —N(R¹⁰)C(NR¹⁰)—, —C(NR¹⁰)N(R¹⁰)—, —N(R¹⁰)C(NR¹⁰)N(R¹⁰)—, —S(O)₂—, —OS(O)—, —S(O)O—, —S(O)—, —OS(O)₂—, —S(O)₂O—, —N(R¹⁰)S(O)₂—, —S(O)₂N(R¹⁰)—, —N(R¹⁰)S(O)—, —S(O)N(R¹⁰)—, —N(R¹⁰)S(O)₂N(R¹⁰)—, and —N(R¹⁰)S(O)N(R¹⁰)—;
R¹ and R² are independently selected from L³, and hydrogen; and C₁₋₁₀ alkyl, C₂₋₁₀ alkenyl, and C₂₋₁₀ alkynyl, each of which is optionally bound to L³ and each of which is optionally substituted with one or more substituents independently selected from halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), and —CN;
R⁴ and R⁸ are independently selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, C₂₋₁₀ alkynyl, each of which is optionally bound to L³ and each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle, and 3- to 12-membered heterocycle, wherein each C₃₋₁₂ carbocycle, and 3- to 12-membered heterocycle in R⁴ and R⁸ is optionally bound to L³ and each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R¹⁰ is independently selected at each occurrence from L³, hydrogen, —NH₂, —C(O)OCH₂C₆H₅; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
L³ is a linker moiety, wherein there is at least one occurrence of L³; and
R¹² is independently selected at each occurrence from halogen, —OR¹⁰, —SR¹⁰, —N(R¹⁰)₂, —C(O)R¹⁰, —C(O)N(R¹⁰)₂, —N(R¹⁰)C(O)R, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), and —CN; C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R¹² is optionally substituted with one or more substituents independently selected from halogen, —OR¹⁰, —SR¹⁰, —N(R¹⁰)₂, —C(O)R¹⁰, —C(O)N(R¹⁰)₂, —N(R¹⁰)C(O)R, —C(O)OR¹⁰, —OC(O)R¹⁰, —S(O)R¹⁰, —S(O)₂R¹⁰, —P(O)(OR¹⁰)₂, —OP(O)(OR¹⁰)₂, —NO₂, ═O, ═S, ═N(R¹⁰), —CN, C₁₋₆ alkyl, C₂₋₆ alkenyl, C₂₋₆ alkynyl;
wherein any substitutable carbon on the benzazepine core is optionally substituted by a substituent independently selected from R¹² or two substituents on a single carbon atom combine to form a 3- to 7-membered carbocycle.

In some embodiments, the compound of Formula (IVA) is represented by Formula (IVB):

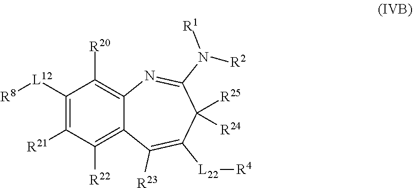

or a pharmaceutically acceptable salt thereof, wherein:
R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; and
R²⁴, and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In some embodiments, R¹ is L³. In some embodiments, R² is L³.

In some embodiments, L¹² is —C(O)N(R¹⁰)—. In some embodiments, R¹⁰ of —C(O)N(R¹⁰)— is selected from hydrogen, C₁₋₆ alkyl, and L³. For example, L¹² may be —C(O)NH—.

In some embodiments, R⁸ is an optionally substituted 5- or 6-membered heteroaryl. R⁸ may be an optionally substituted 5- or 6-membered heteroaryl, bound to L³. In some embodiments, R⁸ is an optionally substituted pyridine, bound to L³.

In some embodiments, L²² is selected from —C(O)—, and —C(O)NR¹⁰—. In certain embodiments, L²² is —C(O)—. In certain embodiments, L²² is —C(O)NR¹⁰—. R¹⁰ of —C(O)NR¹⁰— may be selected from hydrogen, C₁₋₆ alkyl, and -L³. For example, L²² may be —C(O)NH—.

In some embodiments, R⁴ is selected from: —OR¹⁰, and —N(R¹⁰)₂; and C₁₋₁₀ alkyl, C₂₋₁₀ alkenyl, C₂₋₁₀ alkynyl, C₃₋₁₂ carbocycle, 3- to 12-membered heterocycle, aryl, and heteroaryl, each of which is optionally substituted with one or more substituents independently selected from halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl and each of which is further optionally bound to L³. In some embodiments, R⁴ is —N(R¹⁰)₂ and R¹⁰ of —N(R¹⁰)₂ is selected from L³ and hydrogen, and wherein at least one R¹⁰ of —N(R¹⁰)₂ is L³.

In some aspects, the compound of Formula (IVB) is a compound of Formula (IVC):

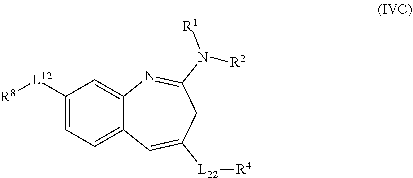

or a pharmaceutically acceptable salt thereof,
wherein:
R¹ and R² are hydrogen;
L²² is —C(O)—;
R⁴—N(R¹⁰)₂;
R¹⁰ is independently selected at each occurrence from hydrogen, —NH₂, —C(O)OCH₂C₆H₅; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
L¹² is —C(O)N(R¹⁰)—*, wherein * represents where L¹² is bound to R⁸;
R⁸ is an optionally substituted fused 5-5, fused 5-6, or fused 6-6 bicyclic heterocycle bound to linker moiety, L³, and wherein optional substituents are independently selected at each occurrence from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle, and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl.

In certain embodiments: R¹⁰ of —N(R¹⁰)₂ is independently selected at each occurrence from methyl, ethyl, propyl, and butyl, any one of which is optionally substituted. In certain embodiments, R¹⁰ of —C(O)N(R¹⁰)—* is hydrogen.

In some embodiments, the compound is further covalently bound to a linker, L³. In some embodiments, L³ is a noncleavable linker. In some embodiments, L³ is a cleavable linker. L³ may be cleavable by a lysosomal enzyme. In some embodiments, the compound is covalently attached to an antibody construct. In some embodiments, the compound is covalently attached to a targeting moiety, optionally through the linker. In some embodiments, the targeting moiety or antibody construct specifically binds to a tumor antigen. In some embodiments, the antibody construct or targeting moiety further comprises a target binding domain.

In some embodiments, L³ is represented by the formula:

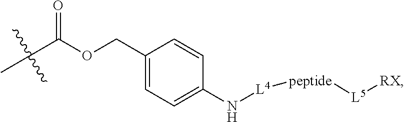

wherein:
L⁴ represents the C-terminus of the peptide and L⁵ is selected from a bond, alkylene and heteroalkylene, wherein L⁵ is optionally substituted with one or more groups independently selected from R³², and RX is a reactive moiety; and
R³² is independently selected at each occurrence from halogen, —OH, —CN, —O-alkyl, —SH, ═O, ═S, —NH₂, —NO₂; and C₁₋₁₀ alkyl, C₂₋₁₀ alkenyl, C₂₋₁₀ alkynyl, each of which is optionally substituted with one or more substituents independently selected from halogen, —OH, —CN, —O-alkyl, —SH, ═O, ═S, —NH₂, —NO₂.

In some embodiments, RX comprises a leaving group. In some embodiments, RX comprises a maleimide. In some embodiments, L³ is further covalently bound to an antibody construct. In some embodiments, the antibody construct is directed against a tumor antigen. In some embodiments, the antibody construct further comprises a target binding domain.

In some embodiments, L³ is represented by the formula:

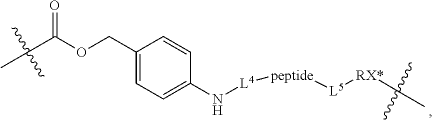

wherein
L⁴ represents the C-terminal of the peptide and
L⁵ is selected from a bond, alkylene and heteroalkylene,
   wherein L⁵ is optionally substituted with one or more groups independently selected from R³²;
RX* comprises a bond, a succinimide moiety, or a hydrolyzed succinimide moiety bound to a residue of an antibody construct,
   wherein

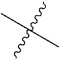

on RX* represents the point of attachment to the residue of the antibody construct; and,
R³² is independently selected at each occurrence from halogen, —OH, —CN, —O-alkyl, —SH, ═O, ═S, —NH₂, —NO₂; and C₁₋₁₀ alkyl, C₂₋₁₀ alkenyl, C₂₋₁₀ alkynyl, each of which is optionally substituted with one or more substituents independently selected from halogen, —OH, —CN, —O-alkyl, —SH, ═O, ═S, —NH₂, —NO₂. In some embodiments, the peptide of L³ comprises Val-Cit or Val-Ala.

In some aspects, the present disclosure provides a compound or salt selected from:

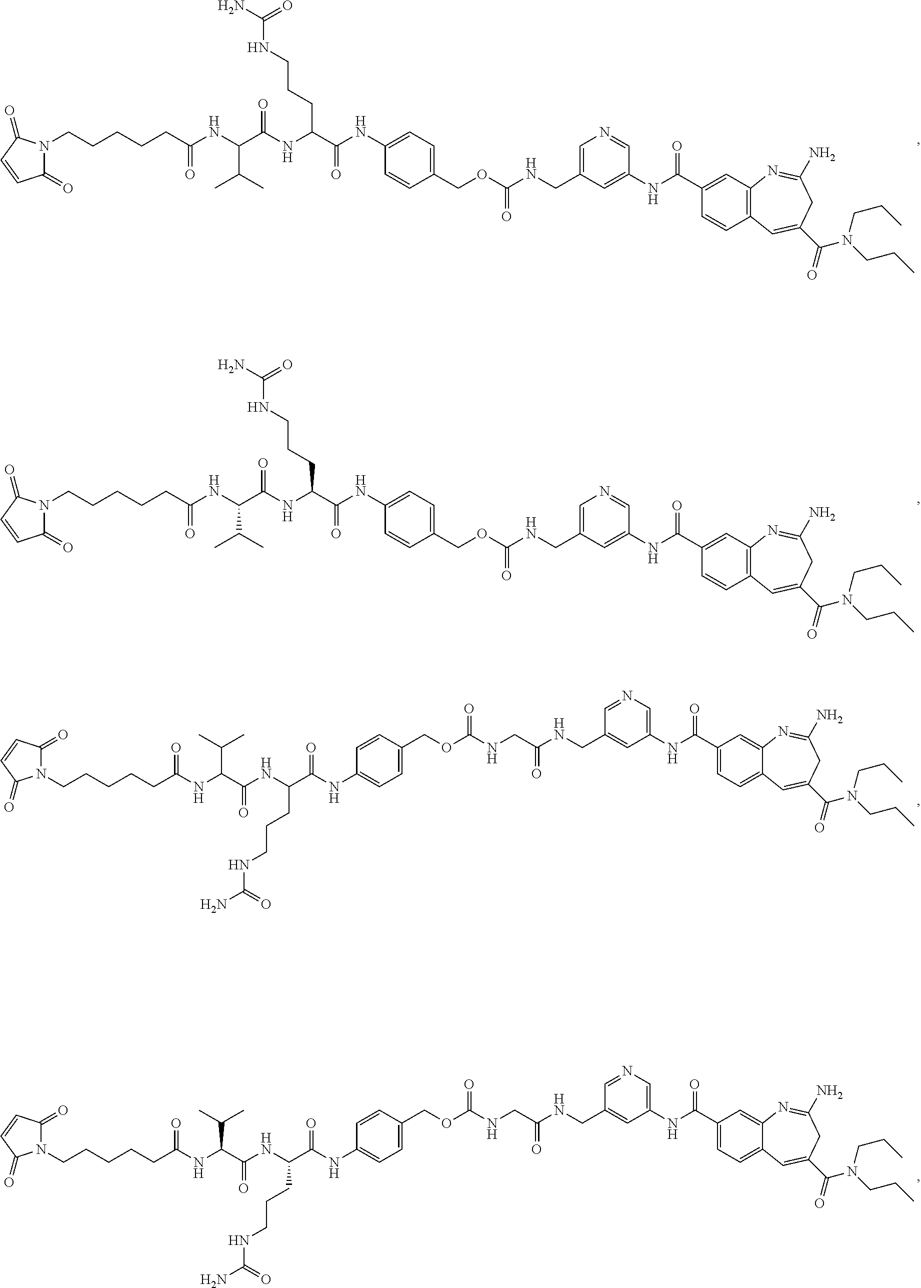

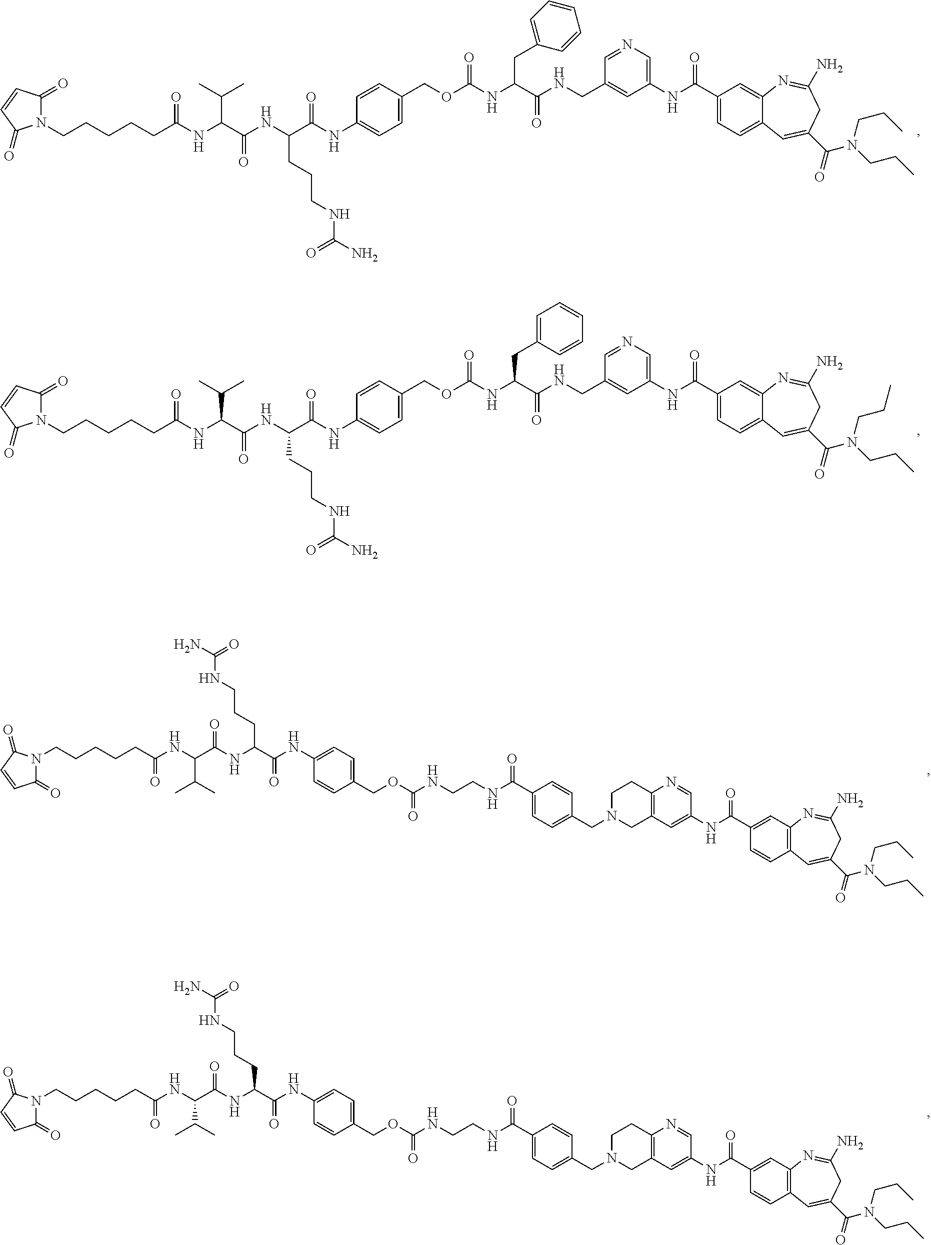

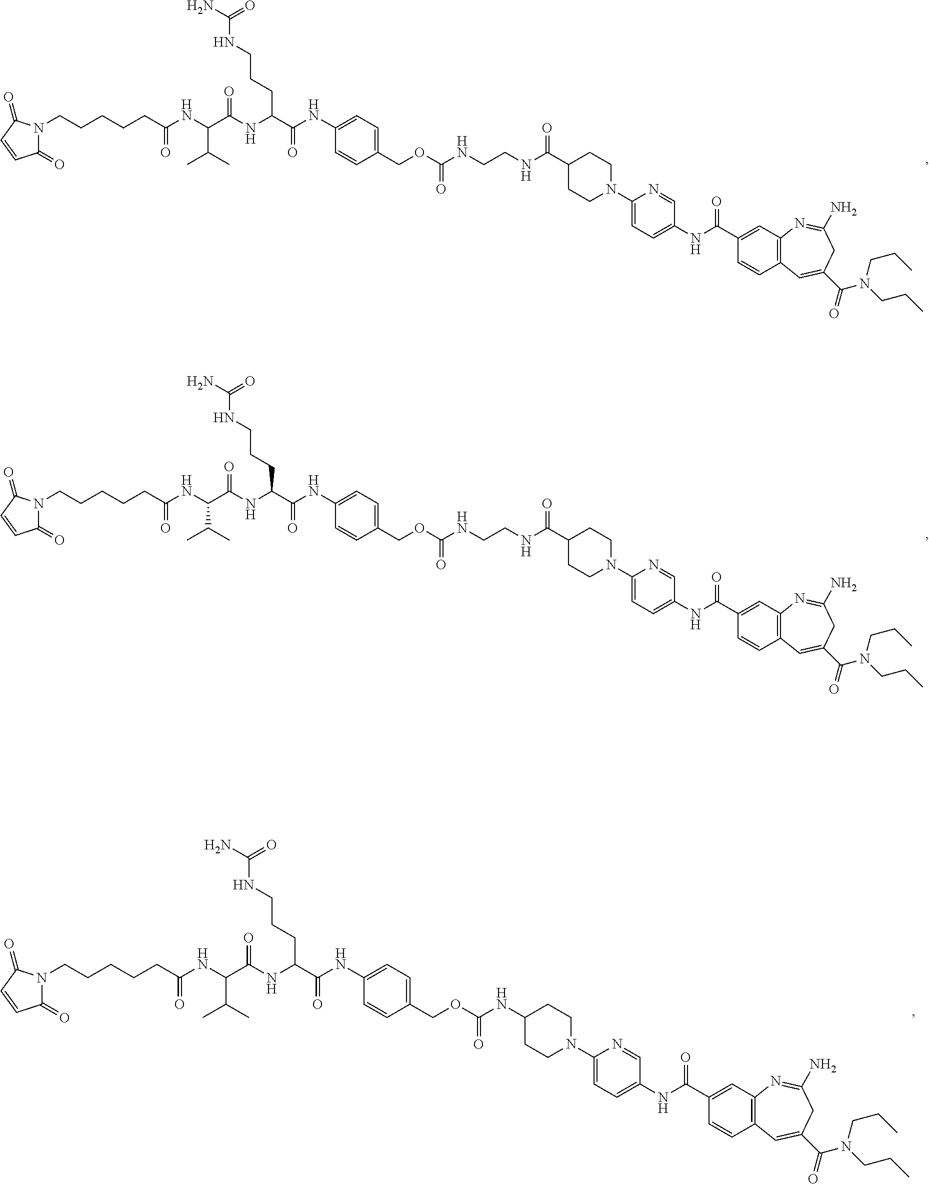 [SMILES: CCCN(CCC)C(=O)C1=Cc2ccc(C(=O)Nc3ccc(N4CCC(C(=O)NCCNC(=O)OCc5ccc(NC(=O)C(CCCNC(N)=O)NC(=O)C(NC(=O)CCCCCN6C(=O)C=CC6=O)C(C)C)cc5)CC4)nc3)cc2N=C(N)C1 ; CCCN(CCC)C(=O)C1=Cc2ccc(C(=O)Nc3ccc(N4CCC(C(=O)NCCNC(=O)OCc5ccc(NC(=O)[C@H](CCCNC(N)=O)NC(=O)[C@@H](NC(=O)CCCCCN6C(=O)C=CC6=O)C(C)C)cc5)CC4)nc3)cc2N=C(N)C1 ; CCCN(CCC)C(=O)C1=Cc2ccc(C(=O)Nc3ccc(N4CCC(NC(=O)OCc5ccc(NC(=O)C(CCCNC(N)=O)NC(=O)C(NC(=O)CCCCCN6C(=O)C=CC6=O)C(C)C)cc5)CC4)nc3)cc2N=C(N)C1]

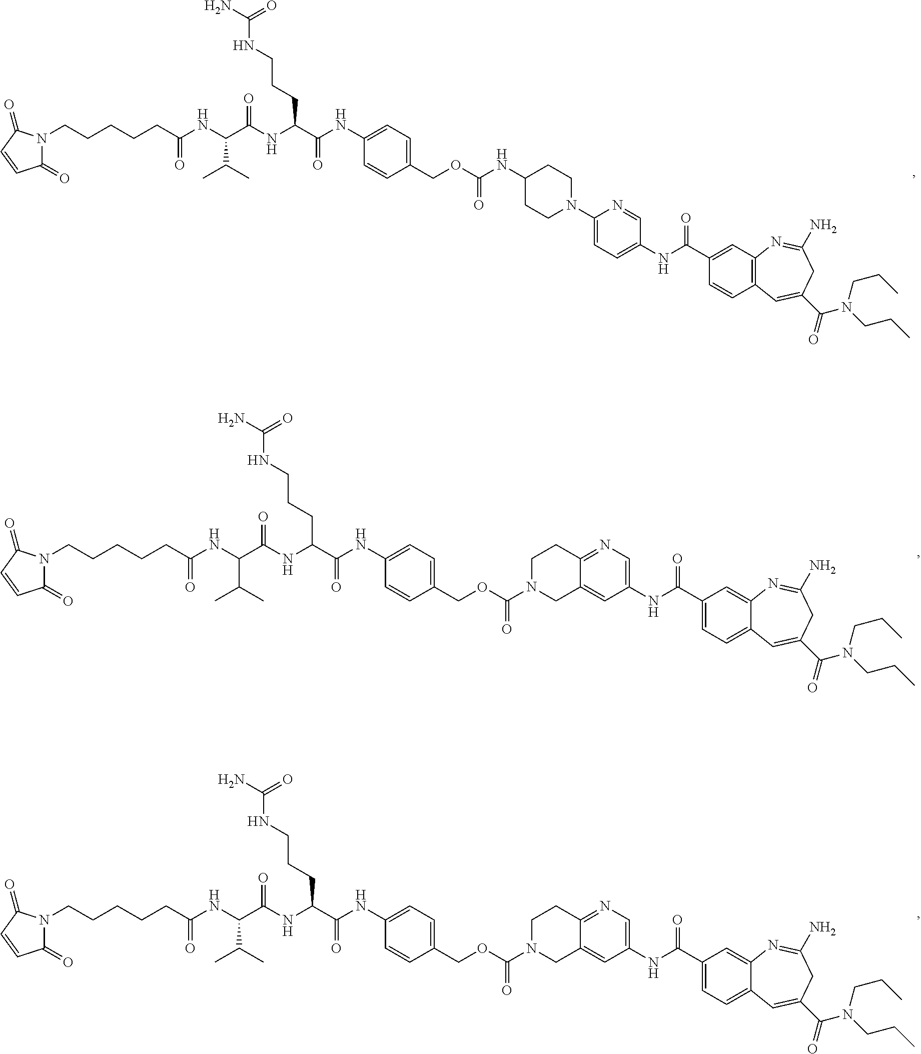 [SMILES: CCCN(CCC)C(=O)C1=Cc2ccc(C(=O)Nc3ccc(N4CCC(NC(=O)OCc5ccc(NC(=O)[C@H](CCCNC(N)=O)NC(=O)[C@@H](NC(=O)CCCCCN6C(=O)C=CC6=O)C(C)C)cc5)CC4)nc3)cc2N=C(N)C1 ; CCCN(CCC)C(=O)C1=Cc2ccc(C(=O)Nc3cnc4c(c3)CN(C(=O)OCc3ccc(NC(=O)C(CCCNC(N)=O)NC(=O)C(NC(=O)CCCCCN5C(=O)C=CC5=O)C(C)C)cc3)CC4)cc2N=C(N)C1 ; CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1]

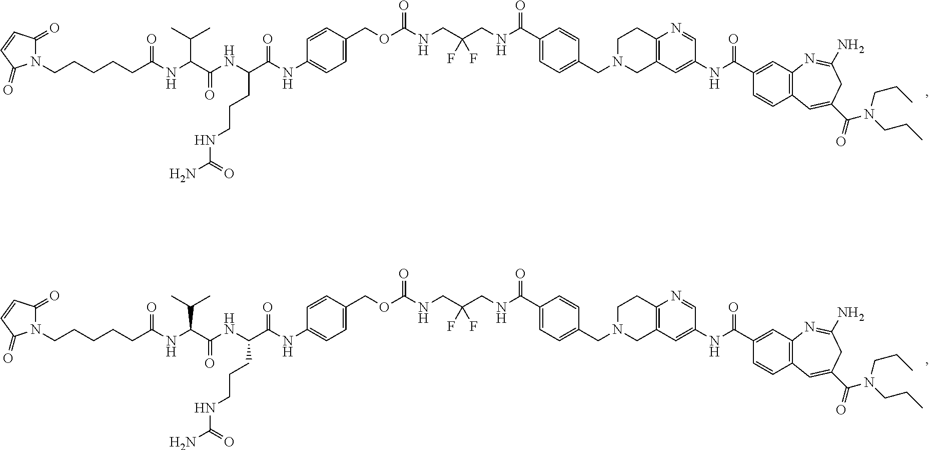 [SMILES: CCCN(CCC)C(=O)C1=Cc2ccc(C(=O)Nc3cnc4c(c3)CN(Cc3ccc(C(=O)NCC(F)(F)CNC(=O)OCc5ccc(NC(=O)C(CCCNC(N)=O)NC(=O)C(NC(=O)CCCCCN6C(=O)C=CC6=O)C(C)C)cc5)cc3)CC4)cc2N=C(N)C1 ; CCCN(CCC)C(=O)C1=Cc2ccc(C(=O)Nc3cnc4c(c3)CN(Cc3ccc(C(=O)NCC(F)(F)CNC(=O)OCc5ccc(NC(=O)[C@H](CCCNC(N)=O)NC(=O)[C@@H](NC(=O)CCCCCN6C(=O)C=CC6=O)C(C)C)cc5)cc3)CC4)cc2N=C(N)C1]

and a salt of any one thereof.

In some aspects, the present disclosure provides a compound or salt selected from:

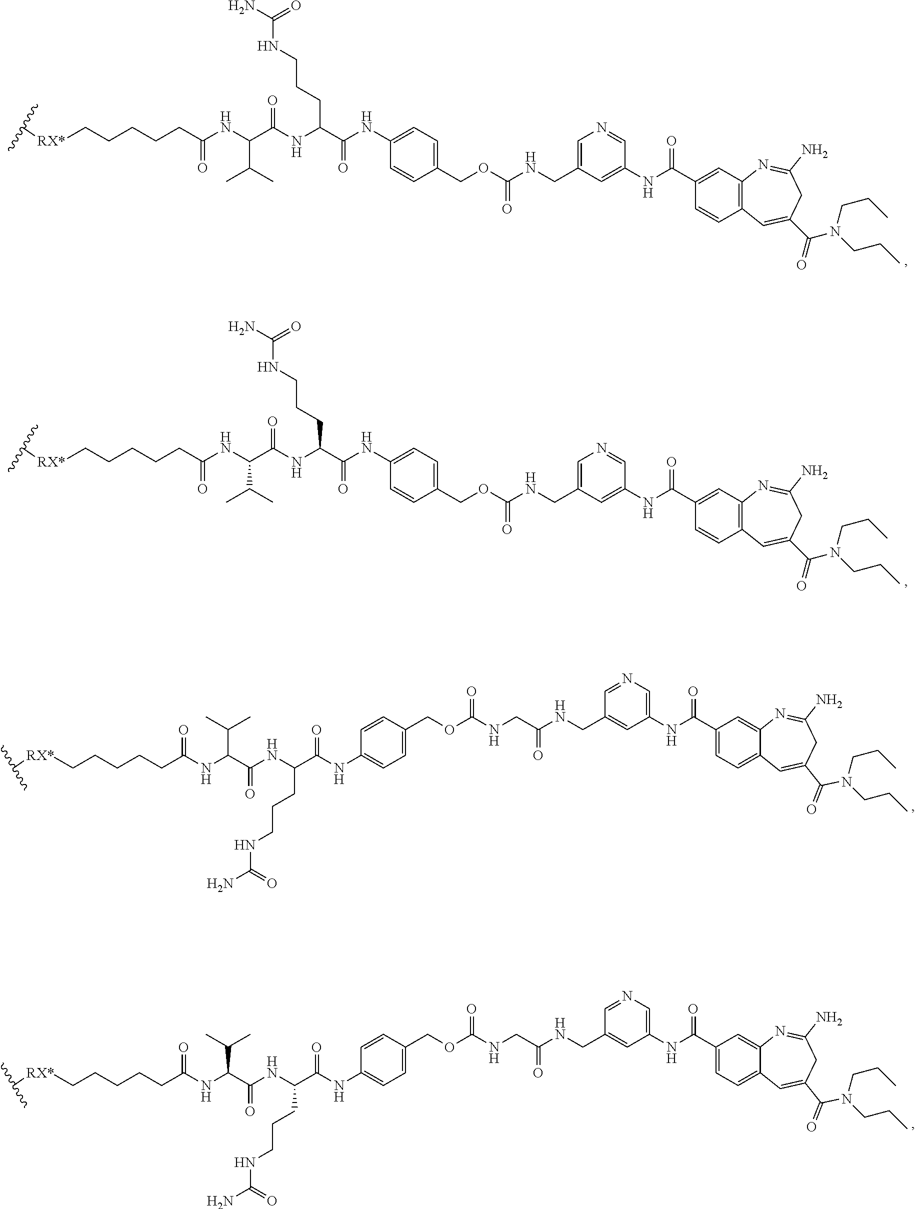

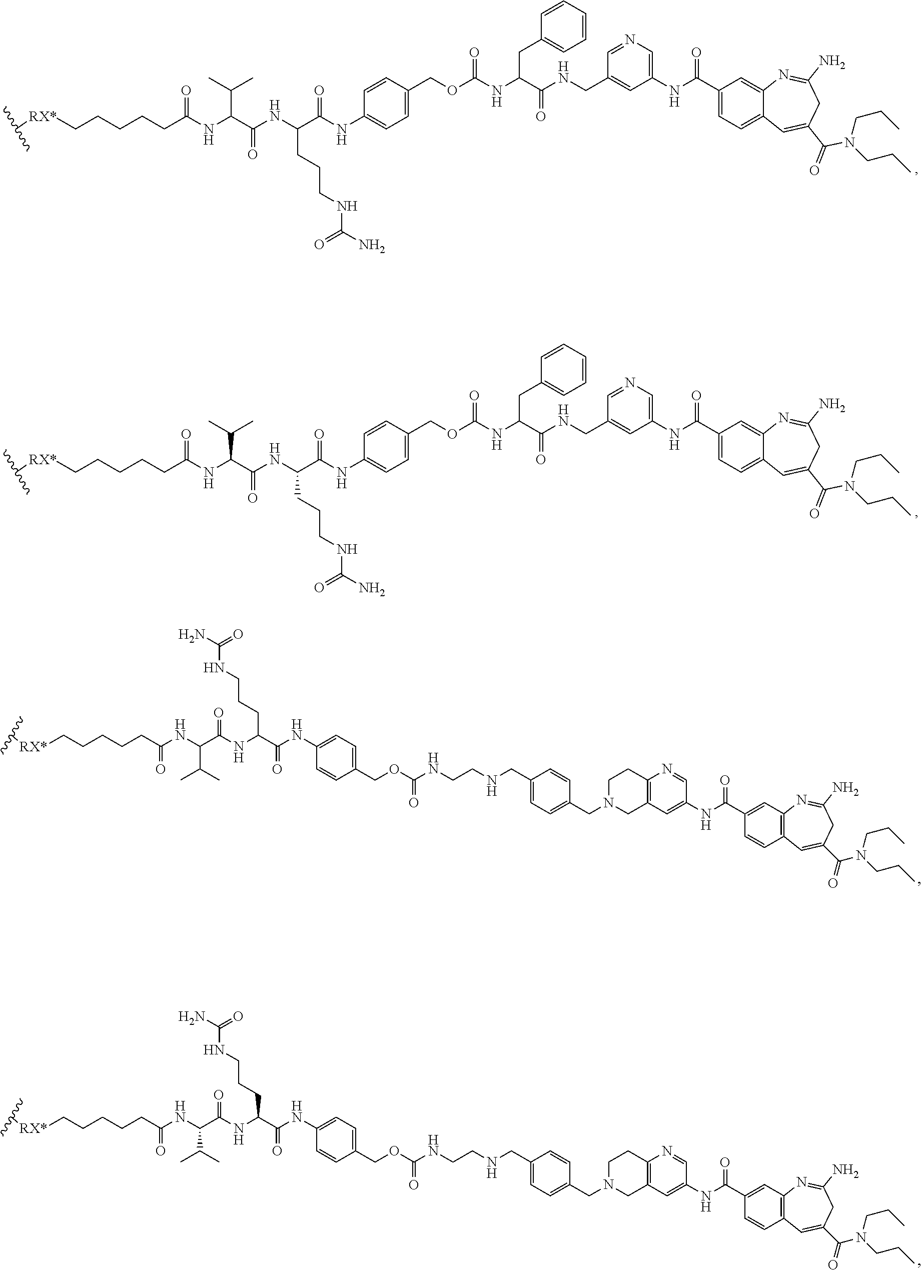

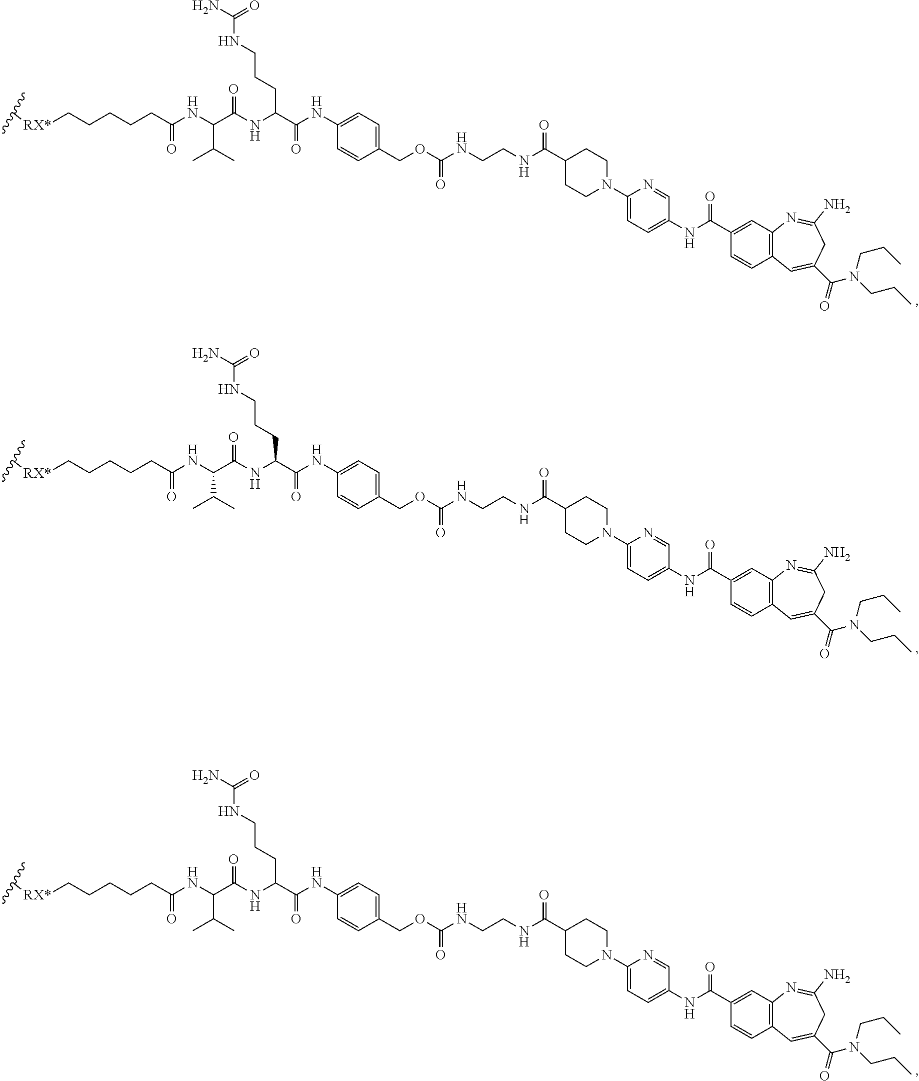

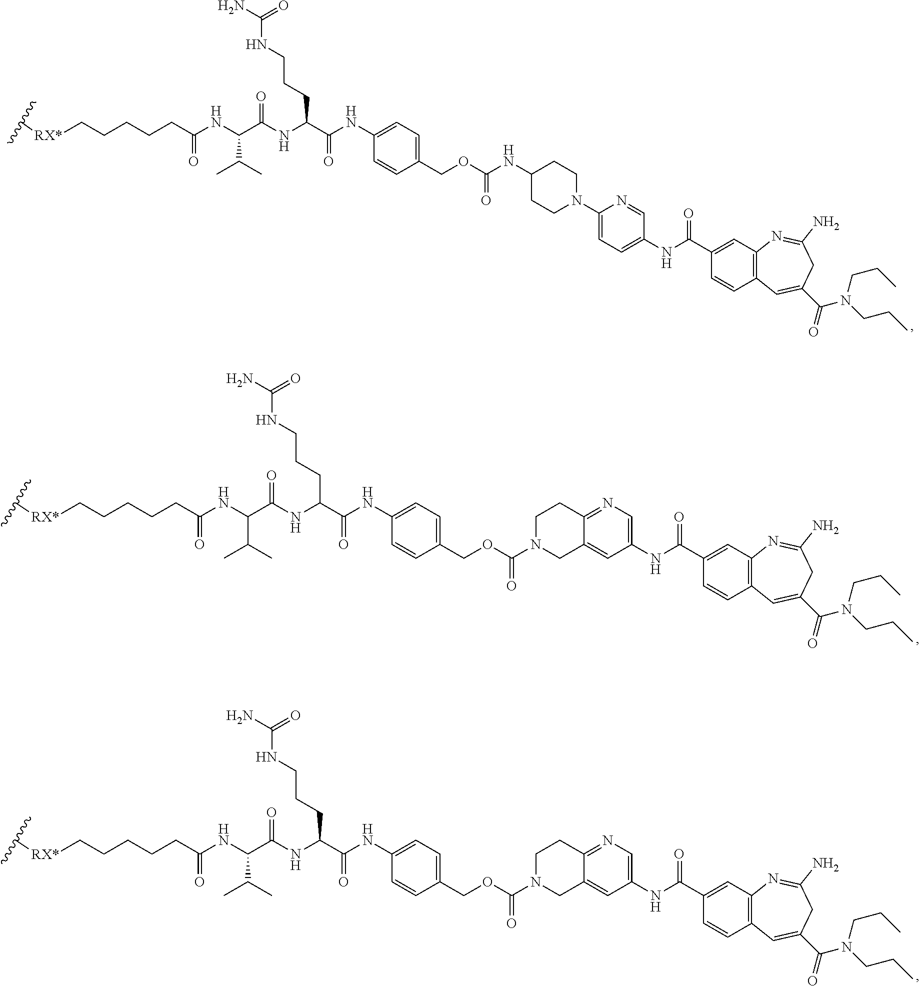

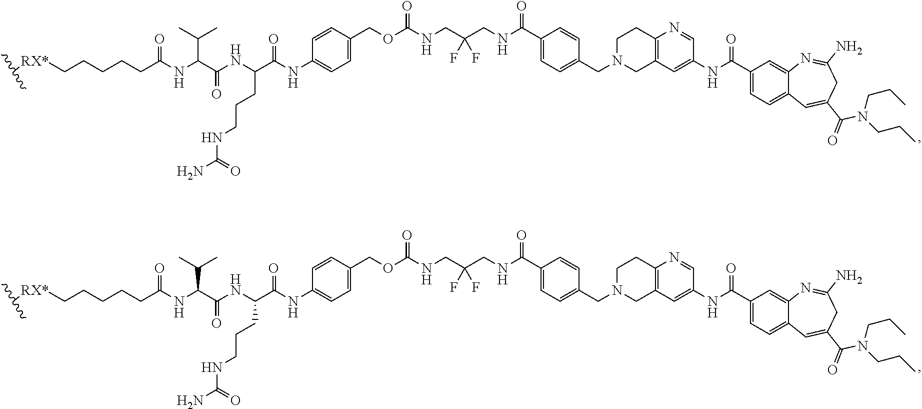

and a salt of any one thereof,
wherein the RX* is a bond, a succinimide moiety, or a hydrolyzed succinimide moiety bound to a residue of an antibody construct,
wherein

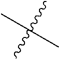

on RX* represents the point of attachment to the residue of the antibody construct.

In some embodiments, L³ is represented by the formula:

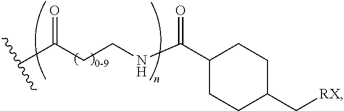

wherein RX comprises a reactive moiety, and n=0-9. In some embodiments, RX comprises a leaving group. In some embodiments, RX comprises a maleimide. In some embodiments, L³ is represented as follows:

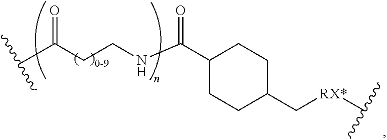

wherein RX* comprises a bond, a succinimide moiety, or a hydrolyzed succinimide moiety bound to a residue of an antibody construct, wherein

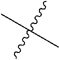

on RX* represents the point of attachment to the residue of the antibody construct, and n=0-9.

In some aspects, the present disclosure provides a compound or salt selected from:

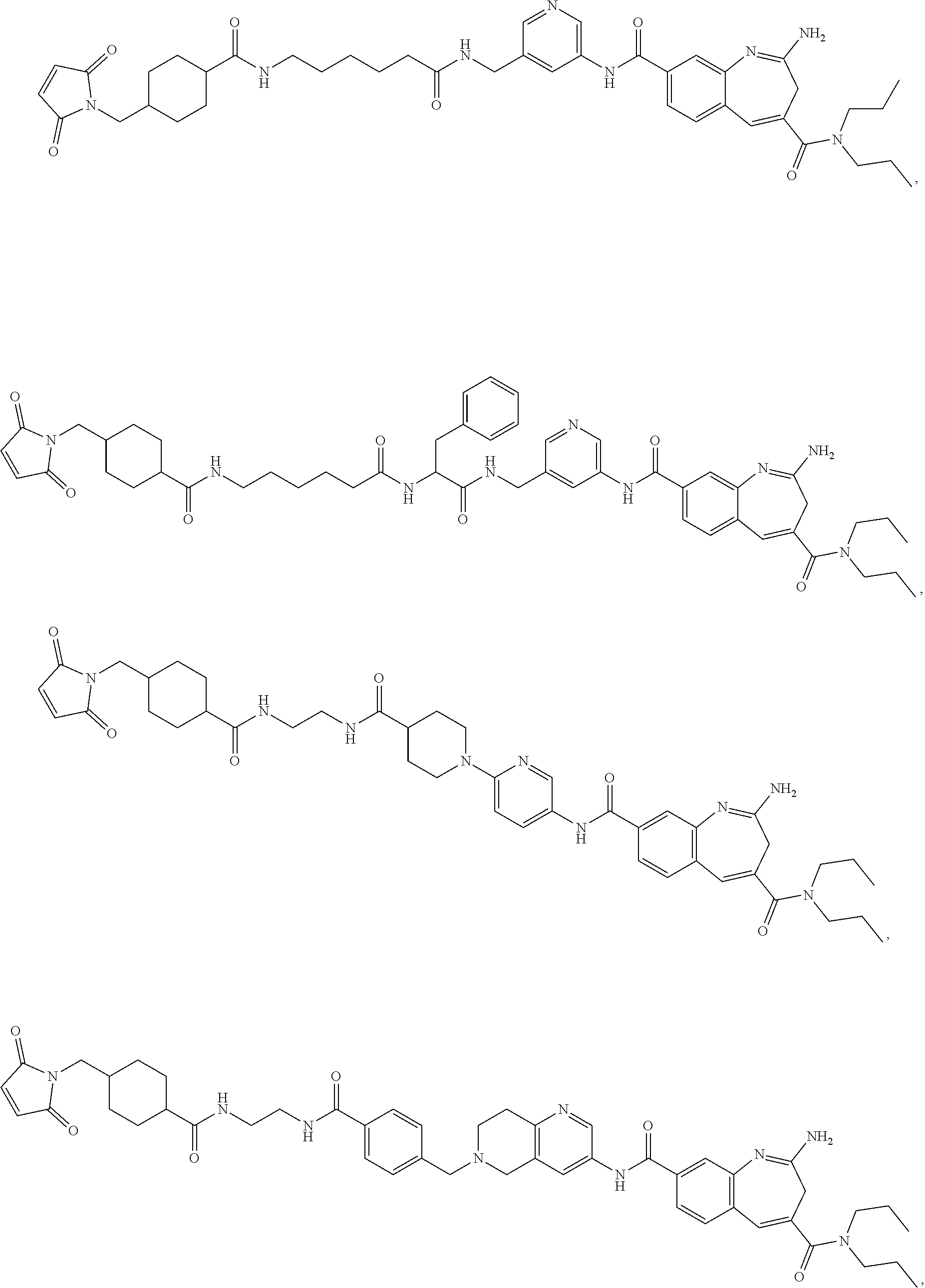 [SMILES: CCCN(CCC)C(=O)C1=Cc2ccc(C(=O)Nc3cncc(CNC(=O)CCCCCNC(=O)C4CCC(CN5C(=O)C=CC5=O)CC4)c3)cc2N=C(N)C1 ; CCCN(CCC)C(=O)C1=Cc2ccc(C(=O)Nc3cncc(CNC(=O)C(Cc4ccccc4)NC(=O)CCCCCNC(=O)C4CCC(CN5C(=O)C=CC5=O)CC4)c3)cc2N=C(N)C1 ; CCCN(CCC)C(=O)C1=Cc2ccc(C(=O)Nc3ccc(N4CCC(C(=O)NCCNC(=O)C5CCC(CN6C(=O)C=CC6=O)CC5)CC4)nc3)cc2N=C(N)C1 ; CCCN(CCC)C(=O)C1=Cc2ccc(C(=O)Nc3cnc4c(c3)CN(Cc3ccc(C(=O)NCCNC(=O)C5CCC(CN6C(=O)C=CC6=O)CC5)cc3)CC4)cc2N=C(N)C1]

and a salt of any one thereof.

In some aspects, the present disclosure provides a compound or salt selected from:

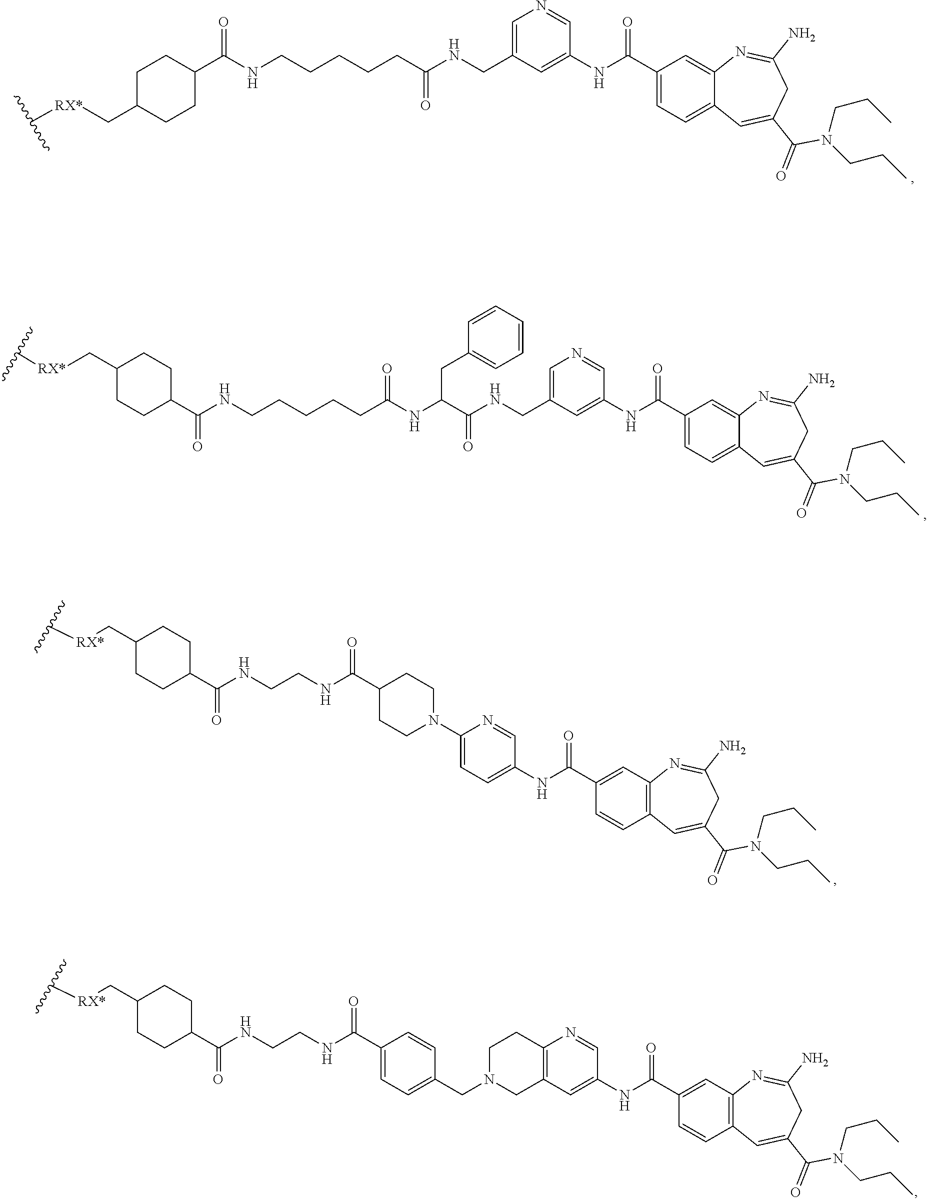

and a salt of any one thereof, wherein the RX* comprises a bond, a succinimide moiety, or a hydrolyzed succinimide moiety bound to a residue of an antibody construct, wherein

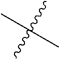

on RX* represents the point of attachment to the residue of the antibody construct.

In some embodiments, RX* comprises a succinamide moiety and is bound to a cysteine residue of an antibody construct. In some embodiments, RX* comprises a hydrolyzed succinamide moiety and is bound to a cysteine residue of an antibody construct.

In some aspects, the present disclosure provides a conjugate represented by the formula:

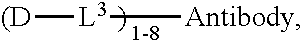

wherein Antibody is an antibody construct, D is a Category A compound or salt disclosed herein, and L³ is a linker moiety.

In some aspects, the present disclosure provides a conjugate represented by the formula:

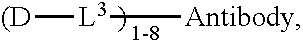

wherein Antibody is an antibody construct and D-L³ is a Category A compound or salt disclosed herein.

In some aspects, the present disclosure provides a pharmaceutical composition, comprising the conjugate disclosed herein and at least one pharmaceutically acceptable excipient.

In some embodiments, the average DAR of the conjugate is from about 2 to about 8, or about 1 to about 3, or about 3 to about 5.

### Compounds of Category B, TLR7 Agonists

In some aspects, the present disclosure provides a compound represented by the structure of Formula (IA):

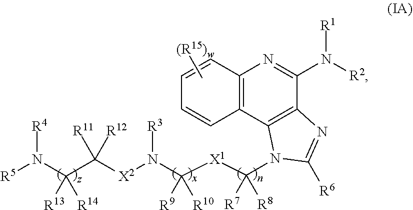

or a pharmaceutically acceptable salt thereof, wherein:
R¹, R², R³, R⁴, and R⁵ are independently selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; or R³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;
R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;
R⁷, R⁸, R⁹, and R¹⁰ are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;
R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;
R¹³ and R¹⁴ are independently selected at each occurrence from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;
R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;
R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;
R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;
X¹ is O, S, or NR¹⁶;
X² is C(O) or S(O)₂;
n is 1, 2, or 3;
x is 1, 2, or 3;
w is 0, 1, 2, 3, or 4; and
z is 0, 1, or 2.

In certain embodiments, for a compound of Formula (IA), wherein X¹ is O. In certain embodiments, for a compound of Formula (IA), n is 2. In certain embodiments, for a compound of Formula (IA), x is 2. In certain embodiments, for a compound of Formula (IA), z is 0. In certain embodiments, for a compound of Formula (IA), z is 1.

In certain embodiments, a compound of Formula (IA) is represented by Formula (IB):

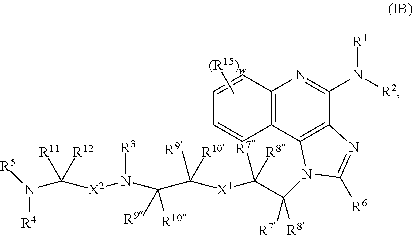

or a pharmaceutically acceptable salt thereof, wherein R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen.

In certain embodiments, a compound of Formula (IA) is represented by Formula (IC):

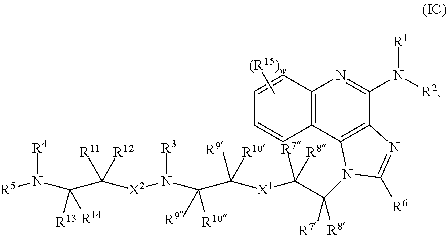

or a pharmaceutically acceptable salt thereof, wherein R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen.

In certain embodiments, for a compound or salt of any one of Formulas (IA), (IB), or (IC), R¹, R², R³, R⁴, and R⁵ are independently selected from hydrogen and C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN.

In certain embodiments, for a compound or salt of any one of Formulas (IA), (IB), or (IC), R¹ and R² are independently selected from hydrogen and C₁₋₆ alkyl. In certain embodiments, for a compound or salt of any one of Formulas (IA), (IB), or (IC), R¹ and R² are each hydrogen.

In certain embodiments, for a compound or salt of any one of Formulas (IA), (IB), or (IC), R³ is selected from hydrogen and C₁₋₆ alkyl optionally substituted with one or more halogens.

In certain embodiments, for a compound or salt of any one of Formulas (IA), (IB), or (IC), R³ is hydrogen.

In certain embodiments, for a compound or salt of any one of Formulas (IA), (IB), or (IC), R⁴ is selected from hydrogen and C₁₋₆ alkyl optionally substituted with one or more halogens.

In certain embodiments, for a compound or salt of any one of Formulas (IA), (IB), or (IC), R⁴ is hydrogen.

In certain embodiments, for a compound or salt of any one of Formulas (IA), (IB), or (IC), R⁵ is selected from hydrogen and C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN. In certain embodiments, for a compound or salt of any one of Formulas (IA), (IB), or (IC), R⁵ is hydrogen.

In certain embodiments, for a compound or salt of any one of Formulas (IA), (IB), or (IC), R⁶ is selected from halogen, —OR²⁰, and —N(R²⁰)₂; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and

R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle.

In certain embodiments, for a compound or salt of any one of Formulas (IA), (IB), or (IC),
R⁶ is C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰; and
R²⁰ is independently selected at each occurrence from hydrogen; C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle.

In certain embodiments, for a compound or salt of any one of Formulas (IA), (IB), or (IC), R⁶ is C₁₋₆ alkyl substituted with —OR²⁰, and R²⁰ is selected from hydrogen and C₁₋₆ alkyl optionally substituted with one or more substituents independently selected from halogen, —OH, and —NH₂.

In certain embodiments, for a compound or salt of any one of Formulas (IA), (IB), or (IC), R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, optionally substituted with one or more substituents independently selected from halogen.

In certain embodiments, for a compound or salt of any one of Formulas (IB) or (IC), wherein R^{7′} and R^{8′} are each hydrogen. In certain embodiments, for a compound or salt of any one of Formulas (IB) or (IC), wherein R^{7″} and R^{8″} are each C₁₋₆ alkyl. In certain embodiments, for a compound or salt of any one of Formulas (IB) or (IC), R^{7″} and R^{8″} are each methyl.

In certain embodiments, for a compound or salt of any one of Formulas (IB) or (IC), R^{9′}, R^{9″}, R^{10′}, and R^{10″} are independently selected at each occurrence from hydrogen and C₁₋₆ alkyl.

In certain embodiments, for a compound or salt of any one of Formulas (IB) or (IC), R^{9′}, R^{9″}, R^{10′}, and R^{10″} are each hydrogen.

In certain embodiments, for a compound or salt of any one of Formulas (IA), (IB), or (IC), R¹¹ and R¹² are independently selected from hydrogen, halogen, —OR²⁰, —SR²⁰, —C(O)N(R²⁰)₂, —N(R²⁰)₂, —C(O)R²⁰, —C(O)OR²⁰, —OC(O)R²⁰; and C₁₋₆ alkyl, optionally substituted with one or more substituents independently selected from halogen, —OR²⁰, —SR²⁰, —C(O)N(R²⁰)₂, —N(R²⁰)₂, —C(O)R²⁰, —C(O)OR²⁰, —OC(O)R²⁰, C₃₋₁₂ carbocycle, and 3- to 12-membered heterocycle.

In certain embodiments, for a compound or salt of any one of Formulas (IA) or (IC), R¹³ and R¹⁴ are independently selected from hydrogen, halogen, —OR²⁰, —SR²⁰, —C(O)N(R²⁰)₂, —N(R²⁰)₂, —C(O)R²⁰, —C(O)OR²⁰, —OC(O)R²⁰; and C₁₋₆ alkyl optionally substituted with one or more substituents independently selected from halogen, —OR²⁰, —SR²⁰, —C(O)N(R²⁰)₂, —N(R²⁰)₂, —C(O)R²⁰, —C(O)OR²⁰, —OC(O)R²⁰, C₃₋₁₂ carbocycle, and 3- to 12-membered heterocycle.

In certain embodiments, for a compound or salt of any one of Formulas (IA), (IB), or (IC), R³ and R¹¹ taken together form an optionally substituted 5- to 6-membered heterocycle.

In certain embodiments, for a compound or salt of any one of Formulas (IA), (IB), or (IC), R¹¹ and R¹² taken together form an optionally substituted C₃₋₆ carbocycle.

In certain embodiments, for a compound or salt of any one of Formulas (IA), (IB), or (IC), X² is C(O).

In certain embodiments, the compound is represented by:

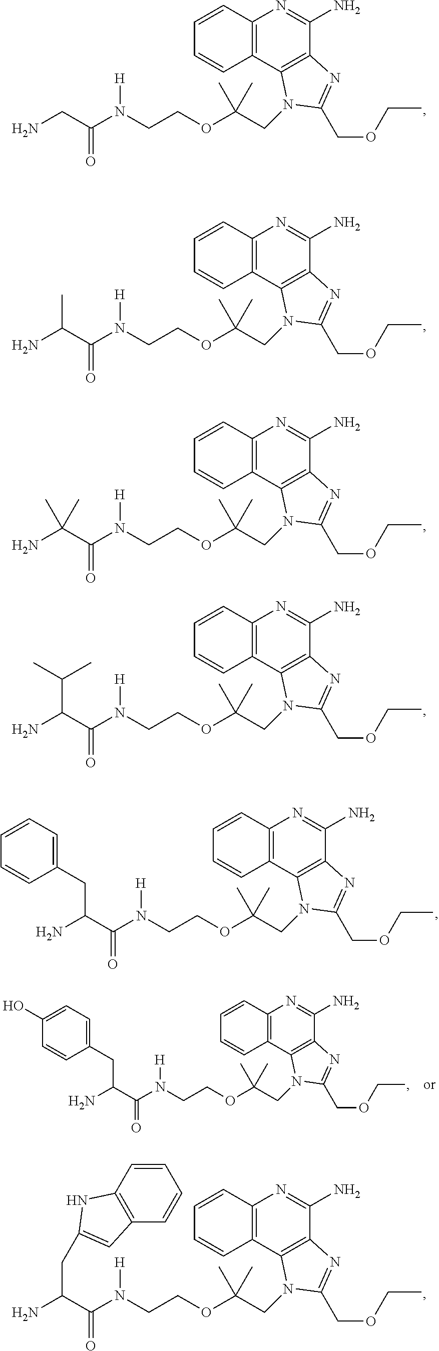

pharmaceutically acceptable salt of any one thereof.

In certain aspects, the disclosure provides a pharmaceutical composition of a compound or pharmaceutically acceptable salt of any one of Formulas (IA), (IB), or (IC), and a pharmaceutically acceptable excipient.

In certain embodiments, for a compound or salt of any one of Formulas (IA), (IB), or (IC), the compound or salt is further covalently bound to a linker, L³.

In certain aspects the disclosure provides a compound represented by Formula (IIA):

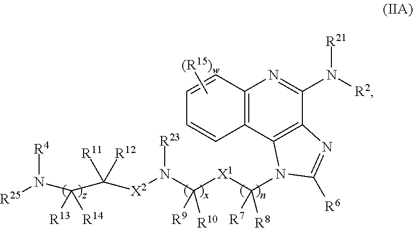

or a pharmaceutically acceptable salt thereof, wherein:
R² and R⁴ are independently selected from hydrogen; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;
R²¹, R²³, and R²⁵ are independently selected from hydrogen; C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and L³; or R²³ and R¹¹ taken together form a 5- to 10-membered heterocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and wherein one of R²¹, R²³, and R²⁵ is L³;
R⁶ is selected from halogen, —OR²⁰, —N(R²⁰)₂, —C(O)N(R²⁰)₂, —C(O)R²⁰, —C(O)OR²⁰, —S(O)R²⁰, and —S(O)₂R²⁰; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;
R⁷, R⁸, R⁹, and R¹⁰ are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen;
R¹¹ and R¹² are independently selected from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, and —CN; and C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; or R¹¹ and R¹² taken together form a C₃₋₆ carbocycle optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN;
R¹³ and R¹⁴ are independently selected at each occurrence from hydrogen, halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, —CN, C₁₋₆ alkyl, C₂₋₆ alkenyl, and C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl;
R¹⁵ is independently selected at each occurrence from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), —CN, C₁₋₆ alkyl, C₂₋₆ alkenyl, and C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;
R¹⁶ is selected from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;
R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle;
L³ is a linker;
X¹ is O, S, or NR¹⁶;
X² is C(O) or S(O)₂;
n is 1, 2, or 3;
x is 1, 2, or 3;
w is 0, 1, 2, 3, or 4; and
z is 0, 1, or 2.

In certain embodiments, for a compound or salt of Formula (IIA), X¹ is O. In certain embodiments, for a compound or salt of Formula (IIA), n is 2. In certain embodiments, for a compound or salt of Formula (IIA), x is 2. In certain embodiments, for a compound or salt of Formula (IIA), z is 0. In certain embodiments, for a compound or salt of Formula (IIA), z is 1.

In certain embodiments, the compound of Formula (IIA) is represented by (IIB) or (IIC):

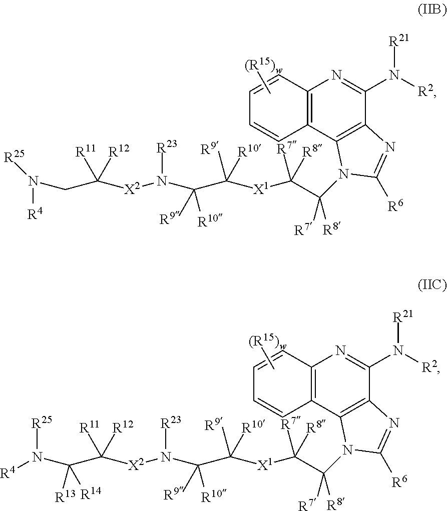

or a pharmaceutically acceptable salt thereof, wherein R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl, C₂₋₆ alkenyl, and C₂₋₆ alkynyl, each of which is optionally substituted with one or more substituents independently selected from halogen.

In certain embodiments, for a compound or salt of any one of Formulas (IIA), (IIB), or (IIC), R² and R⁴ are independently selected from hydrogen and C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN.

In certain embodiments, for a compound or salt of any one of Formulas (IIA), (IIB), or (IIC), R² and R⁴ are independently selected from hydrogen and C₁₋₆ alkyl. In certain embodiments, for a compound or salt of any one of Formulas (IIA), (IIB), or (IIC), R² and R⁴ are each hydrogen.

In certain embodiments, for a compound or salt of any one of Formulas (IIA), (IIB), or (IIC), R²³ is selected from hydrogen and C₁₋₆ alkyl optionally substituted with one or more halogens. In certain embodiments, for a compound or salt of any one of Formulas (IIA), (IIB), or (IIC), R²³ is hydrogen.

In certain embodiments, for a compound or salt of any one of Formulas (IIA), (IIB), or (IIC), R²¹ is selected from hydrogen and C₁₋₆ alkyl optionally substituted with one or more halogens. In certain embodiments, for a compound or salt of any one of Formulas (IIA), (IIB), or (IIC), R²¹ is hydrogen.

In certain embodiments, for a compound or salt of any one of Formulas (IIA), (IIB), or (IIC), R²¹ is L³.

In certain embodiments, for a compound or salt of any one of Formulas (IIA), (IIB), or (IIC), R²⁵ is selected from hydrogen and C₁₋₆ alkyl, optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN. In certain embodiments, for a compound or salt of any one of Formulas (IIA), (IIB), or (IIC), R²⁵ is hydrogen.

In certain embodiments, for a compound or salt of any one of Formulas (IIA), (IIB), or (IIC), R²⁵ is L³.

In certain embodiments, for a compound or salt of any one of Formulas (IIA), (IIB), or (IIC),
R⁶ is selected from halogen, —OR²⁰, and —N(R²⁰)₂; and C₁₋₆ alkyl, C₂₋₆ alkenyl, C₂₋₆ alkynyl, each of which is optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰, —NO₂, ═O, ═S, ═N(R²⁰), and —CN; and
R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle.

In certain embodiments, for a compound or salt of any one of Formulas (IIA), (IIB), or (IIC),
R⁶ is C₁₋₆ alkyl optionally substituted with one or more substituents independently selected from halogen, —OR²⁰, —SR²⁰, —C(O)N(R²⁰)₂, —N(R²⁰)₂, —S(O)R²⁰, —S(O)₂R²⁰, —C(O)R²⁰, —C(O)OR²⁰, —OC(O)R²⁰; and
R²⁰ is independently selected at each occurrence from hydrogen, —NH₂, —C(O)OCH₂C₆H₅; C₁₋₆ alkyl, C₃₋₁₂ carbocycle, and 3- to 12-membered heterocycle, each of which is optionally substituted with one or more substituents independently selected from halogen, —OH, —CN, —NO₂, —NH₂, ═O, ═S, —C(O)OCH₂C₆H₅, —NHC(O)OCH₂C₆H₅, C₁₋₆ alkyl, —C₁₋₆ haloalkyl, —O—C₁₋₆ alkyl, C₂₋₆ alkenyl, C₂₋₆ alkynyl, C₃₋₁₂ carbocycle, and 3- to 12-membered heterocycle.

In certain embodiments, for a compound or salt of any one of Formulas (IIA), (IIB), or (IIC),
R⁶ is C₁₋₆ alkyl substituted with —OR²⁰, and
R²⁰ is selected from hydrogen and C₁₋₆ alkyl, which is optionally substituted with one or more substituents independently selected from halogen, —OH, and —NH₂.

In certain embodiments, for a compound or salt of any one of Formulas (IIB) or (IIC), R^{7′}, R^{7″}, R^{8′}, R^{8″}, R^{9′}, R^{9″}, R^{10′}, and R^{10″} are independently selected at each occurrence from hydrogen and halogen; and C₁₋₆ alkyl optionally substituted with one or more substituents independently selected from halogen.

In certain embodiments, for a compound or salt of any one of Formulas (IIB) or (IIC), R^{7′} and R^{8′} are hydrogen.

In certain embodiments, for a compound or salt of any one of Formulas (IIB) or (IIC), R^{7″} and R^{8″} are C₁₋₆ alkyl.

In certain embodiments, for a compound or salt of any one of Formulas (IIB) or (IIC), R^{7″} and R^{8″} are methyl.

In certain embodiments, for a compound or salt of any one of Formulas (IIB) or (IIC), R^{9′}, R^{9″}, R^{10′}, and R^{10″} are independently selected at each occurrence from hydrogen and C₁₋₆ alkyl.

In certain embodiments, for a compound or salt of any one of Formulas (IIB) or (IIC), R^{9′}, R^{9″}, R^{10′}, and R^{10″} are each hydrogen.

In certain embodiments, for a compound or salt of any one of Formulas (IIA), (IIB), or (IIC), R¹¹ and R¹² are independently selected from hydrogen, halogen, —OR²⁰, —SR²⁰, —C(O)N(R²⁰)₂, —N(R²⁰)₂, —C(O)R²⁰, —C(O)OR²⁰, and —OC(O)R²⁰; and C₁₋₆ alkyl optionally substituted with one or more substituents independently selected from halogen, —OR²⁰, —SR²⁰, —C(O)N(R²⁰)₂, —N(R²⁰)₂, —C(O)R²⁰, —C(O)OR²⁰, —OC(O)R²⁰, C₃₋₁₂ carbocycle, and 3- to 12-membered heterocycle.

In certain embodiments, for a compound or salt of any one of Formulas (IIA) or (IIC), R¹³ and R¹⁴ are independently selected from hydrogen, halogen, —OR²⁰, —SR²⁰, —C(O)N(R²⁰)₂, —N(R²⁰)₂, —C(O)R²⁰, —C(O)OR²⁰, and —OC(O)R²⁰; and C₁₋₆ alkyl optionally substituted with one or more substituents independently selected from halogen, —OR²⁰, —SR²⁰, —C(O)N(R²⁰)₂, —N(R²⁰)₂, —C(O)R²⁰, —C(O)OR²⁰, —OC(O)R²⁰, C₃₋₁₂ carbocycle, and 3- to 12-membered heterocycle.

In certain embodiments, for a compound or salt of any one of Formulas (IIA), (IIB), or (IIC), R²³ and R¹¹ taken together form an optionally substituted 5- to 6-membered heterocycle.

In certain embodiments, for a compound or salt of any one of Formulas (IIA), (IIB), or (IIC), R¹¹ and R¹² taken together form an optionally substituted C₃₋₆ carbocycle.

In certain embodiments, for a compound or salt of any one of Formulas (IIA), (IIB), or (IIC), X² is C(O).

In certain embodiments, for a compound or salt of any one of Formulas (IIA), (IIB), or (IIC), L³ is a cleavable linker. In certain embodiments, for a compound or salt of any one of Formulas (IIA), (IIB), or (IIC), L³ is cleavable by a lysosomal enzyme.

In certain embodiments, for a compound or salt of any one of Formulas (IIA), (IIB), or (IIC), L³ is represented by the formula:

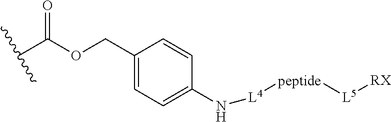

wherein:
L⁴ represents the C-terminus of the peptide and L⁵ is selected from a bond, alkylene and heteroalkylene, wherein L⁵ is optionally substituted with one or more groups independently selected from R³⁰, and RX is a reactive moiety; and
R³⁰ is independently selected at each occurrence from halogen, —OH, —CN, —O-alkyl, —SH, ═O, ═S, —NH₂, —NO₂; and C₁-C₁₀ alkyl, C₂-C₁₀ alkenyl, and C₂-C₁₀ alkynyl, each of which is independently optionally substituted at each occurrence with one or more substituents selected from halogen, —OH, —CN, —O-alkyl, —SH, ═O, ═S, —NH₂, and —NO₂.

In certain embodiments, for a compound or salt of any one of Formulas (IIA), (IIB), or (IIC), RX comprises a leaving group. In certain embodiments, for a compound or salt of any one of Formulas (IIA), (IIB), or (IIC), RX is a maleimide or an alpha-halo carbonyl. In certain embodiments, for a compound or salt of any one of Formulas (IIA), (IIB), or (IIC), the peptide of L³ comprises Val-Cit or Val-Ala.

In certain embodiments, for a compound or salt of any one of Formulas (IIA), (IIB), or (IIC), L³ is represented by the formula:

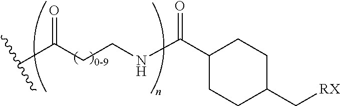

wherein:
RX comprises a reactive moiety; and
n is 0-9.

In certain embodiments, for a compound or salt of any one of Formulas (IIA), (IIB), or (IIC), RX comprises a leaving group. In certain embodiments, for a compound or salt of any one of Formulas (IIA), (IIB), or (IIC), RX is a maleimide or an alpha-halo carbonyl. In certain embodiments, for a compound or salt of any one of Formulas (IIA), (IIB), or (IIC), L³ is further covalently bound to an antibody construct to form a conjugate.

In certain embodiments, the disclosure provides a conjugate represented by the formula:

(D-L³ₙAntibody

wherein:
Antibody is an antibody construct;
n is 1 to 20;
D is a compound or salt of any one of a Category B compound of Formulas (IA), (IB), or (IC); and L³ is a linker moiety; or
D-L³ is a compound or salt of any one of a Category B compound of Formulas (IIA), (IIB), or (IIC).

In certain embodiments, for a conjugate of a compound or salt of any one of Formulas (IA), (IB), (IC), (IIA), (IIB), and (IIC), n is selected from 1 to 8. In certain embodiments, for a conjugate of a compound or salt of any one of Formulas (IA), (IB), (IC), (IIA), (IIB), and (IIC), n is selected from 2 to 5. In certain embodiments, for a conjugate of a compound or salt of any one of Formulas (IA), (IB), (IC), (IIA), (IIB), and (IIC), n is 2.

In certain embodiments, for a compound or salt of any one of Formulas (IIA), (IIB), and (IIC), -L³ is represented by the formula:

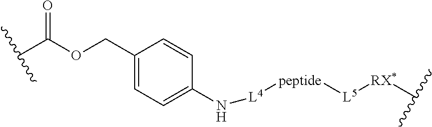

wherein:
L⁴ represents the C-terminus of the peptide and L⁵ is selected from a bond, alkylene and heteroalkylene, wherein L⁵ is optionally substituted with one or more groups independently selected from R³⁰;
RX* is a bond, a succinimide moiety, or a hydrolyzed succinimide moiety bound to a residue of an antibody construct, wherein

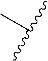

on RX* represents the point of attachment to the residue of the antibody construct; and
R³⁰ is independently selected at each occurrence from halogen, —OH, —CN, —O-alkyl, —SH, ═O, ═S, —NH₂, —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂.

In certain embodiments, for a compound or salt of any one of Formulas (IIA), (IIB), or (IIC), RX* is a succinamide moiety, hydrolyzed succinamide moiety or a mixture thereof and is bound to a cysteine residue of an antibody construct.

In certain embodiments for a compound of Formulas (IIA), (IIB) and (IIC), -L³ is represented by the formula:

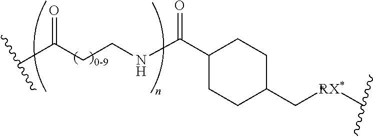

wherein:
RX* is a bond, a succinimide moiety, or a hydrolyzed succinimide moiety bound to a residue of an antibody construct, wherein

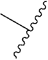

on RX* represents the point of attachment to the residue of the antibody construct; and
n is 0-9.

### Category A and Category B Conjugates

In certain embodiments, the disclosure provides an immune-stimulatory conjugate (or conjugate) of a targeting moiety or an antibody construct and at least one compound of any one of Category A Formulas (IA), (IB), (IIA), (IIB), (IIIA), and (IIIB), each compound optionally attached to the targeting moiety or antibody construct via a linker. In certain embodiments, the disclosure provides an immune-stimulatory conjugate of a targeting moiety or an antibody construct and at least one compound of any one of Category B Formulas (IA), (IB), or (IC), each compound optionally attached to the targeting moiety or antibody construct via a linker. In certain embodiments, the average Drug-to-Antibody Ratio (DAR) of the pharmaceutical composition is selected from 1 to 8.

In certain embodiments, the disclosure provides a pharmaceutical composition suitable for subcutaneous administration, comprising an immune stimulatory conjugate of a compound of any one of Category A Formulas (IA), (IB), (IIA), (IIB), (IIIA), and (IIIB), and a pharmaceutically acceptable excipient. In certain embodiments, the disclosure provides a pharmaceutical composition suitable for subcutaneous administration, comprising an immune stimulatory conjugate of a compound of any one of Category B Formulas (IA), (IB), or (IC), and a pharmaceutically acceptable excipient. In certain embodiments, the average Drug-to-Antibody Ratio (DAR) of the pharmaceutical composition is selected from 1 to 8.

In certain embodiments, the disclosure provides a method for the treatment of a disease treatable by a TLR agonist (e.g., cancer, viral disease) comprising subcutaneously administering an effective amount of a conjugate of a compound of any one of Category A Formulas (IA), (IB), (IIA), (IIB), (IIIA), and (IIIB), or a pharmaceutical composition thereof suitable for subcutaneous administration to a subject in need thereof, while alleviating, sparing, or avoiding toxicity(ies) associated with bolus intravenous administration of the conjugate. In some embodiments, the toxicity that is alleviated, spared, or avoided is anaphylaxis-like toxicity. In certain embodiments, the disclosure provides a method for the treatment of cancer, comprising subcutaneously administering an effective amount of the conjugate of a compound of any one of Category B Formulas (IA), (IB), or (IC), or a pharmaceutical composition thereof suitable for subcutaneous administration to a subject in need thereof, while alleviating, sparing, or avoiding toxicity(ies) associated with bolus intravenous administration of the conjugate. Toxicities that can be alleviated, spared, or avoided include anaphylaxis-like toxicity.

In certain embodiments, the disclosure provides a method for treatment, comprising subcutaneously administering to a subject in need thereof a conjugate of a compound of any one of Category A Formulas (IA), (IB), (IIA), (IIB), (IIIA), and (IIIB), or a pharmaceutical composition thereof suitable for subcutaneous administration, while alleviating, sparing, or avoiding toxicity(ies) associated with bolus intravenous administration of the conjugate. Toxicities that can be alleviated, spared, or avoided include anaphylaxis-like toxicity. In certain embodiments, the disclosure provides a method for treatment, comprising subcutaneously administering to a subject a conjugate of a compound of any one of Category B Formulas (IA), (IB), or (IC) or a pharmaceutical composition thereof suitable for subcutaneous administration, while alleviating, sparing, or avoiding toxicity(ies) associated with bolus intravenous administration of the conjugate. Toxicities that can be alleviated, spared, or avoided include anaphylaxis-like toxicity.

The disclosure provides a conjugate of a compound of any one of Category A Formulas (IA), (IB), (IIA), (IIB), (IIIA), and (IIIB), or a pharmaceutical composition thereof suitable for subcutaneous administration for use in a method of treatment of a subject's body by therapy by subcutaneous administration of the conjugate, while alleviating, sparing, or avoiding toxicity(ies) associated with bolus intravenous administration of the conjugate. Toxicities that can be alleviated, spared, or avoided include anaphylaxis-like toxicity. The disclosure provides a conjugate of a compound of any one of Category B Formulas (IA), (IB), or (IC) or a pharmaceutical composition thereof suitable for subcutaneous administration for use in a method of treatment of a subject's body by therapy, while alleviating, sparing, or avoiding toxicity(ies) associated with bolus intravenous administration of the conjugate. Toxicities that can be alleviated, spared, or avoided include anaphylaxis-like toxicity.

The disclosure provides a method of preparing an antibody conjugate of the formula:

(D-L³ₙAntibody

wherein:
Antibody is an antibody construct;
n is selected from 1 to 20;
L³ is a linker; and
D is selected from a compound or salt of a compound of any one of Category A Formulas (IA), (IB), (IIA), (IIB), (IIIA), and (IIIB) and Category B Formulas (IA), (IB), or (IC),
   comprising contacting D-L³ with an antibody construct.

The disclosure provides a method of preparing an antibody conjugate of the formula:

(D-L³ₙAntibody

wherein:
Antibody is an antibody construct;
n is selected from 1 to 20;
L³ is a linker; and
D is selected from a compound of any one of Category A Formulas (IA), (IB), (IIA), (IIB), (IIIA), and (IIIB) and Category B Formulas (IA), (IB), or (IC),
comprising contacting L³ with the antibody construct to form L³-antibody and contacting L³ antibody with D to form the conjugate.

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present disclosure, whether radioactive or not, are encompassed within the scope of the present disclosure.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present disclosure also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley and Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds described herein may be prodrugs attached to antibody constructs to form conjugates. The term “prodrug” is intended to encompass compounds which, under physiologic conditions, are converted into active compounds, e.g., TLR8 agonists, TLR7 agonists, other TLR agonists, STING agonist, RIG-I-Like receptor agonists, c-type lectin receptors agonists, or cytosolic DNA Sensors agonists. One method for making a prodrug is to include one or more selected moieties which are hydrolyzed or otherwise cleaved under physiologic conditions to reveal the desired molecule. In other embodiments, the prodrug is converted by an enzymatic activity of the host animal such as specific target cells in the host animal.

Prodrug forms of the herein described compounds, wherein the prodrug is metabolized in vivo to produce a compound described herein are included within the scope of the claims. In some cases, some of the herein-described compounds may be a prodrug for another derivative or active compound.

In certain embodiments, an immune-stimulatory compound, such as a TLR8 agonist or TLR7 agonist, is modified as a prodrug with a masking group, such that the TLR8 agonist, TLR7 agonist or other agonist, has limited activity or is inactive until it reaches an environment where the masking group is removed to reveal the active compound. For example, a TLR8 agonist or TLR7 agonist can be covalently modified at an amine involved in binding to the active site of a TLR8 receptor such that the compound is unable to bind the active site of the receptor in its modified (prodrug) form. In such an example, the masking group is removed under physiological conditions, e.g., enzymatic or acidic conditions, specific to the site of delivery, e.g., intracellular or extracellular adjacent to target cells. Masking groups may be removed from the amine of the compound or salt described herein due to the action of lysosomal proteases, e.g., cathepsin and plasmin. These proteases can be present at elevated levels in certain tumor tissues. The masking group may be removed by a lysosomal enzyme. The lysosomal enzyme can be, for example, cathepsin B, cathepsin S, P3-glucuronidase, or 3-galactosidase.

In certain embodiments, an amine masking group inhibits binding of the amine group of the compound with residues of a TLR8 receptor. The amine masking group may be removable under physiological conditions within a cell but remains covalently bound to the amine outside of a cell. Masking groups that may be used to inhibit or attenuate binding of an amine group of a compound with residues of a TLR8 receptor include, for example, peptides and carbamates.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Linkers

The conjugates include a linker(s) that attaches an antibody construct to at least one immune-stimulatory compound, such as a myeloid cell agonist. A linker can be, for example, a cleavable or a non-cleavable linker. A conjugate can comprise multiple linkers. The linkers in a conjugate can be the same linkers or different linkers.

As will be appreciated by skilled artisans, a linker connects an immune-stimulatory compound(s), such as a myeloid cell agonist, to the antibody construct by forming a covalent linkage to the compound at one location and a covalent linkage to the antibody construct at another location. The covalent linkages can be formed by reaction between functional groups on the linker and functional groups on the immune-stimulatory compound and on the antibody construct. As used herein, the expression “linker” can include (i) unattached forms of the linker that can include a functional group capable of covalently attaching the linker to an immune-stimulatory compound and a functional group capable of covalently attached the linker to an antibody construct; (ii) partially attached forms of the linker that can include a functional group capable of covalently attaching the linker to an antibody construct and that can be covalently attached to an immune-stimulatory compound, or vice versa; and (iii) fully attached forms of the linker that can be covalently attached to both an immune stimulatory compound and to an antibody construct. In some specific embodiments, the functional groups on a linker and covalent linkages formed between the linker and an antibody construct can be specifically illustrated as Rx and Rx′, respectively.

A linker can be short or long, and cleavable or non-cleavable. A linker can contain segments that have different characteristics, such as segments of flexibility or segments of rigidity, segments of hydrophilicity, and/or segments of hydrophobicity. A linker can be chemically stable to extracellular environments, for example, chemically stable in the blood stream, and/or may include linkages that are not stable. A linker can include linkages that are designed to cleave and/or immolate or otherwise breakdown specifically or non-specifically inside cells. A cleavable linker can be sensitive to enzymes at a specific site, such as the lysosome or the extracellular space adjacent cancer cells.

A cleavable linker can include a valine-citrulline peptide, a valine-alanine peptide, a phenylalanine-lysine or other peptide, such as a peptide that forms a protease recognition and cleavage site. Such a peptide-containing linker can contain a pentafluorophenyl group. A peptide-containing linker can include a succimide or a maleimide group. A peptide-containing linker can include a para aminobenzoic acid (PABA) group. A peptide-containing linker can include an aminobenzyloxycarbonyl (PABC) group. A peptide-containing linker can include a PABA or PABC group and a pentafluorophenyl group. A peptide-containing linker can include a PABA or PABC group and a succinimide group. A peptide-containing linker can include a PABA or PABC group and a maleimide group.

A non-cleavable linker is generally protease-insensitive and insensitive to intracellular processes. A non-cleavable linker can include a maleimide group. A non-cleavable linker can include a succinimide group. A non-cleavable linker can be maleimido-alkyl-C(O)— linker. A non-cleavable linker can be maleimidocaproyl linker. A maleimidocaproyl linker can be N-maleimidomethylcyclohexane-1-carboxylate. A maleimidocaproyl linker can include a succinimide group. A maleimidocaproyl linker can include pentafluorophenyl group.

A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain a maleimide(s) linked to polyethylene glycol molecules in which the polyethylene glycol can allow for more linker flexibility or can be used lengthen the linker.

A linker can be a (maleimidocaproyl)-(valine-alanine)-(para-aminobenzyloxycarbonyl) linker. A linker can be a (maleimidocaproyl)-(valine-citrulline)-(para-aminobenzyloxycarbonyl) linker. A linker can be a (maleimidocaproyl)-(phenylalanine-lysine)-(para-aminobenzyloxycarbonyl) linker.

A linker can also contain segments of alkylene, alkenylene, alkynylene, polyether, polyester, polyamide, polyamino acids, peptides, polypeptides, cleavable peptides, and/or aminobenzyl-carbamates. A linker can contain a maleimide at one end and an N-hydroxysuccinimidyl ester at the other end. A linker can contain a lysine with an N-terminal amine acetylated, and a valine-citrulline, valine-alanine or phenylalanine-lysine cleavage site. A linker can be a link created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LXPTG recognition motif (SEQ ID NO: 1) to an N-terminal GGG motif to regenerate a native amide bond. The linker created can therefore link to a moiety attached to the LXPTG recognition motif (SEQ ID NO: 1) with a moiety attached to the N-terminal GGG motif. A linker can be a link created between an unnatural amino acid on one moiety reacting with oxime bond that was formed by modifying a ketone group with an alkoxyamine on another moiety. A moiety can be part of a conjugate. A moiety can be part of an antibody construct, such as an antibody. A moiety can be part of an immune-stimulatory compound, such as a myeloid cell agonist. A moiety can be part of a binding domain. A linker can be unsubstituted or substituted, for example, with a substituent. A substituent can include, for example, hydroxyl groups, amino groups, nitro groups, cyano groups, azido groups, carboxyl groups, carboxaldehyde groups, imine groups, alkyl groups, alkenyl groups, alkynyl groups, alkoxy groups, acyl groups, acyloxy groups, amide groups, and ester groups.

A linker can be polyvalent such that it covalently links more than one immune-stimulatory compound to a single site on the antibody construct, or monovalent such that it covalently links a single immune-stimulatory compound to a single site on the antibody construct.

Exemplary polyvalent linkers that may be used to attach many immune-stimulatory compounds to an antibody construct of the conjugate are described. For example, Fleximer® linker technology has the potential to enable high-DAR conjugate with good physicochemical properties. As shown below, the Fleximer® linker technology is based on incorporating molecules into a solubilizing poly-acetal backbone via a sequence of ester bonds. The methodology renders highly-loaded conjugates (DAR up to 20) whilst maintaining good physicochemical properties. This methodology can be utilized with an immune-stimulatory compound as shown in the scheme below, where Drug′ refers to the immune-stimulatory compound.

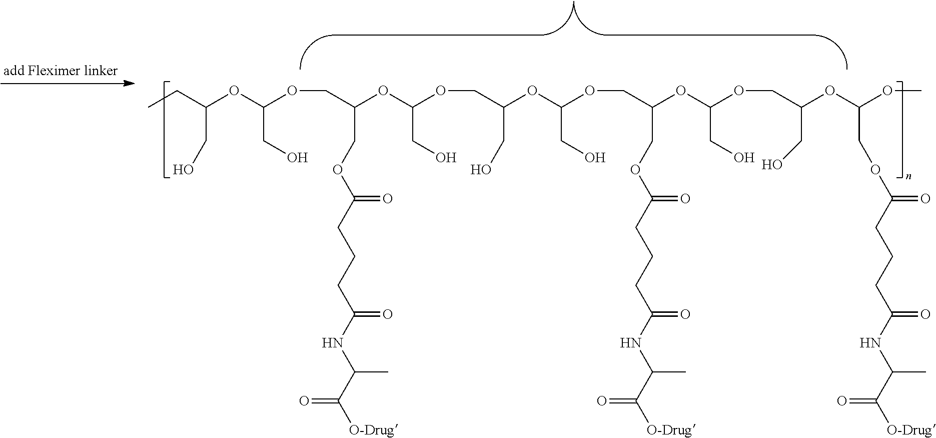

To utilize the Fleximer® linker technology depicted in the scheme above, an aliphatic alcohol can be present or introduced into the immune-stimulatory compound. The alcohol moiety is then attached to an alanine moiety, which is then synthetically incorporated into the Fleximer® linker. Liposomal processing of the conjugate in vitro releases the parent alcohol-containing drug.

By way of example and not limitation, some cleavable and noncleavable linkers that may be included in the conjugates described herein are described below.

Cleavable linkers can be cleavable in vitro and in vivo. Cleavable linkers can include chemically or enzymatically unstable or degradable linkages. Cleavable linkers can rely on processes inside the cell to liberate an immune-stimulatory compound, such as reduction in the cytoplasm, exposure to acidic conditions in the lysosome, or cleavage by specific proteases or other enzymes within the cell. Cleavable linkers can incorporate one or more chemical bonds that are chemically or enzymatically cleavable while the remainder of the linker can be non-cleavable.

A linker can contain a chemically labile group such as hydrazone and/or disulfide group. Linkers comprising chemically labile groups can exploit differential properties between the plasma and some cytoplasmic compartments. The intracellular conditions that can facilitate immune-stimulatory compound release for hydrazine-containing linkers can be the acidic environment of endosomes and lysosomes, while disulfide-containing linkers can be reduced in the cytosol, which can contain high thiol concentrations, e.g., glutathione. The plasma stability of a linker containing a chemically labile group can be increased by introducing steric hindrance using substituents near the chemically labile group.

Acid-labile groups, such as hydrazones, can remain intact during systemic circulation in the blood's neutral pH environment (pH 7.3-7.5) and can undergo hydrolysis and can release an immune-stimulatory compound once the conjugate is internalized into mildly acidic endosomal (pH 5.0-6.5) and lysosomal (pH 4.5-5.0) compartments of the cell. This pH dependent release mechanism can be associated with nonspecific release of the immune-stimulatory compound. To increase the stability of the hydrazone group of the linker, the linker can be varied by chemical modification, e.g., substitution, allowing tuning to achieve more efficient release in the lysosome with a minimized loss in circulation.

Hydrazone-containing linkers can contain additional cleavage sites, such as additional acid-labile cleavage sites and/or enzymatically labile cleavage sites. Conjugates including exemplary hydrazone-containing linkers can include, for example, the following structures:

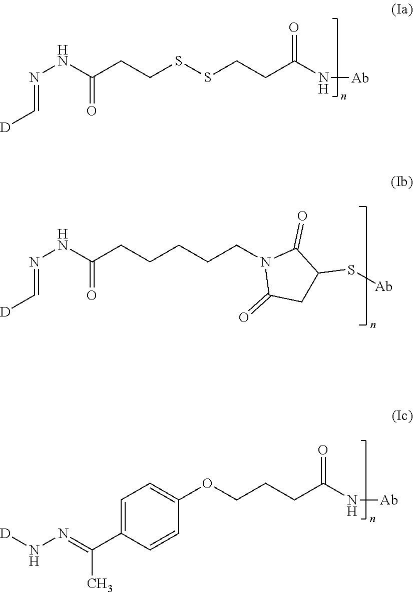

wherein D is an immune-stimulatory compound and Ab is an antibody construct, respectively, and n represents the number of compound-bound linkers (LP) bound to the antibody construct. In certain linkers, such as linker (Ia), the linker can comprise two cleavable groups, a disulfide and a hydrazone moiety. For such linkers, effective release of the unmodified free immune-stimulatory compound can require acidic pH or disulfide reduction and acidic pH. Linkers such as (Ib) and (Ic) can be effective with a single hydrazone cleavage site.

Other acid-labile groups that can be included in linkers include cis-aconityl-containing linkers. cis-Aconityl chemistry can use a carboxylic acid juxtaposed to an amide bond to accelerate amide hydrolysis under acidic conditions.

Cleavable linkers can also include a disulfide group. Disulfides can be thermodynamically stable at physiological pH and can be designed to release an immune-stimulatory compound upon internalization inside cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing linkers can be reasonably stable in circulation, selectively releasing the immune-stimulatory compound in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing linker can be enhanced by chemical modification of the linker, e.g., use of steric hindrance adjacent to the disulfide bond.

Immune-stimulatory conjugates including disulfide-containing linkers can include the following structures:

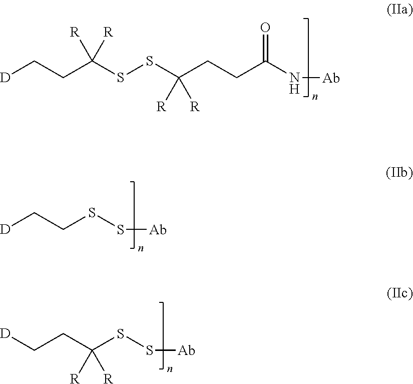

wherein D is an immune-stimulatory compound and Ab is an antibody construct, respectively, n represents the number of compounds bound to linkers bound to the antibody construct and R is independently selected at each occurrence from hydrogen or alkyl, for example. Increasing steric hindrance adjacent to the disulfide bond can increase the stability of the linker. Structures such as (IIa) and (IIc) can show increased in vivo stability when one or more R groups is selected from a lower alkyl such as methyl.

Another type of linker that can be used is a linker that is specifically cleaved by an enzyme. For example, the linker can be cleaved by a lysosomal enzyme. Such linkers can be peptide-based or can include peptidic regions that can act as substrates for enzymes. Peptide based linkers can be more stable in plasma and extracellular milieu than chemically labile linkers.

Peptide bonds can have good serum stability, as lysosomal proteolytic enzymes can have very low activity in blood due to endogenous inhibitors and the unfavorable pH value of blood compared to lysosomes. Release of an immune-stimulatory compound from an antibody construct can occur due to the action of lysosomal proteases, e.g., cathepsin and plasmin. These proteases can be present at elevated levels in certain tumor tissues. A linker can be cleavable by a lysosomal enzyme. The lysosomal enzyme can be, for example, cathepsin B, cathepsin S, β-glucuronidase, or β-galactosidase.

The cleavable peptide can be selected from tetrapeptides such as Gly-Phe-Leu-Gly, Ala-Leu-Ala-Leu, dipeptides such as Val-Cit, Val-Ala, and Phe-Lys, or other peptides. Dipeptides can have lower hydrophobicity compared to longer peptides, depending on the composition of the peptide.

A variety of dipeptide-based cleavable linkers can be used in the immune-stimulatory conjugates described herein.

Enzymatically cleavable linkers can include a self-immolative spacer to spatially separate the immune-stimulatory compound from the site of enzymatic cleavage. The direct attachment of an immune-stimulatory compound to a peptide linker can result in proteolytic release of the immune-stimulatory compound or of an amino acid adduct of the immune-stimulatory compound, thereby impairing its activity. The use of a self-immolative spacer can allow for the elimination of the fully active, chemically unmodified immune-stimulatory compound upon amide bond hydrolysis.

One self-immolative spacer can be a bifunctional para-aminobenzyl alcohol group (PABA), which can link to the peptide through the amino group, forming an amide bond, while amine containing immune-stimulatory compounds can be attached through carbamate functionalities to the benzylic hydroxyl group of the linker (to give a p-amidobenzylcarbamate, PABC). The resulting pro-immune-stimulatory compound can be activated upon protease-mediated cleavage, leading to a 1,6-elimination reaction releasing the unmodified immune-stimulatory compound, carbon dioxide, and remnants of the linker. The following scheme depicts the fragmentation of p-amidobenzyl carbamate and release of the immune-stimulatory compound:

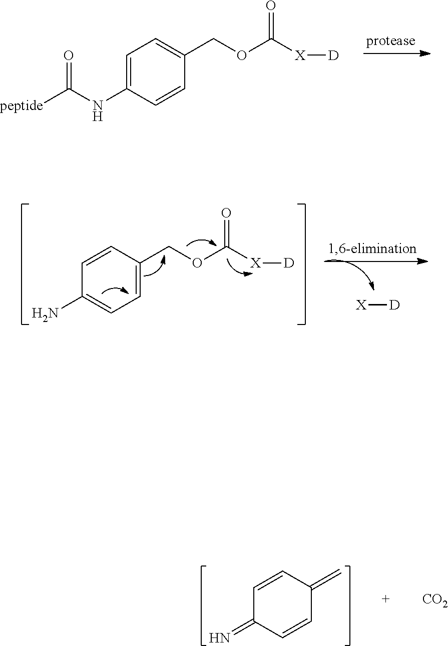

wherein X-D represents the unmodified immune-stimulatory compound and the carbonyl group adjacent “peptide” is part of the peptide. Heterocyclic variants of this self-immolative group have also been described.

An enzymatically cleavable linker can be a β-glucuronic acid-based linker. Facile release of an immune-stimulatory compound can be realized through cleavage of the β-glucuronide glycosidic bond by the lysosomal enzyme β-glucuronidase. This enzyme can be abundantly present within lysosomes and can be overexpressed in some tumor types, while the enzyme activity outside cells can be low. β-Glucuronic acid-based linkers can be used to circumvent the tendency of an immune-stimulatory conjugate to undergo aggregation due to the hydrophilic nature of β-glucuronides. In certain embodiments, β-glucuronic acid-based linkers can link an antibody construct to a hydrophobic immune-stimulatory compound. The following scheme depicts the release of an immune-stimulatory compound (D) from an immune-stimulatory conjugate containing a β-glucuronic acid-based linker:

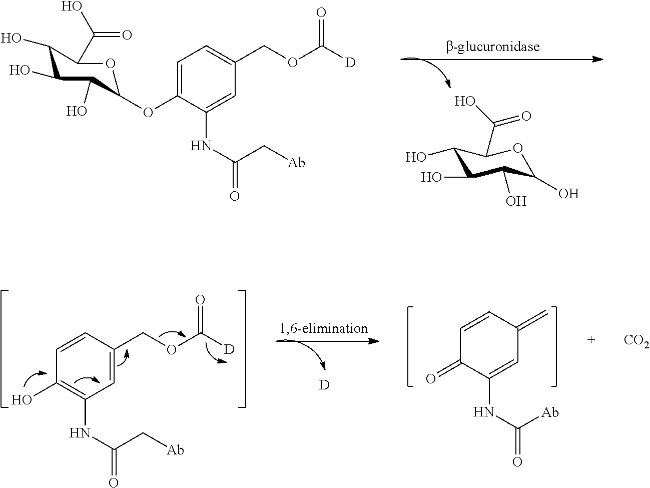

wherein Ab indicates the antibody construct.

A variety of cleavable β-glucuronic acid-based linkers useful for linking drugs such as auristatins, camptothecin and doxorubicin analogues, CBI minor-groove binders, and psymberin to antibodies have been described. These β-glucuronic acid-based linkers may be used in the conjugates described herein. In certain embodiments, the enzymatically cleavable linker is a β-galactoside-based linker. β-Galactoside is present abundantly within lysosomes, while the enzyme activity outside cells is low.

Additionally, immune-stimulatory compounds containing a phenol group can be covalently bonded to a linker through the phenolic oxygen. One such linker relies on a methodology in which a diamino-ethane “Space Link” is used in conjunction with traditional “PABO”-based self-immolative groups to deliver phenols.

Cleavable linkers can include non-cleavable portions or segments, and/or cleavable segments or portions can be included in an otherwise non-cleavable linker to render it cleavable. By way of example only, polyethylene glycol (PEG) and related polymers can include cleavable groups in the polymer backbone. For example, a polyethylene glycol or polymer linker can include one or more cleavable groups such as a disulfide, a hydrazone or a dipeptide.

Other degradable linkages that can be included in linkers can include ester linkages formed by the reaction of PEG carboxylic acids or activated PEG carboxylic acids with alcohol groups on an immune-stimulatory compound, wherein such ester groups can hydrolyze under physiological conditions to release the immune-stimulatory compound. Hydrolytically degradable linkages can include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

A linker can contain an enzymatically cleavable peptide moiety, for example, a linker comprising structural formula (IIIa), (IIIb), (IIIc), or (IIId):

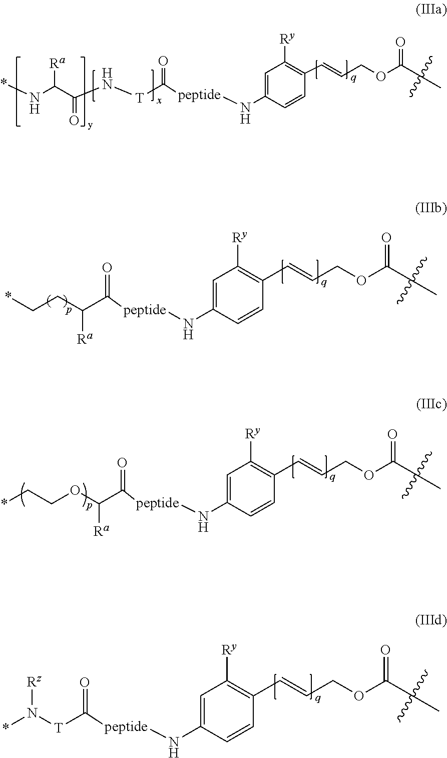

or a pharmaceutically acceptable salt thereof, wherein: “peptide” represents a peptide (illustrated in N→C orientation, wherein peptide includes the amino and carboxy “termini”) that is cleavable by a lysosomal enzyme; T represents a polymer comprising one or more ethylene glycol units or an alkylene chain, or combinations thereof; R^{a} is selected from hydrogen, alkyl, sulfonate and methyl sulfonate; R^{y} is hydrogen or C₁₋₄ alkyl-(O)ᵣ—(C₁₋₄ alkylene)ₛ-G¹ or C₁₋₄ alkyl-(N)—[(C₁₋₄ alkylene)-G¹]₂; R^{z} is C₁₋₄ alkyl-(O)ᵣ—(C₁₋₄ alkylene)ₛ-G²; G¹ is SO₃H, CO₂H, PEG 4-32, or a sugar moiety; G² is SO₃H, CO₂H, or a PEG 4-32 moiety; r is 0 or 1; s is 0 or 1; p is an integer ranging from 0 to 5; q is 0 or 1; x is 0 or 1; y is 0 or 1; [symbol]
represents the point of attachment of the linker to an immune-stimulatory compound; and * represents the point of attachment to the remainder of the linker.

In certain embodiments, the peptide can be selected from natural amino acids, unnatural amino acids or combinations thereof. In certain embodiments, the peptide can be selected from a tripeptide or a dipeptide. In particular embodiments, the dipeptide can comprise L-amino acids and be selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

Exemplary embodiments of linkers according to structural formula (IIIa) are illustrated below (as illustrated, the linkers include a reactive group suitable for covalently linking the linker to an antibody construct):

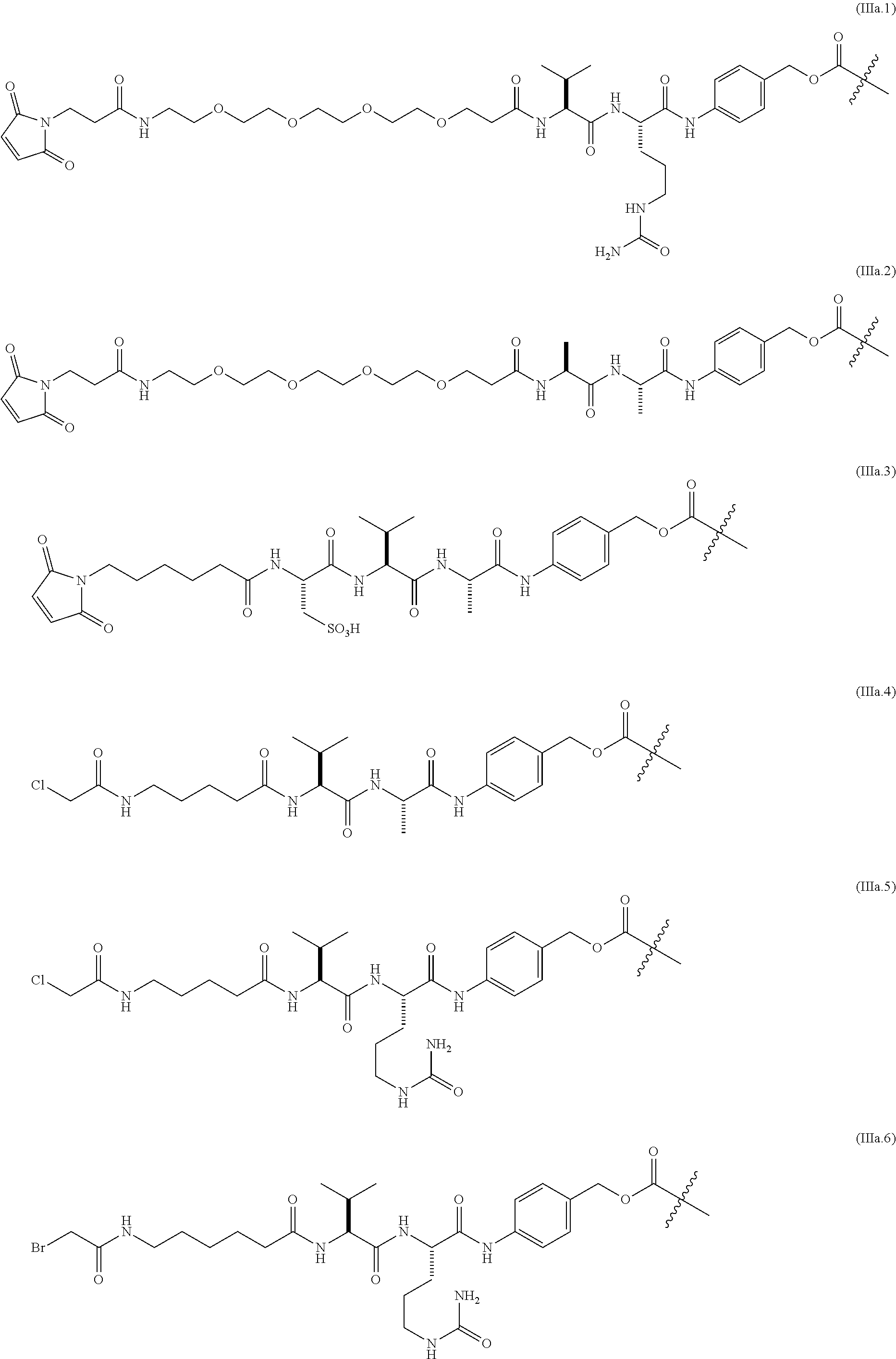

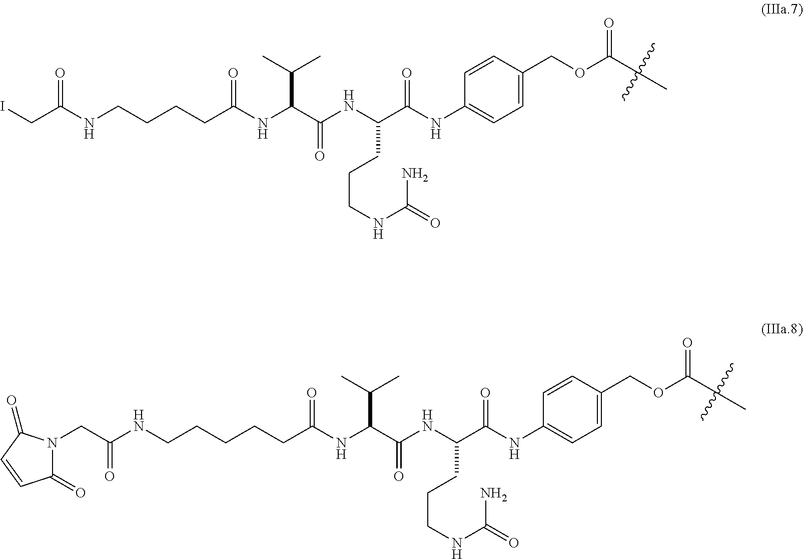 [SMILES: *C(=O)OCc1ccc(NC(=O)[C@H](CCCNC(N)=O)NC(=O)[C@@H](NC(=O)CCCCNC(=O)CI)C(C)C)cc1 ; *C(=O)OCc1ccc(NC(=O)[C@H](CCCNC(N)=O)NC(=O)[C@@H](NC(=O)CCCCCNC(=O)CN2C(=O)C=CC2=O)C(C)C)cc1]

wherein [symbol] indicates an attachment site of a linker to an immune-stimulatory compound.

Exemplary embodiments of linkers according to structural formula (IIIb), (IIIc), or (IIId) that can be included in the conjugates described herein can include the linkers illustrated below (as illustrated, the linkers can include a reactive group suitable for covalently linking the linker to an antibody construct):

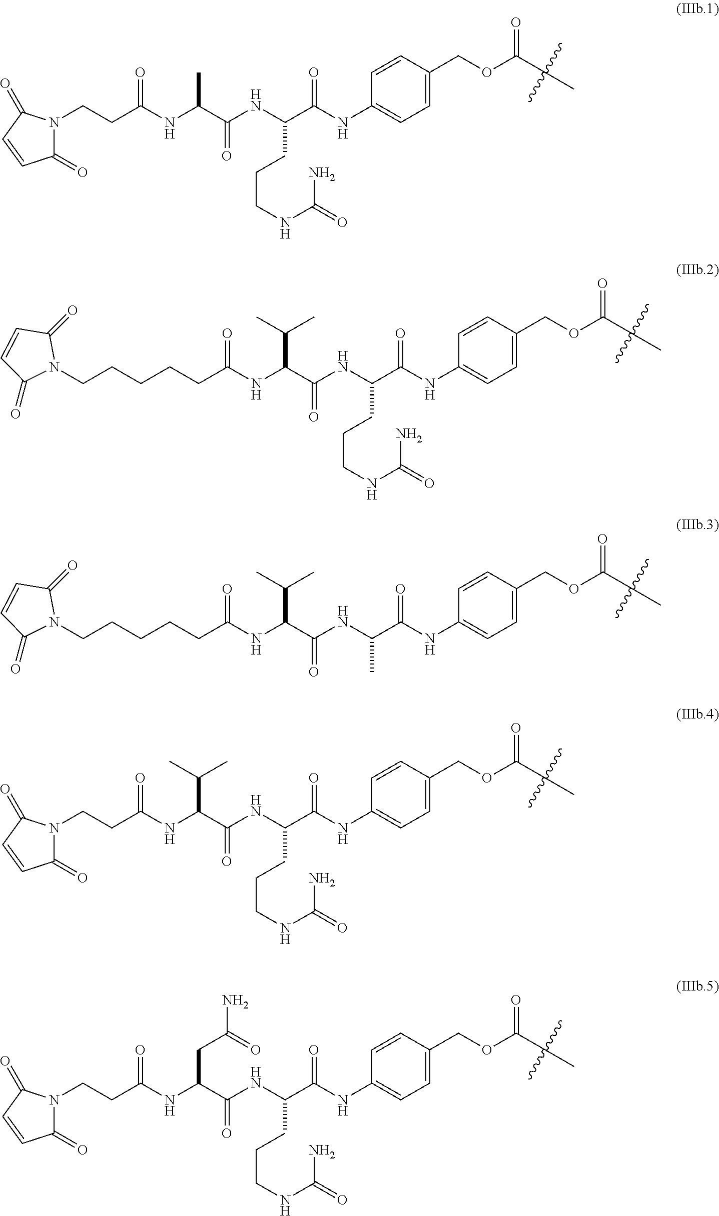 [SMILES: *C(=O)OCc1ccc(NC(=O)[C@H](CCCNC(N)=O)NC(=O)[C@H](C)NC(=O)CCN2C(=O)C=CC2=O)cc1 ; *C(=O)OCc1ccc(NC(=O)[C@H](CCCNC(N)=O)NC(=O)[C@@H](NC(=O)CCCCCN2C(=O)C=CC2=O)C(C)C)cc1 ; *C(=O)OCc1ccc(NC(=O)[C@H](C)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@@H](NC(=O)CCN2C(=O)C=CC2=O)C(C)C)cc1 ; *C(=O)OCc1ccc(NC(=O)[C@H](CCCNC(N)=O)NC(=O)[C@H](CC(N)=O)NC(=O)CCN2C(=O)C=CC2=O)cc1]

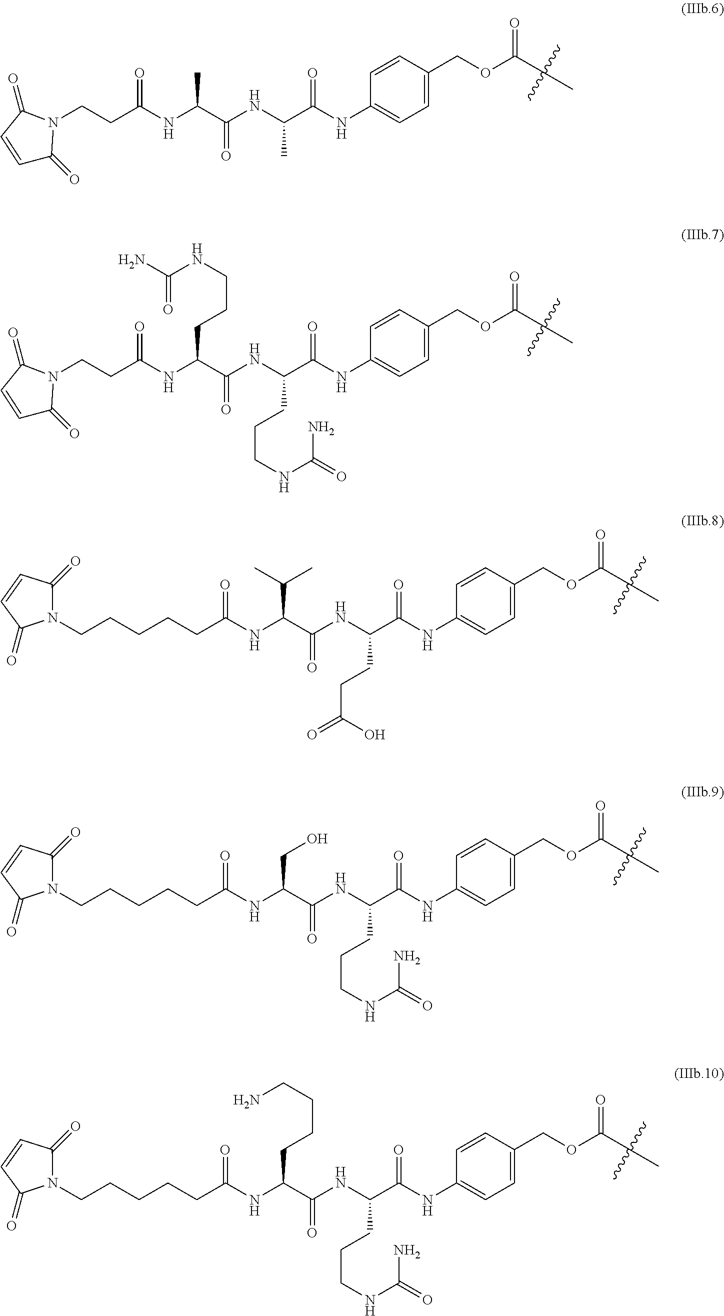 [SMILES: *C(=O)OCc1ccc(NC(=O)[C@H](C)NC(=O)[C@H](C)NC(=O)CCN2C(=O)C=CC2=O)cc1 ; *C(=O)OCc1ccc(NC(=O)[C@H](CCCNC(N)=O)NC(=O)[C@H](CCCNC(N)=O)NC(=O)CCN2C(=O)C=CC2=O)cc1 ; *C(=O)OCc1ccc(NC(=O)[C@H](CCC(=O)O)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@H](CO)NC(=O)CCCCCN2C(=O)C=CC2=O)cc1 ; *C(=O)OCc1ccc(NC(=O)[C@H](CCCNC(N)=O)NC(=O)[C@H](CCCCN)NC(=O)CCCCCN2C(=O)C=CC2=O)cc1]

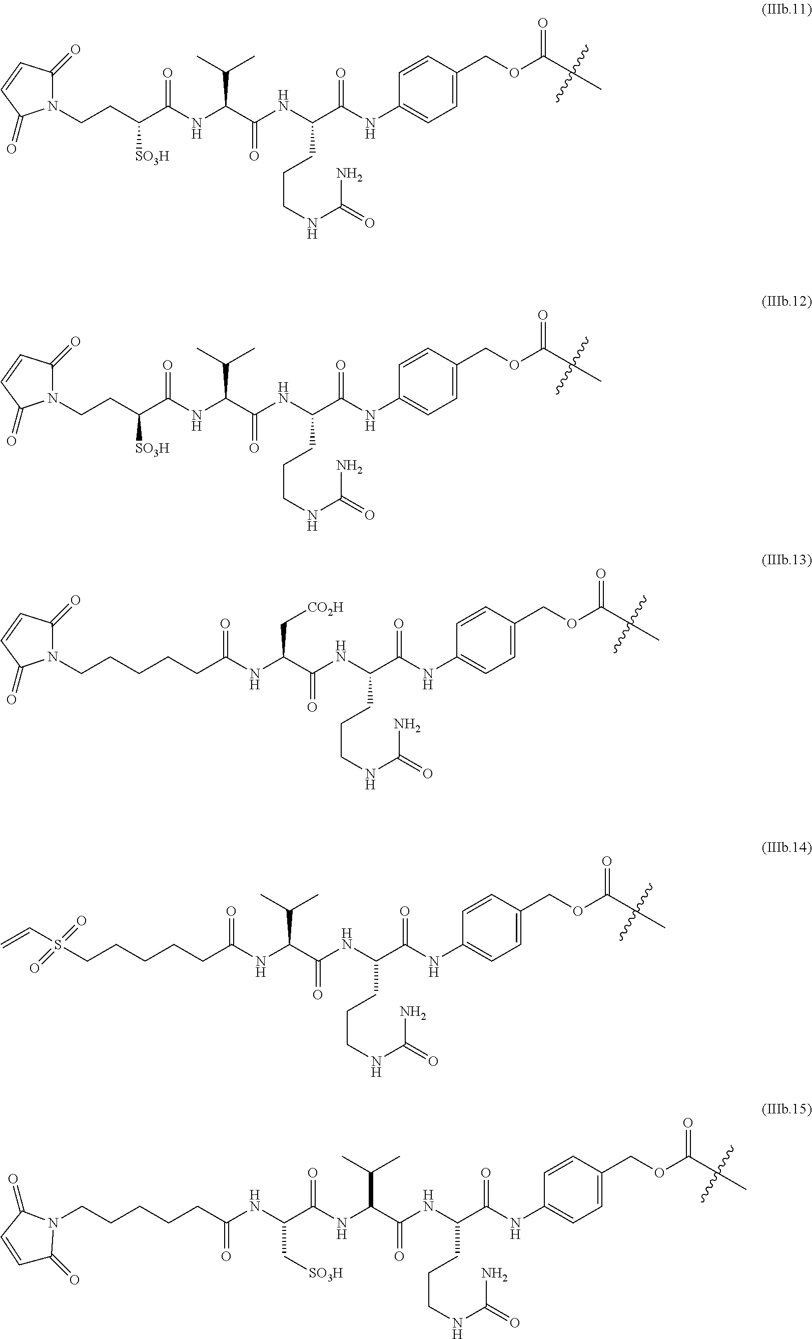 [SMILES: *C(=O)OCc1ccc(NC(=O)[C@H](CCCNC(N)=O)NC(=O)[C@@H](NC(=O)[C@@H](CCN2C(=O)C=CC2=O)S(=O)(=O)O)C(C)C)cc1 ; *C(=O)OCc1ccc(NC(=O)[C@H](CCCNC(N)=O)NC(=O)[C@@H](NC(=O)[C@H](CCN2C(=O)C=CC2=O)S(=O)(=O)O)C(C)C)cc1 ; *C(=O)OCc1ccc(NC(=O)[C@H](CCCNC(N)=O)NC(=O)[C@H](CC(=O)O)NC(=O)CCCCCN2C(=O)C=CC2=O)cc1 ; *C(=O)OCc1ccc(NC(=O)[C@H](CCCNC(N)=O)NC(=O)[C@@H](NC(=O)CCCCCS(=O)(=O)C=C)C(C)C)cc1 ; *C(=O)OCc1ccc(NC(=O)[C@H](CCCNC(N)=O)NC(=O)[C@@H](NC(=O)[C@H](CS(=O)(=O)O)NC(=O)CCCCCN2C(=O)C=CC2=O)C(C)C)cc1]

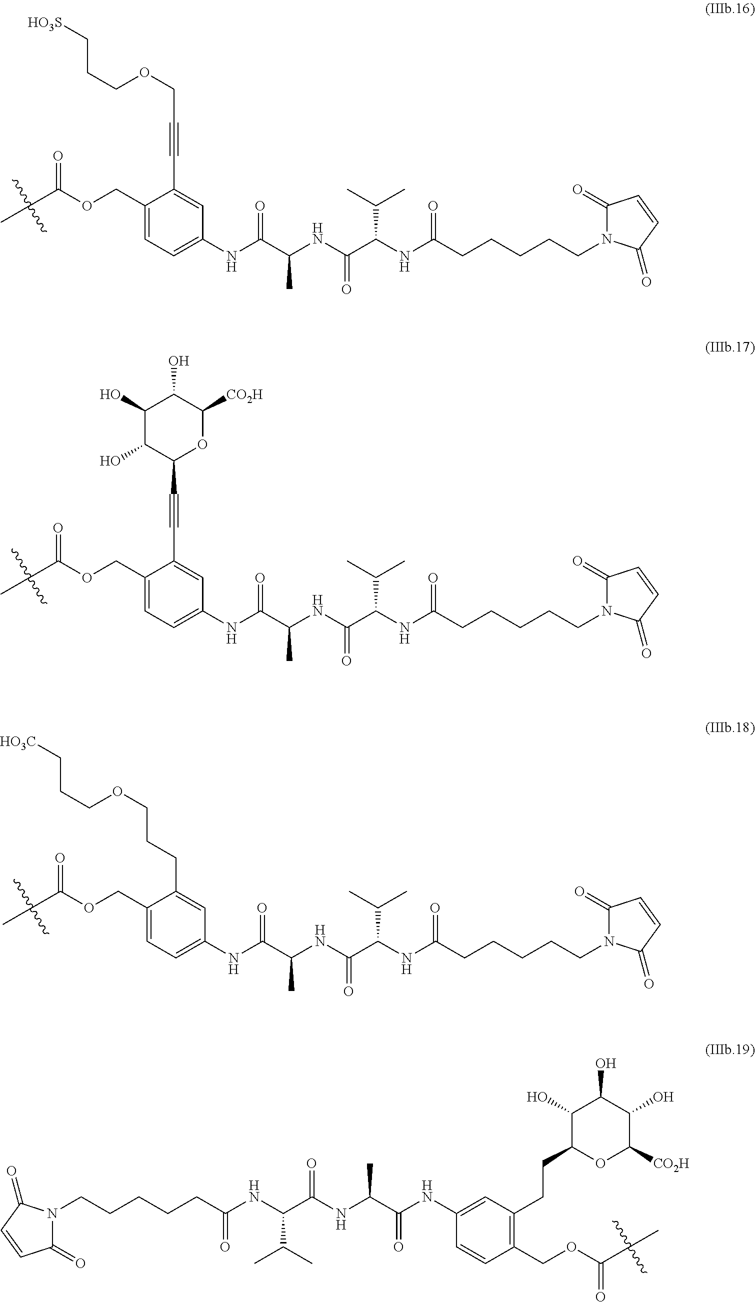

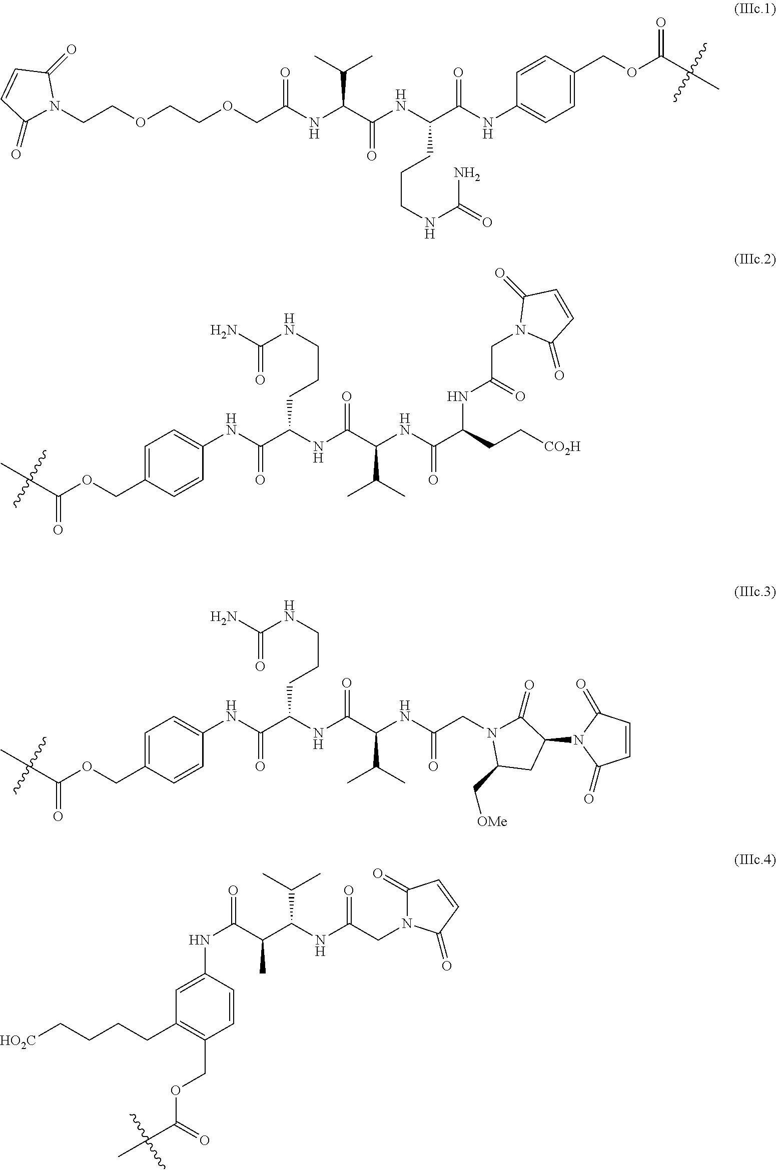 [SMILES: *C(=O)OCc1ccc(NC(=O)[C@H](CCCNC(N)=O)NC(=O)[C@@H](NC(=O)COCCOCCN2C(=O)C=CC2=O)C(C)C)cc1 ; *C(=O)OCc1ccc(NC(=O)[C@H](CCCNC(N)=O)NC(=O)[C@@H](NC(=O)[C@H](CCC(=O)O)NC(=O)CN2C(=O)C=CC2=O)C(C)C)cc1 ; *C(=O)OCc1ccc(NC(=O)[C@H](CCCNC(N)=O)NC(=O)[C@@H](NC(=O)CN2C(=O)[C@@H](N3C(=O)C=CC3=O)C[C@H]2COC)C(C)C)cc1 ; *C(=O)OCc1ccc(NC(=O)[C@H](C)[C@@H](NC(=O)CN2C(=O)C=CC2=O)C(C)C)cc1CCCCC(=O)O]

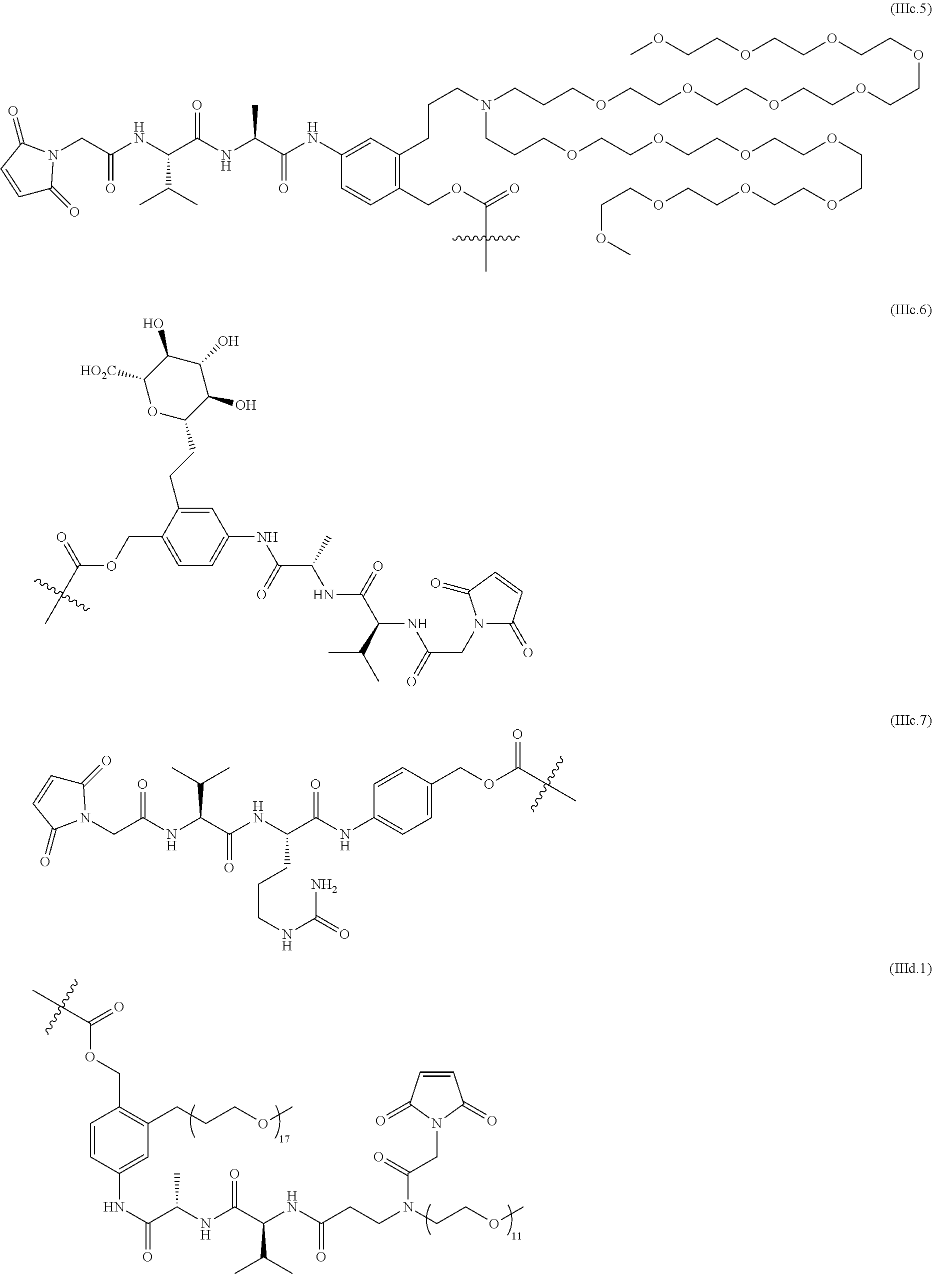

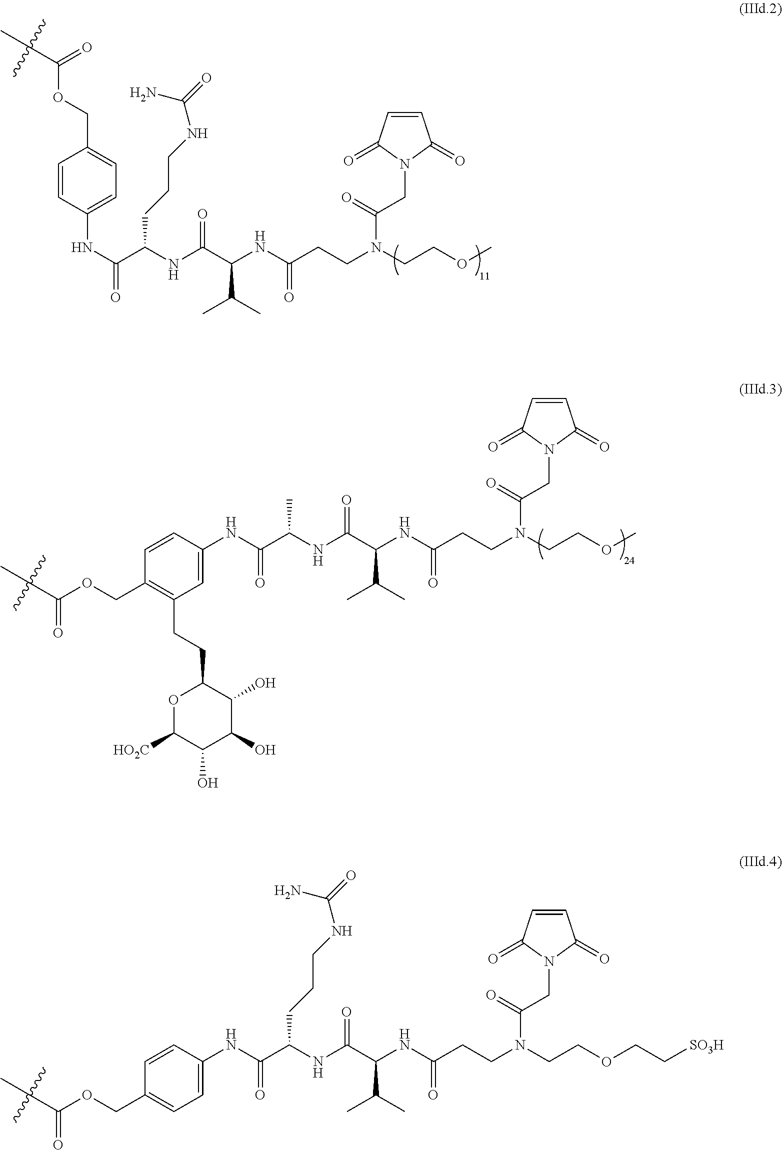

wherein [symbol] indicates an attachment site to an immune-stimulatory compound.

The linker can contain an enzymatically cleavable sugar moiety, for example, a linker comprising structural formula (IVa), (IVb), (IVc), (IVd), or (IVe):

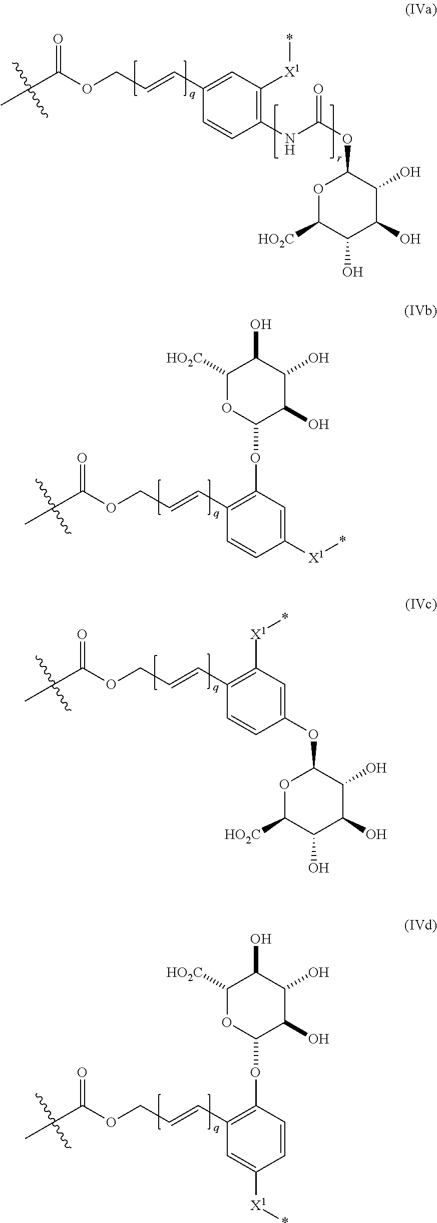

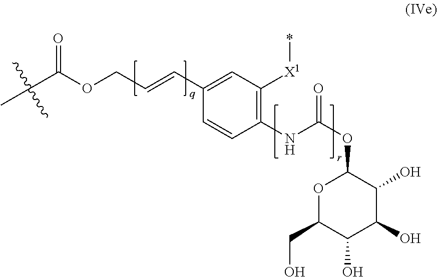

or a pharmaceutically acceptable salt thereof, wherein: q is 0 or 1; r is 0 or 1; X¹ is CH₂, O or NH; [symbol] represents the point of attachment of the linker to an immune-stimulatory compound; and * represents the point of attachment to the remainder of the linker.

Exemplary embodiments of linkers according to structural formula (IVa) that may be included in the immune-stimulatory conjugates described herein can include the linkers illustrated below (as illustrated, the linkers include a group suitable for covalently linking the linker to an antibody construct):

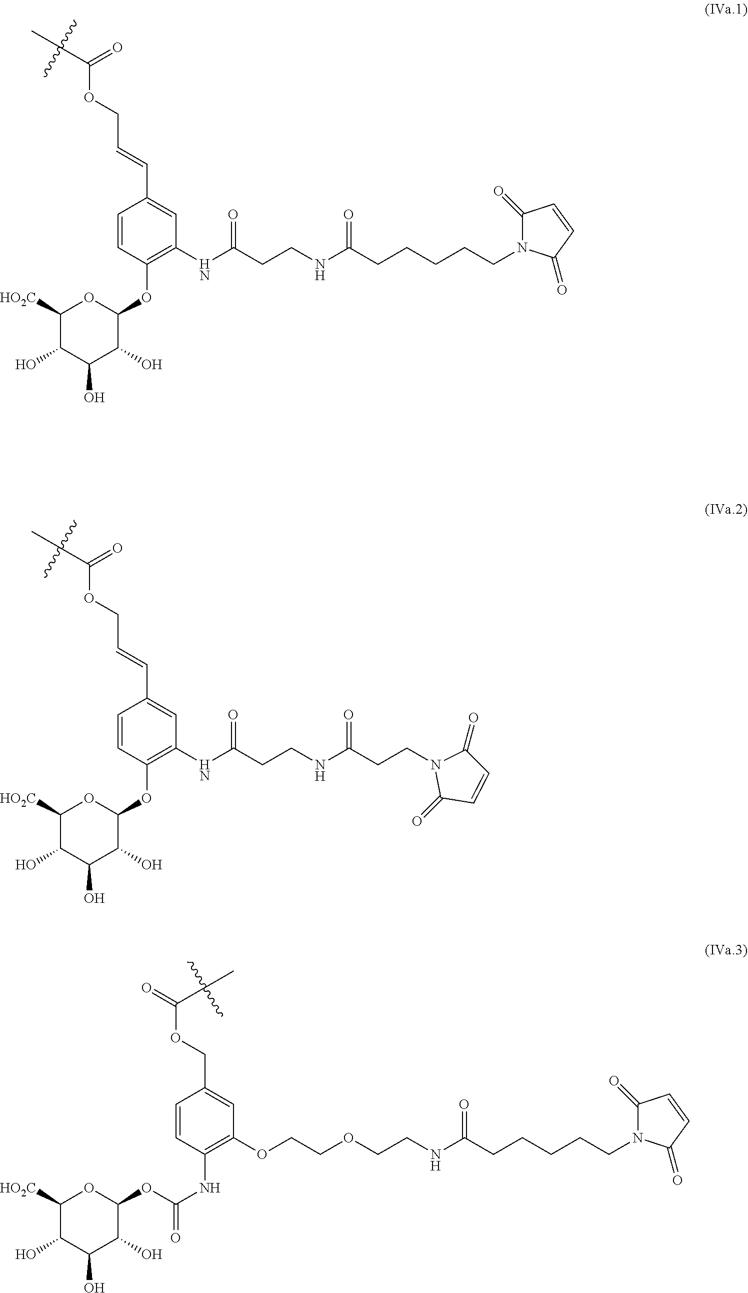

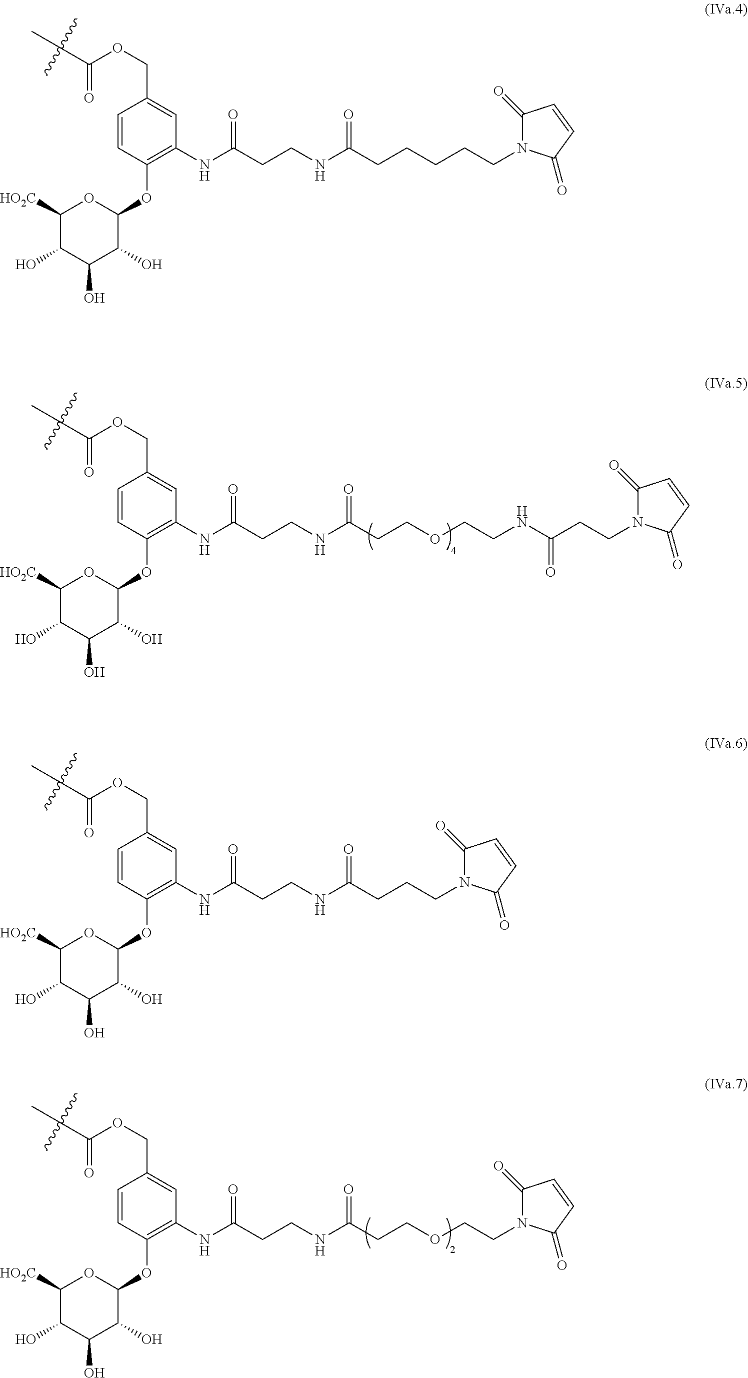

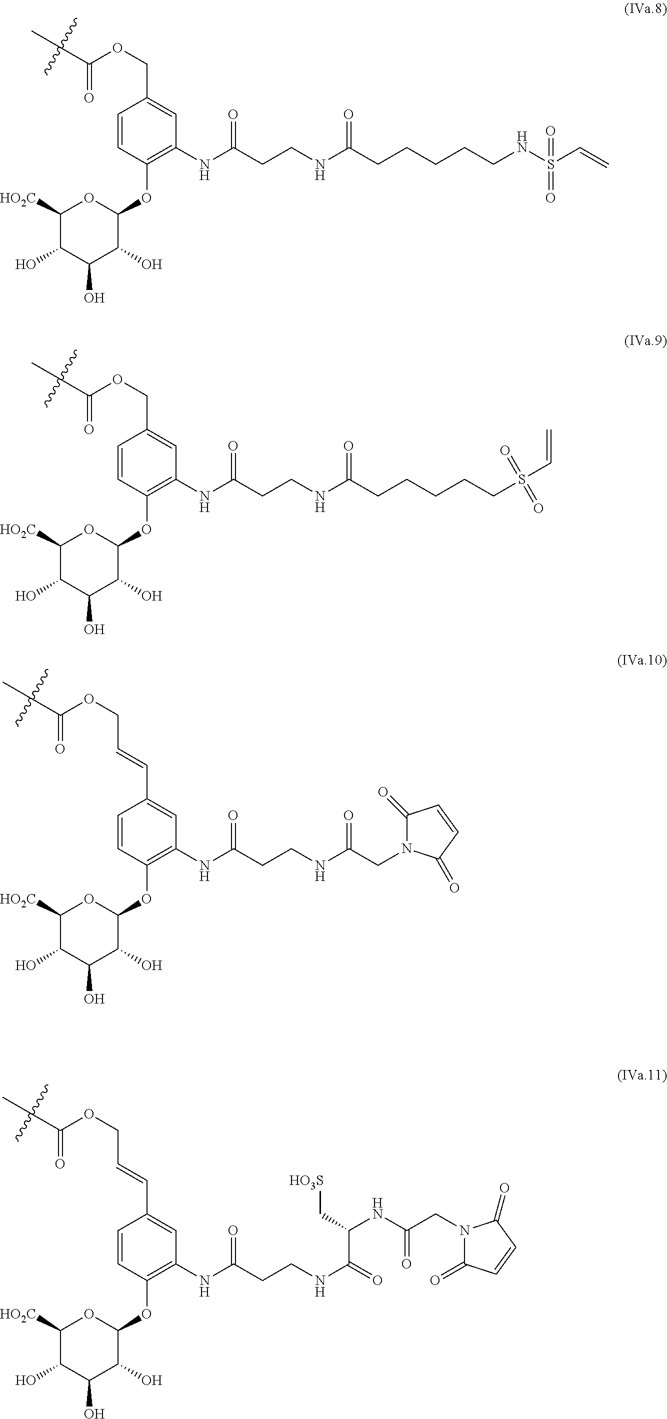

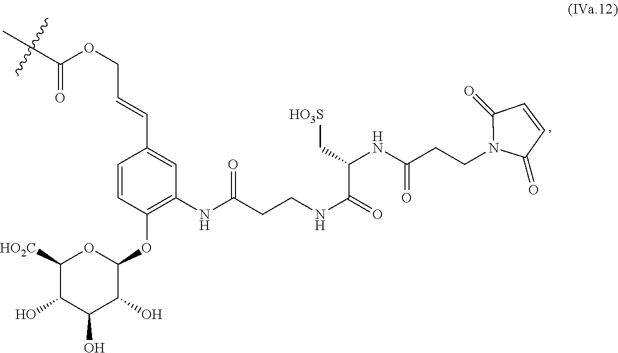

wherein [symbol] represents the point of attachment of a linker to an immune-stimulatory.

Exemplary embodiments of linkers according to structural formula (IVb) that may be included in the conjugates described herein include the linkers illustrated below (as illustrated, the linkers include a group suitable for covalently linking the linker to an antibody construct):

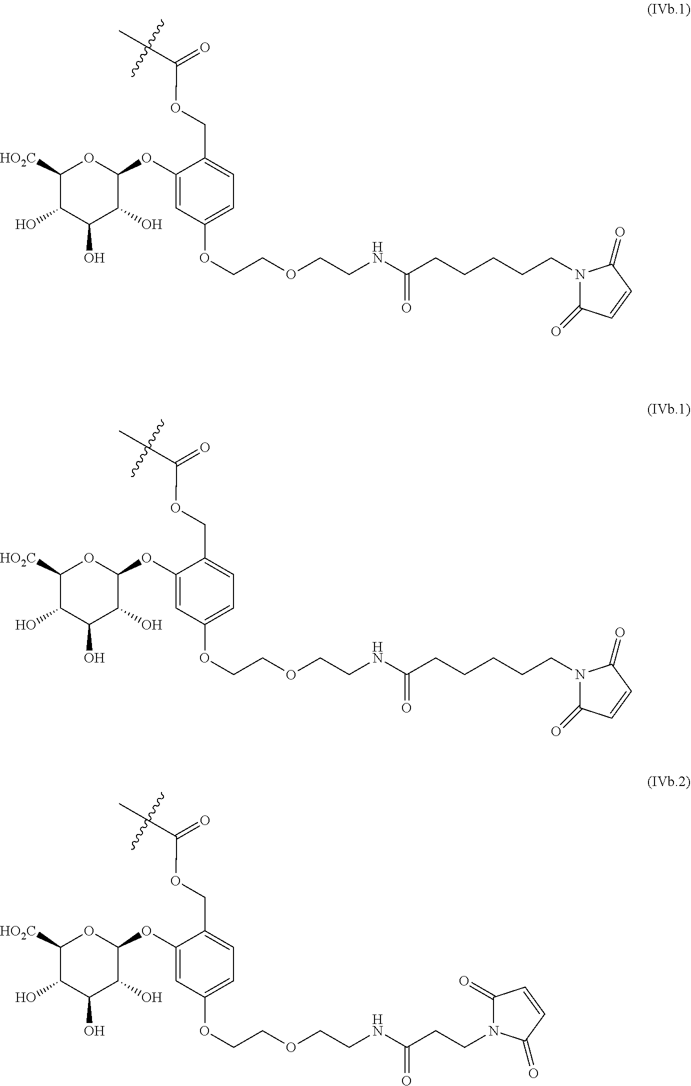

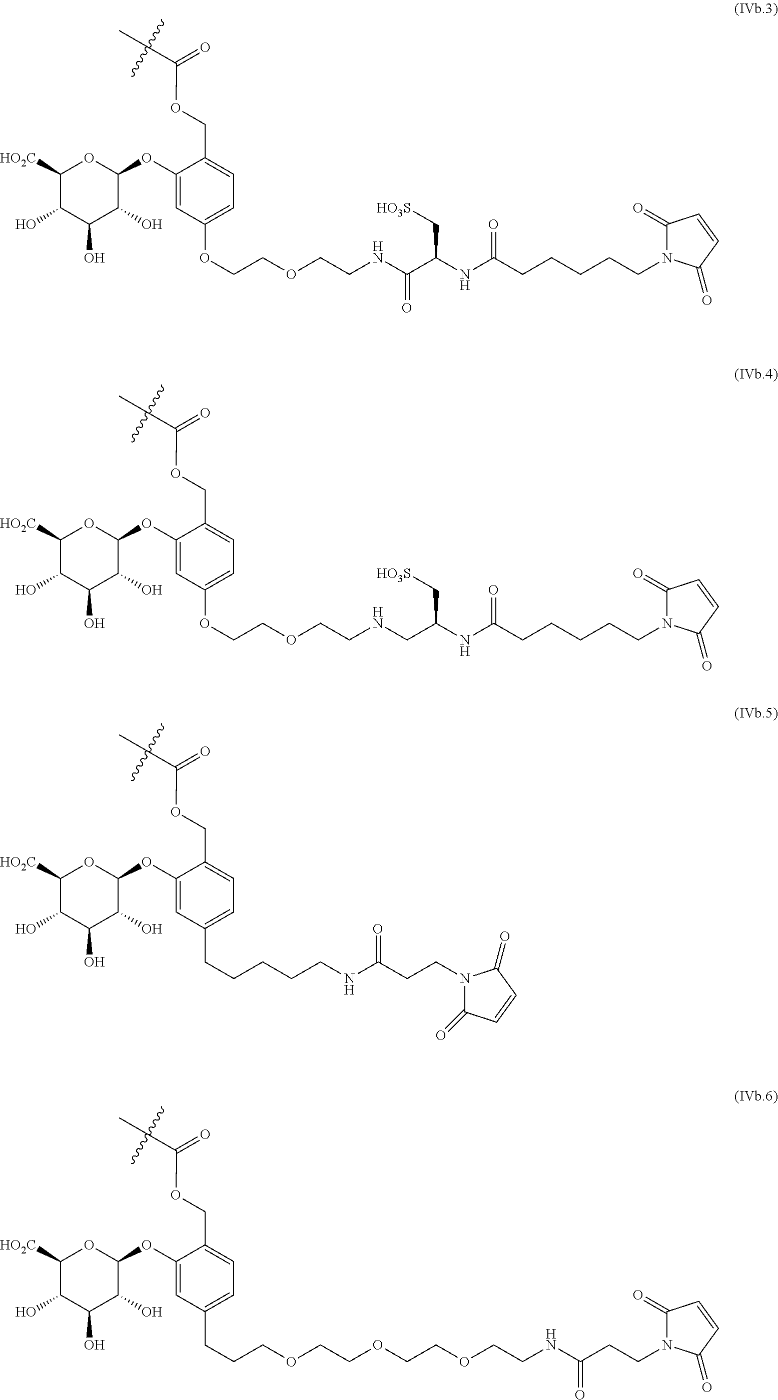

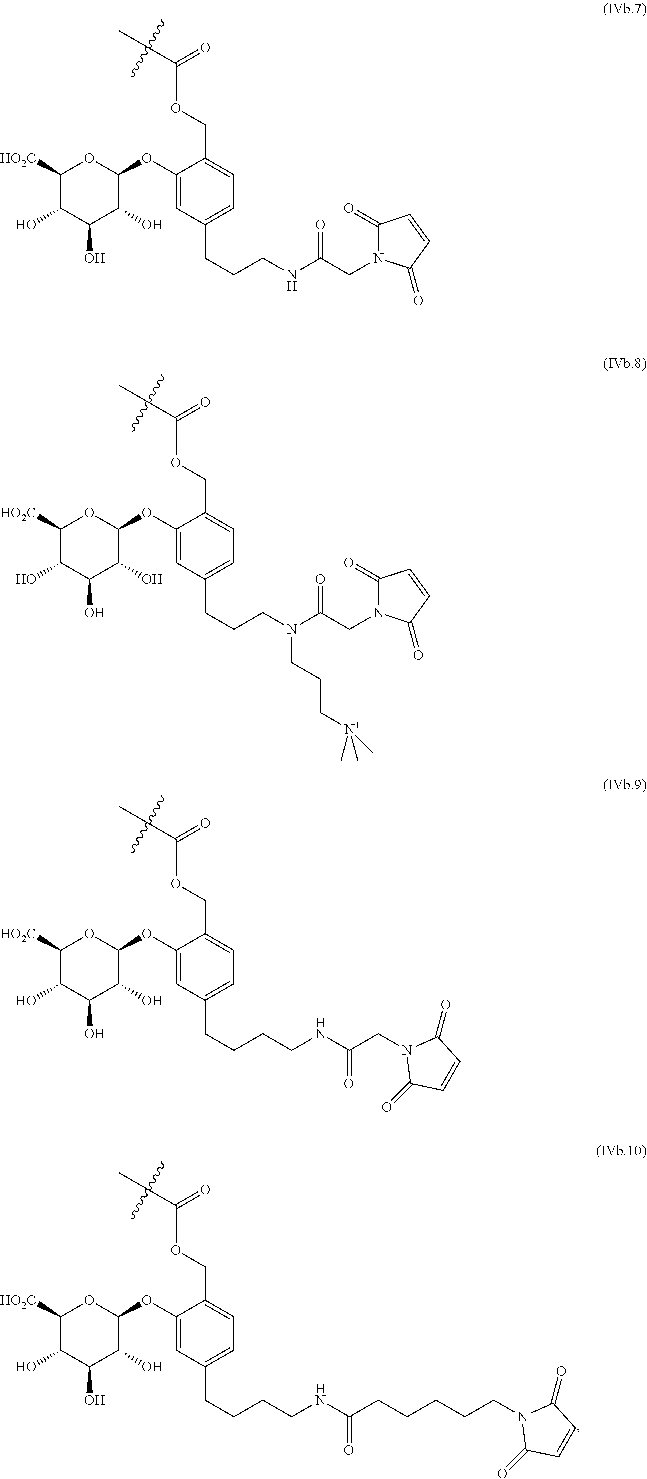

wherein [symbol] represents the point of attachment of a linker to an immune-stimulatory compound.

Exemplary embodiments of linkers according to structural formula (IVc) that may be included in the conjugates described herein include the linkers illustrated below (as illustrated, the linkers include a group suitable for covalently linking the linker to an antibody construct):

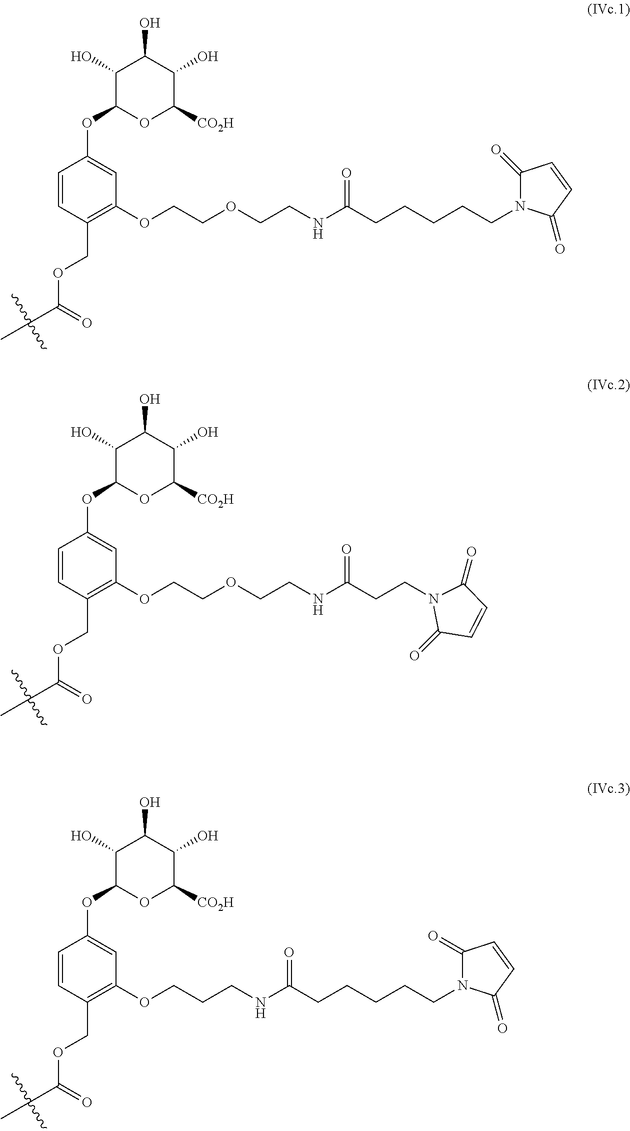

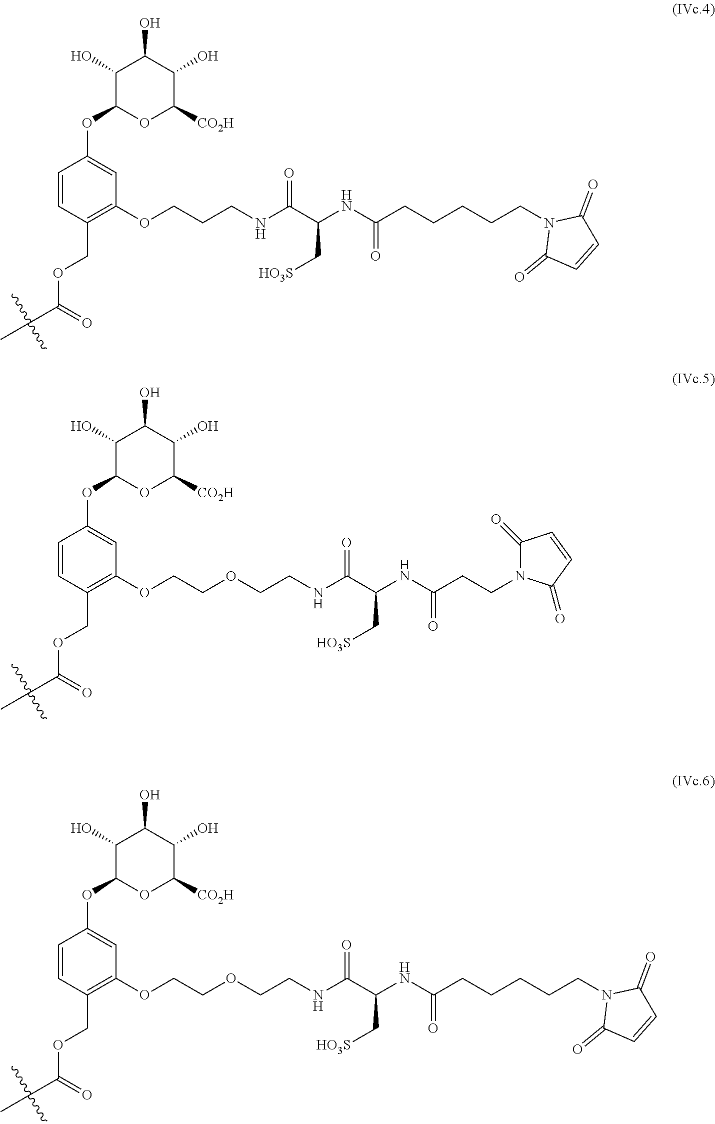

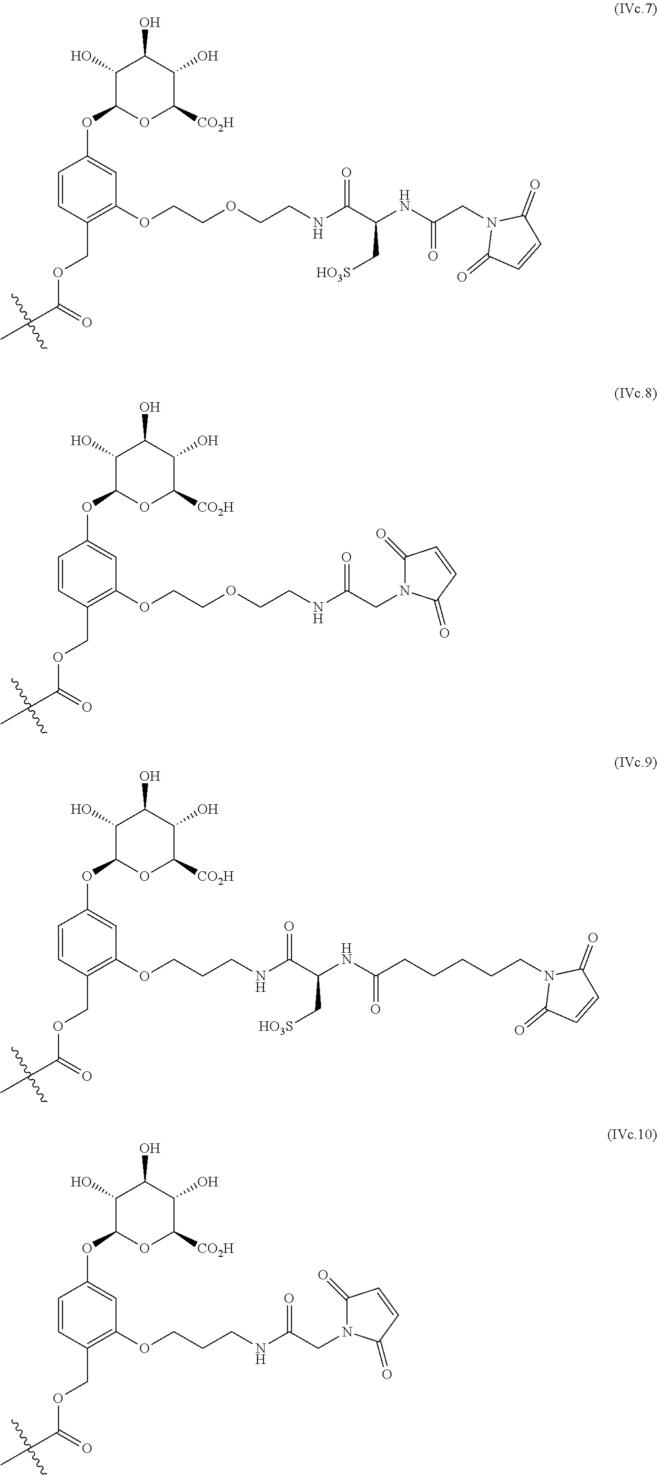

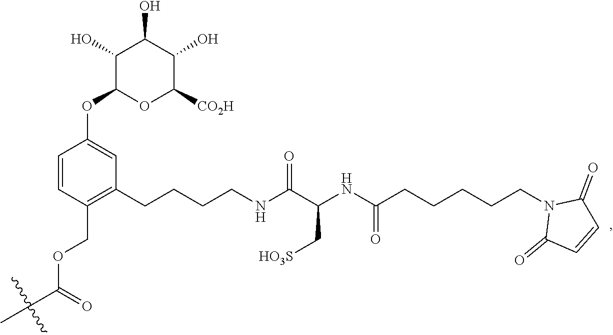

wherein [symbol] represents the point of attachment of a linker to an immune-stimulatory compound.

Exemplary embodiments of linkers according to structural formula (IVd) that may be included in the conjugates described herein include the linkers illustrated below (as illustrated, the linkers include a group suitable for covalently linking the linker to an antibody construct):

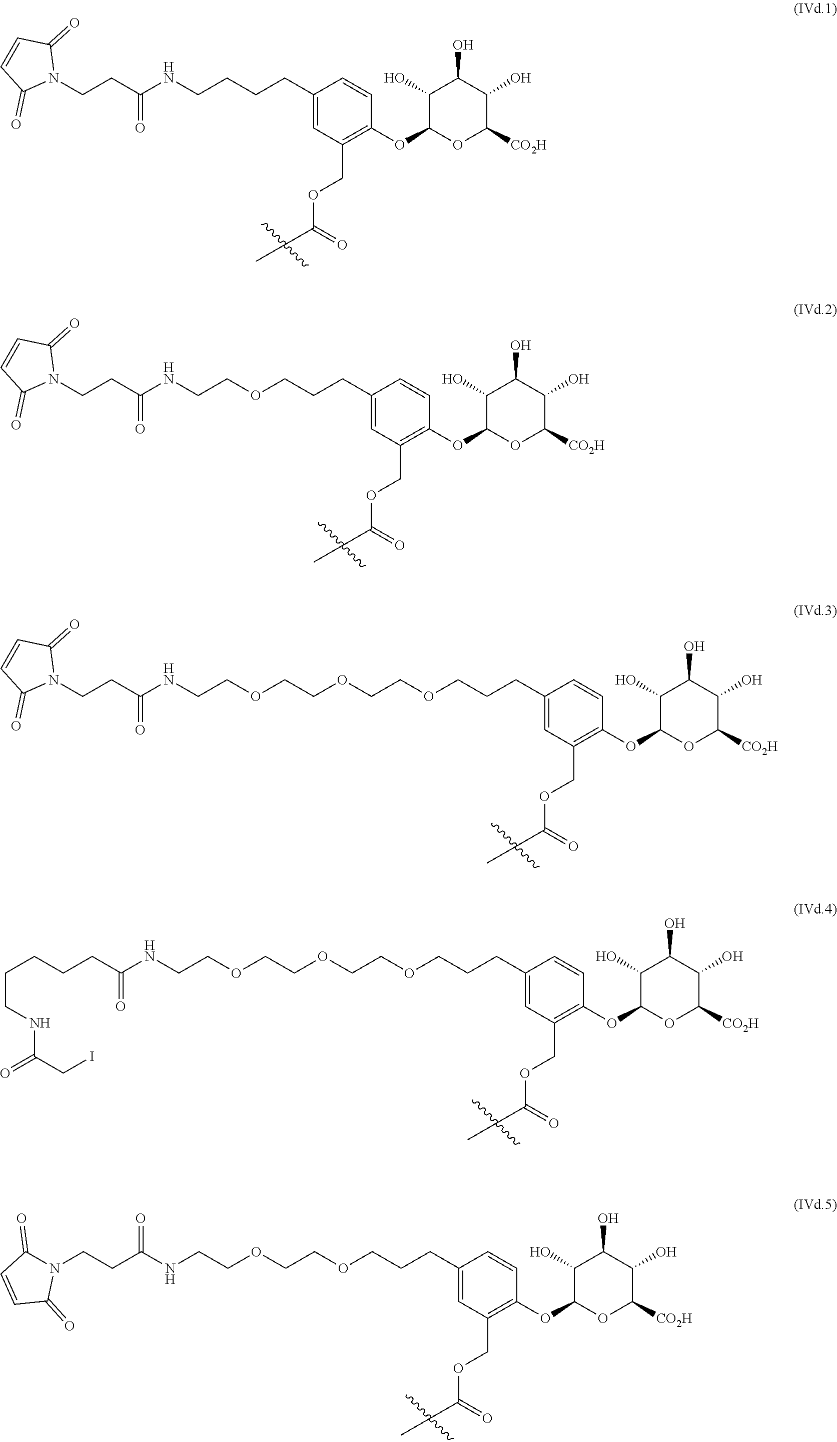

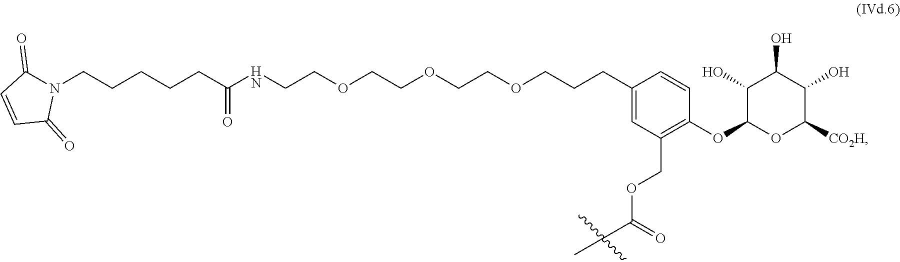

wherein [symbol] represents the point of attachment of a linker to an immune-stimulatory compound.

Exemplary embodiments of linkers according to structural formula (IVe) that may be included in the conjugates described herein include the linkers illustrated below (as illustrated, the linkers include a group suitable for covalently linking the linker to an antibody construct):

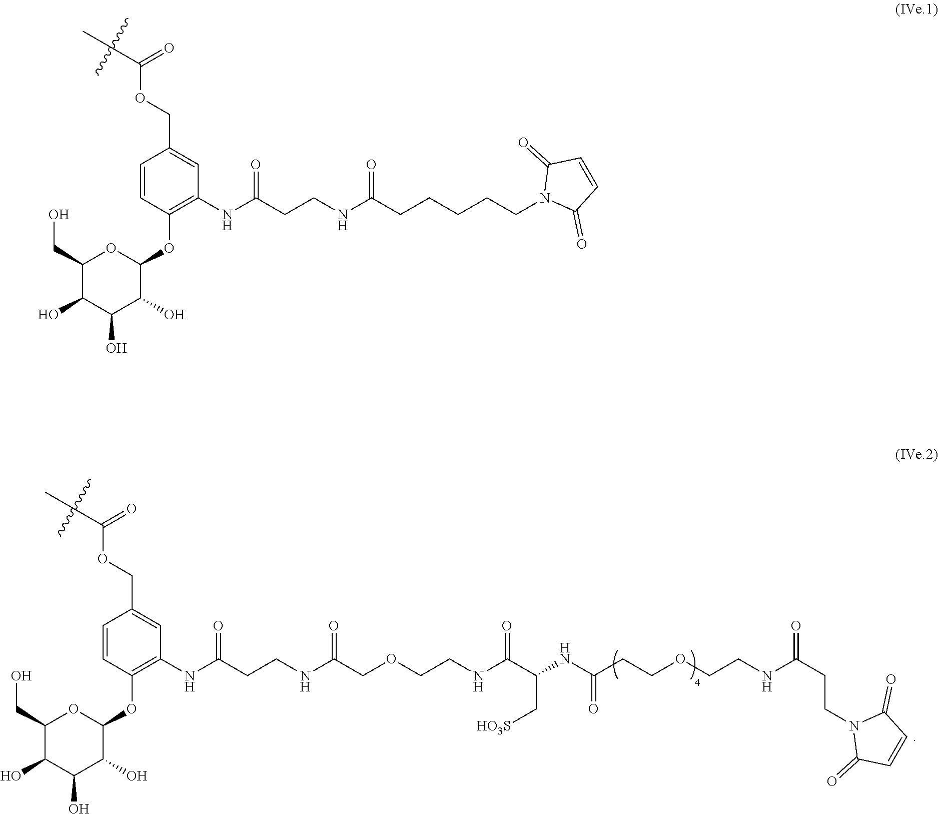

wherein [symbol] represents the point of attachment of a linker to an immune-stimulatory compound.

Although cleavable linkers can provide certain advantages, the linkers comprising the conjugate described herein need not be cleavable. For non-cleavable linkers, the immune-stimulatory compound release may not depend on the differential properties between the plasma and some cytoplasmic compartments. The release of the immune-stimulatory compound can occur after internalization of the immune-stimulatory conjugate via antigen-mediated endocytosis and delivery to lysosomal compartment, where the antibody construct can be degraded to the level of amino acids through intracellular proteolytic degradation. This process can release an immune-stimulatory compound derivative, which is formed by the immune-stimulatory compound, the linker, and the amino acid residue or residues to which the linker was covalently attached. The immune-stimulatory compound derivative from immune-stimulatory conjugates with non-cleavable linkers can be more hydrophilic and less membrane permeable, which can lead to less bystander effects and less nonspecific toxicities compared to immune-stimulatory conjugates with a cleavable linker. Immune-stimulatory conjugates with non-cleavable linkers can have greater stability in circulation than immune-stimulatory conjugates with cleavable linkers. Non-cleavable linkers can include alkylene chains, or can be polymeric, such as, for example, based upon polyalkylene glycol polymers, amide polymers, or can include segments of alkylene chains, polyalkylene glycols and/or amide polymers. The linker can contain a polyethylene glycol segment having from 1 to 6 ethylene glycol units.

The linker can be non-cleavable in vivo, for example, a linker according to the formulations below:

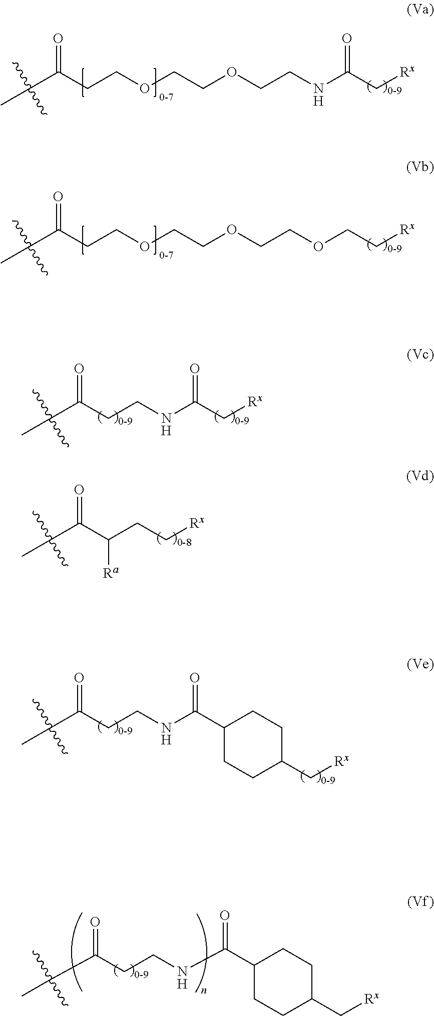

or salts thereof, wherein: R^{a} is selected from hydrogen, alkyl, sulfonate and methyl sulfonate; R^{x} is a reactive moiety including a functional group capable of covalently linking the linker to an antibody construct; and [symbol] represents the point of attachment of the linker to an immune-stimulatory compound.

Exemplary embodiments of linkers according to structural formula (Va)-(Vf) that may be included in the conjugates described herein include the linkers illustrated below (as illustrated, the linkers include a group suitable for covalently linking the linker to an antibody construct, and [symbol] represents the point of attachment of the linker to an immune-stimulatory compound:

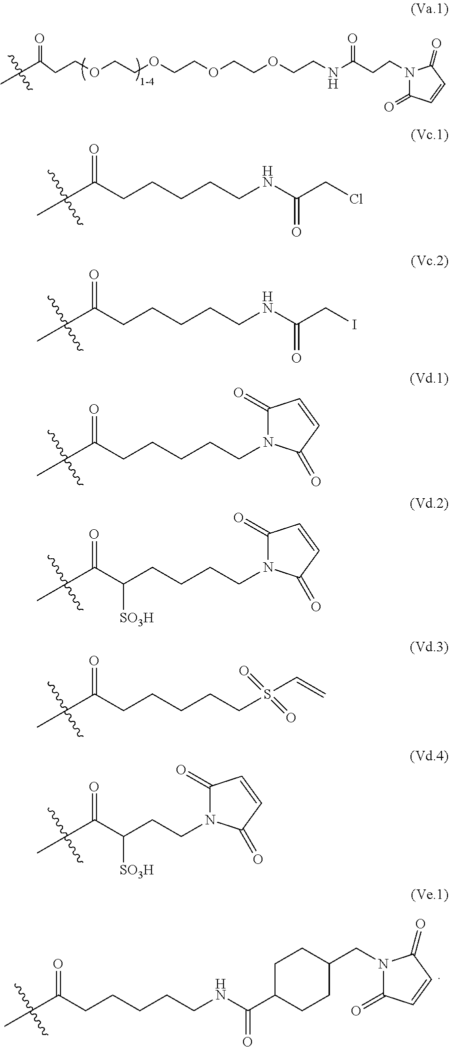

Attachment groups that are used to attach the linkers to an antibody construct can be electrophilic in nature and include, for example, maleimide groups, alkynes, alkynoates, allenes and allenoates, activated disulfides, active esters such as NHS esters and HOBt esters, haloformates, acid halides, alkyl, and benzyl halides such as haloacetamides. There are also emerging technologies related to “self-stabilizing” maleimides and “bridging disulfides” that can be used in accordance with the disclosure.

Maleimide groups are frequently used in the preparation of conjugates because of their specificity for reacting with thiol groups of, for example, cysteine groups of the antibody of a conjugate. The reaction between a thiol group of an antibody and a drug with a linker including a maleimide group proceeds according to the following scheme:

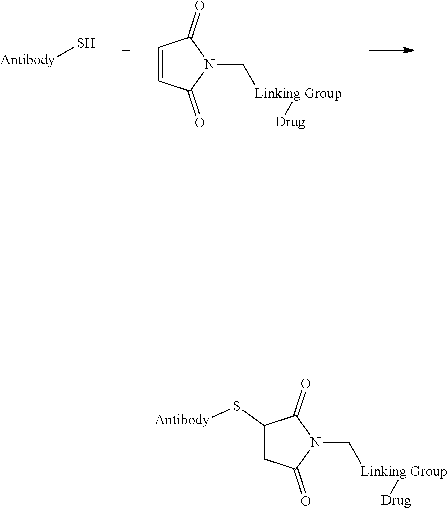

The reverse reaction leading to maleimide elimination from a thio-substituted succinimide may also take place. This reverse reaction is undesirable as the maleimide group may subsequently react with another available thiol group such as other proteins in the body having available cysteines. Accordingly, the reverse reaction can undermine the specificity of a conjugate. One method of preventing the reverse reaction is to incorporate a basic group into the linking group shown in the scheme above. Without wishing to be bound by theory, the presence of the basic group may increase the nucleophilicity of nearby water molecules to promote ring-opening hydrolysis of the succinimide group. The hydrolyzed form of the attachment group is resistant to deconjugation in the presence of plasma proteins. So-called “self-stabilizing” linkers provide conjugates with improved stability. A representative schematic is shown below:

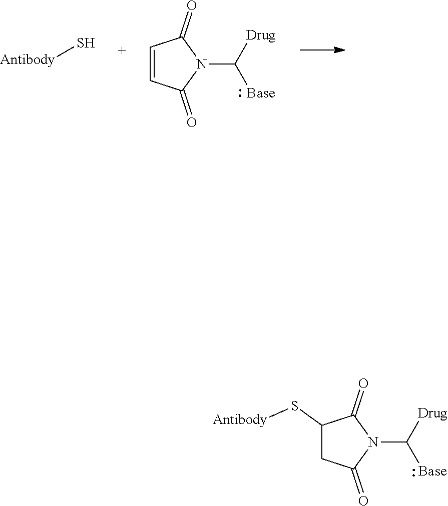

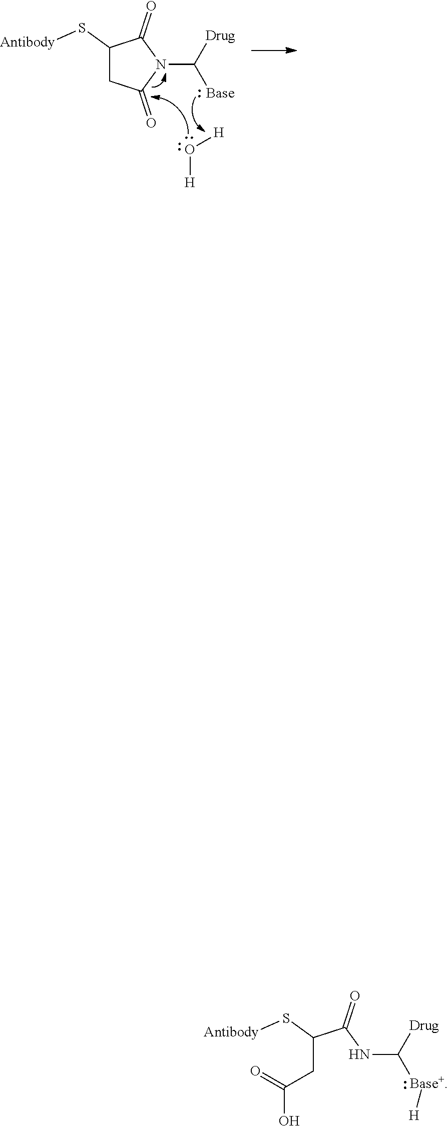

The hydrolysis reaction schematically represented above may occur at either carbonyl group of the succinimide group. Accordingly, two possible isomers may result, as shown below:

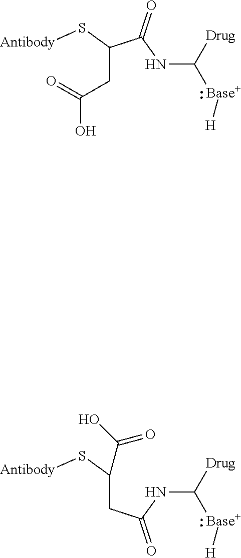

The identity of the base as well as the distance between the base and the maleimide group can be modified to tune the rate of hydrolysis of the thio-substituted succinimide group and optimize the delivery of a conjugate to a target by, for example, improving the specificity and stability of the conjugate.

Bases suitable for inclusion in a linker described herein, e.g., any linker described herein with a maleimide group prior to conjugating to an antibody construct, may facilitate hydrolysis of a nearby succinimide group formed after conjugation of the antibody construct to the linker.

Bases may include, for example, amines (e.g., —N(R²⁶)(R²⁷), where R²⁶ and R²⁷ are independently selected from H and C₁₋₆ alkyl), nitrogen-containing heterocycles (e.g., a 3- to 12-membered heterocycle including one or more nitrogen atoms and optionally one or more double bonds), amidines, guanidines, and carbocycles or heterocycles substituted with one or more amine groups (e.g., a 3- to 12-membered aromatic or non-aromatic cycle optionally including a heteroatom such as a nitrogen atom and substituted with one or more amines of the type —N(R²⁶)(R²⁷), where R²⁶ and R²⁷ are independently selected from H or C₁₋₆ alkyl). A basic unit may be separated from a maleimide group by, for example, an alkylene chain of the form —(CH₂)ₘ—, where m is an integer from 0 to 10. An alkylene chain may be optionally substituted with other functional groups as described herein.

A linker described herein with a maleimide group may include an electron withdrawing group such as, but not limited to, —C(O)R, ═O, —CN, —NO₂, —CX₃, —X, —COOR, —CONR₂, —COR, —COX, —SO₂R, —SO₂OR, —SO₂NHR, —SO₂NR₂, PO₃R₂, —P(O)(CH₃)NHR, —NO, —NR₃⁺, —CR═CR₂, and —C≡CR, where each R is independently selected from H and C₁₋₆ alkyl and each X is independently selected from F, Br, Cl, and I. Self-stabilizing linkers may also include aryl, e.g., phenyl, or heteroaryl, e.g., pyridine, groups optionally substituted with electron withdrawing groups such as those described herein.

Examples of self-stabilizing linkers are provided in, e.g., U.S. Patent Publication Number 2013/0309256, the linkers of which are incorporated by reference herein. It will be understood that a self-stabilizing linker useful in conjunction with immune-stimulatory compounds may be equivalently described as unsubstituted maleimide-including linkers, thio-substituted succinimide-including linkers, or hydrolyzed, ring-opened thio-substituted succinimide-including linkers.

In certain embodiments, a linker comprises a stabilizing linker moiety selected from:

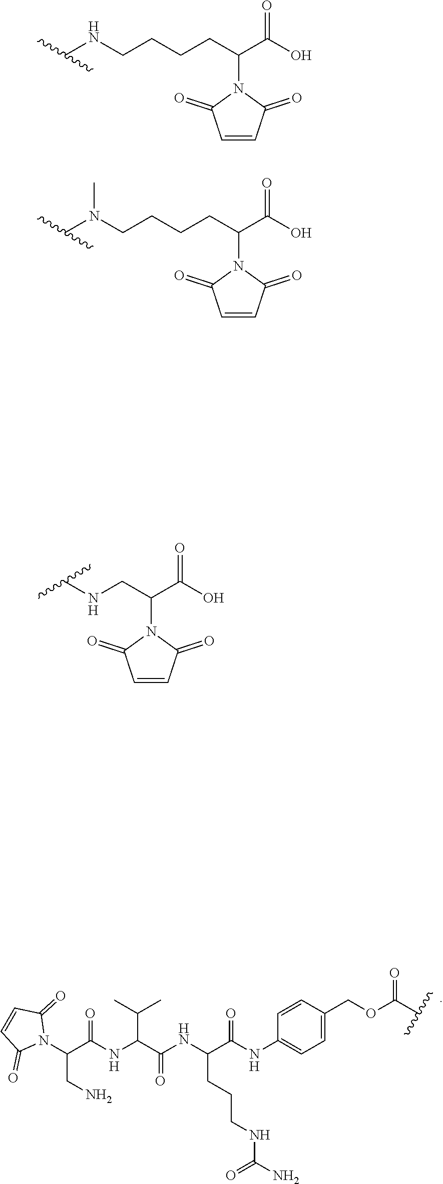

In the scheme provided above, the bottom structure may be referred to as (maleimido)-DPR-Val-Cit-PAB, where DPR refers to diaminopropinoic acid, Val refers to valine, Cit refers to citrulline, and PAB refers to para-aminobenzylcarbonyl. [symbol] represents the point of attachment to an immune-stimulatory compound.

A method for bridging a pair of sulfhydryl groups derived from reduction of a native hinge disulfide bond has been disclosed and is depicted in the schematic below. An advantage of this methodology is the ability to synthesize homogenous DAR4 conjugates by full reduction of IgGs (to give 4 pairs of sulfhydryls from interchain disulfides) followed by reaction with 4 equivalents of the alkylating agent. Conjugates containing “bridged disulfides” are also claimed to have increased stability.

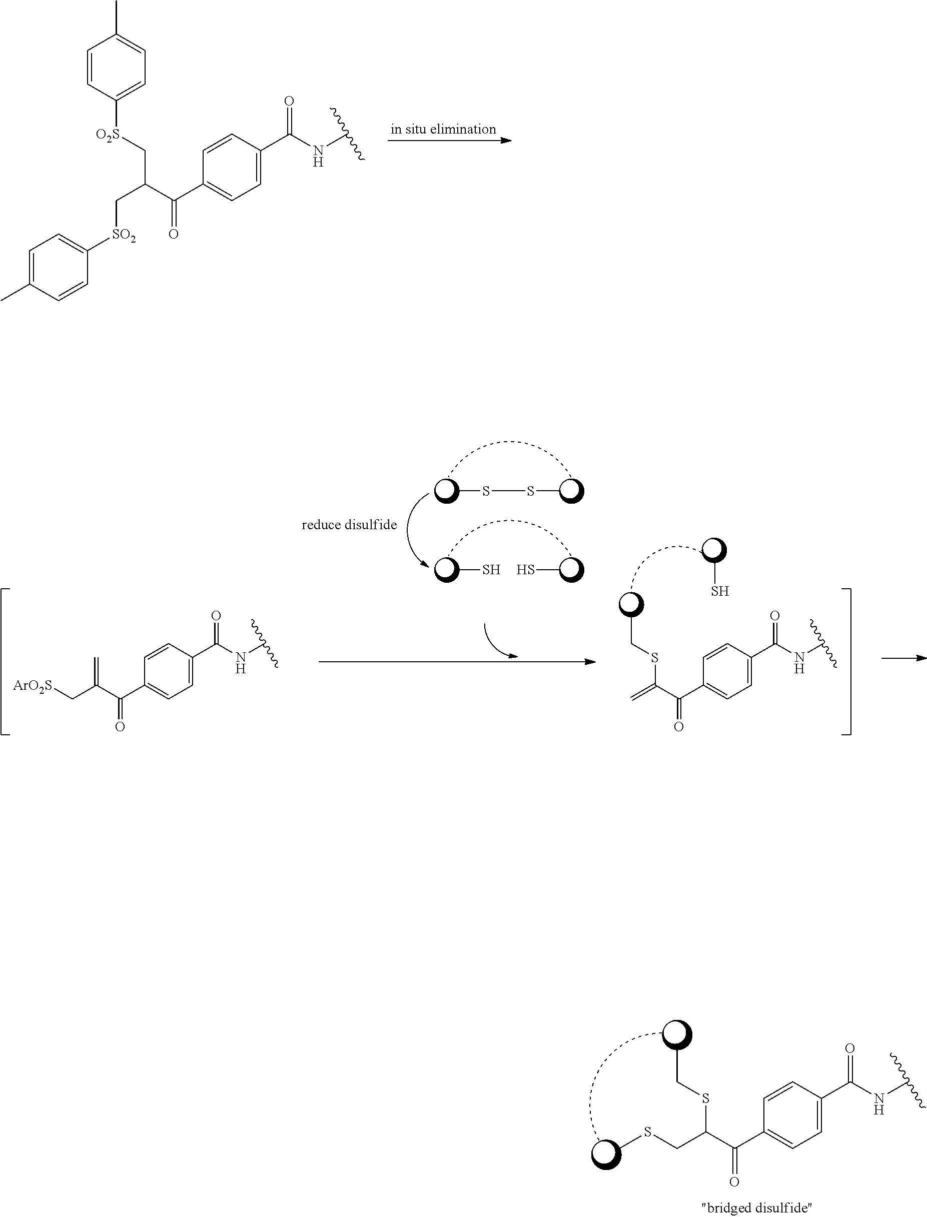

Similarly, as depicted below, a maleimide derivative that is capable of bridging a pair of sulfhydryl groups has been developed.

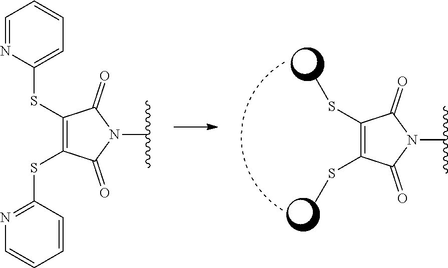 [SMILES: *N1C(=O)C(Sc2ccccn2)=C(Sc2ccccn2)C1=O ; *N1C(=O)C(SC)=C(SC)C1=O]

A linker can contain the following structural formulas (VIa), (VIb), or (VIc):

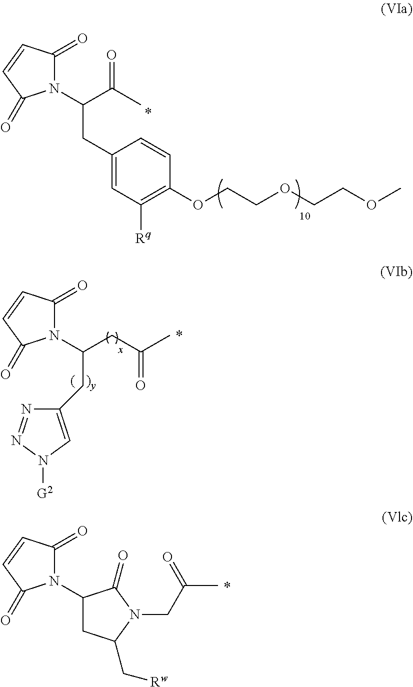

or salts thereof, wherein: R^{q} is H or —O—(CH₂CH₂O)₁₁—CH₃; x is 0 or 1; y is 0 or 1; G² is —CH₂CH₂CH₂SO₃H or —CH₂CH₂O—(CH₂CH₂O)₁₁—CH₃; R^{w} is-O—CH₂CH₂SO₃H or —NH(CO)—CH₂CH₂O—(CH₂CH₂O)₁₂—CH₃; and * represents the point of attachment to the remainder of the linker.

Exemplary embodiments of linkers according to structural formula (VIa) and (VIb) that can be included in the conjugates described herein can include the linkers illustrated below (as illustrated, the linkers can include a group suitable for covalently linking the linker to an antibody construct):

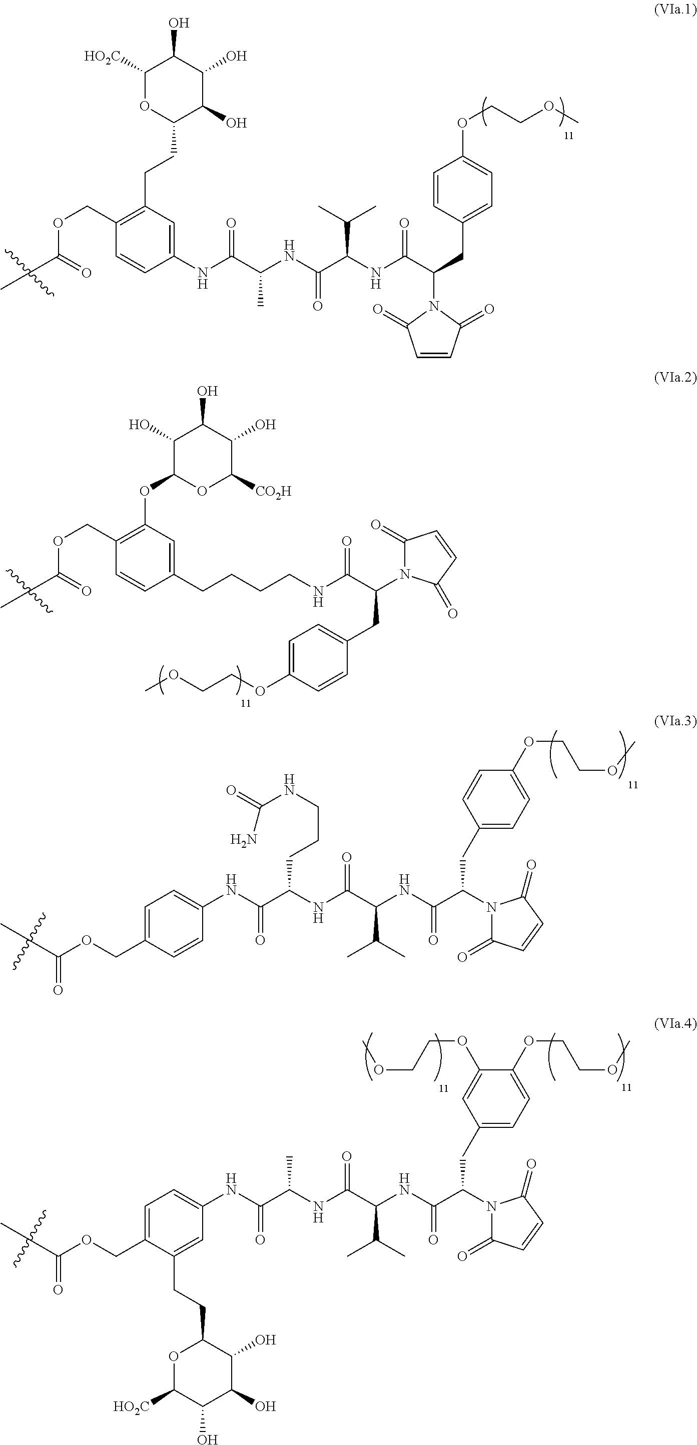

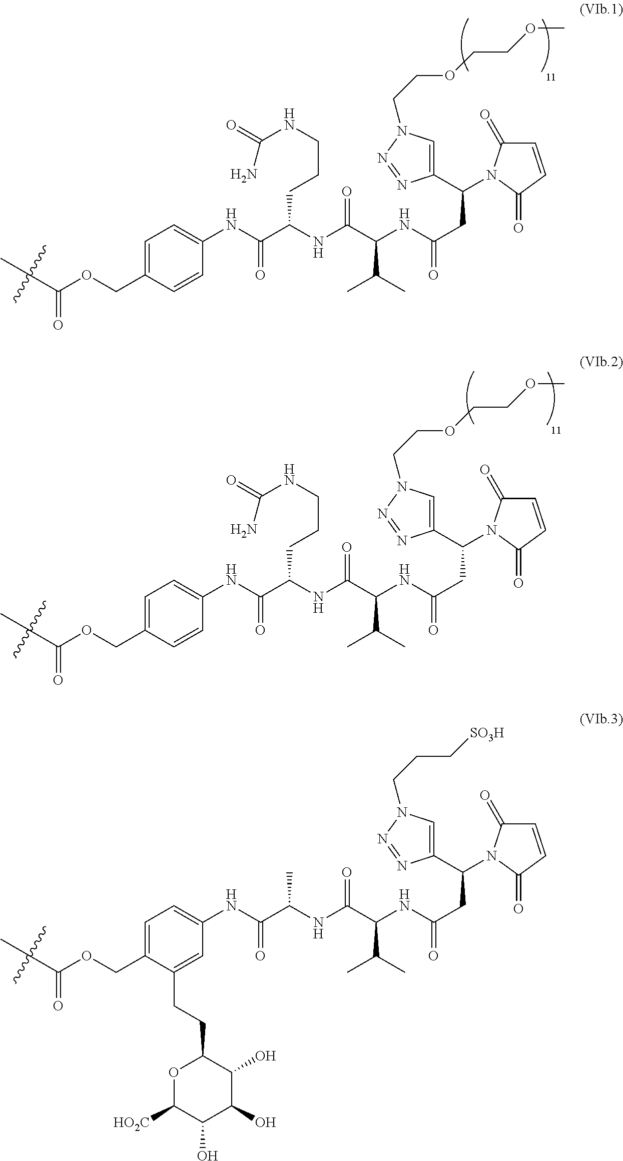

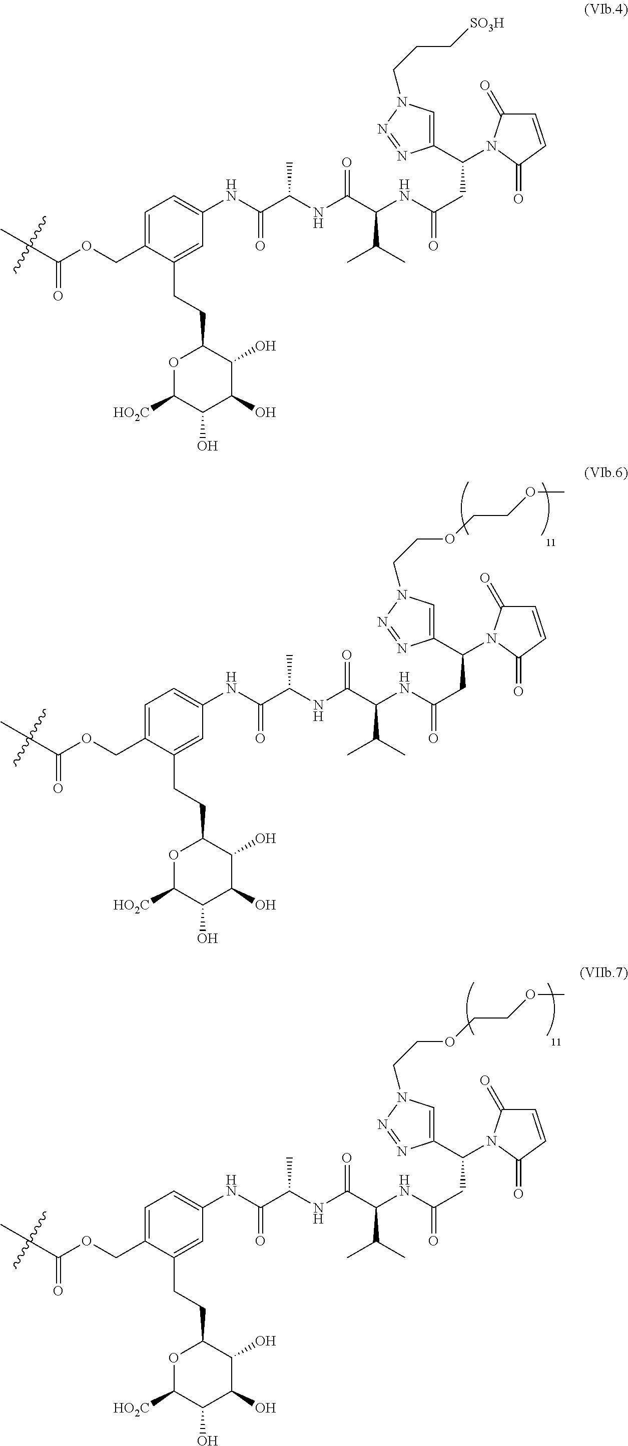

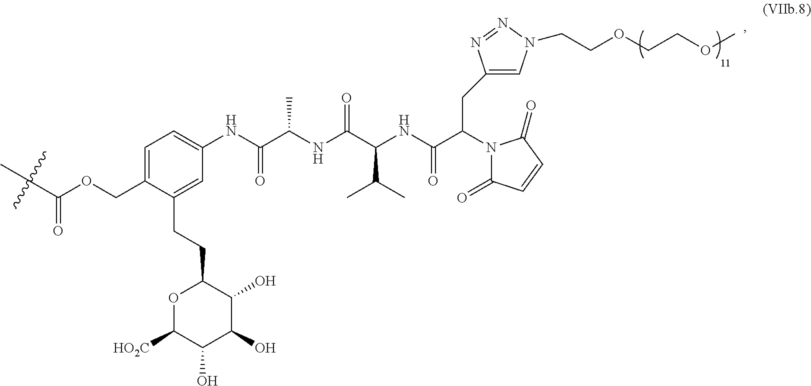

wherein [symbol] represents the point of attachment of the linker to an immune-stimulatory compound.

Exemplary embodiments of linkers according to structural formula (VIc) that can be included in the immune-stimulatory conjugates described herein can include the linkers illustrated below (as illustrated, the linkers can include a group suitable for covalently linking the linker to an antibody construct):

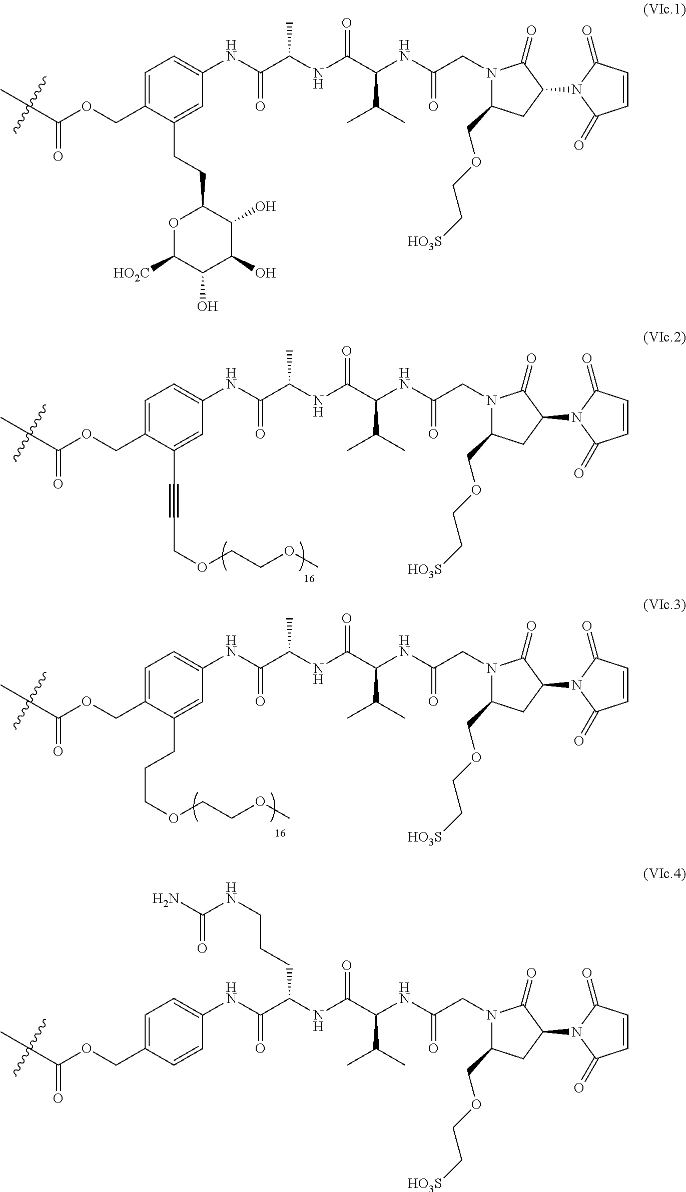

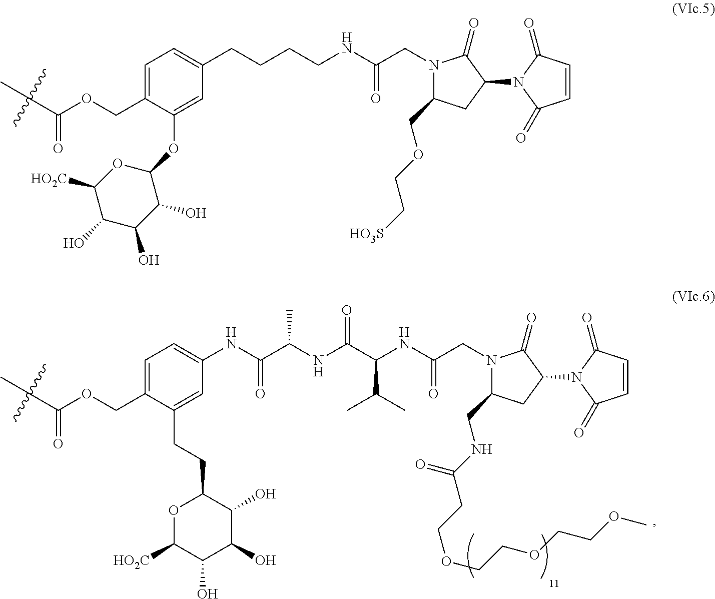

wherein [symbol] represents the point of attachment of the linker to an immune-stimulatory compound.

A linker can be attached to an antibody construct at any suitable position. Factors to be considered in selecting an attachment site include whether the linker is cleavable or non-cleavable, the reactive group of the linker for attachment to the antibody construct, the chemical nature of the immune-stimulatory compound and compatabiltity with reactive sites on the linker and the antibody construct, and the effect of the attachment site on functional activities of the Fc domain. A linker may be attached to a terminus of an amino acid sequence of an antibody construct or can be attached to a side chain of an amino acid of an antibody construct, such as the side chain of a lysine, serine, threonine, cysteine, tyrosine, aspartic acid, glutamine, a non-natural amino acid residue, or glutamic acid residue. A linker may be bound to a terminus of an amino acid sequence of an Fc domain or Fc region of an antibody construct, or may be bound to a side chain of an amino acid of an Fc domain of an antibody construct, such as the side chain of a lysine, serine, threonine, cysteine, tyrosine, aspartic acid, glutamine, a non-natural amino acid residue, or glutamic acid residue.

In some embodiments, a linker is attached to a hinge cysteine of an antibody Fc domain. A linker can be attached to an antibody construct at a light chain constant domain lysine. A linker can be attached to an antibody construct at an engineered cysteine in the light chain. A linker can be attached to an antibody construct at an engineered light chain glutamine. A linker can be attached to an antibody construct at an unnatural amino acid engineered into the light chain. A linker can be attached to an antibody construct at a heavy chain constant domain lysine. A linker can be attached to an antibody construct at an engineered cysteine in the heavy chain. A linker can be attached to an antibody construct at an engineered heavy chain glutamine. A linker can be attached to an antibody construct at an unnatural amino acid engineered into the heavy chain. Amino acids can be engineered into an amino acid sequence of an antibody construct as described herein or as known to the skilled artisan and can be connected to a linker of a conjugate. Engineered amino acids can be added to a sequence of existing amino acids. Engineered amino acids can be substituted for one or more existing amino acids of a sequence of amino acids.

A linker can be attached to an antibody construct via a sulfhydryl group. A linker can be attached to an antibody construct via a primary amine. A linker can be a link created between an unnatural amino acid on an antibody construct reacting with oxime bond that was formed by modifying a ketone group with an alkoxyamine on an immune stimulatory compound.

As is known by skilled artisans, the linker selected for a particular conjugate may be influenced by a variety of factors, including but not limited to, the site of attachment to the antibody construct (e.g., lys, cys or other amino acid residues), structural constraints of the drug pharmacophore and the lipophilicity of the drug. The specific linker selected for a conjugate should seek to balance these different factors for the specific antibody construct/drug combination.

For example, conjugates have been observed to effect killing of bystander antigen-negative cells present in the vicinity of the antigen-positive tumor cells. The mechanism of bystander cell killing by conjugates has indicated that metabolic products formed during intracellular processing of the conjugates may play a role. Neutral cytotoxic metabolites generated by metabolism of the conjugates in antigen-positive cells appear to play a role in bystander cell killing while charged metabolites may be prevented from diffusing across the membrane into the medium, or from the medium across the membrane, and therefore cannot affect bystander killing. In certain embodiments, the linker is selected to attenuate the bystander effect caused by cellular metabolites of the conjugate. In certain embodiments, the linker is selected to increase the bystander effect.

The properties of the linker, or linker-compound, may also impact aggregation of the conjugate under conditions of use and/or storage. Typically, conjugates reported in the literature contain no more than 3-4 drug molecules per antibody molecule. Attempts to obtain higher drug-to-antibody ratios (“DAR”) often failed, particularly if both the drug and the linker were hydrophobic, due to aggregation of the conjugate. In many instances, DARs higher than 3-4 could be beneficial as a means of increasing potency. In instances where an immune-stimulatory compound is more hydrophobic in nature, it may be desirable to select linkers that are relatively hydrophilic as a means of reducing conjugate aggregation, especially in instances where DARs greater than 3-4 are desired. Thus, in certain embodiments, a linker incorporates chemical moieties that reduce aggregation of the conjugates during storage and/or use. A linker may incorporate polar or hydrophilic groups such as charged groups or groups that become charged under physiological pH to reduce the aggregation of the conjugates. For example, a linker may incorporate charged groups such as salts or groups that deprotonate, e.g., carboxylates, or protonate, e.g., amines, at physiological pH.

In particular embodiments, the aggregation of the conjugates during storage or use is less than about 40% as determined by size-exclusion chromatography (SEC). In particular embodiments, the aggregation of the conjugates during storage or use is less than 35%, such as less than about 30%, such as less than about 25%, such as less than about 20%, such as less than about 15%, such as less than about 10%, such as less than about 5%, such as less than about 4%, or even less, as determined by size-exclusion chromatography (SEC).

### Exemplary Syntheses of Myeloid Cell Agonist-Linkers

A myeloid cell agonist-linker compound can be synthesized by various methods before being attached to an antibody construct to form the conjugates as described herein. For example, a can be synthesized as shown in Scheme B1.

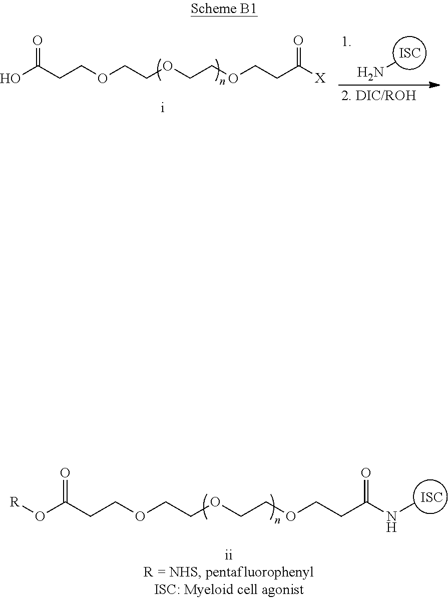

A PEGylated carboxylic acid (i) that has been activated for amide bond formation can be reacted with an appropriately substituted amine containing myeloid cell agonist to afford an intermediate amide. Formation of an activated ester (ii) can be achieved by reaction the intermediate amide-containing carboxylic using a reagent such as N-hydroxysuccinimide or pentafluorophenol in the presence of a coupling agent such as diisopropylcarbodiimide (DIC) to provide compounds (ii).

As another example, myeloid cell agonist-linkers can be synthesized as shown in Scheme B2.

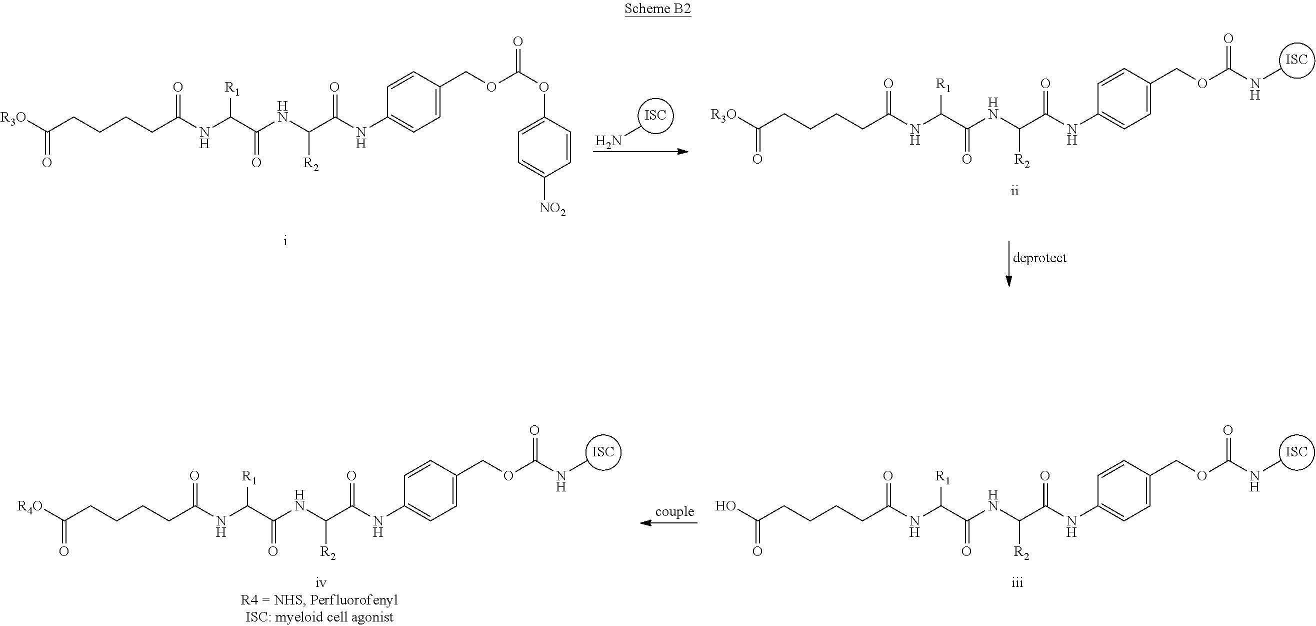

An activated carbonate such as (i) can be reacted with an appropriately substituted amine containing myeloid cell agonist to afford carbamates (ii) which can be deprotected using standard methods based on the nature of the R₃ ester group. The resulting carboxylic acid (iii) can then by coupled with an activating agent such as N-hydroxysuccinimide or pentafluorophenol to provide compounds (iv).

As an additional example, myeloid cell agonist-linker can be synthesized as shown in Scheme B3.

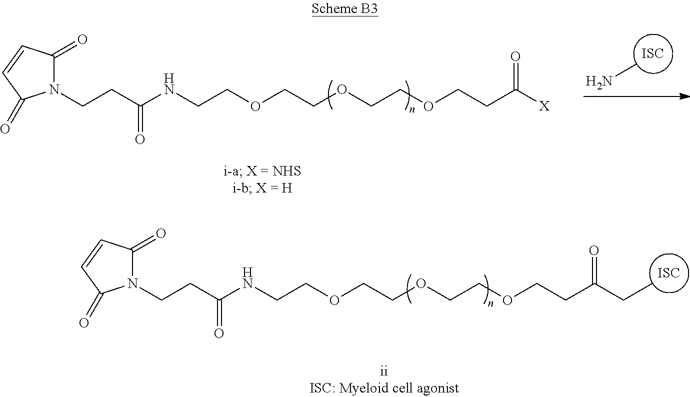

An activated carboxylic ester such as (i-a) can be reacted with an appropriately substituted amine containing myeloid cell agonist to afford amides (ii). Alternatively, carboxylic acids of type (i-b) can be coupled to an appropriately substituted amine containing myeloid cell agonist in the presence of an amide bond forming agent such as dicyclohexycarbodiimde (DCC) to provide the desired myeloid cell agonist-linker.

As an additional example, a myeloid cell agonist-linker can be synthesized as shown in Scheme B4.

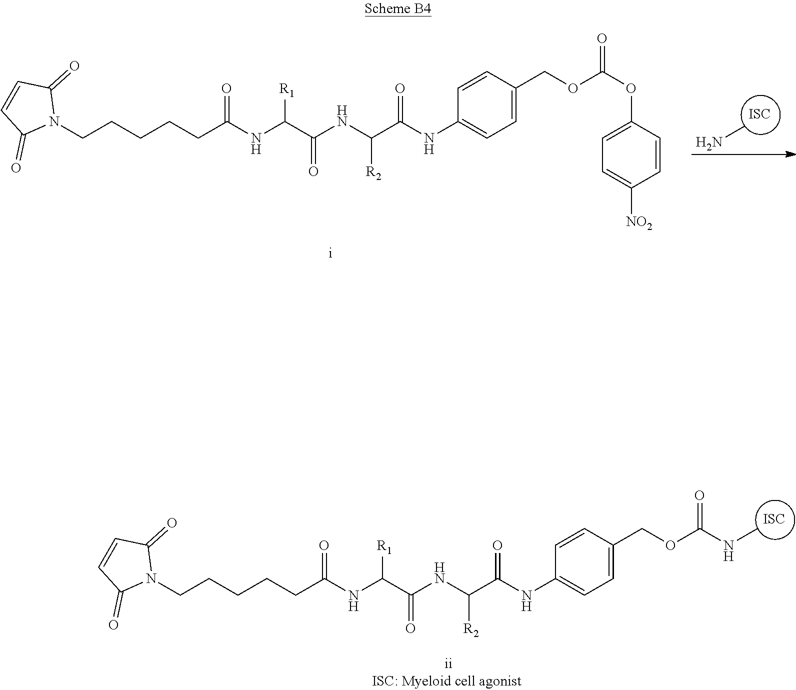

An activated carbonate such as (i) can be reacted with an appropriately substituted amine containing myeloid cell agonist to afford carbamates (ii) as the target myeloid cell agonist.

As an additional example, a myeloid cell agonist-linker can be synthesized as shown in Scheme B5.

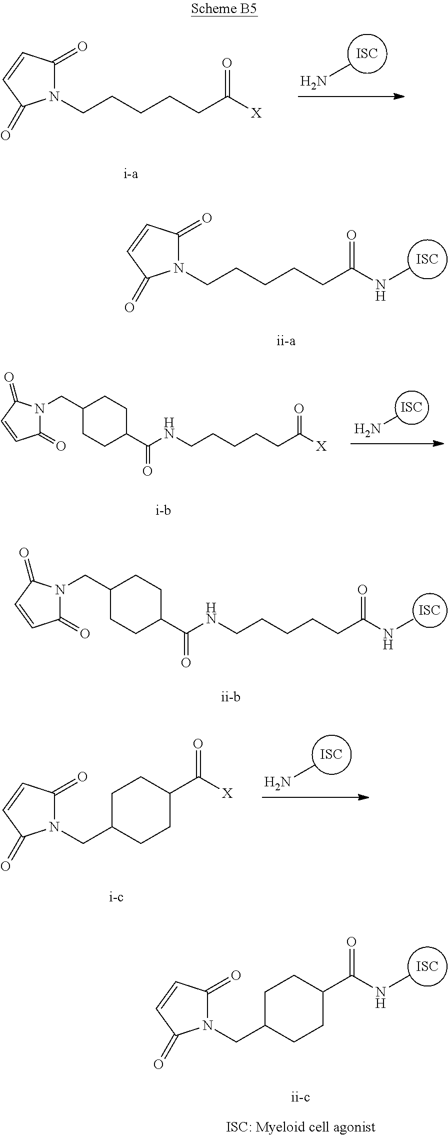

An activated carboxylic acid such as (i-a, i-b, i-c) can be reacted with an appropriately substituted amine containing myeloid cell agonist to afford amides (ii-a, ii-b, ii-c) as the target myeloid cell agonists.

These myeloid cell agonist-linkers can be made by various methods. It is understood that one skilled in the art may be able to make these compounds by similar methods or by combining other methods known to one skilled in the art. It is also understood that one skilled in the art would be able to make, in a similar manner as described herein by using the appropriate starting materials and modifying the synthetic route as needed. Starting materials and reagents can be obtained from commercial vendors or synthesized according to sources known to those skilled in the art or prepared as described herein.

### Conjugates

A conjugate as described herein comprises an antibody construct and at least one linker attached to at least one immune-stimulatory compound, such as a myeloid cell agonist or other agonist (e.g., TLR8 agonist, TLR7 agonist, other TLR agonist, STING agonist, RIG-I-Like receptor agonist, c-type lectin receptors agonist, or cytosolic DNA Sensors agonist). In some aspects, the present disclosure provides a conjugate represented by Formula I:

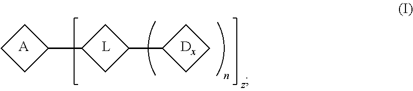

wherein:
A is the antibody construct,
L is the linker;
Dₓ is the immune-stimulatory compound;
n is selected from 1 to 20; and
z is selected from 1 to 20.

In some embodiments, the immune-stimulatory compound is a myeloid cell agonist. In some embodiments, the immune-stimulatory compound is a TLR8 agonist. In some embodiments, the immune-stimulatory compound is a TLR7 agonist. In some embodiments, the immune-stimulatory compound is a TLR3 agonist. In some embodiments, the immune-stimulatory compound is a TLR4 agonist. In some embodiments, the immune-stimulatory compound is a TLR5 agonist. In some embodiments, the immune-stimulatory compound is a TLR9 agonist. In some embodiments, the immune-stimulatory compound is a STING agonist. In some embodiments, the immune-stimulatory compound is a RIG-I-Like receptor agonist. In some embodiments, the immune-stimulatory compound is a c-type lectin receptors agonist. In some embodiments, the immune-stimulatory compound is a cytosolic DNA Sensors agonist.

In some aspects, the present disclosure provides a conjugate comprising at least one immune-stimulatory compound (e.g., a compound or salt thereof), an antibody construct, and at least one linker, wherein each immune-stimulatory compound is linked, i.e., covalently bound, to the antibody construct through a linker. The linker can be selected from a cleavable or non-cleavable linker. In some embodiments, the linker is cleavable. In other embodiments, the linker is non-cleavable. Linkers are further described in the present application in the preceeding section, any one of which can be used to connect an antibody construct to an immune-stimulatory compound.

In a conjugate, the drug loading is represented by z, the number of immune-stimulatory compound-linker molecules per antibody construct, or the number of immune-stimulatory compounds per antibody construct, depending on the particular conjugate. Depending on the context, z can represent the average number of immune-stimulatory compound(-linker) molecules per antibody construct, also referred to the average drug loading. z can range from 1 to 20, from 1-50 or from 1-100. In some conjugates, z is preferably from 1 to 8. In some preferred embodiments, when z represents the average drug loading, z ranges from about 2 to about 5. In some embodiments, z is about 2, about 3, about 4, or about 5. The average number of immune-stimulatory compounds per antibody construct in a preparation of conjugate may be characterized by conventional means such as mass spectroscopy, liquid chromatography/mass spectrometry (LC/MS), HIC, ELISA assay, and HPLC.

A number of conjugates are consistent with the disclosure herein. The conjugates generally comprise an immune-stimulatory compound covalently bound to a targeting moiety or antibody construct that localizes the conjugate to a target tissue, cell population or cell. The targeting moiety can comprise all or part of an antibody variable domain, although alternate targeting moieties are also contemplated. The targeting moiety or antibody construct is covalently attached to each immune-stimulatory compound, either directly or through a linker that tethers the immune-stimulatory compound to the targeting moiety or antibody construct. Antibodies listed herein as well as antibodies to antigens or epitopes thereof listed herein or otherwise known to one of skill in the art are consistent with the conjugates as disclosed herein.

Some exemplary conjugates are as follows. A conjugate can comprise an antibody construct, at least one immune-stimulatory compound, and optionally at least one linker. A conjugate can comprise an antibody construct, at least one TLR7 agonist, and at least one linker. A conjugate can comprise an antibody construct, at least one TLR8 agonist, and at least one linker. A conjugate can comprise an antibody construct, at least one Compound A TLR8 agonist, and at least one linker. A conjugate can comprise an antibody construct, at least one Compound B TLR7 agonist, and at least one linker. A conjugate can comprise an antibody construct, at least one TLR3 agonist, and at least one linker. A conjugate can comprise an antibody construct, at least one TLR4 agonist, and at least one linker. A conjugate can comprise an antibody construct, at least one TLR5 agonist, and at least one linker. A conjugate can comprise an antibody construct, at least one TLR9 agonist, and at least one linker. A conjugate can comprise an antibody construct, at least one STING agonist, and at least one linker. A conjugate can comprise an antibody construct, at least one RIG-I agonist, and at least one linker. A conjugate can comprise an antibody construct, at least one c-type lectin receptor agonist, and at least one linker. A conjugate can comprise an antibody construct, at least one cytosolic DNA Sensors agonist, and at least one linker.

In some embodiments, the immune stimulatory compound is a myeloid cell agonist. A number of myeloid cell agonists are consistent with the disclosure herein such as a TLR8 agonist. Exemplary TLR8 agonists are selected from compounds 1.1-1.2, 1.4-1.20, 1.23-1.27, 1.29-1.46, 1.48, and 1.50-1.67 (Examples). In some embodiments, a myeloid cell agonist-linker compound (Linker-Payload) is selected from any of Linker-Payloads 2.1-2.17 (Examples).

The immune-stimulatory conjugates as described herein can activate, stimulate or augment an immune response against cell of a disease of condition, while sparing, alleviating, or avoiding toxicity(ies) associated with bolus intravenous administration of the immune-stimulatory conjugate. The activation, stimulation or augmentation of an immune response by an immune-stimulatory conjugate, such as a myeloid cell agonist, can be measured in vitro by co-culturing immune cells (e.g., myeloid cells) with cells targeted by the conjugate and measuring cytokine release, chemokine release, proliferation of immune cells, upregulation of immune cell activation markers, and/or ADCC. ADCC can be measured by determining the percentage of remaining target cells in the co-culture after administration of the conjugate with the target cells, myeloid cells, and other immune cells. In some embodiments, an immune-stimulatory conjugate can activate or stimulate immune cell activity, as determined by in vitro assay, such as a cytokine release assay, by detection of activation markers (e.g., MHC class II markers) or other assays known in the art. In some embodiments, an immune-stimulatory conjugate has an EC50 of 100 nM or less, as determine by cytokine release assay. In some embodiments, an immune-stimulatory conjugate has an EC50 of 50 nM or less, as determine by cytokine release assay. In some embodiments, an immune-stimulatory conjugate has an EC50 of 10 nM or less, as determine by cytokine release assay. In some embodiments, an immune-stimulatory conjugate has an EC50 of 1 mM or less.

### Pharmaceutical Formulations

The conjugates described herein are useful as pharmaceutical compositions for administration (e.g., subcutaneous, slow IV infusion) to a subject in need thereof. Pharmaceutical compositions can comprise the conjugates described herein and one or more pharmaceutically acceptable excipients, suitable for subcutaneous administration. A pharmaceutical composition can comprise any conjugate described herein. A pharmaceutical composition can further comprise buffers, carbohydrates, and/or preservatives, as appropriate. Pharmaceutical compositions comprising a conjugate can be manufactured, for example, by lyophilizing the conjugate, mixing, dissolving, emulsifying, encapsulating or entrapping the conjugate. The pharmaceutical compositions can also include the conjugates described herein in a free-base form or pharmaceutically-acceptable salt form.

Methods for formulation of the pharmaceutical compositions can include formulating any of the conjugates described herein with one or more inert, pharmaceutically-acceptable excipients or carriers to form a solid, semi-solid, or liquid composition for subcutaneous administration. Solid compositions can include, for example, powders, and in some aspects, the solid compositions further contain nontoxic, auxiliary substances, for example wetting or emulsifying agents, pH buffering agents, and other pharmaceutically-acceptable additives. Alternatively, the compositions described herein can be lyophilized or in powder form for re-constitution with a suitable vehicle, e.g., sterile pyrogen-free water, before use

The pharmaceutical compositions and formulations can be sterilized. Sterilization can be accomplished by filtration through sterile filtration.

The pharmaceutical compositions described herein can be formulated for administration as an injection, i.e., a subcutaneous injection. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Alternatively, the pharmaceutical compositions described herein can be lyophilized or in powder form for reconstitution with a suitable vehicle, e.g., sterile pyrogen-free water, before use.

Formulations for subcutaneous administration have been described in, for example, WO2018/136412, WO2016/036678, WO2013/173687, WO2013/096835, WO2012/151199, WO2011/147921, WO2011/104381, WO2011/090088, WO2011/017070, WO2011/012637, WO2009/084659, and WO2004/091658, each of which is hereby incorporated by reference in its entirety.

The conjugates can be formulated for subcutaneous administration in a unit dosage form in association with a pharmaceutically acceptable vehicle. Such vehicles can be inherently nontoxic, and non-therapeutic. A vehicle can be water, saline, Ringer's solution, dextrose solution, and 5% human serum albumin. Nonaqueous vehicles such as fixed oils and ethyl oleate can also be used. The vehicle can contain minor amounts of additives such as substances that enhance isotonicity and chemical stability (e.g., buffers and preservatives).

### Therapeutic Applications

The immune-stimulatory conjugates and pharmaceutical compositions thereof are useful in the methods of the present disclosure for treating plurality of different subjects including, but not limited to, a mammal, human, non-human mammal, a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof.

The immune-stimulatory conjugates and pharmaceutical compositions thereof can be used in the methods described herein as a therapeutic, for example, as a treatment that can be administered in an effective regimen to a subject in need thereof to achieve a therapeutic effect, while alleviating, sparing, or avoiding toxicity(ies) associated with bolus repetitive intravenous administration of the conjugate. Toxicities that can be alleviated, spared, or avoided include anaphylaxis-like toxicity. A therapeutic effect can be obtained in a subject by reduction, suppression, remission, alleviation or eradication of a disease state, including, but not limited to, one or more symptoms thereof. A therapeutic effect in a subject having a disease or condition, or exhibiting an early symptom thereof or exhibiting or otherwise suspected of being in or approaching an early stage of a disease or condition, can be obtained by a reduction, a suppression, a prevention, a delay, a remission, an alleviation or an eradication of the condition or disease, or pre-condition or pre-disease state. In various embodiments, the effective regimen results in a Tmax of the immune-stimulatory conjugate of greater than about 4 hours following each administration of the immune-stimulatory conjugate. In some embodiments, the effective regimen results in a Tmax greater than about 6 hours, greater than about 8 hours, greater than about 10 hours, greater than about 12 hours, or greater than about 15 hours following each administration of the immune-stimulatory conjugate.

In certain embodiments, the methods include subcutaneous or intravenous slow-infusion administration of an immune-stimulatory conjugate, or a pharmaceutical composition thereof, to a subject in need thereof in an effective regimen to activate, stimulate or augment an immune response against a disease treatable with a TLR agonist (e.g., cancer or a viral disease). The antibody construct of the conjugate recognizes an antigen associated with the disease or disease state.

In certain embodiments, the methods include subcutaneous or intravenous slow-infusion administration of an immune-stimulatory conjugate, or a pharmaceutical composition thereof, to a subject in need thereof in an effective regimen to activate, stimulate or augment an immune response against cell of a disease of condition. In certain embodiments, the methods include subcutaneous or intravenous slow-infusion administration of an immune-stimulatory conjugate, or a pharmaceutical composition thereof, to a subject in need thereof in an effective regimen to activate, stimulate or augment an immune response against cancer cells, where the cancer cells express a tumor antigen or a tumor associated antigen recognized by the antibody construct of the conjugate. In certain embodiments, the methods include subcutaneous or intravenous slow-infusion administration of an immune-stimulatory conjugate, or a pharmaceutical composition thereof, to a subject in need thereof in an effective regimen to activate, stimulate or augment an immune response against cancer cells expressing a tumor antigen recognized by the antibody construct of the conjugate. In certain embodiments, the methods include subcutaneous or intravenous slow-infusion administration of an immune-stimulatory conjugate, or a pharmaceutical composition thereof, to a subject in need thereof in an effective regimen to activate, stimulate or augment an immune response against cancer cells express a tumor antigen recognized by the antibody construct of the conjugate.

In certain embodiments, the methods include subcutaneous or intravenous slow-infusion administration of an immune-stimulatory conjugate, or a pharmaceutical composition thereof, to a subject in need thereof in an effective regimen to activate, stimulate or augment an immune response against tumor cells of a solid tumor, such as a sarcoma, a carcinoma or lymphoma. The antibody construct of the conjugate recognizes an antigen on the target cells, such as tumor cells. In certain embodiments, the methods include subcutaneous or intravenous slow-infusion administration of an immune-stimulatory conjugate, or a pharmaceutical composition thereof, to a subject in need thereof in an effective regimen to activate, stimulate or augment an immune response against tumor cells of a sarcoma. The antibody construct of the conjugate recognizes an antigen on the sarcoma cells. In certain embodiments, the methods include subcutaneous or intravenous slow-infusion administration of an immune-stimulatory conjugate, or a pharmaceutical composition thereof, to a subject in need thereof in an effective regimen to activate, stimulate or augment an immune response against tumor cells of a carcinoma. The antibody construct of the conjugate recognizes an antigen on the tumor cells. In certain embodiments, the methods include subcutaneous or intravenous slow-infusion administration of an immune-stimulatory conjugate, or a pharmaceutical composition thereof, to a subject in need thereof in an effective regimen to activate, stimulate or augment an immune response against tumor cells of a lymphoma. The antibody construct of the conjugate recognizes an antigen on the tumor cells.

In certain embodiments, the methods include subcutaneous or intravenous slow-infusion administration of an immune-stimulatory conjugate, or a pharmaceutical composition thereof, to a subject in need thereof in an effective regimen to activate, stimulate or augment an immune response against tumor cells of a solid tumor, such as brain, breast, lung, liver, kidney, pancreatic, colorectal, ovarian, head and neck, bone, skin, mesothelioma, bladder, stomach, prostate, thyroid, uterine or cervical/endometrial cells. The antibody construct of the conjugate recognizes an antigen on the tumor cells.

In certain embodiments, the cancer is a HER2 expressing cancer and the methods include subcutaneous or intravenous slow-infusion administration of an immune-stimulatory conjugate, or a pharmaceutical composition thereof, to a subject in need thereof in an effective regimen to activate, stimulate or augment an immune response against cells of the HER2 expressing cancer. In some aspects, the HER2 expressing cancer expresses HER2 at a level of 2+ or 3+ as determined by immunohistochemistry.

In some embodiments, toxicities associated with intravenous administration of immune-stimulatory conjugates that can be spared, alleviated, or avoided are anaphylaxis-like toxicities. Such toxicities can be associated with single or multiple intravenous administrations of an immune-stimulatory conjugate. As used herein, “alleviating” or “to alleviate” a toxicity refers to making the toxicity less severe. The terms “sparing” or “to spare” refer to significantly reducing the toxicity and to reduce harm to the subject.

In some embodiments, toxicities of an anaphylaxis-like response that are associated with intravenous administration of immune-stimulatory conjugates are spared, alleviated, or avoided. An anaphylaxis-like response refers to symptoms such as hypotension, airway constriction, hypothermia and/or vascular leak syndrome, in the absence of significant cytokine release. As used herein, an anaphylaxis-like response is other than classical anaphylaxis, resulting from an IgG or IgE response. In some embodiments, grade 4 or greater anaphylaxis-like adverse events associated with repetitive bolus intravenous administration of an immune-stimulatory conjugate are spared, alleviated, or avoided. In some embodiments, grade 3 or greater anaphylaxis-like adverse events associated with repetitive bolus intravenous administration of an immune-stimulatory conjugate are spared, alleviated, or avoided. In some embodiments, grade 2 or greater anaphylaxis-like adverse events associated with repetitive bolus intravenous administration of an immune-stimulatory conjugate are alleviated, spared, or avoided. In some embodiments, grade 1 or greater anaphylaxis-like adverse events associated with repetitive bolus intravenous administration of an immune-stimulatory conjugate are alleviated, spared, or avoided.

One of ordinary skill in the art would understand that the amount, duration and frequency of administration of a pharmaceutical composition or conjugate described herein to a subject in need thereof depends on several factors including, for example but not limited to, the health of the subject, the specific disease or condition of the subject, the grade or level of a specific disease or condition of the subject, the additional therapeutics the subject is being or has been administered, and the like.

In some aspects of practicing the methods described herein, the immune-stimulatory conjugates are subcutaneously administered or administered by a slow IV infusion in an effective regimen of at least two or at least three cycles. Each cycle can optionally include a resting stage between cycles. Cycles of administration can be of any suitable length. In some embodiments, each cycle is a week (7 days), 10 days, every two weeks (14 days or biweekly), every three week (21 days) or every four weeks (28 days). In some embodiments, each cycle is a month. In some embodiments, at least two doses of the immune-stimulatory conjugate are administered more than 7 days apart, or more than 10 days apart. In some embodiments, at least one dose of the immune-stimulatory conjugate is administered more than 7 days, or more than 10 days, after the initial dose of the immune-stimulatory conjugate.

The dose of immune-stimulatory conjugate or pharmaceutical composition thereof within each cycle is an amount suitable to achieve a therapeutic effect. The dose within a cycle can be a single dose or a split dose (i.e., multiple doses within a cycle). In some embodiments, a split-dose is administered when the volume of the pharmaceutical composition to be administered is greater than is typically administered in a single dose by the selected route. For example, the maximum volume that is typically administered subcutaneously is about 1.5 mL, because greater volumes are believed to be associated with injection site pain and other adverse events at the injection site. Accordingly, in some embodiments, when the amount of the pharmaceutical composition to be administered subcutaneously is greater than about 1.5 mL, a split-dose is administered, meaning the volume is split into smaller volumes of, for example, less than 1.5 mL each, and the smaller volumes are each injected at a different site on the body of the subject. In certain embodiments, the total dose of immune-stimulatory conjugate or pharmaceutical composition thereof within a cycle is from about 0.1 to about 10 mg/kg. In some embodiments, the total dose is from about 0.5 to about 7.5 mg/kg. In some embodiments, the total dose is from about 0.5 to about 5 mg/kg. In some embodiments, the total dose is from about 0.5 to about 4 mg/kg. In some embodiments, the total dose is from about 0.5 to about 3.5 mg/kg. In some embodiments, the total dose is from about 0.5 to about 2 mg/kg.

The methods disclosed herein, using the immune stimulatory conjugates disclosed herein, relate to sequential administration (e.g., sequential subcutaneous administration) of a plurality of doses of immune stimulatory conjugates. This sequential administration avoids toxicities associated with repetitive bolus administration of the immune stimulatory conjugates. In some aspects, the immune stimulatory conjugates are administered in an effective regimen that results in a Tmax of the immune-stimulatory conjugate in the subject of greater than about 4 hours following each administration of the immune-stimulatory conjugate. In some aspects, the Tmax is reached at or prior to about 72 hours, at or prior to about 48 hours, at or prior to about 30 hours, at or prior to about 24 hours, or at or prior to about 16 hours.

Immune stimulatory compounds, particularly delivered as constituents of conjugates as discussed generally herein, stimulate or induce targeted activation of a particular immune response pathway localized to a particular target, by conjugation to an antibody construct, such as an antibody variable domain, or other targeting moiety that differentially binds to a particular antibody construct target by selectively binding to that target.

Application of such conjugates shows substantial benefit in directing a subject's own immune response to cells of a particular site of disease or disorder, such as cells associated with the disease or disorder. Activating or stimulating an immune response directed to targeted cells facilitates the reduction, inhibition of proliferation, inhibition of growth, inhibition of progression, inhibition of metastasis or otherwise inhibition up to and including, in some cases, clearance of the targeted cells. Thus, in some cases, a targeted immune response activation or stimulation leads to inhibition of disease progression, or alleviation of at least one symptom of a manifest disease in a patient, up to and, in some cases, including complete elimination of from one symptom to an entire disease state in a subject.

Nonetheless, administration of immune stimulatory conjugates is not without some risk. As disclosed herein, bolus repetitive intravenous administration of immune-stimulatory conjugates can result in elicitation of an unwanted or unintended immune response, such as an anaphylaxis-like response. Such a toxicity may be characterized by certain symptoms including, in various cases, one or more of a drop in body temperature, a drop in blood pressure, restriction of the airways, a rapid and weakening pulse, and in some cases, death.

Risk of such toxicities is increased when an immune stimulatory conjugate is administered in a dosage regimen comprising multiple bolus intravenous administrations in series. Intravenous bolus administration of an immune stimulatory conjugate to a subject in a second dose increases a risk that, in addition to or prior to eliciting a targeted immune response directed to a particular disease or disorder or cells thereof, the subject may experience a toxicity, such as an anaphylaxis-like toxicity.

Accordingly, disclosed herein are treatment regimens that reduce or eliminate toxicities associated with repetitive bolus intravenous administration of immune stimulatory conjugates such as, but not in all cases limited to, the immune stimulatory conjugates disclosed herein. Such toxicities include anaphylaxis-like toxicity.

Some such treatment regimens can include, for example, a first subcutaneous or intravenous slow-infusion administration of an immune stimulatory conjugate, such as those disclosed herein, so as to elicit an initial targeted immune response as desired, against a particular target, such as a tumor cell or population of cells exhibiting an epitope to which an immune stimulatory compound is targeted through specific binding of the antibody construct of the immune stimulatory conjugate

The treatment regimens then can include, for example, a second administration of an immune stimulatory conjugate through subcutaneous or intravenous slow-infusion administration to spare or alleviate toxicity(ies) associated with intravenous administration of the immune-stimulatory conjugate, while the immune stimulatory conjugate effects its targeted immune stimulatory effect at a particular site of a disease or disorder or cells thereof. As disclosed herein, such as second administration comprises subcutaneous or intravenous slow-infusion administration of the immune stimulatory conjugate. As disclosed in the examples below, it is observed that subcutaneous or intravenous slow-infusion administration of a second dose of an immune stimulatory conjugate to a subject having already received a first dose of the immune stimulatory conjugate alleviates, reduces or, in some cases, reduces or minimizes toxicities associated with bolus repetitive intravenous administration of the conjugate, characterized by an anaphylaxis-like response/toxicity that is often deleterious to the subject.

In various treatment regimens disclosed herein, subcutaneous or intravenous slow-infusion administration of a second dose is incorporated into a treatment regimen comprising prior administration of a first dose of the immune stimulatory conjugate by subcutaneous administration. In these cases, the terms ‘first’ and ‘second’ dose are intended to indicate timing of administration relative to one another, but do not necessarily indicate timing or relative position of a dose in a treatment regimen overall.

Second dose subcutaneous or intravenous slow-infusion administration is temporally distinct from a one or more ‘first’ administrations, such that a first dose or cycle of an immune stimulatory conjugate is separated by, for example, days or longer, is followed by a longer duration before administration of a second dose through subcutaneous or intravenous slow-infusion delivery. A second and subsequent subcutaneous or intravenous slow-infusion administration is often part of a regular series of administration events, comprising a first subcutaneous or intravenous slow-infusion administration and subsequent administrations such as subcutaneous or intravenous slow-infusion dosing at regular or irregular intervals.

In some embodiments, B cells are deplated prior to administration of the immune-stimulatory conjugate. In some embodiments, an immune stimulatory conjugate is administered with a B-cell depleting agent. The B-cell depleting agent may be administered prior to, at the same time as, or after the immune stimulatory conjugate. The B-cell depleting agent may be administered, for example, within 14 days, within 7 days, within 1 day, within 24, 12, 6, 4, 3, 2, or 1 hour of the first administration of the immune-stimulatory conjugate. B-cell depleting agents include, but are not limited to, anti-CD20 antibodies, anti-CD19 antibodies, anti-CD22 antibodies, anti-BLyS antibodies, TACI-Ig, BR3-Fc, and anti-BR3 antibodies. Nonlimiting exemplary B-cell depleting agents include rituximab, ocrelizumab, ofatumumab, epratuzumab, MEDI-51 (anti-CD19 antibody), belimumab, BR3-Fc, AMG-623, and atacicept.

In some embodiments, the immune-stimulatory conjugate is administered with an agent that mitigates an anaphylactic-like toxicity. Nonlimiting exemplary agents that mitigate an anaphylactic-like toxicity include epinephrine, an antihistamine, a cortisone, and a beta-agonist. Administration may be, for example, within 1 hour or within minutes of administration of the immune-stimulatory conjugate.

Methods of administration as disclosed herein are consistent with the use of a broad range of immune-stimulatory conjugates of immune-stimulatory compounds attached to antibody constructs or other targeting moieties. In particular, the methods disclosed herein are well suited for use with immune stimulatory conjugates, such as immune stimulatory conjugates that direct an immune response in a subject to a particular disorder or disease location, cell type or cell. Accordingly, practice of some methods herein comprises selection of a suitable subject such as a subject to be subjected to or undergoing an treatment with an immune stimulatory conjugate that directs an immune stimulatory compound of the conjugate to a particular disorder or disease site, cell type or cell. Often, the subject is selected for practice of the method due to having at least one symptom of a disease or disorder, or projected to develop at least one symptom of a disease or disorder (such as a subject in remission and at risk for relapse), suitable for treatment by an immune stimulatory conjugate as disclosed herein. Some diseases are selected not based upon or not based solely on disease type, but upon detection or presence of a suitable epitope on a tumor, cell type or particular cell that facilitates localization of an immune-stimulatory conjugate to the epitope.

Subcutaneous administration of immune stimulatory conjugate or slow IV infusion administration consistent with the disclosure herein is performed so as to spare or alleviate toxicities or avoid toxicities associated with repetitive bolus intravenous administration of the conjugate, such as an anaphylaxis-like response. A number of timing regimens are consistent with the second dose administration following first dose administration, such as administration of a second dose no more than 7, 8, 9, 10, 11, 12, 13, 14, 15, 16, 17, 18, 19, 20, 21, 22, 23, or 24 days after a first dose. Alternately, some dosage regimens comprise subcutaneous administration of a second dose at least 1, 2, 3, 4, 5, 6, 7, 8, 9, 10, 11, 12, 13, 14, 15, 16, 17, 18, 19, 20, 21, 22, 23, or 24 days after administration of a first dose.

Similarly, a number of dosage amounts are consistent with the methods disclosed herein. Typically, administration of a second dose and subsequent doses are at a level about or the same as that of a first dose. A second dose can variously greater than, equal to or less than a first dose. For example, a second dose is selected so as to be at least 10%, at least 20%, at least 30%, at least 40%, at least 50%, at least 60%, at least 70%, at least 80%, at least 90%, or at least 95% of a first dose. Alternately, a second dose is selected so as to be at most 10%, at most 20%, at most 30%, at most 40%, at most 50%, at most 60%, at most 70%, at most 80%, at most 90%, or at most 95% of a first dose. Similarly, a second dose is, in some cases, selected to be greater than a first dose, such as at least 125%, at least 150%, at least 200%, at least 300%, or at least 400% of a first dose. Similarly, a second dose is, in some cases, selected to be greater than a first dose, such as at most 125%, at most 150%, at most 200%, at most 300%, or at most 400% of a first dose.

Dosage is often determined for a subject relative to an attribute of the subject, such as subject weight. Exemplary dosage amounts (e.g., subcutaneous dosage amounts) range from less than 1 mg/kg to 1, 2, 3, 4, 5, 6, 7, 8, 9, to 10, and also contemplate values intermediate to those listed in the aforementioned range of values. Exemplary doses in various treatment regimens include, for example, a first 1 mg/kg dose, a second 1 mg/kg dose administered three weeks later on day 21 and subsequent 1 mg/kg doses administered every three weeks; a first 2 mg/kg dose, a second 2 mg/kg dose administered three weeks later on day 21 and subsequent 2 mg/kg doses administered every three weeks; a first 3 mg/kg dose, a second 3 mg/kg dose administered three weeks later on day 21 and subsequent 3 mg/kg doses administered every three weeks; a first 4 mg/kg dose, a second 4 mg/kg dose administered three weeks later on day 21 and subsequent 4 mg/kg doses administered every three weeks; or a first 5 mg/kg dose, a second 5 mg/kg dose administered three weeks later on day 21 and subsequent 5 mg/kg doses administered every three weeks; in PBS buffer or other pharmaceutical formulation, suitable for subcutaneous or intravenous slow-infusion administration. Additional exemplary doses in various treatment regimens include, for example, a first 1 mg/kg dose, a second 1 mg/kg dose administered two weeks later on day 14 and subsequent 1 mg/kg doses administered every two weeks; a first 2 mg/kg dose, a second 2 mg/kg dose administered two weeks later on day 14 and subsequent 2 mg/kg doses administered every two weeks; a first 3 mg/kg dose, a second 3 mg/kg dose administered two weeks later on day 14 and subsequent 3 mg/kg doses administered every two weeks; a first 4 mg/kg dose, a second 4 mg/kg dose administered two weeks later on day 21 and subsequent 4 mg/kg doses administered every two weeks; and a first 5 mg/kg dose, a second 5 mg/kg dose administered two weeks later on Day 14 and subsequent 5 mg/kg doses administered every two weeks; in PBS buffer or other pharmaceutical formulation suitable for subcutaneous or intravenous slow-infusion administration. Other exemplary doses in various treatment regimens include, for example, a first 1 mg/kg dose, a second 1 mg/kg dose administered four weeks later on day 28 and subsequent 1 mg/kg doses administered every four weeks; a first 2 mg/kg dose, a second 2 mg/kg dose administered four weeks later on day 28 and subsequent 2 mg/kg doses administered every four weeks; a first 3 mg/kg dose, a second 3 mg/kg dose administered four weeks later on day 28 and subsequent 3 mg/kg doses administered every four weeks; a first 4 mg/kg dose, a second 4 mg/kg dose administered four weeks later on day 28 and subsequent 4 mg/kg doses administered every four weeks; and a first 5 mg/kg dose, a second 5 mg/kg dose administered four weeks later on Day 28 and subsequent 5 mg/kg doses administered every four weeks; in PBS buffer or other pharmaceutical formulation suitable for subcutaneous or intravenous slow-infusion administration. One of skill in the art understands that alternatives within these ranges or outside of these ranges but differing by, for example, no greater than 10%, no greater than 20%, no greater than 30%, no greater than 40%, or no greater than 50% from the upper or lower values in these ranges are also contemplated.

Methods disclosed herein often comprise monitoring a subject following administration of a first dose, a second dose, or one or more additional doses. A number of monitoring approaches are consistent with the disclosure herein. Monitoring is generally directed toward detection of at least one symptom or adverse event or at least one indicator of an increased risk of an anaphylaxis-like response. Exemplary monitoring comprises at least one monitoring process selected from a list comprising monitoring blood cell count, body temperature, skin discoloration, subject alertness or other indicator of anaphylaxis-like response.

Administration regimens as disclosed herein optionally comprise a ‘test dose’ prior to or subsequent to a second dose such as the second subcutaneous or intravenous slow-infusion dose described above. A test dose comprises administration of an immune stimulatory conjugate at a level below a selected level suitable of elicitation of a targeted immune stimulatory effect of the conjugate but sufficient to be indicative of an anaphylaxis-like response that may arise from, for example, administration of a second dose by intravenous administration. Administration of a test dose is often accompanied by monitoring of the subject for symptoms such as change in body temperature, breathing, heart rate or blood pressure, or other indication disclosed herein or otherwise associated with an anaphylaxis-like response.

Accordingly, methods herein one or more of the elements of selecting a subject in need of an immune-stimulatory treatment directed to an antigen such as a tumor antigen, administering a dosage regimen comprising subcutaneous or intravenous slow-infusion administration of an immune-stimulatory conjugate, monitoring for a response such as an anaphylaxis-like response, and observing alleviation of at least one symptom associated with a disorder.

In various cases, the immune-stimulatory conjugate comprises benzazepine. In some cases, the immune-stimulatory conjugate is a TLR8 agonist. In certain embodiments, the TLR8 agonist is benzazepine, an imidazoquinoline, a thiazoloquinoline, an aminoquinoline, an aminoquinazoline, a pyrido[3,2-d]pyrimidine-2,4-diamine, a pyrimidine-2,4-diamine, a 2-aminoimidazole, an 1-alkyl-1H-benzimidazol-2-amine, a tetrahydropyridopyrimidine, a pyrido[3,2-d]pyrimidine, a dihydropyrimidinyl benzazepine carboxamide, a benzo[b]azepine, benzazepine dicarboxamide derivatives with a tertiary amide, benzazepine dicarboxamide derivatives with a secondary amide, a quinazoline, a pyrido[3,2-d]pyrimidine, a diamino-pyrimidine, an amino-quinazoline, a heterocyclic-substituted 2-amino-quinazoline, a diamino-pyrimidine, a piperidino-pyrimidine, an alkylamino-pyrimidine, an 8-substituted benzoazepine, an amino-diazepine, an amino-benzo-diazepine, an amido-indole, an amido-benzimidazole, a phenyl sulfonamide, a dihydropteridinone, a fused amino-pyrimidine, a quinazoline, a pyrido-pyrimidine, an amino-substituted benzazepine, a pyrrolo-pyridine, an imidazo-pyridine derivatives, an amino-benzazepine, and a ssRNA. In certain embodiments, a TLR8 agonist is selected from the group consisting of a benzazepine, an imidazoquinoline, a thiazoloquinoline, an aminoquinoline, an aminoquinazoline, a pyrido[3,2-d]pyrimidine-2,4-diamine, a pyrimidine-2,4-diamine, a 2-aminoimidazole, an 1-alkyl-1H-benzimidazol-2-amine, a tetrahydropyridopyrimidine, a pyrido[3,2-d]pyrimidine, a dihydropyrimidinyl benzazepine carboxamide, a benzo[b]azepine, benzazepine dicarboxamide derivatives with a tertiary amide, benzazepine dicarboxamide derivatives with a secondary amide, a quinazoline, a pyrido[3,2-d]pyrimidine, a diamino-pyrimidine, an amino-quinazoline, a heterocyclic-substituted 2-amino-quinazoline, a diamino-pyrimidine, a piperidino-pyrimidine, an alkylamino-pyrimidine, an 8-substituted benzoazepine, an amino-diazepine, an amino-benzo-diazepine, an amido-indole, an amido-benzimidazole, a phenyl sulfonamide, a dihydropteridinone, a fused amino-pyrimidine, a quinazoline, a pyrido-pyrimidine, an amino-substituted benzazepine, a pyrrolo-pyridine, an imidazo-pyridine derivatives, and an amino-benzazepine, and is other a ssRNA. In some embodiments, a TLR8 agonist is a non-naturally occurring compound. Examples of TLR8 agonists include motolimod, resiquimod, 3M-051, 3M-052, MCT-465, IMO-4200, VTX-763, VTX-1463, and the TLR8 modulator compounds disclosed in US20180086755 (Gilead, pyrido[3,2-d]pyrimidine derivatives), WO2017216054 (Roche, dihydropyrimidinyl benzazepine carboxamide derivatives), WO2017190669 (Shanghai De Novo Pharmatech, benzo[b]azepine derivatives), WO2016142250 (Roche, benzazepine dicarboxamide derivatives), WO2017202704 (Roche, benzazepine dicarboxamide derivatives with a tertiary amide), WO2017202703 (Roche, benzazepine dicarboxamide derivatives with a secondary amide), US20170071944 (Gilead, quinazoline and pyrido[3,2-d]pyrimdine derivatives), US20140045849 (Janssen, diamino-pyrimidine derivatives), US20140073642 (Janssen, amino-quinazoline derivatives), WO2014056953 (Janssen, pyrrolo[3,2-d]pyrimidine derivatives), WO2014076221 (Janssen, heterocyclic substituted 2-amino-quinazoline derivatives), WO2014128189 (Janssen, diamino-pyrimidine derivatives), US20140350031 (Janssen, piperidino-pyrimidine derivatives), WO2014023813 (Janssen, alkyl-aminopyrimidine derivatives), US20080234251 (Array Biopharma, 8-substituted benzoazepine derivatives), US20080306050 (Array Biopharma, amino-diazepine derivatives), US20100029585 (VentiRx Pharma, amino-benzazepine derivatives), US20110092485 (VentiRx Pharma, amino-benzazepine derivatives), US20110118235 (VentiRx Pharma, amino-benzazepine derivatives), US20120082658 (VentiRx Pharma, amino-benzazepine VTX-378), US20120219615 (VentiRx Pharma), US20140066432 (VentiRx Pharma, amino-benzazepine VTX-2337), US20140088085 (VentiRx Pharma, amino-benzazepine and amino-benzo-diazepine derivatives), US20140275167 (Novira Therapeutics, amido-indole and amido-benzimidazole derivatives), and US20130251673 (Novira Therapeutics, phenyl sulfonamide derivatives), and these publications are incorporated by reference herein. Further examples of TLR8 modulators include compounds disclosed in US2016/0108045 (Gilead, dihydropteridinone derivatives), US2018/0065938 (Gilead, fused amino-pyrimidine derivatives), US2018/0263985 (Gilead, quinazoline and pyrido-pyrimidine derivatives), WO2017/046112 (Roche, amino-substituted benzazepine derivatives), WO2016/096778 (Roche, amino-substituted benzazepine derivatives), US2019/0016808 (Birdie Biopharmaceuticals, pyrrolo- or imidazo-pyridine derivatives or amino-benzazepine derivatives), and these publications are incorporated by reference herein. In some embodiments, the TLR8 agonist comprises the structure:

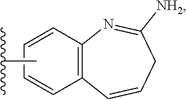

wherein the structure is optionally substituted at any position other than the —NH₂ position. In some embodiments, a TLR8 agonist has an EC50 value of 500 nM or less by PBMC assay measuring TNFalpha production. In some embodiments, a TLR8 agonist has an EC50 value of 100 nM or less by PBMC assay measuring TNFalpha production. In some embodiments, a TLR8 agonist has an EC50 value of 50 nM or less by PBMC assay measuring TNFalpha production. In some embodiments, a TLR8 agonist has an EC50 value of 10 nM or less by PBMC assay measuring TNFalpha production.

In some cases, the immune-stimulatory conjugate comprises a TLR7 agonist. In certain embodiments, the TLR7 agonist is selected from an imidazoquinoline, an imidazoquinoline amine, a thiazoquinoline, an aminoquinoline, an aminoquinazoline, a pyrido[3,2-d]pyrimidine-2,4-diamine, a pyrimidine-2,4-diamine, a 2-aminoimidazole, an 1-alkyl-1H-benzimidazol-2-amine, a tetrahydropyridopyrimidine, a heteroarothiadiazide-2,2-dioxide, a benzonaphthyridine, a thieno[3,2-d]pyrimidine, a 4-amino-imidazoquinoline, an imidazo-pyridinone, an imidazo-pyrimidinone, a purine, a fused pyrimidine-lactam, an imidazo[4,5-c]quinoline-4-amine, an imidazo[4,5-c]quinoline, a pyrimidine, a benzazepine, an imidazo-pyridine, a pyrrolo-pyrimidine, a 2-amino-quinazoline, a guanosine analog, an adenosine analog, a thymidine homopolymer, an ssRNA, CpG-A, PolyG10, and PolyG3. In certain embodiments, the TLR7 agonist is selected from an imidazoquinoline, an imidazoquinoline amine, a thiazoquinoline, an aminoquinoline, an aminoquinazoline, a pyrido[3,2-d]pyrimidine-2,4-diamine, a pyrimidine-2,4-diamine, a 2-aminoimidazole, an 1-alkyl-1H-benzimidazol-2-amine, a tetrahydropyridopyrimidine, a heteroarothiadiazide-2,2-dioxide, a benzonaphthyridine, a thieno[3,2-d]pyrimidine, a 4-amino-imidazoquinoline, an imidazo-pyridinone, an imidazo-pyrimidinone, a purine, a fused pyrimidine-lactam, an imidazo[4,5-c]quinoline-4-amine, an imidazo[4,5-c]quinoline, a pyrimidine, a benzazepine, an imidazo-pyridine, a pyrrolo-pyrimidine, and a 2-amino-quinazoline, but is other than a guanosine analog, an adenosine analog, a thymidine homopolymer, an ssRNA, CpG-A, PolyG10, and PolyG3. In some embodiments, a TLR7 agonist is a non-naturally occurring compound. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the TLR7 modulator compounds disclosed in US20160168164 (Janssen, thieno[3,2-d]pyrimidine derivatives), US 20150299194 (Roche, 4-amino-imidazoquinoline derivatives), US20110098248 (Gilead Sciences, imidazo-pyridinone, imidazo-pyrimidinone, and purines derivatives), US20100143301 (Gilead Sciences, fused pyrimidine-lactam derivatives), and US20090047249 (Gilead Sciences, purine derivatives), and these publications are incorporated by reference herein. Further examples of TLR7 modulators include compounds disclosed in WO2018/009916 (Stanford University/Bolt Biotherapeutics, imidazo[4,5-c]quinolin-4-amine derivatives), WO2018/112108 (Bolt Biotherapeutics, imidazo[4,5-c]quinoline, pyrimidine, benzazepine, imidazo-pyridine, pyrrolo-pyrimidine, and purine derivatives), US2019/0055247 (Bristol-Myers Squibb, purine derivatives), WO2018/198091 (Novartis, pyrrolo-pyrimidine derivatives), US2017/0121421 (Novartis, pyrrolo-pyrimidine derivatives), U.S. Pat. No. 10,253,003 (Janssen, 2-amino-quinazoline derivatives), and U.S. Pat. No. 10,233,184 (Roche, imidazo-pyrimidinone derivatives), and these publications are incorporated by reference herein. In some embodiments, a TLR7 agonist has an EC50 value of 500 nM or less by PBMC assay measuring TNFalpha or IFNalpha production. In some embodiments, a TLR7 agonist has an EC50 value of 100 nM or less by PBMC assay measuring TNFalpha or IFNalpha production. In some embodiments, a TLR7 agonist has an EC50 value of 50 nM or less by PBMC assay measuring TNFalpha or IFNalpha production. In some embodiments, a TLR7 agonist has an EC50 value of 10 nM or less by PBMC assay measuring TNFalpha or IFNalpha production.

Other immune-stimulatory compounds disclosed elsewhere herein are also consistent with the methods disclosed herein.

Immune-stimulatory compounds in some cases comprise an antibody or antibody domain as disclosed elsewhere herein.

In some cases, alleviation of at least one symptom associated with the disorder comprises reduced tumor growth. In some cases, alleviation of at least one symptom associated with the disorder comprises tumor arrest.

### General Synthetic Schemes and Examples

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

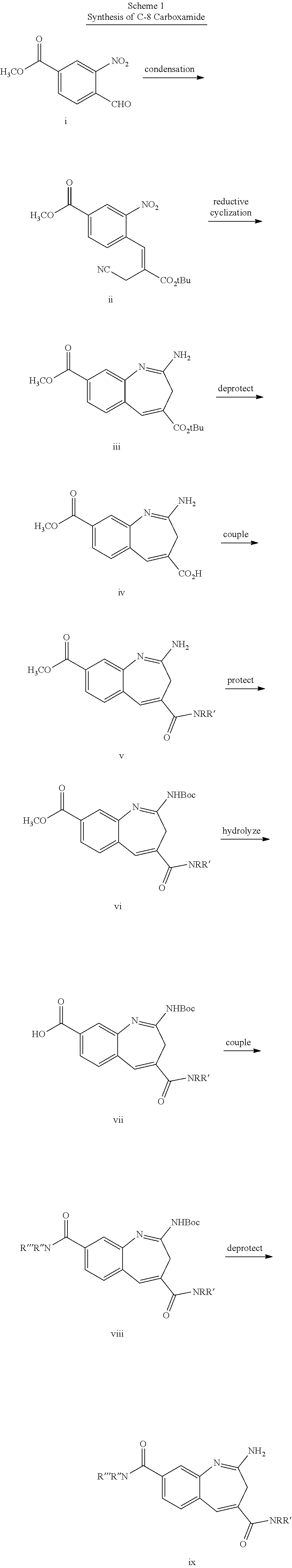

React an aldehyde (i) with an appropriately Wittig reagent, such as tert-butyl 3-cyano-2-(triphenylphosphorylidene)propanoate, at elevated temperatures to afford an olefin (ii), which undergoes reductive cyclization by treating the olefin (ii) with a reducing agent, such as iron powder in hot acetic acid, to afford azepines (iii). Deprotect the C-4 ester group by using a strong acid such as HCl to give compounds (iv), which is in turn coupled with a substituted amine using a coupling agent, such as BOP reagent. Protect the 2-amino substituent of compounds (v) with a tert-butoxycarbonyl group. Hydrolyze the resulting compounds (vi) with reagents such as LiOH in a mixture of THF and methanol to afford compounds (vii). Convert the C-8 carboxylic acid of (vii) to the amide group using known reagents such as HBTU and a tertiary amine base. Acid-mediated deprotection of compounds (viii) using a reagent such as TFA in dichloromethane provides the target compounds (ix).

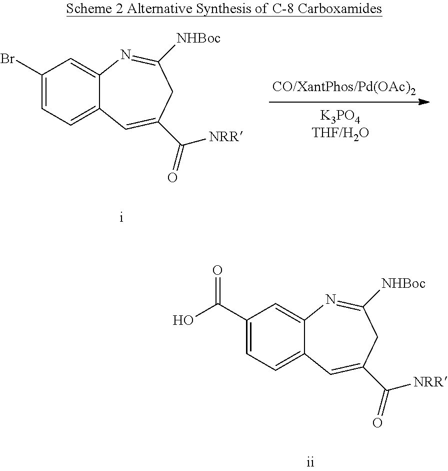

React (i) under standard conditions used for the carbonylation of aryl halides such as carbon monoxide, a palladium catalyst such as Pd(OAc)₂ and a ligand such as 4,5-bis(diphenylphosphino)-9,9-dimethylxanthene (XantPhos) and a base such as potassium phosphate in a mixture of THF and water to provide carboxylic acids (ii). Conversion to final products can then be carried out in a manner similar to that described in Scheme 1 (vii→ix).

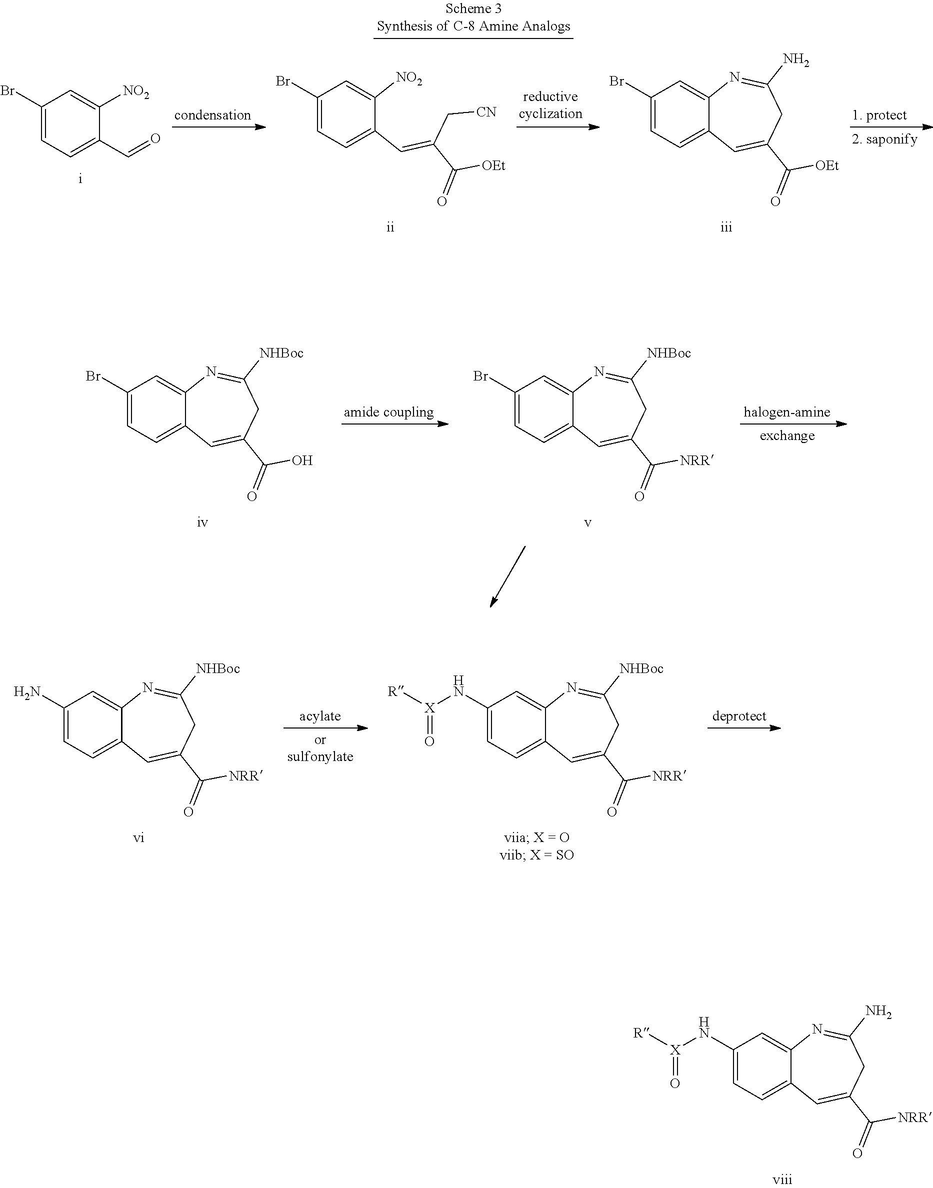

React an aldehyde (i) with an appropriately Wittig reagent, such as ethyl 3-cyano-2-(triphenylphosphorylidene)propanoate, at ambient temperature to afford an olefin (ii), which undergoes reductive cyclization by treating the olefin (ii) with a reducing agent, such as iron powder in hot acetic acid, to afford azepines (iii). Protect the C-2 amine group by using Boc anhydride to give compounds (iii), which is in turn saponified with an alkaline metal hydroxide such as LiOH to afford the carboxylic acid which is coupled with a substituted amine using a coupling agent, such as BOP reagent to provide compounds (iv). Convert the C-8 carboxylic acid of (v) to the amide group using known reagents such as EDCI/HOBT and a tertiary amine base. Halogen-amine exchange can be effected using standard methodology such as copper-mediated or palladium-catalyzed couplings (benzophenone imine/Pd(II)) to provide C-8 anilines (vi). Functionalization of amines (vi) by acylation or sulfonylation provides anilides (X═C) or sulfonamides (X═SO) compounds (vii). Alternatively, compounds (vii) can be prepared directly through a palladium-mediated coupling of bromide (v) and an appropriately substituted amide or sulfonamide. Acid-mediated deprotection of compounds (vii) using a reagent such as TFA in dichloromethane provides the target compounds (viii).

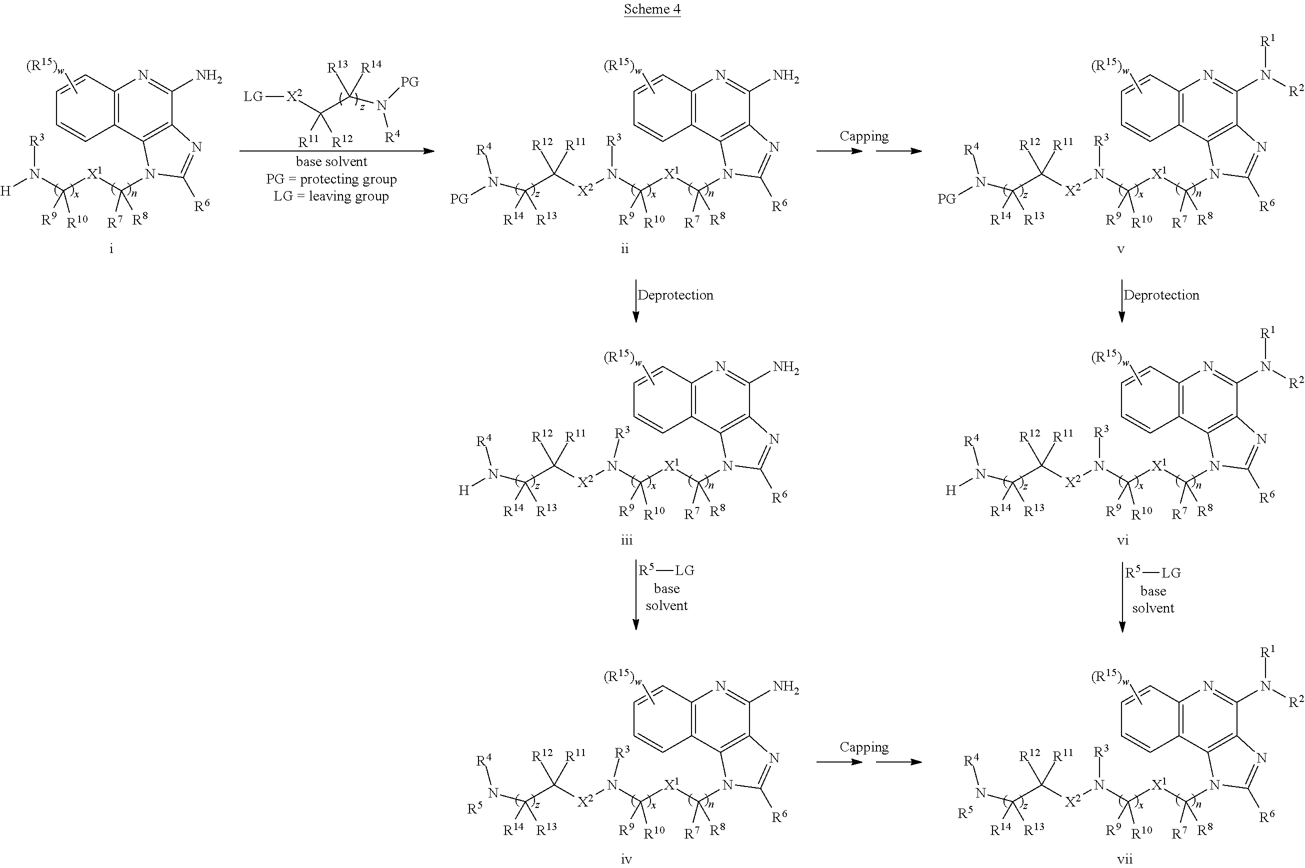

A 4-amino imidazoquinoline (i) with a pendent amino-functionality may be acylated, or alkylated, when treated with an appropriate electrophile in the presence of an appropriate base in an appropriate solvent, to give compounds of type (ii). Subsequent deprotection of a protecting group (PG), if applicable, results in the generation of compound (iii), containing a free amine which may be functionalized in an analogous fashion to the first step of this sequence (i→ii). Alternatively, the 4-amino compound (ii) may be capped via treatment with an appropriate electrophile to provide access to compounds of type (v). Compounds of type (v) can be converted to compounds of type (vii) just as compounds of type (ii) are converted to (iv). In some instances, compounds of type (iv) may be modified directly to access compounds of type (vii), via treatment with an appropriate electrophile in the presence of an appropriate base in an appropriate solvent.

### Scheme 5

### Synthesis of Linker-Payloads

A linker-payload (LP) can be synthesized by various methods. For example, LP compounds can be synthesized as shown in Scheme 5-1.

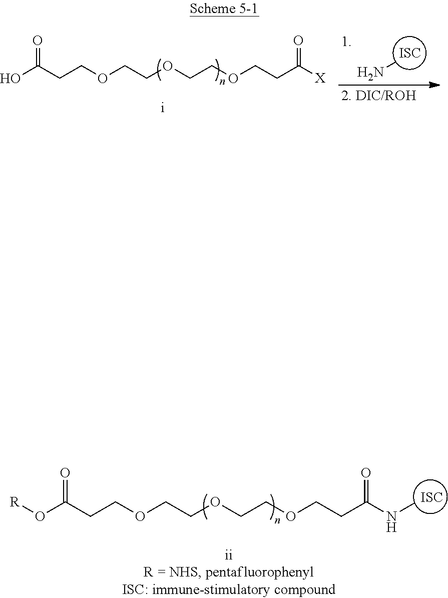

A PEGylated carboxylic acid (i) that has been activated for amide bond formation can be reacted with an appropriately substituted amine containing immune-stimulatory compound to afford an intermediate amide. Formation of an activated ester (ii) can be achieved by reaction the intermediate amide-containing carboxylic using a reagent such as N-hydroxysuccinimide or pentafluorophenol in the presence of a coupling agent such as diisopropylcarbodiimide (DIC) to provide compounds (ii).

An LP can be synthesized as shown in Scheme 5-2.

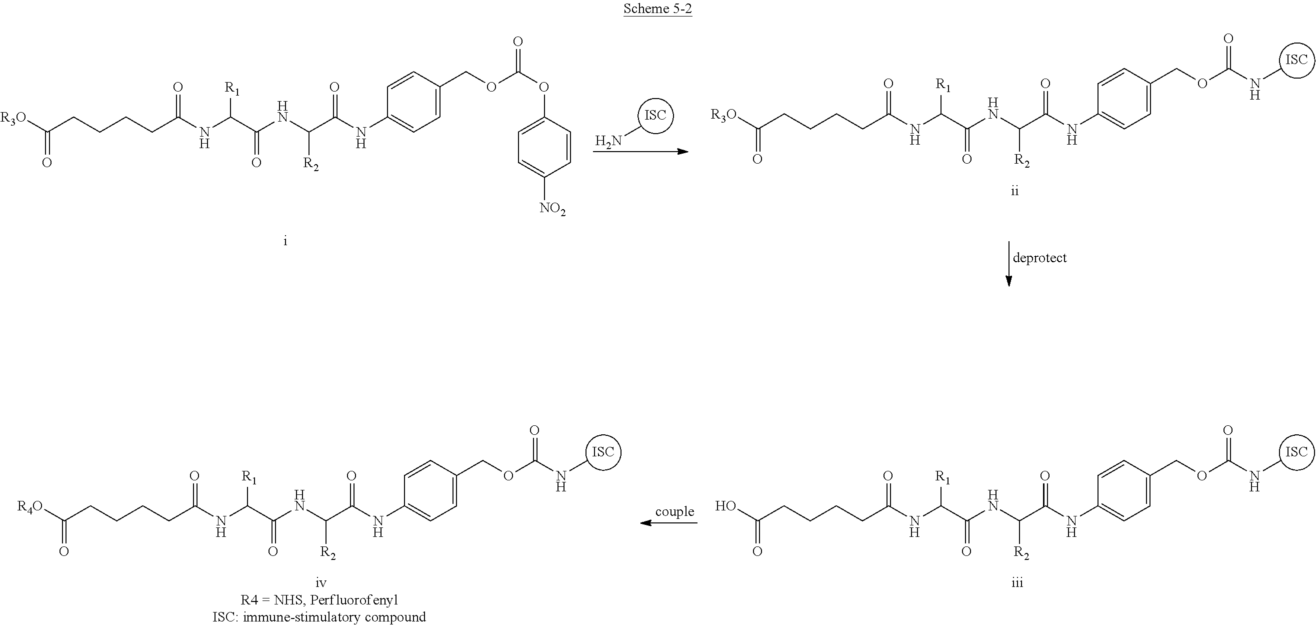

An activated carbonate such as (i) can be reacted with an appropriately substituted amine containing immune-stimulatory compound to afford carbamates (ii) which can be deprotected using standard methods based on the nature of the R₃ ester group. The resulting carboxylic acid (iii) can then by coupled with an activating agent such as N-hydroxysuccinimide or pentafluorophenol to provide compounds (iv).

An LP compound can be synthesized as shown in Scheme 5-3.

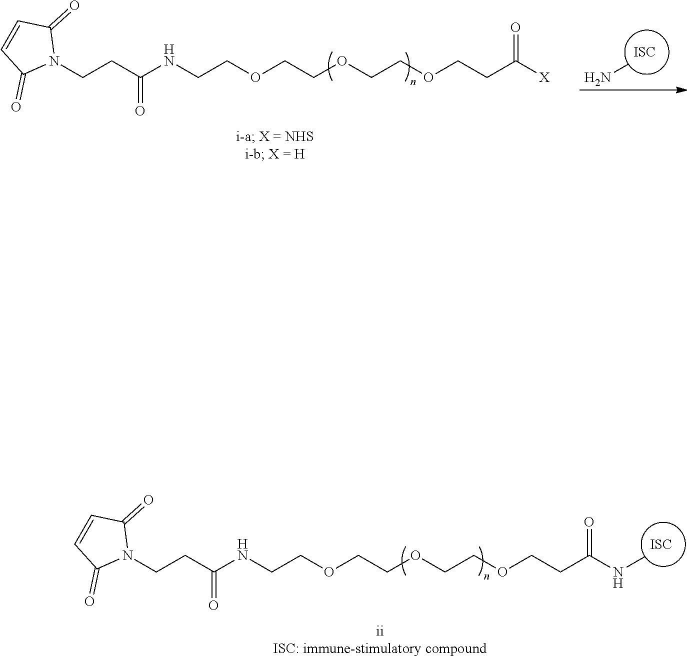

An activated carboxylic ester such as (i-a) can be reacted with an appropriately substituted amine containing immune-modstimulatory compound to afford amides (ii). Alternatively, carboxylic acids of type (i-b) can be coupled to an appropriately substituted amine containing immune-stimulatory compound in the presence of an amide bond forming agent such as dicyclohexycarbodiimde (DCC) to provide the desired LP.

An LP compound can be synthesized by various methods such as that shown in Scheme 5-4.

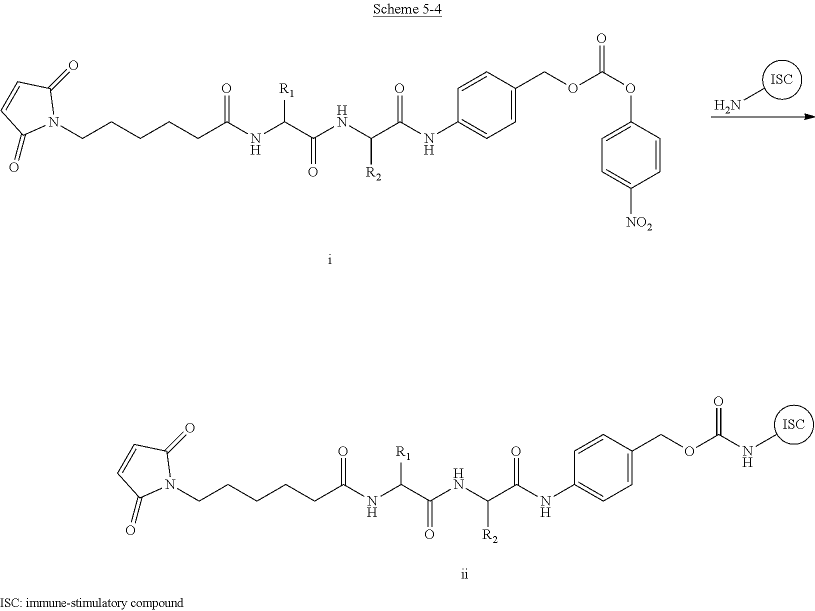

An activated carbonate such as (i) can be reacted with an appropriately substituted amine containing immune-modstimulatory compound to afford carbamates (ii) as the target ISC.

An LP compound can also be synthesized as shown in Scheme 5-5.

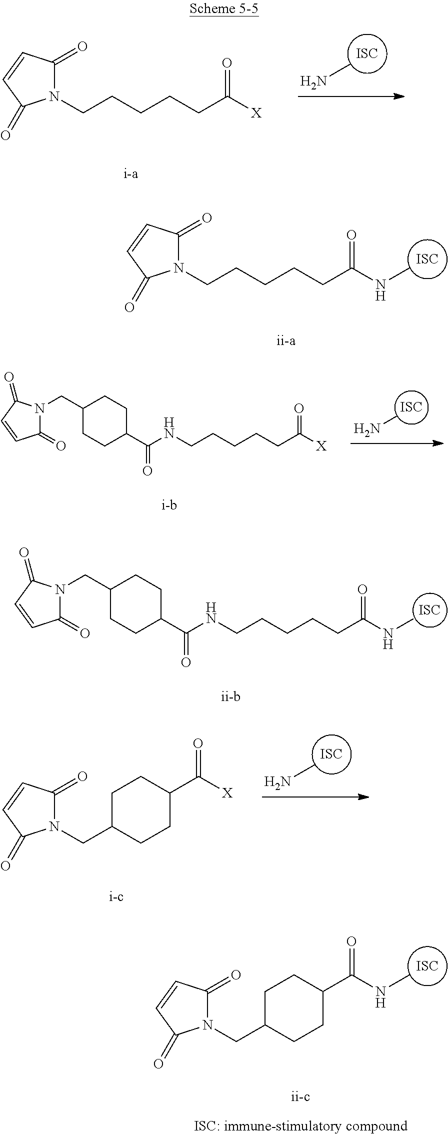

An activated carboxylic acid such as (i-a, i-b, i-c) can be reacted with an appropriately substituted amine containing immune-stimulatory compound to afford amides (ii-a, ii-b, ii-c) as the target linkered payloads (LPs).

Further understanding of the disclosure is found through reference to the following numbered embodiments. 1. A method for alleviating unwanted toxicity associated with intravenous administration of an immune-stimulatory conjugate, comprising: subcutaneously administering to a subject in need thereof an effective regimen of an immune-stimulatory conjugate comprising (a) a targeting moiety that specifically binds to a tumor antigen or a tumor associated antigen and (b) an immune-stimulatory compound; whereby a toxicity of intravenous administration of the conjugate is alleviated, as compared with intravenous administration of the conjugate, and the toxicity is selected from a hematopoietic toxicity, an anaphylaxis-like toxicity and cytokine release syndrome. 2. A method for alleviating an adverse event associated with intravenous administration of an immune-stimulatory conjugate, comprising: subcutaneously administering to a subject in need thereof an effective regimen of an immune-stimulatory conjugate comprising (a) a targeting moiety that specifically binds to a tumor antigen or a tumor associated antigen and (b) an immune-stimulatory compound; whereby a hematopoietic toxicity, an anaphylaxis-like toxicity or cytokine release syndrome associated with intravenous administration of the conjugate is spared in the subject. 3. A method for increasing the tolerability of treatment with an immune activating conjugate, comprising: subcutaneously administering to a subject in need thereof an effective regimen of an immune-stimulatory conjugate comprising (a) a targeting moiety that specifically binds to a tumor antigen or a tumor associated antigen and (b) an immune-stimulatory compound; wherein a total dose administered in the effective regimen is greater than a tolerated dose of the conjugate by intravenous administration and whereby development of hematopoietic toxicity, an anaphylaxis-like toxicity or cytokine release syndrome is spared in the subject, as compared with intravenous administration of the conjugate. 4. The method of any of embodiments 1-3, wherein the conjugate is represented by Formula (I):

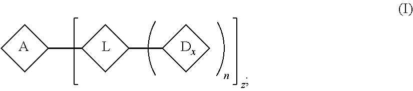

wherein: A is the targeting moiety, optionally an antibody construct having at least one antigen binding domain and an Fc domain, L is a linker; Dₓ is the immune-stimulatory compound; n is selected from 1 to 20; and z is selected from 1 to 20.5. The method of embodiment 4, wherein the antigen binding domain specifically binds to a tumor antigen. 6. The method of any one of embodiments 1-5, wherein the tumor antigen is a sarcoma antigen or a carcinoma antigen. 7. The method of any one of embodiments 1-6, wherein the tumor antigen is a carcinoma antigen. 8. The method of embodiment 7, wherein the carcinoma antigen is selected from the group consisting of HER2, TROP2, LIV-1, MUC16, CEACAM1, CEACAM3, CEACAM4, CEACAM5, CEACAM6, CEACAM7, CEACAM8, CEACAM16, CEACAM18, CEACAM19, CEACAM20, CEACAM21, URLC10, NY-ESO-1, GAA, OFA, cyclin B1, WT-1, CEF, VEGRR1, VEGFR2, TTK, MUC1, HPV16E7, CEA, IMA910, KOC1, SL-701, MART-1, gp100, tyrosinase, GSK2302050A, survivin, MAGE-3.1, MAGE-10.A2, OVA BiP, gp209-2M, melan-A, NA17.A2, KOC1, CO16, DEPDC1, MPHOSPH1, MAGE12, ONT-10, GD2L, GD3L, GSK2302032A, URLC10, CDCA1, TF, rsPSMA, PSA, MUC-2, TERT, HPV16, HPV18, STF-II, G17DT, ICT-107, Dex2, hTERT, PAP, and tyrosinase related peptide 2 (TRP2). 9. The method of any one of embodiments 1-6, wherein the tumor antigen is a sarcoma antigen. 10. The method of embodiment 9, wherein the sarcoma antigen is selected LRRC15. 11. The method of any one of embodiments 1-6 wherein the tumor antigen is a selected from one of the following antigens: (i) an antigen present on lung cancer selected from the group consisting of mesothelin, HER2, EGFR, PD-L1, MSLN, LY6K, CD56, PTK7, FOLR1, DLL3, SLC34A2, CECAM5, MUC16, LRRC15, ADAM12, EGFRvIII, LYPD3, EFNA4 and MUC1; (ii) an antigen present on liver cancer selected from the group consisting of GPC3, EPCAM, CECAM5; (iii) an antigen present on kidney cancer selected from the group consisting of HAVCR1, ENPP3, CDH6, CD70, and cMET; (iv) an antigen present on pancreatic cancer selected from the group consisting of PTK7, MUC16, MSLN, LRRC15, ADAM12, EFNA4, MUC5A and MUC1; (v) an antigen present on colorectal cancer selected from the group consisting of EPHB2, TMEM238, CECAM5, LRRC15, ADAM12, EFNA4 and GPA33; (vi) an antigen present on ovarian cancer selected from the group consisting of MUC16, MUC1, MSLN, FOLR1, sTN, VTCN1, HER2, PTK7, FAP, TMEM238, LRRC15, CLDN6, SLC34A2 and EFNA4; (vii) an antigen present on head and neck cancer selected from the group consisting of LY6K, PTK7, LRRC15, ADAM12, LYPD3, EFNA4 and TNC; (viii) an antigen present on bone cancer selected from the group consisting of EPHA2, LRRC15, ADAM12, GPNMB, TP-3 and CD248; (ix) an antigen present on mesothelioma, MSLN; (x) an antigen present on bladder cancer selected from the group consisting of LY6K, PTK7, UPK1B, UPK2, TNC, Nectin4, SLITRK6, LYPD3, EFNA4 and HER2; (xi) an antigen present on stomach cancer selected from the group consisting of HER2, EPHB2, TMEM238, CECAM5 and EFNA4; (xii) an antigen present on prostate cancer selected from the group consisting of PSMA, FOLH1, PTK7, STEAP, TMEFF2 (TENB2), OR51E2, SLC30A4 and EFNA4; (xiii) an antigen present on thyroid cancer, PTK7; (xiv) an antigen present on uterine cancer selected from the group consisting of LY6K, PTK7, EPHB2, FOLR1, ALPPL2, MUC16 and EFNA4; (xv) an antigen present on cervical/endometrial cancer selected from the group consisting of LY6K, PTK7, MUC16, LYPD3, EFNA4 and MUC1; and (xvi) an antigen present on breast cancer selected from the group consisting of HER2, TROP2, LIV-1, CDH3 (p-cadherin), MUC1, Sialo-epitope CA6, PTK7, GPNMB, LAMP-1, LRRC15, ADAM12, EPHA2, TNC, LYPD3, EFNA4 and CLDN6. 12. The method of embodiment 11, wherein the tumor antigen is an antigen present on breast cancer selected from the group consisting of HER2, TROP2, LIV-1, CDH3 (p-cadherin), MUC1, Sialo-epitope CA6, PTK7, GPNMB, LAMP-1, LRRC15, ADAM12, EPHA2, TNC, LYPD3, EFNA4 and CLDN6. 13. The method of any one of embodiments 1-12, wherein the immune-stimulatory compound is a myeloid cell agonist. 14. The method of embodiment 13, wherein the myeloid cell agonist is a TLR7 agonist. 15. The method of embodiment 14, wherein the TLR7 agonist is selected from the group consisting of imidazoquinoline, an imidazoquinoline amine, a thiazoquinoline, an aminoquinoline, an aminoquinazoline, a pyrido[3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, benzonaphthyridine, and a compound of Category B Formulas (IA), (IB), and (IC). 16. The method of embodiment 14, wherein the TLR7 agonist is selected from the group consisting of GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20160168164 (Janssen), US 20150299194 (Roche), US20110098248 (Gilead Sciences), US20100143301 (Gilead Sciences), and US20090047249 (Gilead Sciences). 17. The method of embodiment 13, wherein the myeloid cell agonist is a TLR8 agonist. 18. The method of embodiment 17, wherein the TLR8 agonist is selected from the group consisting of a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, and a compound of Category A Formulas (IA), (IB), (IIA), (IIB), (IIIA), and (IIIB). 19. The method of embodiment 17, wherein the TLR8 agonist is selected from the group consisting of motolimod, resiquimod, 3M-051, 3M-052, MCT-465, IMO-4200, VTX-763, VTX-1463, and the compounds disclosed in US20180086755 (Gilead), WO2017216054 (Roche), WO2017190669 (Shanghai De Novo Pharmatech), WO2017202704 (Roche), WO2017202703 (Roche), WO20170071944 (Gilead), US20140045849 (Janssen), US20140073642 (Janssen), WO2014056953 (Janssen), WO2014076221 (Janssen), WO2014128189 (Janssen), US20140350031 (Janssen), WO2014023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics) and compounds 1.1-1.2, 1.4-1.20, 1.23-1.27, 1.29-1.46, 1.48, and 1.50-1.67. 20. The method of any one of embodiments 1-19, wherein the Fc domain is an IgG region. 21. The method of embodiment 20, wherein the Fc domain is an IgG1 Fc region. 22. The method of any one of embodiments 1-21, wherein the Fc domain is an Fc domain variant comprising one or more amino acid substitutions in an IgG region as compared to an amino acid sequence of a wild-type IgG region. 23. The method of embodiment 22, wherein the Fc domain variant has increased affinity to one or more Fcγ receptors as compared to the wild-type IgG region. 24. The method of any one of embodiments 1-23, wherein the toxicity is heme toxicity comprising a decrease in platelet or red blood cells. 25. The method of embodiment 24, wherein platelet levels do not decrease below 50,000 cells/uL following the administration of the conjugate, and preferably do not decrease below 100,000 cells/uL. 26. The method of embodiment 24, wherein red blood cell levels do not decrease below 4 million cells/uL following the administration of the conjugate. 27. The method of any one of embodiments 1-26, wherein the toxicity is anaphylaxis-like toxicity characterized by hypotension, airway constriction, hypothermia and/or vascular leak syndrome, and at least one of hypotension, airway constriction, hypothermia and/or vascular leak syndrome is reduced, as compared to intravenous administration of the conjugate. 28. The method of embodiment 27, wherein the subject does not experience heme toxicity or anaphylaxis-like toxicity greater than grade 1 following subcutaneous administration of the conjugate. 29. The method of any one of embodiments 1-28, wherein the total dose of the conjugate administered per cycle of the regimen is from about 0.5 to about 7.5 mg/kg. 30. The method of embodiment 29, wherein the total dose of the conjugate is from about 0.5 to about 5 mg/kg. 31. The method of embodiment 30, wherein the total dose of the conjugate is from about 0.5 to about 4 mg/kg. 32. The method of embodiment 31, wherein the total dose of the conjugate is from about 0.5 to about 3.5 mg/kg. 33. The method of any one of embodiments 1-32, wherein the conjugate is administered as a split dose. 34. A method of eliciting targeted immune stimulation in a subject, comprising the steps of selecting the subject for treatment that expresses a tumor antigen at the site for targeted immune stimulation; administering a first dose of an immune-stimulatory conjugate to the subject; wherein the first dose is administered subcutaneously; administering a second dose of the immune-stimulatory conjugate to the subject, wherein the second dose is administered subcutaneously; and monitoring for a toxicity associated with intravenous administration of the conjugate, and the toxicity is selected from heme toxicity, anaphylaxis-like toxicity and cytokine release syndrome; and observing a targeted immune response in the subject. 35. The method of embodiment 34, wherein the immune-stimulatory conjugate comprises an antibody construct comprising an antigen binding variable domain that specifically binds to an epitope of the tumor antigen. 36. The method of any one of embodiments 1-34, wherein the toxicity is hematopoietic toxicity or an anaphylaxis-like toxicity. 37. The method of embodiment 34, comprising administering a test dose to the subject and monitoring for a symptom of a toxicity. 38. The method of embodiment 34, wherein selecting a subject comprises identifying a target tissue in the subject presenting a tumor antigen suitable for targeting of the immune-stimulatory conjugate in the subject. 39. The method of embodiment 34, wherein the tumor antigen is a carcinoma antigen. 40. The method of embodiment 34, wherein the immune-stimulatory conjugate is administered at a dose of about 0.5 to about 7.5 mg/kg. 41. The method of embodiment 40, wherein the immune-stimulatory conjugate is administered at a dose of about about 0.5 to about 5 mg/kg. 42. The method of any one of embodiments 1-41, wherein the immune-stimulatory conjugate is administered in at least two cycles, each cycle comprising a period of two weeks, three weeks for four week and wherein the total first dose of the conjugate administered per cycle is from about 0.5 to about 7.5 mg/kg. 43. The method of embodiment 42, wherein the total dose of the conjugate administered per cycle is from about 0.5 to about 5 mg/kg. Embodiments are presented numbered but are variously related to all of the other embodiments listed as well as other elements recited herein.

### EXAMPLES

The following examples are included to further describe some embodiments of the present disclosure and should not be used to limit the scope of the disclosure.

### Example 1: Synthesis of 2-amino-N⁴,N⁴-dipropyl-N⁸-(1,2,3,4-tetrahydroquinolin-7-yl)-3H-benzo[b]azepine-4,8-dicarboxamide TFA Salt (Compound 1.1)

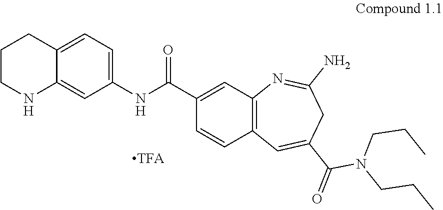 [SMILES: CCCN(CCC)C(=O)C1=Cc2ccc(C(=O)Nc3ccc4c(c3)NCCC4)cc2N=C(N)C1]

Step A: Preparation of Int 1.1a

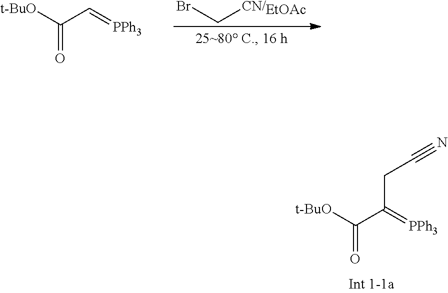

Bromoacetonitrile (8.60 g, 71.7 mmol, 4.78 mL) was added to a solution of tert-butyl (triphenylphosphorylidine)acetate (45.0 g, 119 mmol, 1.00 eq) in EtOAc (260 mL) at 25° C. The reaction was heated at 80° C. for 16 h after which time TLC (DCM:MeOH=10:1; R_{f}=0.4) and LCMS showed the reaction was complete. The mixture was cooled, filtered and washed with EtOAc (200 mL) and concentrated to afford crude Int 1.1a as a red solid which was used directly without purification.

Step B: Preparation of Int 1.1b

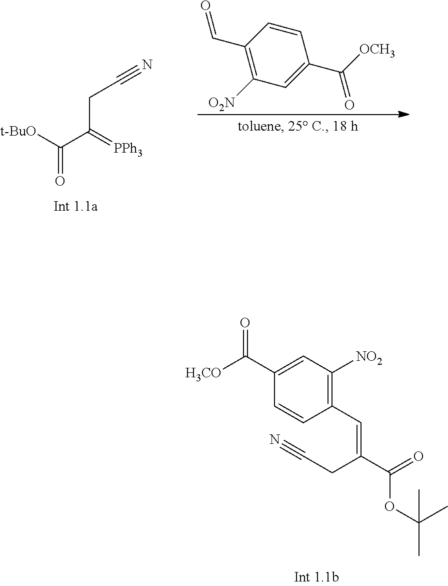

A solution of Int 1.1a (11.4 g, 54.4 mmol, 1.00 eq) and methyl 4-formyl-3-nitrobenzoate (24.8 g, 59.8 mmol, 1.10 eq) in toluene (200 mL) was stirred at 25° C. for 18 h. TLC (petroleum ether:EtOAc=1:2) showed the reaction was completed and the mixture was concentrated to afford crude product which was purified by silica gel chromatography (petroleum ether:EtOAc=10:1 to 8:1 to 4:1) to give Int 1.1b (11.3 g) as yellow solid. ¹H NMR (CDCl₃) δ 8.86 (d, J=1.3 Hz, 1H), 8.40 (dd, J=7.9, 1.3 Hz, 1H), 8.11 (s, 1H), 7.54 (d, J=7.9 Hz, 1H), 3.97-4.05 (m, 3H), 3.27 (s, 2H), 1.60 ppm (s, 9H).

Step C: Preparation of Int 1.1c

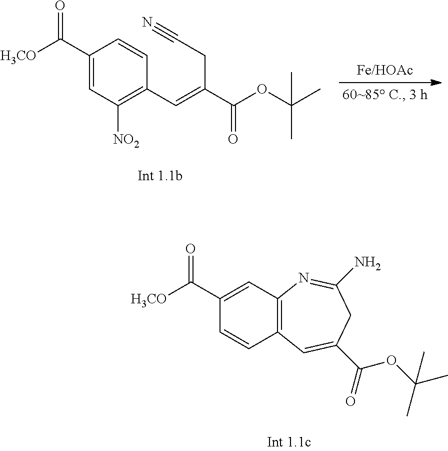 [SMILES: COC(=O)c1ccc(/C=C(\CC#N)C(=O)OC(C)(C)C)c([N+](=O)[O-])c1 ; COC(=O)c1ccc2c(c1)N=C(N)CC(C(=O)OC(C)(C)C)=C2]

Iron powder (6.79 g, 122 mmol) was added to a solution of Int 1.1b (23.4 g, 20.3 mmol, 1.00 eq) in glacial acetic acid (230 mL) at 60° C. The mixture was stirred at 85° C. for 3 h. TLC (petroleum ether:EtOAc=1:2; R_{f}=0.43) showed the reaction was completed and the mixture was cooled, filtered, washed with acetic acid (100 mL×2) and concentrated. The crude residue was diluted with EtOAc (100 mL) and washed with aq. NaHCO₃ (50 mL×3) and dried over Na₂SO₄, filtered and concentrated. The residue was purified by silica gel chromatography to afford 15.9 g of the Int 1.1c as yellow solid. ¹H NMR (CDCl₃) δ 7.95 (s, 1H), 7.76 (dd, J=8.2, 1.5 Hz, 1H), 7.70 (s, 1H), 7.46 (d, J=8.2 Hz, 1H), 3.93 (s, 3H), 2.99 (s, 2H), 1.56 (s, 9H).

Step D: Preparation of Int 1.1d

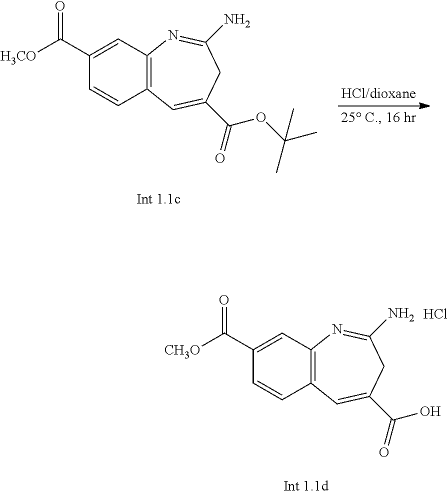

A solution of Int 1.1c (8.00 g, 25.3 mmol) in HCl/dioxane (160 mL) was stirred at 25° C. for 16 h after which time LCMS showed the reaction to be complete. The mixture was concentrated to afford 12.5 g of Int 1.1d as light yellow solid which was used directly without purification. ¹H NMR (DMSO-d₆) δ 13.43 (br s, 1H), 13.00 (br s, 1H), 10.20 (s, 1H), 9.22 (s, 1H), 7.96 (s, 1H), 7.85-7.92 (m, 2H), 7.78-7.83 (m, 1H), 3.90 (s, 3H), 3.52 (s, 2H).

Step E: Preparation of Int 1.1e

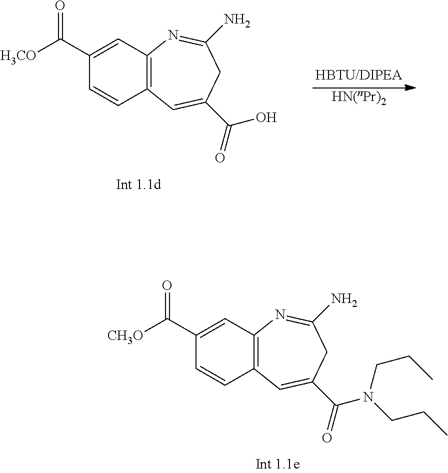

5.0 g (13.3 mmol) of HBTU and 7.7 mL (44.4 mmol) of DIPEA were added to a solution containing 3.3 g (11.1 mmol) of Int 1.1d in 60 mL of DMF at 0° C. After 5 minutes, 2.2 g (21.7 mmol) of di-n-propylamine was added and the reaction was stirred to room temperature overnight. The reaction was quenched with 20 mL of saturated NH₄Cl and then 20 mL of water. The mixture was extracted with EtOAc (3×30 mL) and the combined organic extracts were washed with brine (2×) then dried over Na₂SO₄. After removal of the drying agent and concentration of the EtOAc solution, the residue was purified on silica gel (80 g column; 0% to 20% methanol/DCM) to afford 3.0 g of Int 1.1e. ¹H NMR (CDCl₃) δ 7.92 (d, J=1.5 Hz, 1H), 7.86 (dd, J=8.2, 1.5 Hz, 1H), 7.38 (d, J=8.2 Hz, 1H), 6.89 (s, 1H), 3.92 (s, 3H), 3.39 (t, J=7.5 Hz, 4H), 3.22 (s, 2H), 1.68 (m, 4H), 0.91 (bs, 6H). ESI, m/z 343 [M+H].

Step F. Preparation of Int 1.1f

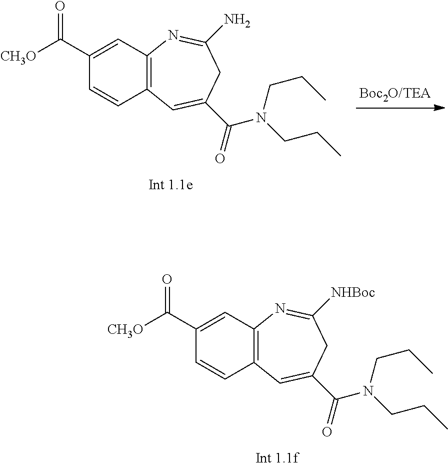

A solution containing 1.8 g (5.3 mmol) of Int 1.1e in 30 mL of dichloromethane was cooled to 0° C. and treated with 2.2 mL (7.9 mmol) of TEA and then 1.7 g (7.9 mmol) of Boc₂O. The reaction mixture was stirred to room temperature overnight and then quenched with 10 mL of water. The layers were separated and the aqueous was back extracted with dichloromethane (3×30 mL). The combined organic extracts were washed with brine and dried over Na₂SO₄. The solvent was removed and the residue was purified by silica gel chromatography (80 g column; 0% to 75% EtOAc/Hexanes) to afford the desired Int 1.1f as a white solid.

Step G: Preparation of Int 1.1g

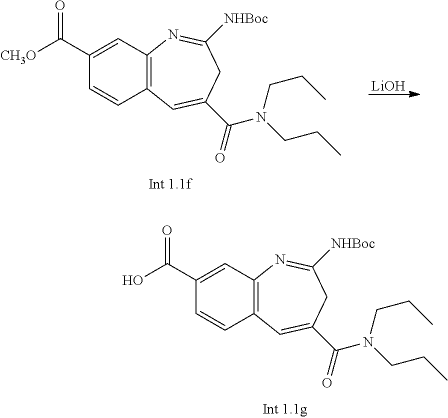

A solution containing 500 mg (1.13 mmol) of Int 1.1f in 10 mL of a 1:1 mixture of THF and water was cooled to 0° C. and treated with 1.7 mL (1.7 mmol) of 1N LiOH. After stirring for 16 h, ice chips were added, followed by enough 5% citric acid solution to effect a precipitate (pH-5.5). The resulting mixture was washed three times with EtOAc and the combined organic extracts were washed with brine and dried over Na₂SO₄. The solution was evaporated to afford 419 mg of Int 1.1g as a pale yellow solid, which was used without purification.

Step H. Preparation of Compound 1.1

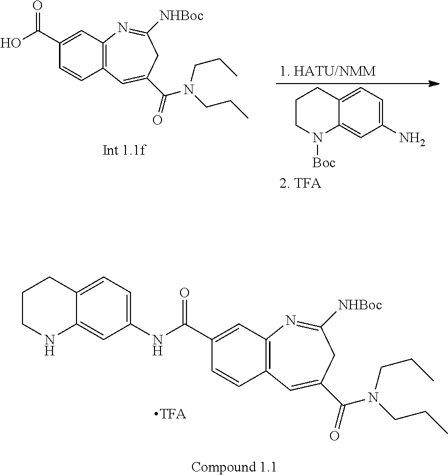 [SMILES: CCCN(CCC)C(=O)C1=Cc2ccc(C(=O)O)cc2N=C(NC(=O)OC(C)(C)C)C1 ; CC(C)(C)OC(=O)N1CCCc2ccc(N)cc21 ; CCCN(CCC)C(=O)C1=Cc2ccc(C(=O)Nc3ccc4c(c3)NCCC4)cc2N=C(NC(=O)OC(C)(C)C)C1]

46 mg (0.12 mmol) of HATU was added to a solution containing 43 mg (0.10 mmol) of Int 1.1f in 1.0 mL of DMF. The reaction mixture was stirred for 5 minutes and then treated with 30 mg (0.12 mmol) of 7-N-Boc-amino-1,2,3,4-tetrahydroquinoline and 0.022 mL (0.20 mmol) of NMM. The reaction mixture was stirred for 16 h then treated with 5 mL of saturated NH₄Cl solution and 5 mL of water. The resulting mixture was extracted three times with EtOAc and the combined organics were washed with brine then dried over Na₂SO₄. After evaporation of the solvent, the crude oil was dissolved in 3 mL of DCM and then cooled to 0° C. Then, 0.6 mL of TFA was added to the mixture. The mixture was stirred for 4 h, evaporated and the resulting residue was purified by reverse phase chromatography to afford the TFA salt of Compound 1.1 as a white solid. ¹H NMR (CD₃OD) δ 7.96 (s, 1H), 7.95 (s, 1H), 7.85 (d, J=2.4 Hz, 1H), 7.79 (d, J=8.8 Hz, 1H), 7.38 (d, J=7.5 Hz, 1H), 7.25 (d, J=7.5 Hz, 1H), 7.10 (s, 1H), 3.55 (t, J=7.5 Hz, 6H), 3.33 (m, 2H), 2.90 (t, J=6.6 Hz, 2H), 2.10 (m, 1H), 1.69 (m, 4H), 0.77 (bs, 6H). LCMS [M+H]=460.25.

### Example 2: Myeloid Cell Agonist Benzazepine Compounds

Table 1 shows benzazepine compounds that are myeloid cell agonists. Compounds 1.2-1.67 can be prepared in manner similar to that used for the synthesis of Compound 1.1 (Example 2) by using Intermediate 1.1f and an appropriately substituted amine, methods described in the following examples, or other methods known to the skilled artisan.

TABLE 1
Compounds 1.1-1.67

| Cmpd | Structure and IUPAC Name | ¹H NMR | M + 1 |
|---|---|---|---|
| 1.1 | 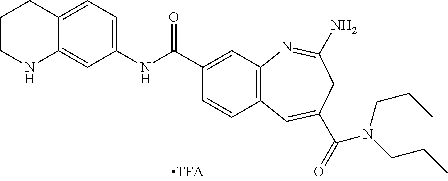 [SMILES: CCCN(CCC)C(=O)C1=Cc2ccc(C(=O)Nc3ccc4c(c3)NCCC4)cc2N=C(N)C1] | (CD₃OD) δ 7.96 (s, 1H), 7.95 (s, 1H), 7.85 (d, J = 2.4 Hz, 1H), 7.79 (d, J = 8.8 Hz, 1H), 7.38 (d, J = 7.5 Hz, 1H), 7.25 (d, J = 7.5 Hz, 1H), 7.10 (s, 1H), 3.55 (t, | 460.3 |
| | | J = 7.5 Hz, 6H), | |
| | | 3.33 (m, | |
| | | 2H), 2.90 (t, | |
| | | J = 6.6 Hz, | |
| | | 2H), 2.10 (m, 1H), | |
| | | 1.69 (m, 4H), | |
| | | 0.77 (bs, 6H). | |
| | 2-amino-N⁴,N⁴-dipropyl-N⁸-(1,2,3,4-tetrahydroquinolin-7- | | |
| | yl)-3H-benzo[b]azepine-4,8-dicarboxamide TFA salt | | |
| 1.2 | 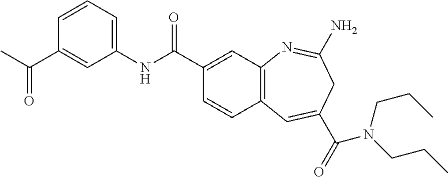 [SMILES: CCCN(CCC)C(=O)C1=Cc2ccc(C(=O)Nc3cccc(C(C)=O)c3)cc2N=C(N)C1] | (CD₃OD) δ 8.35 (s, 1H), 7.97 (dd, J = 1.5,8.0 Hz, 1H), 7.79 (dd, J = 1.5, 8.8 Hz, 1H), 7.75 (d, J = 1.5 Hz, 1H), 7.63 (dd, J = 1.5, 7.5 Hz, 1H), 7.51 (m, 2H), 6.92 (s, | 446.9 |
| | | 1H), 3.43 (t, | |
| | | J = 7.5 Hz, | |
| | | 4H), 2.63 (s, | |
| | | 3H), 1.70 | |
| | | (m, 4H), 0.96 | |
| | | (bs, 3H), | |
| | | 0.87 (bs, 3H). | |
| | N⁸-(3-acetylphenyl)-2-amino-N⁴,N⁴-dipropyl-3H- | | |
| | benzo[b]azepine-4,8-dicarboxamide | | |
| 1.3 | 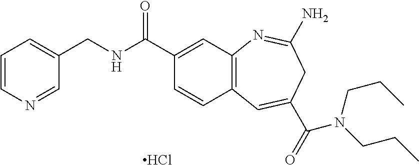 [SMILES: CCCN(CCC)C(=O)C1=Cc2ccc(C(=O)NCc3cccnc3)cc2N=C(N)C1 ; Cl] | (CD₃OD) δ 8.93 (s, 1H), 8.80 (d, J = 5.5 Hz, 1H), 8.68 (d, J = 8.5 Hz, 1H), 8.11 (m, 1H), 7.97 (d, J = 1.5 Hz, 1H), 7.89 (dd, J = 1.5, 7.5 Hz, 1H), | 419.9 |
| | | 7.67 (d, J = | |
| | | 7.5 Hz, 1H), | |
| | | 7.08 (s, 1H), | |
| | | 4.84 (s, | |
| | | 2H), 3.44 (bs, 4H), | |
| | | 3.25 (s, 2H), 1.69 | |
| | | (q, J = 7.5 Hz, | |
| | | 4H), 0.92 (bs, | |
| | | 3H), 0.90 (bs, 3H). | |
| | 2-amino-N⁴,N⁴-dipropyl-N⁸-(pyridin-3-ylmethyl)-3H- | | |
| | benzo[b]azepine-4,8-dicarboxamide HCl salt | | |
| 1.4 | 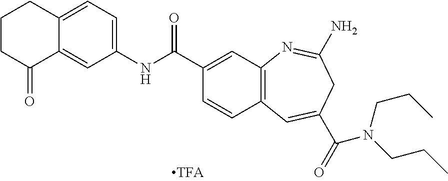 [SMILES: CCCN(CCC)C(=O)C1=Cc2ccc(C(=O)Nc3ccc4c(c3)C(=O)CCC4)cc2N=C(N)C1] | (CD₃OD) δ 8.27 (s, 1H), 7.97 (m, 3H), 7.71 (d, J = 7.5 Hz, 1H), 7.37 (d, J = 7.5 Hz, 1H), 7.11 (s, 1H), 3.55 (m, 4H), 3.28 (s, 2H), 3.00 (t, J = 7.5 Hz, 2H), 2.69 (t, J = 7.5 Hz, | 473.2 |
| | | 2H), 2.15 | |
| | | (m, 2H), 1.70 (q, | |
| | | J = 7.5 Hz, 4H), | |
| | | 0.98 (bs, 6H). | |
| | 2-amino-N⁸-(8-oxo-5,6,7,8-tetrahydronaphthalen-2-yl)- | | |
| | N⁴,N⁴-dipropyl-3H-benzo[b]azepine-4,8-dicarboxamide | | |
| | trifluoroacetate salt | | |
| 1.5 | 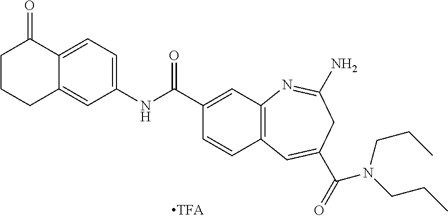 [SMILES: CCCN(CCC)C(=O)C1=Cc2ccc(C(=O)Nc3ccc4c(c3)CCCC4=O)cc2N=C(N)C1] | (CD₃OD) δ 7.99 (m, 3H), 7.85 (s, 1H), 7.71 (m, 2H), 7.15 (s, 1H), 3.50 (m, 4H), 3.30 (s, 2H), 3.03 (t, J = 7.5 Hz, 2H), 2.65 (t, J = 7.5 Hz, 2H), 2.15 (m, 2H), 1.73 (q, J = | 473.1 |
| | | 7.5 Hz, 4H), | |
| | | 0.97 (bs, 6H). | |
| | 2-amino-N⁸-(5-oxo-5,6,7,8-tetrahydronaphthalen-2-yl)- | | |
| | N⁴,N⁴-dipropyl-3H-benzo[b]azepine-4,8-dicarboxamide | | |
| | trifluoroacetate salt | | |
| 1.6 | 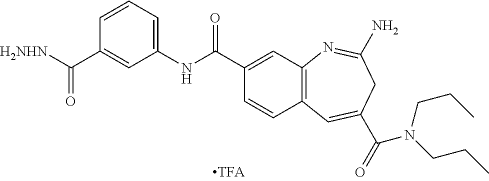 | (CD₃OD) δ 8.41 (s, 1H), 7.99 (m, 2H), 7.67 (m, 2H), 7.57 t, J = 8.0 Hz, 1H), 7.12 (s, 1H), 3.65 (m, 5H), 1.66 (m, 4H), 0.96 (bs, 6H). | |
| | 2-amino-N⁸-(3-(hydrazinecarbonyl)phenyl)-N⁴,N⁴- | | |
| | dipropyl-3H-benzo[b]azepine-4,8-dicarboxamide | | |
| | trifluoroacetate salt | | |
| 1.7 | 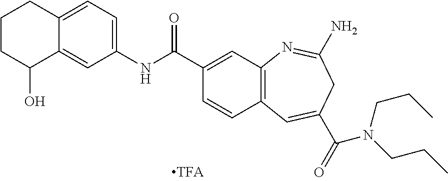 [SMILES: CCCN(CCC)C(=O)C1=Cc2ccc(C(=O)Nc3ccc4c(c3)C(O)CCC4)cc2N=C(N)C1] | (DMSO-d₆) δ 10.1 (s, 1H), 7.86 (s, 1H), 7.72 (s, 1H), 7.56 (d, J = 7.7 Hz, 1H), 7.49 (d, J = 7.5 Hz, 1H), 7.38 (d, J = 7.5 Hz, 1H), 7.00 (d, J = 8.1 Hz, | 475.2 |
| | | 1H), 6.87 (s, | |
| | | 1H), 6.77 (s, | |
| | | 1H), 5.10 | |
| | | (bs, 1H), 4.54 | |
| | | (s, 1H), | |
| | | 3.28 (m, 4H), | |
| | | 3.28 (s, | |
| | | 2H), 2.66 (m, 4H), | |
| | | 1.88 (m, 2H), | |
| | | 1.66-1.32 (m, 6H), | |
| | | 0.87 (bs, 6H). | |
| | 2-amino-N⁸-(8-hydroxy-5,6,7,8-tetrahydronaphthalen-2- | | |
| | yl)-N⁴,N⁴-dipropyl-3H-benzo[b]azepine-4,8- | | |
| | dicarboxamide trifluoroacetate salt | | |
| 1.8 | 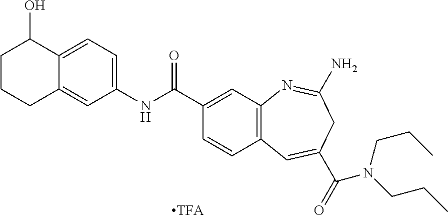 [SMILES: CCCN(CCC)C(=O)C1=Cc2ccc(C(=O)Nc3ccc4c(c3)CCCC4O)cc2N=C(N)C1] | (CD₃CN) δ 14.0 (bs, 1H), 11.0 (bs, 1H), 8.86 (s, 1H), 7.87 (s, 1H), 7.85 (d, J = 7.7 Hz, 1H), 7.62 (m, 2H), 7.42 (d, J = 7.5 Hz, 1H), 6.98 (s, 1H), 6.77 (s, 1H), 4.68 (s, 1H), 3.28 (m, 4H), 3.15 (m, 4H), | 475.2 |
| | | 2.76 (m, 2H), | |
| | | 1.88 (m, | |
| | | 3H), 1.61 (m, 4H), | |
| | | 0.92 (bs, 6H). | |
| | 2-amino-N⁸-(5-hydroxy-5,6,7,8-tetrahydronaphthalen-2- | | |
| | yl)-N⁴,N⁴-dipropyl-3H-benzo[b]azepine-4,8- | | |
| | dicarboxamide trifluoroacetate salt | | |
| 1.9 | 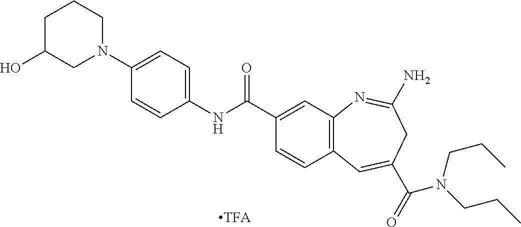 [SMILES: CCCN(CCC)C(=O)C1=Cc2ccc(C(=O)Nc3ccc(N4CCCC(O)C4)cc3)cc2N=C(N)C1] | (CD₃OD)□δ 9.98 (s, 1H), 7.63 (m, 2H), 7.44 (d, J = 8.4 Hz, 1H), 7.39 (d, J = 8.4 Hz, 1H), 6.90 (m, 3H), 6.75 (s, 1H), 4.77 (d, J = 8.4 Hz, 1H), 3.56 (m, 2H), 3.44 (m, 1H), 2.70-2.50 (m, 3H), 1.88 (m, | |
| | | 1H), 1.70 (m, 1H), | |
| | | 1.60 (m, 4H), | |
| | | 1.22 (m, | |
| | | 2H), 0.85 (bs, 6H). | |
| | 2-amino-N⁸-(4-(3-hydroxypiperidin-1-yl)phenyl)-N⁴,N⁴- | | |
| | dipropyl-3H-benzo[b]azepine-4,8-dicarboxamide | | |
| | trifluoroacetate salt | | |
| 1.10 | 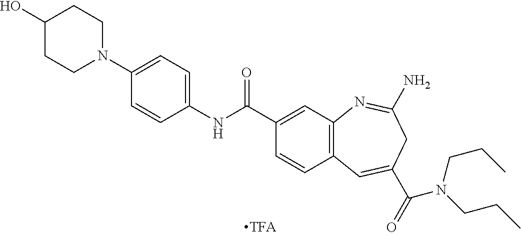 [SMILES: CCCN(CCC)C(=O)C1=Cc2ccc(C(=O)Nc3ccc(N4CCC(O)CC4)cc3)cc2N=C(N)C1] | (CD₃OD) δ 7.95 (m, 4H), 7.71 (d, J = 8.0 Hz, 1H), 7.55 (d, J = 8.8 Hz, 2H), 7.10 (s, 1H), 4.05 (m, 1H), 3.80 (m, 2H), 3.50 (m, 4H), 3.33 (s, 2H), 2.25 (m, 2H), 1.97 (m, 2H), 1.73 (q, J = 7.5 Hz, | |
| | | 4H), 0.97 (bs, 6H). | |
| | 2-amino-N⁸-(4-(4-hydroxypiperidin-1-yl)phenyl)-N⁴,N⁴- | | |
| | dipropyl-3H-benzo[b]azepine-4,8-dicarboxamide | | |
| | trifluoroacetate salt | | |
| 1.11 | 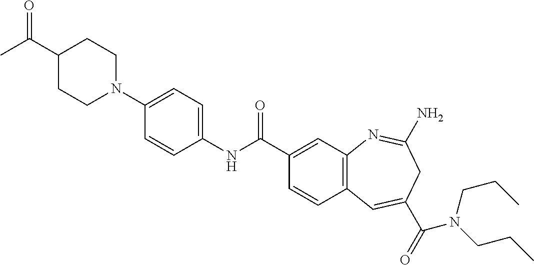 [SMILES: CCCN(CCC)C(=O)C1=Cc2ccc(C(=O)Nc3ccc(N4CCC(C(C)=O)CC4)cc3)cc2N=C(N)C1] | (CD₃OD) δ 7.95 (m, 4H), 7.71 (d, J = 8.0 Hz, 1H), 7.55 (d, J = 8.8 Hz, 2H), 7.10 (s, 1H), 4.05 (m, 1H), 3.80 (m, 2H), 3.50 (m, 4H), 3.33 (s, 2H), 2.25 (m, 2H), 2.15 (s, 3H), 1.97 (m, 2H), 1.73 (q, J = 7.5 Hz, 4H), 0.97 (bs, 6H). | |
| | N⁸-(4-(4-acetylpiperidin-1-yl)phenyl)-2-amino-N⁴,N⁴- | | |
| | dipropyl-3H-benzo[b]azepine-4,8-dicarboxamide | | |
| 1.12 | 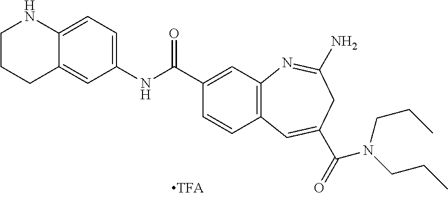 [SMILES: CCCN(CCC)C(=O)C1=Cc2ccc(C(=O)Nc3ccc4c(c3)CCCN4)cc2N=C(N)C1] | (CD₃OD) δ 7.95 (m, 2H), 7.71 (m, 3H), 7.18 (m, 1H), 7.11 (s, 1H), 3.43 (m, 4H), 3.50 (m, 2H), 3.28 (s, 2H), 2.96 (t, J = 7.5 Hz, 2H), 2.15 (m, 2H), 1.73 (q, J = 7.5 Hz, 4H), 0.97 (bs, 6H). | 460.2 |
| | 2-amino-N⁴,N⁴-dipropyl-N⁸-(1,2,3,4-tetrahydroquinolin-6- | | |
| | yl)-3H-benzo[b]azepine-4,8-dicarboxamide | | |
| | trifluoroacetate salt | | |
| 1.13 | 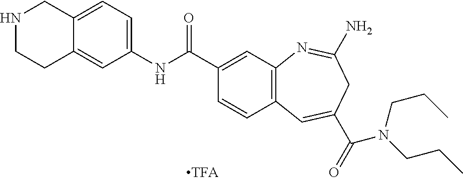 [SMILES: CCCN(CCC)C(=O)C1=Cc2ccc(C(=O)Nc3ccc4c(c3)CCNC4)cc2N=C(N)C1] | (CD₃OD) δ 7.95 (m, 2H), 7.71-7.61 (m, 3H), 7.27 (d, J = 8.4 Hz, 1H), 7.13 (s, 1H), 4.38 (s, 2H), 3.58-3.45 (m, 6H), 3.40 (s, 2H), 3.15 (t, J = 6.6 Hz, 2H), 1.71 (q, J = 7.5 Hz, | 460.2 |
| | | 4H), 0.96 (bs, 6H). | |
| | 2-amino-N⁴,N⁴-dipropyl-N⁸-(1,2,3,4- | | |
| | tetrahydroisoquinolin-6-yl)-3H-benzo[b]azepine-4,8- | | |
| | dicarboxamide trifluoroacetate salt | | |
| 1.14 | 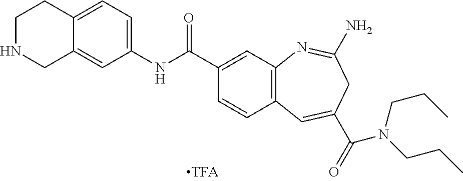 [SMILES: CCCN(CCC)C(=O)C1=Cc2ccc(C(=O)Nc3ccc4c(c3)CNCC4)cc2N=C(N)C1] | (CD₃OD) δ 7.95 (m, 2H), 7.71-7.61 (m, 3H), 7.27 (d, J = 8.4 Hz, 1H), 7.13 (s, 1H), 4.39 (s, 2H), 3.58- 3.45 (m, 6H)? 3.40 (s, 2H), 3.14 (t, J = 6.6 Hz, 2H), 1.74 | 460.2 |
| | | (q, J = 7.5 Hz, | |
| | | 4H), 0.95 (bs, 6H). | |
| | 2-amino-N⁴,N⁴-dipropyl-N⁸-(1,2,3,4-tetrahydro- | | |
| | isoquinolin-7-yl)-3H-benzo[b]azepine-4,8-dicarboxamide | | |
| | trifluoroacetate salt | | |
| 1.15 | 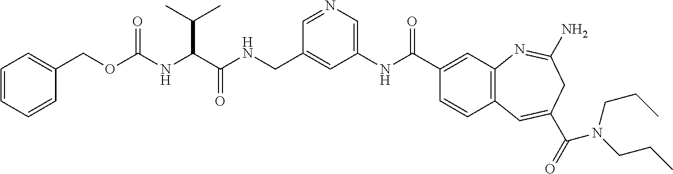 [SMILES: CCCN(CCC)C(=O)C1=Cc2ccc(C(=O)Nc3cncc(CNC(=O)[C@@H](NC(=O)OCc4ccccc4)C(C)C)c3)cc2N=C(N)C1] | (CD₃OD) δ 8.80 (d, J = 2.1 Hz, 1H), 8.29 (s, 1H), 8.21 (s, 1H), 7.72 (s, 1H), 7.58 (dd, J = 1.5, 8.2 Hz, 1H), 7.33-7.23 (m, 5H), 6.90 (s, 1H), 5.11 (d, J = 6.8 Hz, | 668.3 |
| | | 2H), 4.44 (s, | |
| | | 2H), 3.98 (d, | |
| | | J = 7.0 Hz, | |
| | | 1H), 3.43 (m, 4H), | |
| | | 2.11 (m, 1H), | |
| | | 1.66 (m, | |
| | | 4H), 1.0-0.95 | |
| | | (m, 12H). | |
| | benzyl (S)-(1-(((5-(2-amino-4-(dipropyl-carbamoyl)-3H- | | |
| | benzo[b]azepine-8-carboxamido)pyridin-3- | | |
| | yl)methyl)amino)-3-methyl-1-oxobutan-2-yl)carbamate | | |
| 1.16 | 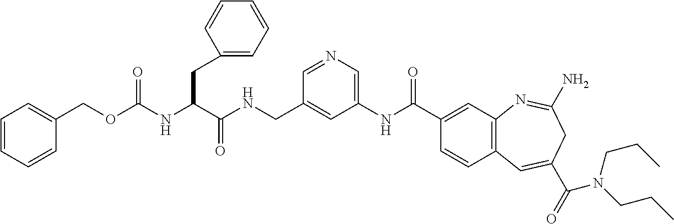 [SMILES: CCCN(CCC)C(=O)C1=Cc2ccc(C(=O)Nc3cncc(CNC(=O)[C@H](Cc4ccccc4)NC(=O)OCc4ccccc4)c3)cc2N=C(N)C1] | (CD₃OD) δ 8.80 (d, J = 2.1 Hz, 1H), 8.21 (s, 1H), 8.11 (s, 1H), 7.72 (s, 1H), 7.61 (dd, J = 1.5, 8.2 Hz, 1H), 7.45 (d, = 8.2 Hz, H), 7.33- 7.11 (m, 10H), 6.90 (s, 1H), 5.00 (q, J = 12.6 Hz, 2H), 4.35 | 716.3 |
| | | (m, 3H), 3.43 | |
| | | (m, 4H), | |
| | | 3.12 (m, 1H), | |
| | | 2.89 (m, | |
| | | 2H), 1.66 (m, | |
| | | 4H), 1.0- | |
| | | 0.85 (m, 6H). | |
| | benzyl (S)-(1-(((5-(2-amino-4-(dipropyl-carbamoyl)-3H- | | |
| | benzo[b]azepine-8-carboxamido)pyridin-3- | | |
| | yl)methyl)amino)-1-oxo-3-phenylpropan-2-yl)carbamate | | |
| 1.17 | 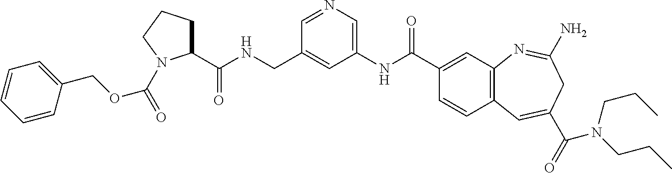 [SMILES: CCCN(CCC)C(=O)C1=Cc2ccc(C(=O)Nc3cncc(CNC(=O)[C@@H]4CCCN4C(=O)OCc4ccccc4)c3)cc2N=C(N)C1] | (CD₃OD) δ 8.82 (d, J = 2.1 Hz, 1H), 8.69 (s, 1H), 8.33-8.21 (m, 2H), 7.70 (d, J = 17 Hz, 1H), 7.57 (dd, J = 1.5, 8.2 Hz, 1H), 7.45 (d, J = 8.1 Hz, 1H), 7.40-7.21 | 666.5 |
| | | (m, 5H), 6.90 (s, | |
| | | 1H), 5.00 (q, | |
| | | J = 12.6 Hz, | |
| | | 2H), 4.49 (s, | |
| | | 1H), 4.35 (m, 2H), | |
| | | 3.63-3.53 (m, 2H), | |
| | | 3.45 (m, 4H), | |
| | | 2.85 (m, | |
| | | 1H), 2.31 (m, 1H), | |
| | | 2.10-1.86 (m, 3H), | |
| | | 1.65 (m, 4H), 1.0- | |
| | | 0.85 (m, 6H). | |
| | benzyl (S)-2-(((5-(2-amino-4-(dipropyl-carbamoyl)-3H- | | |
| | benzo[b]azepine-8-carboxamido)pyridin-3- | | |
| | yl)methyl)carbamoyl)pyrrolidine-1-carboxylate | | |
| 1.18 | 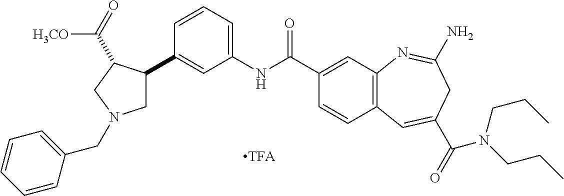 [SMILES: CCCN(CCC)C(=O)C1=Cc2ccc(C(=O)Nc3cccc([C@H]4CN(Cc5ccccc5)C[C@@H]4C(=O)OC)c3)cc2N=C(N)C1] | (CD₃OD) δ□ 7.96 (m, 2H), 7.89 (bs, 1H), 7.70 (d, 8.2 Hz, 2H), 7.55 (m, 6H), 7.42 (t, J = 7.5 Hz, 1H), 7.22 (d, J = 7.0 Hz, 1H), 7.11 (s, 1H), 4.53 (s, 2H), 3.90 (m, 3H), | 622.2 |
| | | 3.70 (s, 3H), | |
| | | 3.51 (m, 4H), | |
| | | 3.37 (s, | |
| | | 2H), 1.70 (q, | |
| | | J = 7.5 Hz, | |
| | | 4H), 1.0-0.85 | |
| | | (m, 6H). | |
| | methyl (3R,4S)-4-(3-(2-amino-4-(dipropylcarbamoyl)-3H- | | |
| | benzo[b]azepine-8-carboxamido)phenyl)-1- | | |
| | benzylpyrrolidine-3-carboxylate trifluoroacetate salt | | |
| 1.19 | 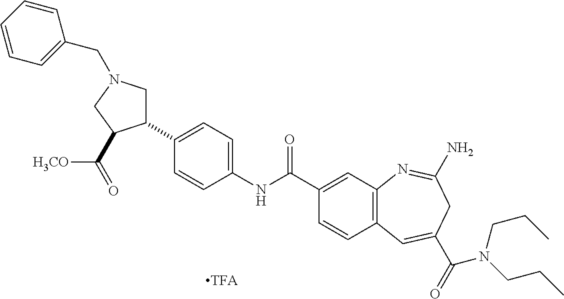 | (CD₃OD) δ□ 7.95 (m, 2H), 7.75 (d, 8.2 Hz, 2H), 7.69 (d, J = 8.5 Hz, 1H), 7.75 (m, 1H), 7.51 (m, 5H), 7.40 (d, J = 7.5 Hz, 1H), 7.10 (s, 1H), 4.51 (s, 2H), 3.90 (m, 3H), 3.68 (s, 3H), 3.51 (m, 4H), 3.37 (s, 2H), 1.70 (q, J = 7.5 Hz, 4H), 0.99-0.92 (m, 6H). | 622.2 |
| | methyl (3R,4S)-4-(4-(2-amino-4-(dipropyl-carbamoyl)- | | |
| | 3H-benzo[b]azepine-8-carboxamido)phenyl)-1- | | |
| | benzylpyrrolidine-3-carboxylate trifluoroacetate salt | | |
| 1.20 | 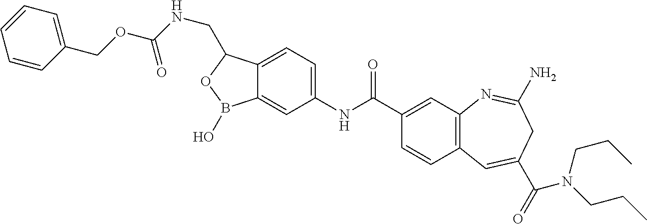 [SMILES: CCCN(CCC)C(=O)C1=Cc2ccc(C(=O)Nc3ccc4c(c3)B(O)OC4CNC(=O)OCc3ccccc3)cc2N=C(N)C1] | | 624.3 |
| | benzyl ((6-(2-amino-4-(dipropylcarbamoyl)-3H- | | |
| | benzo[b]azepine-8-carboxamido)-1-hydroxy-1,3- | | |
| | dihydrobenzo[c][1,2]oxaborol-3-yl)methyl)carbamate | | |
| 1.21 | 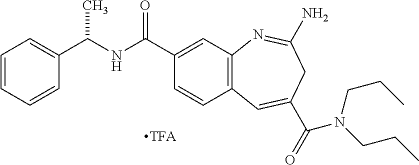 [SMILES: CCCN(CCC)C(=O)C1=Cc2ccc(C(=O)N[C@@H](C)c3ccccc3)cc2N=C(N)C1] | (CD₃OD) δ 7.82 (d, 8.1 Hz, 1H), 7.81 (s, 1H), 7.45 (d, J = 8.1 Hz, 2H), 7.34 (t, J = 7.5 Hz, 2H), 7.27 (d, J = 7.5 Hz, 1H), 7.07 (s, | 433.2 |
| | | 1H), 5.25 | |
| | | (q, J = 7.0 Hz, | |
| | | 1H), 3.45 | |
| | | (m, 4H), 3.33 | |
| | | (s, 2H), 1.74 (q, | |
| | | J = 7.5 Hz, 4H), | |
| | | 1.52 (d, J = | |
| | | 7.1 Hz, 3H), | |
| | | 0.94 (bs, 3H), | |
| | | 0.91 (bs, 3H). | |
| | (S)-2-amino-N⁸-(1-phenylethyl)-N⁴,N⁴-dipropyl-3H- | | |
| | benzo[b]azepine-4,8-dicarboxamide trifluoroacetate salt | | |
| 1.22 | 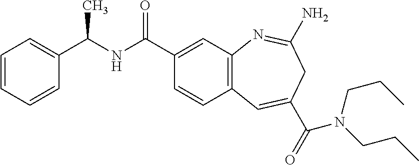 [SMILES: CCCN(CCC)C(=O)C1=Cc2ccc(C(=O)N[C@H](C)c3ccccc3)cc2N=C(N)C1] | (CD₃OD) δ 7.82 (d, 8.1 Hz, 1H), 7.81 (s, 1H), 7.45 (d, J = 8.1 Hz, 2H), 7.34 (t, J = 7.5 Hz, 2H), 7.27 (d, J = 7.5 Hz, 1H), 7.07 (s, | 433.2 |
| | | 1H), 5.25 | |
| | | (q, J = 7.0 Hz, | |
| | | 1H), 3.45 | |
| | | (m, 4H), | |
| | | 3.33 (s, 2H), | |
| | | 1.74 (q, J = | |
| | | 7.5 Hz, 4H), | |
| | | 1.52 (d, J = | |
| | | 7.1 Hz, 3H), | |
| | | 0.94 (bs, 3H), | |
| | | 0.91 (bs, 3H). | |
| | (R)-2-amino-N⁸-(1-phenylethyl)-N⁴,N⁴-dipropyl-3H- | | |
| | benzo[b]azepine-4,8-dicarboxamide trifluoroacetate salt | | |
| 1.23 | 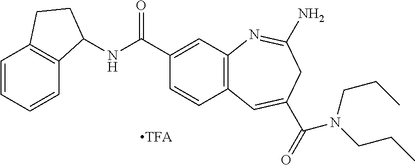 [SMILES: CCCN(CCC)C(=O)C1=Cc2ccc(C(=O)NC3CCc4ccccc43)cc2N=C(N)C1] | (CD₃OD) δ 7.89 (s, 1H), 7.81 (dd, J = 1.8, 8.2 Hz, 1H), 7.61 (d, J = 8.2 Hz, 1H), 7.34 (t, J = 7.5 Hz, 2H), 7.21 (m, 2H), 7.07 (s, 1H), 5.65 | 445.1 |
| | | (q, J = 7.8 | |
| | | Hz, 1H), 3.48 | |
| | | (m, 4H), | |
| | | 3.28 (s, 2H), | |
| | | 3.01 (m, 1H), | |
| | | 2.95 (m, | |
| | | 1H), 2.62 (m, | |
| | | 1H), 2.02 (m, | |
| | | 1H), 1.68 (q, | |
| | | J = 7.4 Hz, | |
| | | 4H), 0.94 (bs, | |
| | | 3H), 0.91 (bs, 3H). | |
| | 2-amino-N⁸-(2,3-dihydro-1H-inden-1-yl)-N⁴,N⁴-dipropyl- | | |
| | 3H-benzo[b]azepine-4,8-dicarboxamide trifluoroacetate | | |
| | salt | | |
| 1.24 | 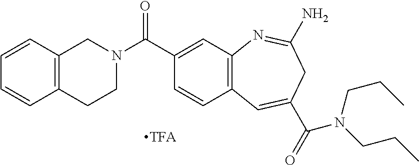 [SMILES: CCCN(CCC)C(=O)C1=Cc2ccc(C(=O)N3CCc4ccccc4C3)cc2N=C(N)C1] | (CD₃OD) δ 7.64 (d, J = 8.4 Hz, 1H), 7.44 (m, 2H), 7.23 (m, 4H), 7.06 (s, 1H), 4.64, (m, 1H), 4.00 (m, 1H), 3.71 (m, 1H), 3.48 (m, 4H). 3.28 (s, 2H), 3.01 | 445.1 |
| | | (m, 1H), 2.95 | |
| | | (m, 1H), | |
| | | 2.62 (m, 1H), | |
| | | 1.98 (s, | |
| | | 1H), 1.71 (q, | |
| | | J = 7.4 Hz, | |
| | | 4H), 1.01 (bs, 3H), | |
| | | 0.95 (bs, 3H). | |
| | 2-amino-N,N-dipropyl-8-(1,2,3,4-tetrahydroisoquinoline- | | |
| | 2-carbonyl)-3H-benzo[b]azepine-4-carboxamide | | |
| | trifluoroacetate salt | | |
| 1.25 | 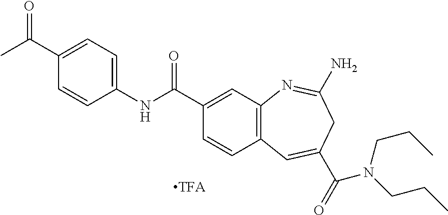 [SMILES: CCCN(CCC)C(=O)C1=Cc2ccc(C(=O)Nc3ccc(C(C)=O)cc3)cc2N=C(N)C1] | (CD₃OD) δ 8.04 (d, J = 8.4 Hz, 1H), 7.94 (d, J = 8.0 Hz, 2H), 7.90 (d, J = 8.8 Hz, 2H), 7.69 (d, J = 8.0 Hz, 1H), 7.10 (s, 1H), 3.43 (m, 4H), 3.28 (s, 2H), 2.60 (s, 3H), 1.71 (q, J = 7.5 Hz, 4H), 0.96 (bs, 3H), | 447.2 |
| | | 0.92 (bs, 3H). | |
| | N⁸-(4-acetylphenyl)-2-amino-N⁴,N⁴-dipropyl-3H- | | |
| | benzo[b]azepine-4,8-dicarboxamide trifluoroacetate salt | | |
| 1.26 | 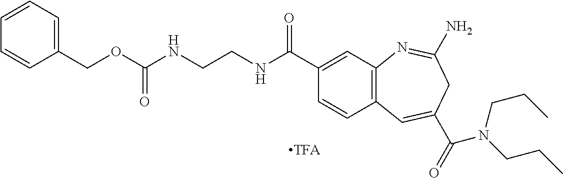 [SMILES: CCCN(CCC)C(=O)C1=Cc2ccc(C(=O)NCCNC(=O)OCc3ccccc3)cc2N=C(N)C1] | (CD₃OD) δ 7.80 (d, J = 1.5 Hz, 1H), 7.74 (dd, J = 1.5, 8.0 Hz, 2H), 7.60 (d, J = 8.0 Hz, 1H), 7.31-7.23 (m, 5H), 7.07 (s, 1H), 5.06 (s, 2H), 3.53-3.38 (m, | 505.8 |
| | | 8H), 3.28 (s, | |
| | | 2H), 1.69 | |
| | | (q, J = 7.5 Hz, | |
| | | 4H), 0.95 | |
| | | (bs, 3H), 0.91 | |
| | | (bs, 3H). | |
| | benzyl (2-(2-amino-4-(dipropylcarbamoyl)-3H- | | |
| | benzo[b]azepine-8-carboxamido)ethyl)carbamate | | |
| | trifluoroacetate salt | | |
| 1.27 | 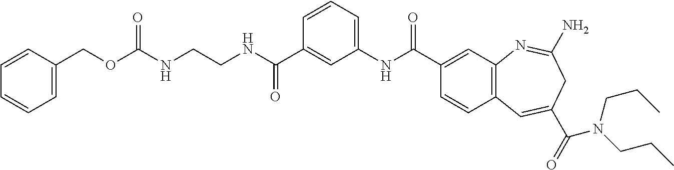 [SMILES: CCCN(CCC)C(=O)C1=Cc2ccc(C(=O)Nc3cccc(C(=O)NCCNC(=O)OCc4ccccc4)c3)cc2N=C(N)C1] | (CD₃OD) δ 7.82-7.75 (m, 4H), 7.74 (d, J = 8.0 Hz, 2H), 7.60 (d, J = 8.0 Hz, 1H), 7.45 (m, 1H), 7.31-7.23 (m, 5H), 7.07 (s, 1H), 5.06 (s, 2H), 3.53- | 625.4 |
| | | 3.38 (m, | |
| | | 8H), 3.28 (s, | |
| | | 2H), 1.69 | |
| | | (q, J = 7.5 Hz, | |
| | | 4H), 0.95 | |
| | | (bs, 3H), 0.91 | |
| | | (bs, 3H). | |
| | benzyl (2-(3-(2-amino-4-(dipropylcarbamoyl)-3H- | | |
| | benzo[b]azepine-8- | | |
| | carboxamido)benzamido)ethyl)carbamate | | |
| 1.28 | 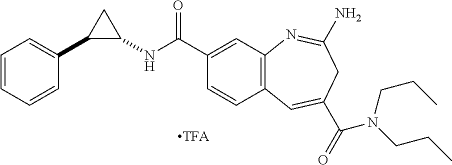 [SMILES: CCCN(CCC)C(=O)C1=Cc2ccc(C(=O)N[C@H]3C[C@@H]3c3ccccc3)cc2N=C(N)C1] | (CD₃OD) δ 7.83 (s, 1H), 7.79 (d, 8.1 Hz, 1H), 7.61 (d, J = 8.1 Hz, 2H), 7.27 (t, J = 7.5 Hz, 2H), 7.19 (d, J = 7.5 Hz, 1H), 7.06 (s, 1H), 3.45 | 444.8 |
| | | (m, 4H), | |
| | | 3.28 (s, 2H), | |
| | | 3.00 (m, 1H), | |
| | | 2.21 (m, | |
| | | 1H), 1.68 (q, | |
| | | J = 7.5 Hz, | |
| | | 4H), 1.35 (m, 2H), | |
| | | 0.96 (bs, 3H), | |
| | | 0.91 (bs, 3H). | |
| | 2-amino-N⁸-((1S,2R)-2-phenylcyclopropyl)-N⁴,N⁴- | | |
| | dipropyl-3H-benzo[b]azepine-4,8-dicarboxamide | | |
| | trifluoroacetate salt | | |
| 1.29 | 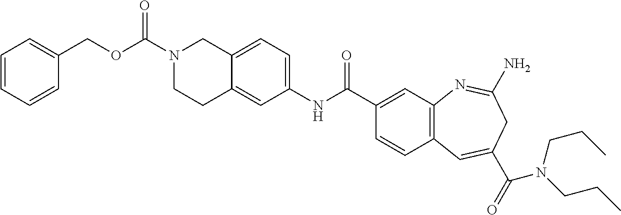 [SMILES: CCCN(CCC)C(=O)C1=Cc2ccc(C(=O)Nc3ccc4c(c3)CCN(C(=O)OCc3ccccc3)C4)cc2N=C(N)C1] | (CD₃OD) δ 7.95 (m, 2H), 7.71-7.61 (m, 3H), 7.27 (d, J = 8.4 Hz, 1H), 7.23 (m, 5H), 7.13 (s, 1H), 5.06 (s, 2H), 4.38 (s, 2H), 4.11 (s, 2H), 3.58 (t, J = 7.5 Hz, 2H), 3.28 (s, 2H), 3.15 (t, J = 6.6 Hz, 2H), 1.71 (q, | 594.4 |
| | | J = 7.5 Hz, 4H), | |
| | | 0.96 (bs, 6H). | |
| | benzyl 6-(2-amino-4-(dipropylcarbamoyl)-3H- | | |
| | benzo[b]azepine-8-carboxamido)-3,4-dihydroisoquinoline- | | |
| | 2(1H)-carboxylate | | |
| 1.30 | 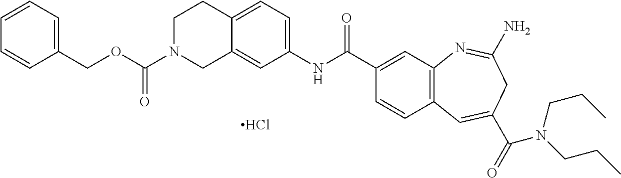 [SMILES: CCCN(CCC)C(=O)C1=Cc2ccc(C(=O)Nc3ccc4c(c3)CN(C(=O)OCc3ccccc3)CC4)cc2N=C(N)C1 ; Cl] | (CD₃OD) δ 7.95 (m, 2H), 7.71-7.61 (m, 3H), 7.27 (d, J = 8.4 Hz, 1H), 7.23 (m, 5H), 7.13 (s, 1H), 5.06 (s, 2H), 4.38 (s, 2H), 4.11 (s, 2H), 3.58 (t, J = 7.5 Hz, H), 3.28 (s, | 594.4 |
| | | 2H), 3.15 (t, | |
| | | J = 6.6 Hz, | |
| | | 2H), 1.71 (q, | |
| | | J = 7.5 Hz, 4H), | |
| | | 0.96 (bs, 6H). | |
| | benzyl 7-(2-amino-4-(dipropylcarbamoyl)-3H- | | |
| | benzo[b]azepine-8-carboxamido)-3,4-dihydroisoquinoline- | | |
| | 2(1H)-carboxylate HCl salt | | |
| 1.31 | 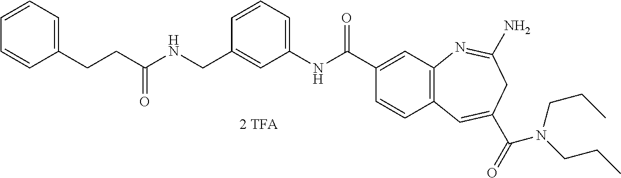 [SMILES: CCCN(CCC)C(=O)C1=Cc2ccc(C(=O)Nc3cccc(CNC(=O)CCc4ccccc4)c3)cc2N=C(N)C1 ; "2 TFA"] | (CD₃OD) δ 9.15 (s, 1H), 8.63 (bs, 1H), 8.42 (s, 1H), 8.29 (s, 1H), 8.02- 7.99 (m, 2H), 7.71 (d, J = 8.5 Hz, 1H), 7.23-7.10 (m, 6H), 4.45 (s, 2H), 3.44 | 566.3 |
| | | (m, 4H), 3.37 (s, | |
| | | 2H), 2.94 (t, J = | |
| | | 7.5 Hz, 2H), | |
| | | 2.57 (t, J = | |
| | | 7.5 Hz, 2H), | |
| | | 1.61 (q, J = | |
| | | 7.5 Hz, 4H), | |
| | | 0.96 (bs, 3H), | |
| | | 0.91 (bs, 3H). | |
| | 2-amino-N⁸-(3-((3-phenylpropanamido)methyl)phenyl)- | | |
| | N⁴,N⁴-dipropyl-3H-benzo[b]azepine-4,8-dicarboxamide | | |
| | bis TFA salt | | |
| 1.32 | 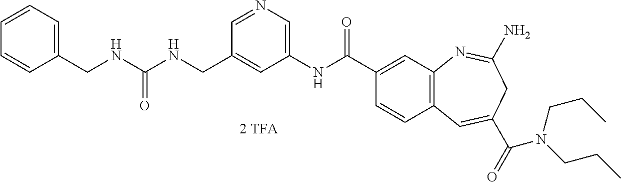 [SMILES: CCCN(CCC)C(=O)C1=Cc2ccc(C(=O)Nc3cncc(CNC(=O)NCc4ccccc4)c3)cc2N=C(N)C1 ; "2 TFA"] | (CD₃OD) δ 9.15 (s, 1H), 8.47 (s, 1H), 8.42 (s, 1H), 8.29 (s, 1H), 8.02-7.99 (m, 2H), 7.72 (d, J = 8.0 Hz, 1H), 7.33 (m, 4H), 7.22 (m, 1H), 7.12 (s, 1H), 4.45 (s, 2H), 4.33 (s, 2H), | 568.3 |
| | | 3.54 (m, 4H), | |
| | | 3.37 (s, | |
| | | 2H), 1.71 (q, | |
| | | J = 7.5 Hz, | |
| | | 4H), 0.97 (bs, 3H), | |
| | | 0.92 (bs, 3H). | |
| | 2-amino-N⁸-(5-((3-benzylureido)methyl)pyridin-3-yl)- | | |
| | N⁴,N⁴-dipropyl-3H-benzo[b]azepine-4,8-dicarboxamide | | |
| | bis TFA salt | | |
| 1.33 | 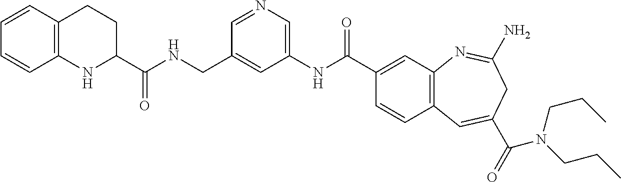 [SMILES: CCCN(CCC)C(=O)C1=Cc2ccc(C(=O)Nc3cncc(CNC(=O)C4CCc5ccccc5N4)c3)cc2N=C(N)C1] | (CD₃OD) δ 8.77 (s, 1H), 8.42 (s, 1H), 8.22 (s, 1H), 8.19 (m, 1H), 7.71 (s, 1H), 7.59 (dd, J = 8.1, 1.8 Hz, 1H), 7.44 (d, J = 8.1 Hz, 1H), 6.99- 6.84 (m, | 593.3 |
| | | 3H), 6.66 (d, | |
| | | J = 8.0 Hz, | |
| | | 1H), 6.55 (t, | |
| | | J = 7.3 Hz, | |
| | | 1H), 4.51 (s, | |
| | | 2H), 4.00 (t, | |
| | | J = 5.2 Hz, | |
| | | 1H), 3.44 (m, 4H), | |
| | | 2.85 (s, 2H), | |
| | | 2.74 (m, | |
| | | 1H), 2.51 (m, 1H), | |
| | | 2.25 (m, 1H), | |
| | | 1.91 (m, | |
| | | 1H), 1.67 (m, 4H), | |
| | | 0.96 (bs, 3H), | |
| | | 0.91 (bs, 3H). | |
| | 2-amino-N⁴,N⁴-dipropyl-N⁸-(5-((1,2,3,4- | | |
| | tetrahydroquinoline-2-carboxamido)methyl)pyridin-3-yl)- | | |
| | 3H-benzo[b]azepine-4,8-dicarboxamide | | |
| 1.34 | 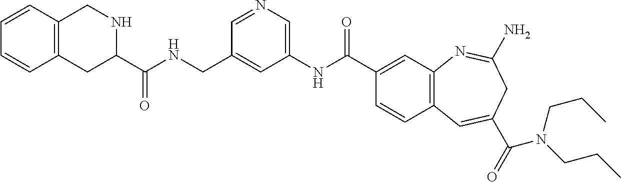 [SMILES: CCCN(CCC)C(=O)C1=Cc2ccc(C(=O)Nc3cncc(CNC(=O)C4Cc5ccccc5CN4)c3)cc2N=C(N)C1] | (CD₃OD) δ 8.80 (d, J = 2.4 Hz, 1H), 8.28 (d, J = 2.1 Hz, 1H), 8.25 (t, J = 2.1 Hz, 1H), 7.72 (d, J = 1.9 Hz, 1H), 7.61 (dd, J = 1.9, 8.1 Hz, 1H), 7.47 (d, J = 8.2 Hz, 1H), | 594.4 |
| | | 7.13 (m, 3H), | |
| | | 7.05 (m, | |
| | | 1H), 6.90 (s, | |
| | | 1H), 4.50 | |
| | | (s, 2H), 4.05 (q, | |
| | | J = 6.1 Hz, | |
| | | 2H), 3.63 | |
| | | (dd, J = 4.7, | |
| | | 10.5 Hz, | |
| | | 1H), 3.43 (m, 4H), | |
| | | 3.05 (dd, J = 4.7, | |
| | | 16.0 Hz, 1H), | |
| | | 3.02 (m, | |
| | | 1H), 2.83 (d, | |
| | | J = 16.6 Hz, | |
| | | 1H), 1.71 | |
| | | (m, 4H), 1.0-0.85 | |
| | | (m, 6H). | |
| | 2-amino-N⁴,N⁴-dipropyl-N⁸-(5-((1,2,3,4- | | |
| | tetrahydroisoquinoline-3-carboxamido)-methyl)pyridin-3- | | |
| | yl)-3H-benzo[b]azepine-4,8-dicarboxamide | | |
| 1.35 | 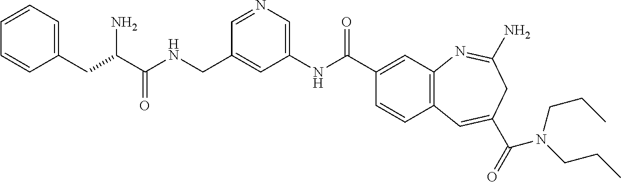 [SMILES: CCCN(CCC)C(=O)C1=Cc2ccc(C(=O)Nc3cncc(CNC(=O)[C@@H](N)Cc4ccccc4)c3)cc2N=C(N)C1] | (CD₃OD) δ 8.78 (d, J = 2.3 Hz, 1H), 8.15 (s, 1H), 8.11 (s, 1H), 7.72 (s, 1H), 7.61 (dd, J = 1.5, 8.2 Hz, 1H), 7.45 (d, = 8.2 Hz, 1H), 7.23- 7.15 (m, 5H), 6.90 (s, | 582.2 |
| | | 1H), 4.44 (q, | |
| | | J = 12.6 Hz, | |
| | | 2H), 3.63 (t, | |
| | | J = 7.5 Hz, | |
| | | 1H), 3.43 (m, | |
| | | 4H), 2.99 (m, 1H), | |
| | | 2.89 (m, 2H), | |
| | | 1.66 (m, 4H), | |
| | | 1.0-0.85 (m, 6H). | |
| | (S)-2-amino-N⁸-(5-((2-amino-3- | | |
| | phenylpropanamido)methyl)pyridin-3-yl)-N⁴,N⁴-dipropyl- | | |
| | 3H-benzo[b]azepine-4,8-dicarboxamide | | |
| 1.36 | 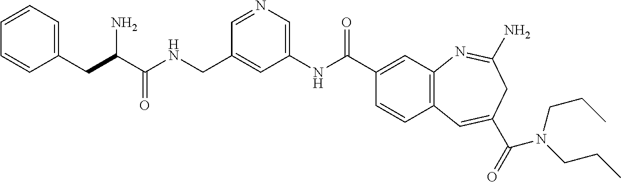 [SMILES: CCCN(CCC)C(=O)C1=Cc2ccc(C(=O)Nc3cncc(CNC(=O)[C@H](N)Cc4ccccc4)c3)cc2N=C(N)C1] | (CD₃OD) δ 8.78 (d, J = 2.3 Hz, 1H), 8.15 (s, 1H), 8.11 (s, 1H), 7.72 (s, 1H), 7.61 (dd, J = 1.5, 8.2 Hz, 1H), 7.45 (d, = 8.2 Hz, 1H), 7.23- 7.15 (m, 5H), 6.90 (s, 1H), 4.41 (d, | 582.2 |
| | | J = 15.0 Hz, | |
| | | 1H), 4.34 | |
| | | (d, J = 15.0 | |
| | | Hz, 1H), 3.63 (t, | |
| | | J = 7.5 Hz, 1H), | |
| | | 3.43 (m, 4H), | |
| | | 2.99 (m, | |
| | | 1H), 2.89 (m, 2H), | |
| | | 1.66 (m, 4H), | |
| | | 1.0-0.85 (m, 6H). | |
| | (R)-2-amino-N⁸-(5-((2-amino-3-phenyl-propanamido)- | | |
| | methyl)pyridine-3-yl)-N⁴,N⁴-dipropyl-3H- | | |
| | benzo[b]azepine-4,8-dicarboxamide | | |
| 1.37 | 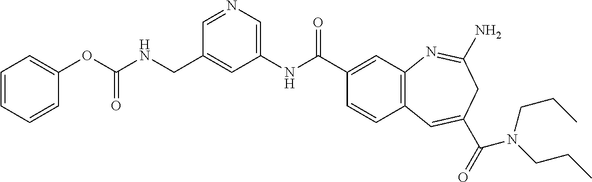 [SMILES: CCCN(CCC)C(=O)C1=Cc2ccc(C(=O)Nc3cncc(CNC(=O)Oc4ccccc4)c3)cc2N=C(N)C1] | (CD₃OD) δ 8.85 (d, J = 2.3 Hz, 1H), 8.35 (s, 1H), 8.31 (s, 1H), 7.72 (s, 1H), 7.95 (m, 2H), 7.72 (d, = 8.5 Hz, 1H), 7.41 (m, 2H), 7.21 (t, J = 7.0 Hz, 1H), 7.15 (d, | 555.2 |
| | | J = 7.5 Hz, | |
| | | 1H), 7.09 (s, | |
| | | 1H), 4.49 (s, | |
| | | 2H), 3.49 | |
| | | (m, 4H), 1.70 | |
| | | (m, 4H), | |
| | | 1.0-0.85 (m, 6H). | |
| | Phenyl ((5-(2-amino-4-(dipropylcarbamoyl)-3H- | | |
| | benzo[b]azepine-8-carboxamido)pyridin-3- | | |
| | yl)methyl)carbamate | | |
| 1.38 | 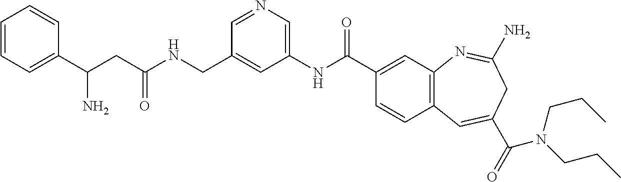 [SMILES: CCCN(CCC)C(=O)C1=Cc2ccc(C(=O)Nc3cncc(CNC(=O)CC(N)c4ccccc4)c3)cc2N=C(N)C1] | (CD₃OD) δ 8.75 (d, J = 2.1 Hz, 1H), 8.15 (s, 1H), 8.11 (s, 1H), 7.72 (d, J = 2.0 Hz, 1H), 7.61 (dd, J = 1.5, 8.2 Hz, 1H), 7.47 (d, = 8.2 Hz, 1H), 7.33- 7.15 (m, 5H), | 582.2 |
| | | 6.90 (s, 1H), | |
| | | 4.41 (m, | |
| | | 3H), 3.43 (m, 4H), | |
| | | 2.89 (m, 2H), | |
| | | 2.67 (m, | |
| | | 2H), 1.66 (m, 4H), | |
| | | 1.0-0.85 (m, 6H). | |
| | 2-amino-N⁸-(5-((3-amino-3-phenyl-propanamido)methyl)- | | |
| | pyridin-3-yl)-N⁴,N⁴-dipropyl-3H-benzo[b]azepine-4,8- | | |
| | dicarboxamide | | |
| 1.39 | 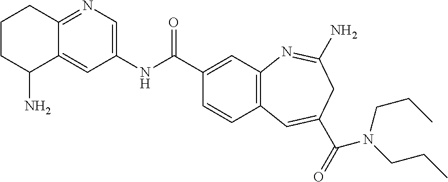 [SMILES: CCCN(CCC)C(=O)C1=Cc2ccc(C(=O)Nc3cnc4c(c3)C(N)CCC4)cc2N=C(N)C1] | (DMSO-d₆) δ 10.3 (s, 1H), 8.68 (s, 1H), 8.26 (s, 1H), 7.68 (s, 1H), 7.50 (d, J = 8.4 Hz, 1H), 7.41 (d, J = 8.4 Hz, 1H), 6.89 (bs, 2H), 6.78 (s, 1H), 3.81 (m, 1H), 3.43 (m, 4H), | 475.3 |
| | | 2.75 (m, | |
| | | 4H), 1.99 (m, | |
| | | 2H), 1.75 (m, | |
| | | 1H), 1.61 (m, | |
| | | 5H), 0.88 (bs, 6H). | |
| | 2-amino-N⁸-(5-amino-5,6,7,8-tetrahydro-quinolin-3-yl)- | | |
| | N⁴,N⁴-dipropyl-3H-benzo-[b]azepine-4,8-dicarbox-amide | | |
| 1.40 | 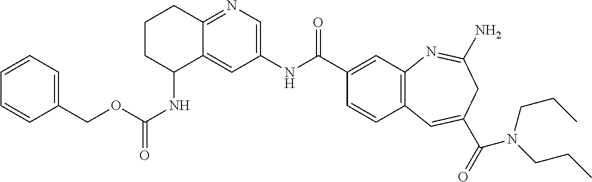 [SMILES: CCCN(CCC)C(=O)C1=Cc2ccc(C(=O)Nc3cnc4c(c3)C(NC(=O)OCc3ccccc3)CCC4)cc2N=C(N)C1] | (CD₃OD) δ 9.25 (d, J = 2.1 Hz, 1H), 8.55 (s, 1H), 8.11 (s, 1H), 8.00 (d, J = 2.0 Hz, 1H), 7.61 (dd, J = 1.5, 8.2 Hz, 1H), 7.33-7.15 (m, 5H), 7.10 (s, 1H), 5.25 (m, | 609.3 |
| | | 4H), 5.05 (m, 1H), | |
| | | 3.63-3.55 (m, 4H), | |
| | | 3.12 (m, 2H), | |
| | | 2.22 (m, | |
| | | 2H), 1.98 (m, 2H), | |
| | | 1.66 (m, 4H), | |
| | | 1.0-0.85 (m, 6H). | |
| | Benzyl (3-(2-amino-4-(dipropylcarbamoyl)-3H- | | |
| | benzo[b]azepine-8-carboxamido)-5,6,7,8- | | |
| | tetrahydroquinolin-5-yl)carbamate | | |
| 1.41 | 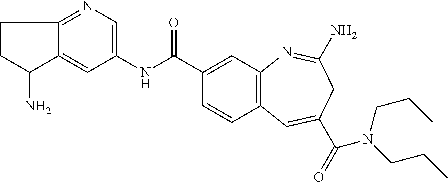 [SMILES: CCCN(CCC)C(=O)C1=Cc2ccc(C(=O)Nc3cnc4c(c3)C(N)CC4)cc2N=C(N)C1] | (CD₃OD) δ 8.75 (d, J = 2.1 Hz, 1H), 8.55 (s, 1H), 7.70 (s, 1H), 7.61 (d, J = 2.0 Hz, 1H), 7.50 (dd, J = 1.5, 8.2 Hz, 1H), 6.90 (s, 1H), 4.70 (m, 1H), 3.63-3.55 (m, | 461.4 |
| | | 4H), 3.20-2.95 | |
| | | (m ,2H), 2.75 | |
| | | (m, 1H), 2.02 (m, | |
| | | 1H), 1.66 (m, | |
| | | 4H), 1.0-0.85 | |
| | | (m, 6H). | |
| | 2-amino-N⁸-(5-amino-6,7-dihydro-5H- | | |
| | cyclopenta[b]pyridin-3-yl)-N⁴,N⁴-dipropyl-3H- | | |
| | benzo[b]azepine-4,8-dicarboxamide | | |
| 1.42 | 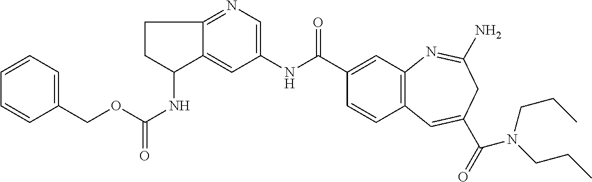 [SMILES: CCCN(CCC)C(=O)C1=Cc2ccc(C(=O)Nc3cnc4c(c3)C(NC(=O)OCc3ccccc3)CC4)cc2N=C(N)C1] | (CD₃OD) δ 8.72 (d, J = 2.1 Hz, 1H), 8.11 (s, 1H), 8.05 (s, 1H), 8.00 (d, J = 2.0 Hz, 1H), 7.61 (dd, J = 1.5, 8.2 Hz, 1H), 7.33-7.15 (m, 5H), 7.66 (d, 1H), 7.44-7.20 (m, 5H), | 595.4 |
| | | 7.10 (s, 1H), | |
| | | 5.25 (m, | |
| | | 1H), 5.15 (s, | |
| | | 1H), 3.63-3.55 (m, | |
| | | 4H), 3.10-2.95 (m, | |
| | | 2H), 2.00 1.66 (m, | |
| | | 4H), 1.0-0.85 | |
| | | (m, 6H). | |
| | Benzyl (3-(2-amino-4-(dipropylcarbamoyl)-3H- | | |
| | benzo[b]azepine-8-carboxamido)-6,7-dihydro-5H- | | |
| | cyclopenta[b]pyridin-5-yl)carbamate | | |
| 1.43 | 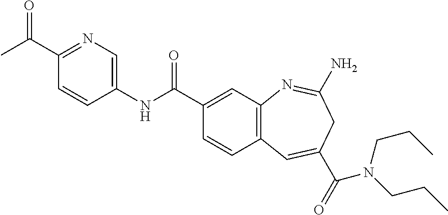 [SMILES: CCCN(CCC)C(=O)C1=Cc2ccc(C(=O)Nc3ccc(C(C)=O)nc3)cc2N=C(N)C1] | (DMSO-d₆) δ 12.3 (s, 1H), 10.9 (s, 1H), 9.89 (s, 1H), 9.17 9s, 1H), 9.06 (s, 1H), 8.45 (d, J = 8.8 Hz, 1H), 8.05- 7.95 (m, 3H), 7.77 (d, J = 8.0 Hz, 1H), 7.05 (s, 1H), 3.44 (m, 6H), 2.60 (s, 3H), 1.65 (m, | 448.2 |
| | | 4H), 0.90 (m, 6H). | |
| | N⁸-(6-acetylpyridin-3-yl)-2-amino-N⁴,N⁴-dipropyl-3H- | | |
| | benzo[b]azepine-4,8-dicarboxamide | | |
| 1.44 | 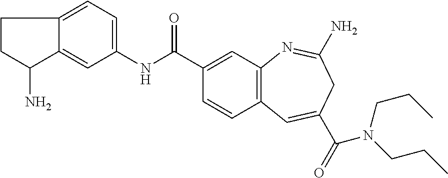 [SMILES: CCCN(CCC)C(=O)C1=Cc2ccc(C(=O)Nc3ccc4c(c3)C(N)CC4)cc2N=C(N)C1] | (DMSO-d₆) δ 10.1 (s, 1H), 7.90 (s, 1H), 7.75 (s, 1H), 7.50- 7.40 (m, 3H), 7.17 (d, J = 8.4 Hz, 1H), 6.90 (bs, 1H), 6.68 (s, 1H), 4.25 (m, 1H), 3.50-3.30 (m, | 460.3 |
| | | 6H), 2.85-2.65 (m, | |
| | | 4H), 2.40 (m, 1H), | |
| | | 1.65-1.55 (m, 5H), | |
| | | 0.85 (bs, 6H). | |
| | 2-amino-N⁸-(3-amino-2,3-dihydro-1H-inden-5-yl)-N⁴,N⁴- | | |
| | dipropyl-3H-benzo[b]azepine-4,8-dicarboxamide | | |
| 1.45 | 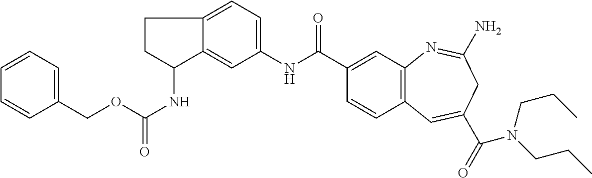 [SMILES: CCCN(CCC)C(=O)C1=Cc2ccc(C(=O)Nc3ccc4c(c3)C(NC(=O)OCc3ccccc3)CC4)cc2N=C(N)C1] | (DMSO-d₆) δ 10.1 (s, 1H), 7.72-7.55 (m, 3H), 7.50-7.40 (m, 5H), 7.17 (d, J = 8.4 Hz, 1H), 6.90 (bs, 1H), 6.88 (s, 1H), 5.15 (m, 3H), 3.40 (m, 4H), 2.85-2.65 (m, 4H), 2.40 (m, 1H), 1.80 (m, | 594.3 |
| | | 1H), 1.65-1.55 (m, | |
| | | 4H), 0.85 (bs, 6H). | |
| | Benzyl (6-(2-amino-4-(dipropylcarbamoyl)-3H- | | |
| | benzo[b]azepine-8-carboxamido)-2,3-dihydro-1H-inden-1- | | |
| | yl)carbamate | | |
| 1.46 | 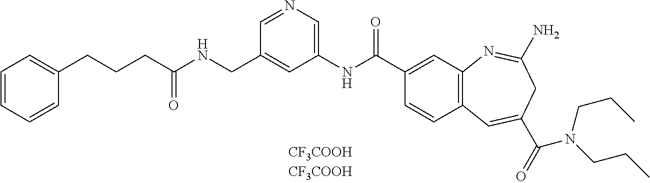 [SMILES: CCCN(CCC)C(=O)C1=Cc2ccc(C(=O)Nc3cncc(CNC(=O)CCCc4ccccc4)c3)cc2N=C(N)C1] | (CD₃OD) δ 9.15 (d, J = 2.1 Hz, 1H), 8.51 (s, 1H), 8.43 (s, 1H), 8.00 (s, 1H), 7.96 (dd, J = 8.4, 2.1 Hz, 1H), 7.71 (d, J = 8.5 Hz, 1H), 7.25-7.11 (m, 6H), 4.50 (s, 2H), 3.46 (m, | 581.2 |
| | | 4H), 3.37 (s, 2H), | |
| | | 2.64 (t, | |
| | | J = 7.5 Hz, 2H), | |
| | | 2.31 (t, J = 7.5 Hz, | |
| | | 2H), 1.95 (m, | |
| | | 2H), 1.69 (m, 4H), | |
| | | 0.96 (bs, 3H), 0.92 | |
| | | (bs, 3H). | |
| | 2-amino-N⁸-(5-((4-phenylbutanamido)methyl)pyridin-3- | | |
| | yl)-N⁴,N⁴-dipropyl-3H-benzo[b]azepine-4,8- | | |
| | dicarboxamide bis TFA salt | | |
| 1.47 | 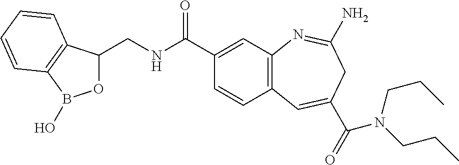 [SMILES: CCCN(CCC)C(=O)C1=Cc2ccc(C(=O)NCC3OB(O)c4ccccc43)cc2N=C(N)C1] | (CD₃OD) δ 7.78 (d, J = 1.5 Hz, 1H), 7.73 (dd, J = 1.5, 8.5 Hz, 1H), 7.66 (d, J = 7.0 Hz, 1H), 7.62 (d, J = 8.5 Hz 1H), 7.47 (m, 2H), 7.37 (m, 1H), 7.07 (s, 1H), 5.44 | 473.2 |
| | | (dd, J = 3.5, | |
| | | 8.5 Hz, 1H), | |
| | | 4.00 (dd, J = 3.5, | |
| | | 14.0 Hz, 1H), 3.6- | |
| | | 3.4 (m, 7H), 1.69 | |
| | | (m, 4H), | |
| | | 0.95 (bs, 3H), 0.91 | |
| | | (bs, 3H). | |
| | 2-amino-N⁸-((1-hydroxy-1,3-dihydro- | | |
| | benzo[c][1,2]oxaborol-3-yl)methyl)-N⁴,N⁴-dipropyl-3H- | | |
| | benzo[b]azepine-4,8-dicarboxamide | | |
| 1.48 | 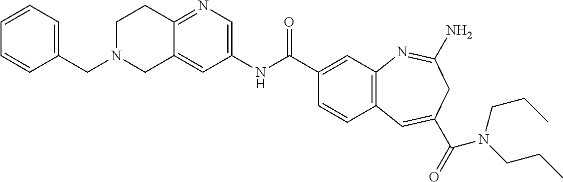 [SMILES: CCCN(CCC)C(=O)C1=Cc2ccc(C(=O)Nc3cnc4c(c3)CN(Cc3ccccc3)CC4)cc2N=C(N)C1] | (CD₃OD) δ 9.33 (s, 1H), 8.89 (s, 1H), 8.09 (s, 1H), 8.05 (d, J = 8.4 Hz, 1H), 7.74 (d, J = 8.4 Hz, 1H), 7.31- 7.22 (m, 5H), 7.12 (s, 1H), 4.40 (s, 2H), 4.37 (s, 2H), 3.48 (m, 4H), 3.38-3.28 | 551.3 |
| | | (m, 8H), 1.71 (q, | |
| | | J = 7.5 Hz, 4H), | |
| | | 0.97-0.92 (bs, 6H). | |
| | 2-amino-N⁸-(6-benzyl-5,6,7,8-tetrahydro-1,6- | | |
| | naphthyridin-3-yl)-N⁴,N⁴-dipropyl-3H-benzo[b]azepine- | | |
| | 4,8-dicarboxamide | | |
| 1.49 | 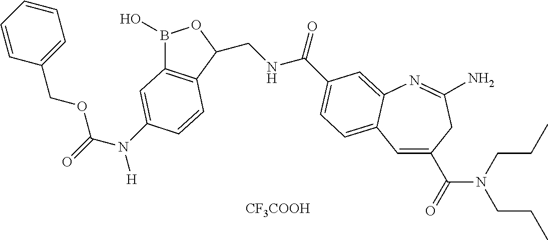 [SMILES: CCCN(CCC)C(=O)C1=Cc2ccc(C(=O)NCC3OB(O)c4cc(NC(=O)OCc5ccccc5)ccc43)cc2N=C(N)C1 ; "CF3COOH"] | (DMSO-d₆) δ 12.0 (s, 1H), 9.82 (s, 1H), 9.29 (s, 1H), 8.98 (s, 1H), 8.92 (m, 1H), 7.88-7.83 (m, 3H), 7.65 (d, J = 8.5 Hz, 1H), 7.50 (dd, J = 8.0, .0 Hz, 1H), 7.45-7.33 (m, 6H), 7.01 (s, 1H), 5.30 (m, 1H), 5.15 (s, 2H), 3.70 | 624 |
| | | (m, 1H), 4.40- | |
| | | 3.30 (m, | |
| | | 5H), 1.58 (m, 4H), | |
| | | 0.89 (bs, 3H), | |
| | | 0.80 (bs, 3H). | |
| | benzyl (3-((2-amino-4-(dipropylcarbamoyl)-3H- | | |
| | benzo[b]azepine-8-carboxamido)methyl)-1-hydroxy-1,3- | | |
| | dihydrobenzo[c][1,2]oxaborol-6-yl)carbamate TFA salt | | |
| 1.50 | 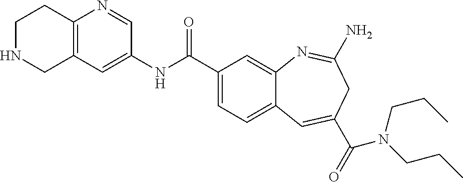 [SMILES: CCCN(CCC)C(=O)C1=Cc2ccc(C(=O)Nc3cnc4c(c3)CNCC4)cc2N=C(N)C1] | (CD₃OD) δ 8.68 (s, 1H), 8.00 (s, 1H), 7.70 (s, 1H), 7.58 (dd, J = 8.4, 2.1 Hz, 1H), 7.47 (d, J = 8.4 Hz, 1H), 6.91 (s, 1H), 4.08 (s, 2H), 3.38- 3.28 (m, 6H), 2.95 (t, J = | 461 |
| | | 3.0 Hz, 2H), | |
| | | 1.71 (q, J = | |
| | | 7.5 Hz, 4H), | |
| | | 0.97-0.92 (bs, 6H). | |
| | 2-amino-N⁴,N⁴-dipropyl-N⁸-(5,6,7,8-tetrahydro-1,6- | | |
| | naphthyridin-3-yl)-3H-benzo[b]azepine-4,8- | | |
| | dicarboxamide | | |
| 1.51 | 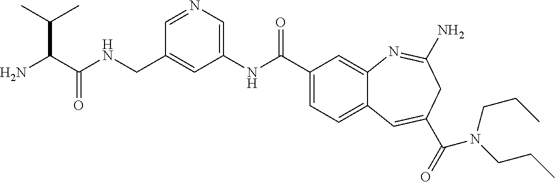 [SMILES: CCCN(CCC)C(=O)C1=Cc2ccc(C(=O)Nc3cncc(CNC(=O)[C@@H](N)C(C)C)c3)cc2N=C(N)C1] | (CD₃OD) δ 8.78 (d, J = 2.3 Hz, 1H), 8.33 (s, 1H), 8.31 (s, 1H), 7.72 (s, 1H), 7.57 (dd, J = 1.5, 8.2 Hz, 1H), 7.45 (d, = 8.4 Hz, 1H), 6.90 (s, 1H), 4.48 (s, 2H), 3.43 (m, | 534 |
| | | 4H), 2.00 (m, | |
| | | 1H), 1.66 (m, | |
| | | 4H), 1.0- | |
| | | 0.85 (m, 12H). | |
| | (S)-2-amino-N⁸-(5-((2-amino-3- | | |
| | methylbutanamido)methyl)pyridin-3-yl)-N⁴,N⁴-dipropyl- | | |
| | 3H-benzo[b]azepine-4,8-dicarboxamide | | |
| 1.52 | 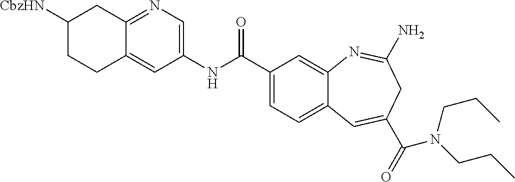 [SMILES: CCCN(CCC)C(=O)C1=Cc2ccc(C(=O)Nc3cnc4c(c3)CCC(NC(=O)OCc3ccccc3)C4)cc2N=C(N)C1] | (DMSO-d₆) δ 10.3 (s, 1H), 8.70 (s, 1H), 7.98 (s, 1H), 7.66 (s, 1H), 7.52-7.35 (m, 6H), 6.90 (bs, 1H), 6.88 (s, 1H), 5.15 (s, 2H), 3.80 (m, 1H), 3.43 (m, 4H), 3.00-2.65 (m, 6H), 2.02 (m, 1H), 1.65- | 609.3 |
| | | 1.55 (m, 4H), | |
| | | 0.85 (bs, 6H). | |
| | benzyl (3-(2-amino-4-(dipropylcarbamoyl)-3H- | | |
| | benzo[b]azepine-8-carboxamido)-5,6,7,8- | | |
| | tetrahydroquinolin-7-yl)carbamate | | |
| 1.53 | 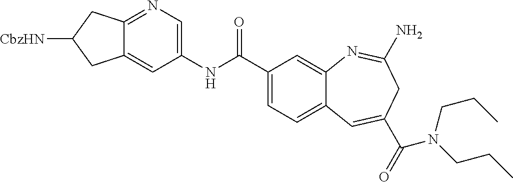 [SMILES: CCCN(CCC)C(=O)C1=Cc2ccc(C(=O)Nc3cnc4c(c3)CC(NC(=O)OCc3ccccc3)C4)cc2N=C(N)C1] | (CD₃OD) δ 9.05 (m, 1H), 8.45 (m, 1H), 7.98 (m, 1H), 7.66 (m, 1H), 7.22-7.35 (m, 5H), 7.10 (s, 1H), 5.09 (s, 2H), 4.73 (m, 1H), 3.43 (m, 4H), 3.00- 2.65 (m, 2H), 1.72- | 595.3 |
| | | 1.62 (m, 4H), | |
| | | 0.85 (bs, 6H). | |
| | benzyl (3-(2-amino-4-(dipropylcarbamoyl)-3H- | | |
| | benzo[b]azepine-8-carboxamido)-6,7-dihydro-5H- | | |
| | cyclopenta[b]pyridin-6-yl)carbamate | | |
| 1.54 | 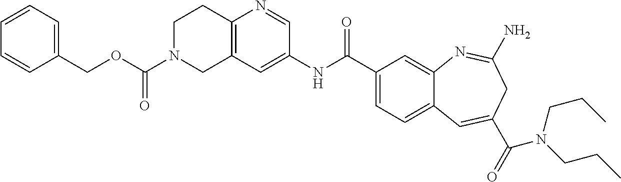 [SMILES: CCCN(CCC)C(=O)C1=Cc2ccc(C(=O)Nc3cnc4c(c3)CN(C(=O)OCc3ccccc3)CC4)cc2N=C(N)C1] | (CD₃OD) δ 8.70 (s, 1H), 8.05 (s, 1H), 7.93 (m, 2H), 7.66 (d, J = 7.8 Hz, 1H), 7.42-7.31 (m, 5H), 7.08 (s, 1H), 5.19 (s, 2H), 4.73 (m, 2H), 3.85 (bs, 2H), 3.43 (m, 4H), 3.00- 2.95 (m, | 595 |
| | | 2H), 1.72- | |
| | | 1.62 (m, 4H), | |
| | | 0.85 (bs, 6H). | |
| | benzyl 3-(2-amino-4-(dipropylcarbamoyl)-3H- | | |
| | benzo[b]azepine-8-carboxamido)-7,8-dihydro-1,6- | | |
| | naphthyridine-6(5H)-carboxylate | | |
| 1.55 | 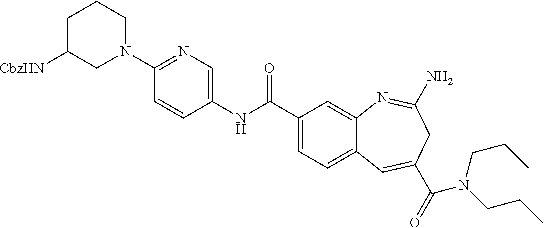 [SMILES: CCCN(CCC)C(=O)C1=Cc2ccc(C(=O)Nc3ccc(N4CCCC(NC(=O)OCc5ccccc5)C4)nc3)cc2N=C(N)C1] | (DMSO-d₆) δ 12.2 (bs, 1H), 10.2 (s, 1H), 8.50 (s, 1H), 8.00- 7.75 (m, 3H), 7.65 (d, J = 7.8 Hz, 1H), 7.43-7.25 (m, 5H), 7.01 (s, 1H), 6.82 (d, J = 8.8 Hz, 1H), 5.04 (s, 2H), 4.21 (d, J = 12 Hz, | 638.3 |
| | | 1H), 4.04 (d, | |
| | | J = 12 Hz, | |
| | | 1H), 3.55-3.00 | |
| | | (m, 7H), 2.80- | |
| | | 2.70 (m, 2H), | |
| | | 2.00-1.40 (m, 8H), | |
| | | 0.85 (bs, 6H). | |
| | benzyl (1-(5-(2-amino-4-(dipropylcarbamoyl)-3H- | | |
| | benzo[b]azepine-8-carboxamido)pyridin-2-yl)piperidin-3- | | |
| | yl)carbamate | | |
| 1.56 | 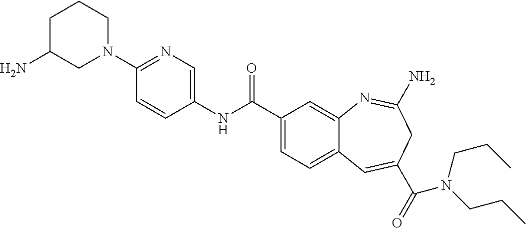 [SMILES: CCCN(CCC)C(=O)C1=Cc2ccc(C(=O)Nc3ccc(N4CCCC(N)C4)nc3)cc2N=C(N)C1] | (DMSO-d₆) δ 10.2 (s, 1H), 8.48 (s, 1H), 7.98 (d, J = 7.2 Hz, 1H), 7.65 (s, 1H), 7.45 (m, 2H), 6.82 (d, J = 8.2 Hz, 1H), 4.21 (d, J = 12 Hz, 1H), 3.94 (d, J = 12 Hz, 1H), 2.80-2.70 (m, 4H), 2.00-1.40 (m, 10H), 0.85 | 504.2 |
| | | (bs, 6H). | |
| | 2-amino-N⁸-(6-(3-aminopiperidin-1-yl)pyridin-3-yl)- | | |
| | N⁴,N⁴-dipropyl-3H-benzo[b]azepine-4,8-dicarboxamide | | |
| 1.57 | 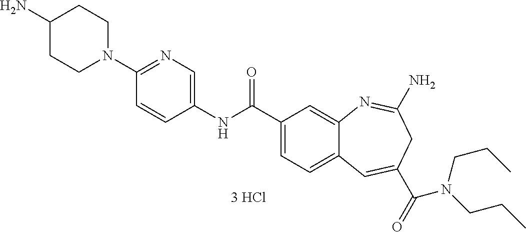 [SMILES: CCCN(CCC)C(=O)C1=Cc2ccc(C(=O)Nc3ccc(N4CCC(N)CC4)nc3)cc2N=C(N)C1] | (CD₃OD) δ 8.65 (s, 1H), 8.16 (m, 1H), 8.00-7.96 (m, 2H), 7.70 (d, J = 8.0 Hz, 1H), 7.32 (m, 1H), 7.11 (s, 1H), 4.33 (d, J = 13.5 Hz, 2H), 3.47- 3.40 (m, 5H), 2.17 (m 2H), 1.72 (m, 6H), 0.94 (m, 6H). | 504.6 |
| | 2-amino-N⁸-(6-(4-aminopiperidin-1-yl)pyridin-3-yl)- | | |
| | N⁴,N⁴-dipropyl-3H-benzo[b]azepine-4,8-dicarboxamide | | |
| | tris HCl salt | | |
| 1.58 | 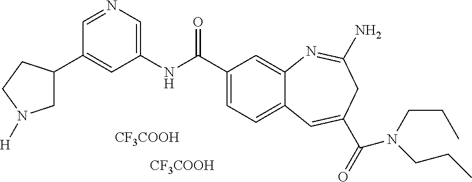 [SMILES: CCCN(CCC)C(=O)C1=Cc2ccc(C(=O)Nc3cncc(C4CCNC4)c3)cc2N=C(N)C1 ; "CF3COOH" ; "CF3COOH"] | (CD₃OD) δ 8.84 (J = 1.5 Hz, 1H), 8.42 (d, J = 1.5 Hz, 1H), 8.37 (d, J = 1.5 Hz, 1H), 8.01- 7.98 (m, 2H), 7.71 (d, J = 8.0 Hz, 1H), 7.11 (s, 1H), 4.80 (m, 1H), 3.83 (m, 1H), 7.73- | 475 |
| | | 3.60 (m, 2H), 3.52- | |
| | | 3.44 (m, 2H), | |
| | | 2.57 (m, | |
| | | 1H), 2.18 (m, 1H), | |
| | | 1.71 (q, J = | |
| | | 7.5 Hz, 4H), | |
| | | 0.97 (bs, 3H), 0.92 | |
| | | (bs, 3H). | |
| | 2-amino-N⁴,N⁴-dipropyl-N⁸-(5-(pyrrolidin-3-yl)pyridin-3- | | |
| | yl)-3H-benzo[b]azepine-4,8-dicarboxamide | | |
| 1.59 | 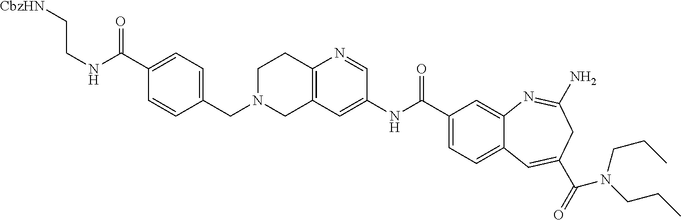 [SMILES: CCCN(CCC)C(=O)C1=Cc2ccc(C(=O)Nc3cnc4c(c3)CN(Cc3ccc(C(=O)NCCNC(=O)OCc5ccccc5)cc3)CC4)cc2N=C(N)C1] | (CD₃OD) δ 8.71 (J = 1.5 Hz, 1H), 8.05 (bs, 1H), 7.95 (m, 2H), 7.87 (m, 2H), 7.70 (d, J = 9.0 Hz, 1H), 7.57 (d, J = 7.5 Hz, 1H), 7.32-7.25 (m, 5H), 7.11 (s, 1H), 5.06 (s, 2H), 3.51- 3.46 (m, | 771 |
| | | 6H), 3.37 (m, | |
| | | 4H), 1.69 (q, | |
| | | J = 7.5 Hz, 4H), | |
| | | 0.96 (bs, 3H), | |
| | | 0.92 (bs, 3H). | |
| | benzyl (2-(4-((3-(2-amino-4-(dipropylcarbamoyl)-3H- | | |
| | benzo[b]azepine-8-carboxamido)-7,8-dihydro-1,6- | | |
| | naphthyridin-6(5H)-yl)methyl)benzamido)ethyl)carbamate | | |
| 1.60 | 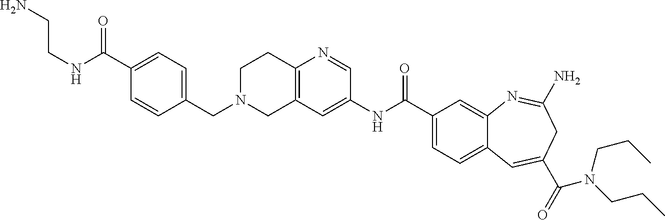 [SMILES: CCCN(CCC)C(=O)C1=Cc2ccc(C(=O)Nc3cnc4c(c3)CN(Cc3ccc(C(=O)NCCN)cc3)CC4)cc2N=C(N)C1] | (CD₃OD) δ 8.65 (J = 2.5 Hz, 1H), 7.95 (J = 2.5 Hz, 1H), 7.85 (d, J = 8.5 Hz, 2H), 7.68 (d, J = 2.0 Hz, 1H), 7.58-7.53 (m, 3H), 7.44 (d, J = 8.5 Hz, 1H), 6.89 (s, 1H), 3.82 (s, 2H), 3.70 (s, 2H), 3.53 (t, | 637.6 |
| | | J = 6.0 Hz, | |
| | | 2H), 3,42 (m, | |
| | | 4H), 3.00-2.89 | |
| | | (m, 6H), 1.67 | |
| | | (m, 4H), 0.95- | |
| | | 0.87 (m, 6H). | |
| | 2-amino-N⁸-(6-(4-((2-aminoethyl)carbamoyl)benzyl)- | | |
| | 5,6,7,8-tetrahydro-1,6-naphthyridin-3-yl)-N⁴,N⁴-dipropyl- | | |
| | 3H-benzo[b]azepine-4,8-dicarboxamide | | |
| 1.61 | 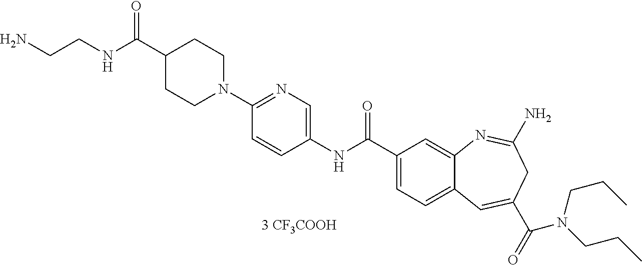 [SMILES: CCCN(CCC)C(=O)C1=Cc2ccc(C(=O)Nc3ccc(N4CCC(C(=O)NCCN)CC4)nc3)cc2N=C(N)C1] | (CD₃OD) δ 8.57 (d, J = 2.5 Hz, 1H), 8.06 (dd, J = 8.0, 2.5 Hz, 1H), 7.96 (s, 1H), 7.94 (d, J = 8.0 Hz, 1H), 7.70 (d, J = 8.0 Hz, 1H), 7.21 (d, J = 8.0 Hz, 1H), 7.11 (s, 1H), 4.25 (d, J = 13.5 Hz, 2H), 3.48- 3.44 (m, 6H), 3.17 (m, 2H), 3.06 (t, | 575.6 |
| | | J = 6.0 Hz, | |
| | | 2H), 2.57 (m, 1H), | |
| | | 1.99-1.95 (m, 2H), | |
| | | 1.82-1.79 (m, 2H), | |
| | | 1.73-1.66 (m, 4H), | |
| | | 0.97 (bs, 3H), 0.91 | |
| | | (bs, 3H). | |
| | 2-amino-N⁸-(6-(4-((2-aminoethyl)carbamoyl)piperidin-1- | | |
| | yl)pyridin-3-yl)-N⁴,N⁴-dipropyl-3H-benzo[b]azepine-4,8- | | |
| | dicarboxamide tris TFA salt | | |
| 1.62 | 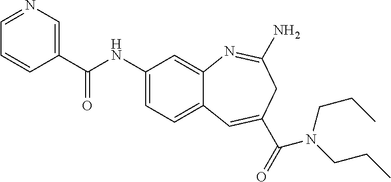 [SMILES: CCCN(CCC)C(=O)C1=Cc2ccc(NC(=O)c3cccnc3)cc2N=C(N)C1] | | |
| | 2-amino-8-(nicotinamido)-N,N-dipropyl-3H- | | |
| | benzo[b]azepine-4-carboxamide | | |
| 1.63 | 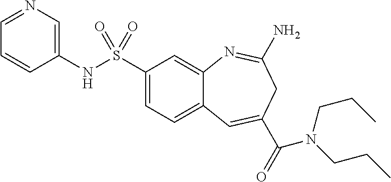 [SMILES: CCCN(CCC)C(=O)C1=Cc2ccc(S(=O)(=O)Nc3cccnc3)cc2N=C(N)C1] | | |
| | 2-amino-N,N-dipropyl-8-(N-(pyridin-3-yl)sulfamoyl)-3H- | | |
| | benzo[b]azepine-4-carboxamide | | |
| 1.64 | 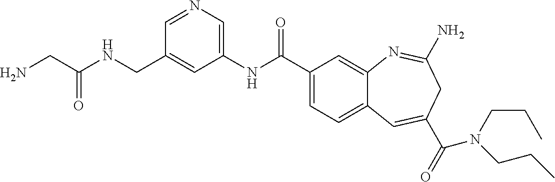 [SMILES: CCCN(CCC)C(=O)C1=Cc2ccc(C(=O)Nc3cncc(CNC(=O)CN)c3)cc2N=C(N)C1] | 1H NMR (DMSO-d₆) δ 10.4 (s, 1H), 8.85 (d, J = 2.4 Hz, 1H), 8.44 (t, J = 6.0 Hz, 1H), 8.23 (d, 2.0 Hz, 1H), 8.13 (d, t, J = 2.0 Hz, 1H), 7.68 (d, J = 2.0 Hz, 1H), 7.50 (dd, J = | 492.3 |
| | | 2.0, 8.0 Hz, 1H), | |
| | | 7.41 (d, J = | |
| | | 8.0 Hz, 1H), | |
| | | 6.91 (bs, 2H), | |
| | | 6.79 (s, 1H), 4.33 | |
| | | (d, J = 5.6 Hz, | |
| | | 1H), 3.33 (m, 2H), | |
| | | 3.15 (s, 1H), | |
| | | 2.73 (s, | |
| | | 1H), 1.78 (bs, 1H), | |
| | | 1.56 (m, 4H), | |
| | | 0.84 (bs, 6H). | |
| | 2-amino-N⁸-(5-((2-aminoacetamido)methyl)pyridin-3-yl)- | | |
| | N⁴,N⁴-dipropyl-3H-benzo[b]azepine-4,8-dicarboxamide | | |
| 1.65 | 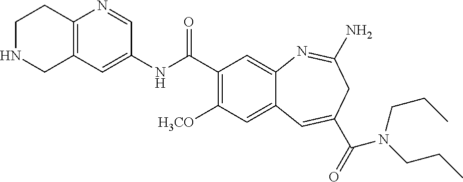 [SMILES: CCCN(CCC)C(=O)C1=Cc2cc(OC)c(C(=O)Nc3cnc4c(c3)CNCC4)cc2N=C(N)C1] | | |
| | 2-amino-7-methoxy-N⁴,N⁴-dipropyl-N⁸-(5,6,7,8- | | |
| | tetrahydro-1,6-naphthyridin-3-yl)-3H-benzo[b]azepine- | | |
| | 4,8-dicarboxamide | | |
| 1.66 | 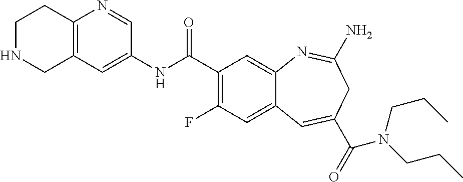 [SMILES: CCCN(CCC)C(=O)C1=Cc2cc(F)c(C(=O)Nc3cnc4c(c3)CNCC4)cc2N=C(N)C1] | | |
| | 2-amino-7-fluoro-N⁴,N⁴-dipropyl-N⁸-(5,6,7,8-tetrahydro- | | |
| | 1,6-naphthyridin-3-yl)-3H-benzo[b]azepine-4,8- | | |
| | dicarboxamide | | |
| 1.67 | 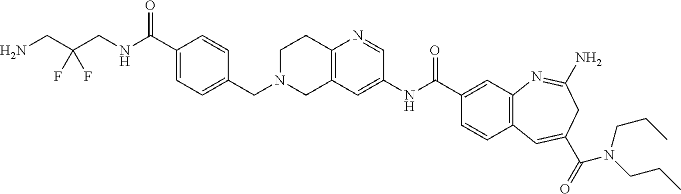 [SMILES: CCCN(CCC)C(=O)C1=Cc2ccc(C(=O)Nc3cnc4c(c3)CN(Cc3ccc(C(=O)NCC(F)(F)CN)cc3)CC4)cc2N=C(N)C1] | | |
| | 2-amino-N⁸-(6-(4-((3-amino-2,2- | | |
| | difluoropropyl)carbamoyl)benzyl)-5,6,7,8-tetrahydro-1,6- | | |
| | naphthyridin-3-yl)-N⁴,N⁴-dipropyl-3H-benzo[b]azepine- | | |
| | 4,8-dicarboxamide | | |

### Example 3: Synthesis of 8-Substituted Anilides: Preparation of 2-amino-8-(nicotinamido)-N,N-dipropyl-3H-benzo[b]azepine-4-carboxamide (Compound 1.62)

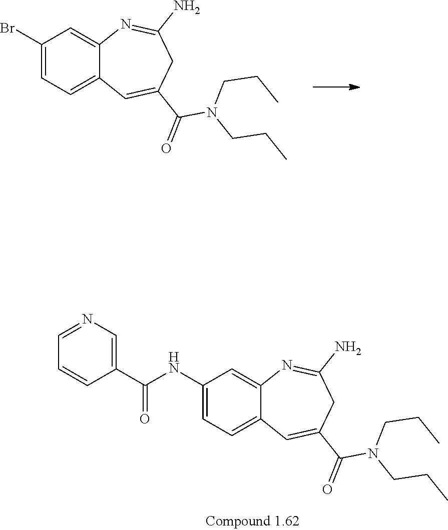 [SMILES: CCCN(CCC)C(=O)C1=Cc2ccc(Br)cc2N=C(N)C1 ; CCCN(CCC)C(=O)C1=Cc2ccc(NC(=O)c3cccnc3)cc2N=C(N)C1]

Step A: Preparation of Compound 1.62

To a solution containing 46 mg (0.10 mmol) of tert-butyl (8-bromo-4-(dipropylcarbamoyl)-3H-benzo[b]azepin-2-yl)carbamate in 5 mL of DMF was added 65 mg (0.20 mmol) of Cs₂CO₃ and 15 mg (0.12 mmol) of nicotinamide. The solution was degassed then treated with 18 mg (0.2 equiv.) of [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate (BrettPhos Pd G3) and 11 mg (0.2 equiv.) of 2-(dicyclohexylphosphino)3,6-dimethoxy-2′,4′,6′-triisopropyl-1,1′-biphenyl (BrettPhos) and heated at 90° C. for 12 h. The reaction mixture was cooled and chromatographed by preparative HPLC to afford 6 mg of the desired coupled and deprotected compound as an off-white solid. ¹H NMR (DMSO-d₆) δ 10.4 (s, 1H), 9.10 (d, J=1.6 Hz, 1H), 8.76 (d, J=8.0 Hz, 1H), 8.28 (d, J=8.0 Hz, 1H), 7.55 (m, 1H), 7.52 (s, 1H), 7.36 (d, J=8.2 Hz, 1H), 7.27 (d, J=8.0 Hz, 1H), 6.80 (bs, 1H), 6.68 (s, 1H), 3.44 (m, 4H), 2.69 (m, 1H), 1.54 (m, 4H), 0.89 (bs, 6H). LCMS (M+H)=406.2.

### Example 4: Synthesis of 8-Substituted Sulfonamides: Preparation of 2-amino-N,N-dipropyl-8-(N-(pyridin-3-yl)sulfamoyl)-3H-benzo[b]azepine-4-carboxamide (Compound 1.63)

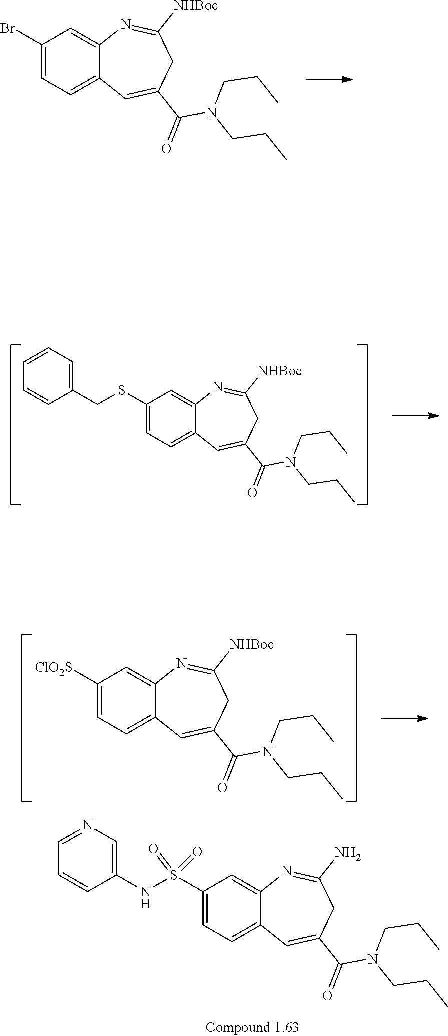

Step A: Preparation of Compound 1.63

To a solution containing 460 mg (1.0 mmol) of tert-butyl (8-bromo-4-(dipropylcarbamoyl)-3H-benzo[b]azepin-2-yl)carbamate in 50 mL of dioxane was added 210 mg (2.0 mmol) of N,N-diisopropylethylamine and 140 mg (1.2 mmol) of benzylthiol. The solution was degassed then treated with 180 mg (0.20 mmol) of Pd₂(dba)₃ and 116 mg (0.20 mmol) of 4,5-bis(diphenylphosphino)-9,9-dimethylxanthene (XantPhos) and heated at 90° C. for 6 h. The reaction mixture was cooled and filtered through diatomaceous earth, then chromatographed by reverse phase chromatography to afford 250 mg of the desired thiol ether, which was immediately dissolved in DCM (20 ml) and acetic acid (0.5 ml). The resulting solution was cooled in an ice water bath and 1,3-dichloro-5,5-dimethy 2-imidazolidinedione (197 mg, 1.0 mmol) was added. After 2 h the mixture was extracted with DCM and brine and the organics were dried and evaporated. The residue was dissolved in MeCN and treated with 1-methyl-1H-imidazole and 3-aminopyridine at 0° C. and stirred to room temperature over 2 h. The solution was extracted with brine and dried over Na₂SO₄. The residue was then dissolved in 4 mL of DCM and treated with 1 mL of TFA and stirred for 2 h. Evaporation of the solvent and purification by reverse phase HPLC afforded 30 mg of the desired compound 1.63. ¹H NMR (DMSO-d₆) δ 10.5 (bs, 1H), 8.32 (s, 1H), 8.25 (d, J=2.0 Hz, 1H), 7.54 (d, 8.0 Hz, 1H), 7.52 (d, J=8.0 Hz, 1H), 7.45 (s, 1H), 7.22 (dd, J=8.0, 2.0 Hz, 1H), 7.07 (m, 2H), 6.73 (s, 1H), 3.30 (m, 4H), 2.95 (s, 2H), 2.11 (s, 1H), 1.54 (m, 4H), 0.85 (bs, 6H). LCMS (M+H)=442.1.

### Example 5: Synthesis of Linker-Modified Payloads (LP): Preparation of 4-((S)-2-((S)-2-(6-(2,5-dioxo-2,5-dihydro-1H-pyrrol-1-yl)hexanamido)-3-methylbutanamido)-5-ureidopentanamido)benzyl ((5-(2-amino-4-(dipropyl-carbamoyl)-3H-benzo[b]azepine-8-carboxamido)pyridin-3-yl)methyl)carbamate (Compound-Linker 2.1)

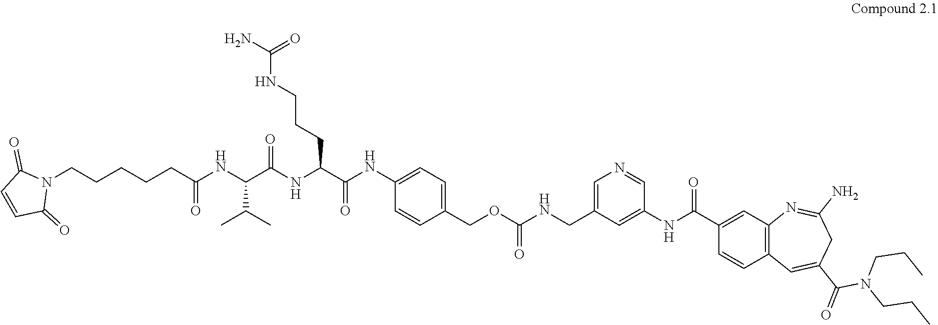 [SMILES: CCCN(CCC)C(=O)C1=Cc2ccc(C(=O)Nc3cncc(CNC(=O)OCc4ccc(NC(=O)[C@H](CCCNC(N)=O)NC(=O)[C@@H](NC(=O)CCCCCN5C(=O)C=CC5=O)C(C)C)cc4)c3)cc2N=C(N)C1]

Step A: Preparation of Compound 2.1

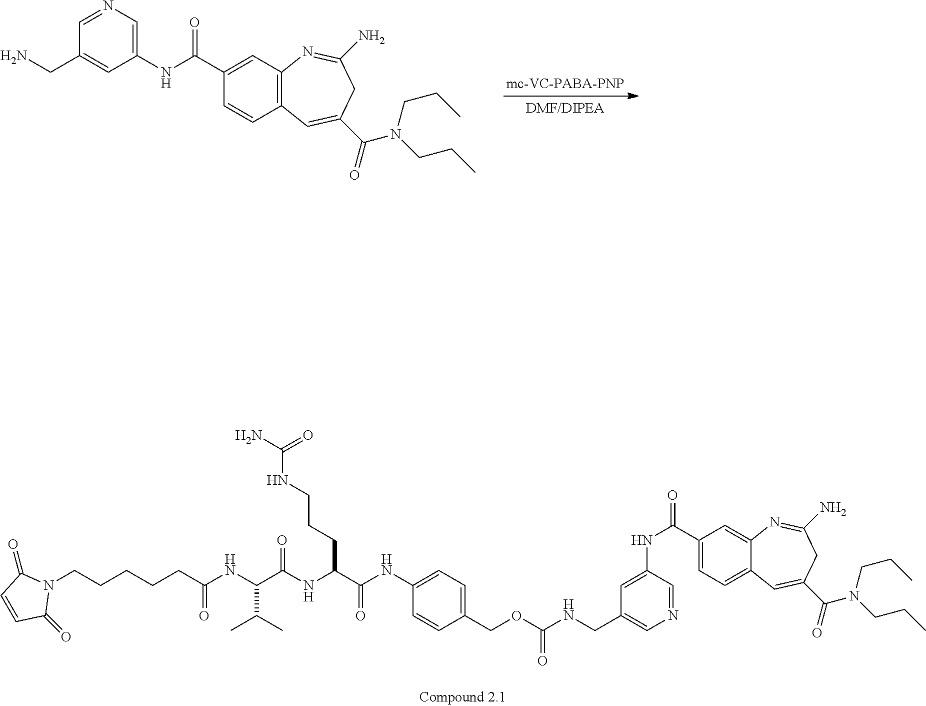 [SMILES: CCCN(CCC)C(=O)C1=Cc2ccc(C(=O)Nc3cncc(CN)c3)cc2N=C(N)C1 ; CCCN(CCC)C(=O)C1=Cc2ccc(C(=O)Nc3cncc(CNC(=O)OCc4ccc(NC(=O)[C@H](CCCNC(N)=O)NC(=O)[C@@H](NC(=O)CCCCCN5C(=O)C=CC5=O)C(C)C)cc4)c3)cc2N=C(N)C1]

54 mg (0.07 mmol) of MC-Val-Cit-PAB-PNP (CAS No. 159857-81-5) was added to a solution containing 40 mg (0.07 mmol) of 2-amino-N⁸-(5-(aminomethyl)pyridin-3-yl)-N⁴,N⁴-dipropyl-3H-benzo[b]azepine-4,8-dicarboxamide in 1.0 mL of DMF and 32 μL (0.18 mmol) of DIPEA. The reaction mixture was stirred for 16 h then purified directly by reverse phase chromatography (no TFA). The clean fractions were lyophilized to afford 60 mg (71%) of the desired product which was dissolved in 5 mL of DCM and treated with 1 mL of TFA at room temperature. The mixture was stirred for 45 minutes and then evaporated. The resulting residue was purified by reverse phase chromatography (no TFA) to afford 34 mg (62%) of Compound-Linker 2.1 as a white solid. ¹H NMR (CD₃OD) δ 8.81 (s, 1H), 8.25 (s, 1H), 8.21 (s, 1H), 7.72 (s, 1H), 7.58 (m, 2H), 7.45 (d, J=8.2 Hz, 2H), 7.33 (d, J=8.4 Hz, 2H), 6.91 (s, 1H), 6.75 (s, 2H), 5.08 (s, 2H), 4.49 (m, 1H), 4.39 (m, 2H), 4.14 (d, J=6.5 Hz, 1H), 3.47 (t, J=7.1 Hz, 2H), 3.42 (m, 4H), 3.15 (m, 1H), 3.10 (m, 1H), 2.27 (t, J=7.4 Hz, 2H), 2.05 (m, 1H), 1.88 (m, 1H), 1.75-1.52 (m, 13H), 1.31 (m, 2H), 0.97 (t, J=6.5 Hz, 6H). LCMS [M+H]=1033.

### Example 6: Synthesis of Linker-Modified Payloads (LP) with Myeloid Cell Agonists: Preparation of 2-amino-N⁸-(5-((6-(4-((2,5-dioxo-2,5-dihydro-1H-pyrrol-1-yl)methyl)cyclohexane-1-carboxamido)hexanamido)methyl)pyridin-3-yl)-N⁴,N⁴-dipropyl-3H-benzo[b]azepine-4,8-dicarboxamide (Compound-Linker 2.2)

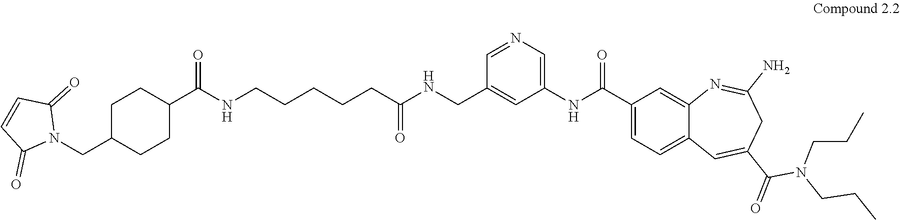 [SMILES: CCCN(CCC)C(=O)C1=Cc2ccc(C(=O)Nc3cncc(CNC(=O)CCCCCNC(=O)C4CCC(CN5C(=O)C=CC5=O)CC4)c3)cc2N=C(N)C1]

Step A: Preparation of Compound 2.2

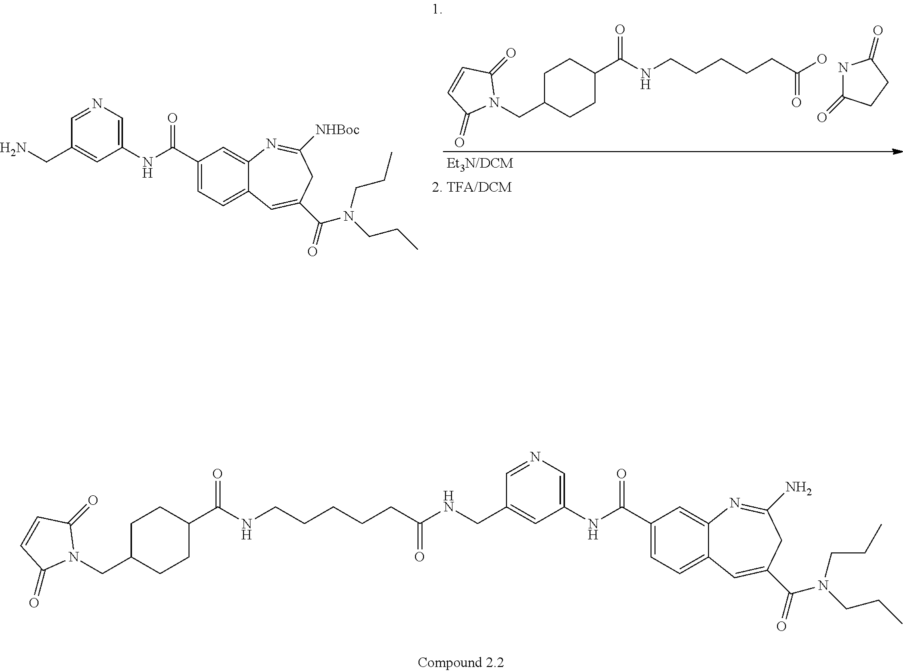

50 mg (0.11 mmol) of N-succinimidyl 6-[[4-(maleimidomethyl)cyclohexyl]carboxamido]caproate (CAS No. 125559-00-4) was added to a solution containing 60 mg (0.11 mmol) of 2-amino-N⁸-(5-(aminomethyl)pyridin-3-yl)-N⁴,N⁴-dipropyl-3H-benzo[b]azepine-4,8-dicarboxamide in 2.0 mL of DCM and 15 μL (0.11 mmol) of triethylamine. The reaction mixture was stirred for 16 h and then purified directly by reverse phase chromatography (no TFA). The clean fractions were lyophilized to afford the desired product which was dissolved in 5 mL of DCM and treated with 1 mL of TFA at room temperature. The mixture was stirred for 2 h and then evaporated. The resulting residue was purified by reverse phase chromatography (no TFA) to afford 49 mg of Compound-Linker 2.2 as a white solid. ¹H NMR (CD₃OD) δ 8.78 (s, 1H), 8.25 (s, 2H), 7.70 (d, J=1.8 Hz, 1H), 7.58 (dd, J=1.8, 8.1 Hz, 1H), 7.46 (d, J=8.3 Hz, 1H), 6.91 (s, 1H), 6.77 (s, 2H), 4.42 (s, 2H), 3.43 (m, 4H), 3.13 (t, J=6.9 Hz, 2H), 2.85 (d, J=16.6 Hz, 1H), 2.29 (t, J=7.3 Hz, 2H), 2.05 (m, 1H), 1.8-1.6 (m, 12H), 1.51 (m, 1H), 1.37 (m, 4H), 1.11-0.84 (m, 9H). LCMS (M+H)=767.

### Example 7: Synthesis of Linker-Modified Payloads (LP)

### Example 7A: Preparation of 2-amino-N⁸-(5-((6-(4-((2,5-dioxo-2,5-dihydro-1H-pyrrol-1-yl)methyl)cyclohexane-1-carboxamido)hexanamido)methyl)pyridin-3-yl)-N⁴,N⁴-dipropyl-3H-benzo[b]azepine-4,8-dicarboxamide (Compound-Linker 2.3)

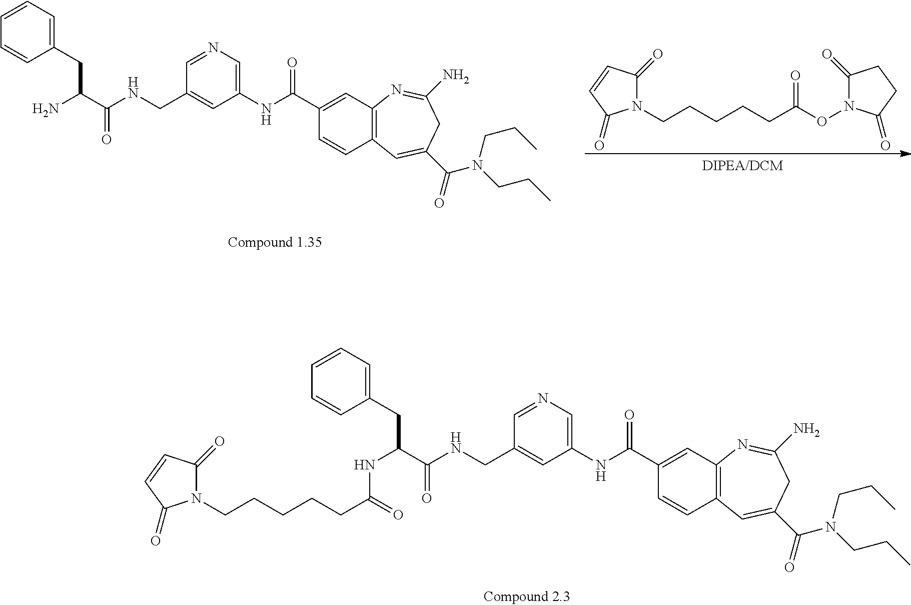

A solution containing 58 mg (0.10 mmol) of Compound 1.35 and 30 mg (0.1 mmol) of 2,5-dioxopyrrolidin-1-yl 6-(2,5-dioxo-2,5-dihydro-1H-pyrrol-1-yl)hexanoate in 2 mL of DCM was treated with 0.07 mL (0.4 mmol) of DIPEA and the reaction was stirred for 4 h at room temperature. The reaction mixture was purified without work-up by reverse phase chromatography to provide 28 mg of Compound-Linker 2.3 as a white solid. ¹H NMR (CD₃OD) δ 8.81 (d, J=2.3 Hz, 1H), 8.19 (d, J=1.9 Hz, 1H), 8.08 (t, J=2.1 Hz, 1H), 7.90 (m, 2H), 7.64 (dd, J=1.9, 8.1 Hz, 1H), 7.25-7.15 (m, 5H), 7.06 (s, 1H), 6.77 (s, 2H), 4.62-4.57 (m, 3H), 4.39 (s, 2H), 3.45-3.40 (m, 4H), 3.39 (t, J=7.5 Hz, 2H), 3.10 (m, 1H), 2.90 (m, 1H), 2.16 (t, J=7.5 Hz, 2H), 1.70 (m, 4H), 1.50 (m, 4H), 1.10 (m, 4H), 0.95 (m, 6H). LCMS (M+H)=775.8. The following compound-linkers 2.4 to 2.7 could be prepared in a manner similar to that described for Compound-Linker 2.3 above by reacting Compound 1.35 with an appropriately substituted linker group.

Compound-Linker 2.4

### (S)-2-amino-N⁸-(5-((2-(6-(4-((2,5-dioxo-2,5-dihydro-1H-pyrrol-1-yl)methyl)cyclohexane-1-carboxamido)hexanamido)-3-phenylpropanamido)methyl)pyridin-3-yl)-N⁴,N⁴-dipropyl-3H-benzo[b]azepine-4,8-dicarboxamide

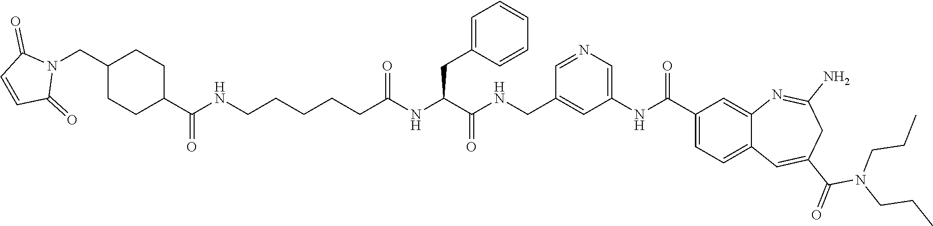 [SMILES: CCCN(CCC)C(=O)C1=Cc2ccc(C(=O)Nc3cncc(CNC(=O)[C@H](Cc4ccccc4)NC(=O)CCCCCNC(=O)C4CCC(CN5C(=O)C=CC5=O)CC4)c3)cc2N=C(N)C1]

From succinimidyl-4-(N-maleimidomethyl)cyclohexane-1-carboxy-(6-amidocaproate) (LC-smcc) to afford a white solid. ¹H NMR (CD₃OD) δ 8.79 (d, J=2.0 Hz, 1H), 8.17 (d, J=2.0 Hz, 1H), 8.09 (t, J=2.0 Hz, 1H), 7.78 (s, 1H), 7.69 (m, 1H), 7.55 (m, 1H), 7.25-7.15 (m, 5H), 6.96 (s, 1H), 6.79 (s, 2H), 4.62-4.57 (m, 1H), 4.38 (s, 2H), 3.45-3.40 (m, 6H), 3.14 (m, 1H), 3.05 (t, J=7.5 Hz, 2H), 2.90 (m, 1H), 2.18 (t, J=7.5 Hz, 2H), 2.10 (m, 1H), 1.80-1.60 (m, 10H), 1.50-1.30 (m, 6H), 1.20-1.10 (m, 3H), 0.95 (m, 6H). LCMS (M+H)=914.9.

Compound-Linker 2.5

### (S)-2-amino-N⁸-(5-(4-benzyl-24-(2,5-dioxo-2,5-dihydro-1H-pyrrol-1-yl)-3,6,22-trioxo-9,12,15,18-tetraoxa-2,5,21-triazatetracosyl)pyridin-3-yl)-N⁴,N⁴-dipropyl-3H-benzo[b]azepine-4,8-dicarboxamide

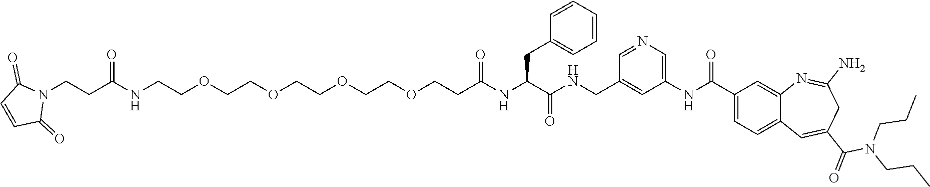 [SMILES: CCCN(CCC)C(=O)C1=Cc2ccc(C(=O)Nc3cncc(CNC(=O)[C@H](Cc4ccccc4)NC(=O)CCOCCOCCOCCOCCNC(=O)CCN4C(=O)C=CC4=O)c3)cc2N=C(N)C1]

From (α-maleimidopropionyl-ω-succinimidyl-4(ethylene glycol)) (mal-PEG4-NHS) to afford a white solid. ¹H NMR (CD₃OD) δ 8.91 (d, J=2.0 Hz, 1H), 8.24 (d, J=2.0 Hz, 1H), 8.15 (t, J=2.0 Hz, 1H), 8.01-7.98 (m, 2H), 7.72 (d, 8.0 Hz, 1H), 7.25-7.15 (m, 5H), 7.12 (s, 1H), 6.78 (s, 2H), 4.60 (m, 1H), 4.43 (s, 2H), 3.73 (t, J=7.5 Hz, 2H), 3.70-3.40 (m, 20H), 3.39 (s, 2H), 3.15 (m, 1H), 2.95 (m, 1H), 2.45 (t, J=7.5 Hz, 2H), 1.70 (q, J=7.5 Hz, 4H), 0.97-0.91 (m, 6H). LCMS (M+H)=980.9.

Compound-Linker 2.6

### (S)-2-amino-N⁸-(5-((2-(4-(4-(2,5-dioxo-2,5-dihydro-1H-pyrrol-1-yl)phenyl)butanamido)-3-phenylpropanamido)methyl)pyridin-3-yl)-N⁴,N⁴-dipropyl-3H-benzo[b]azepine-4,8-dicarboxamide, Trifluoroacetate Salt

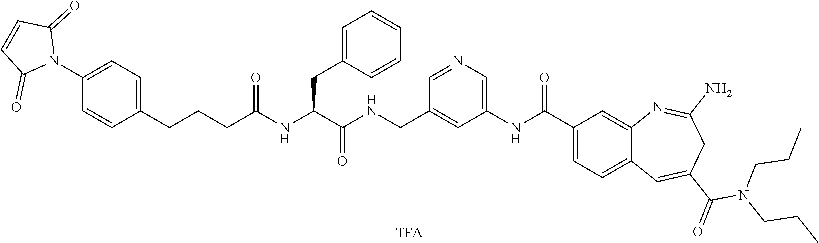 [SMILES: CCCN(CCC)C(=O)C1=Cc2ccc(C(=O)Nc3cncc(CNC(=O)[C@H](Cc4ccccc4)NC(=O)CCCc4ccc(N5C(=O)C=CC5=O)cc4)c3)cc2N=C(N)C1]

From succinimidyl 4-(p-maleimidophenyl)butyrate (SMPB NHS ester) to afford a white solid. ¹H NMR (CD₃OD) δ 8.95 (d, J=2.0 Hz, 1H), 8.63 (d, J=2.0 Hz, 1H), 8.28 (s, 1H), 8.24 (m, 2H), 7.98 (m, 2H), 7.70 (d, J=9.0 Hz, 1H), 7.25-7.15 (m, 9H), 7.16 (s, 1H), 6.94 (s, 2H), 4.60 (m, 1H), 4.51-4.37 (m, 2H), 3.15 (m, 1H), 2.91 (m, 1H), 2.51 (t, J=7.5 Hz, 2H), 2.22 (m, 2H), 1.81 (t, J=7.5 Hz, 2H), 1.70 (q, J=7.5 Hz, 4H), 0.95 (m, 6H). LCMS (M+H)=823.8.

Compound-Linker 2.7

### 4-((S)-2-((S)-2-(6-(2,5-dioxo-2,5-dihydro-1H-pyrrol-1-yl)hexanamido)-3-methylbutanamido)-5-ureidopentanamido)benzyl ((S)-1-(((5-(2-amino-4-(dipropylcarbamoyl)-3H-benzo[b]azepine-8-carboxamido)pyridin-3-yl)methyl)amino)-1-oxo-3-phenylpropan-2-yl)carbamate

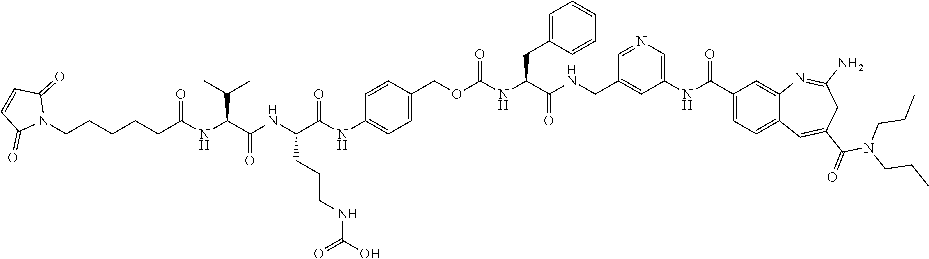 [SMILES: CCCN(CCC)C(=O)C1=Cc2ccc(C(=O)Nc3cncc(CNC(=O)[C@H](Cc4ccccc4)NC(=O)OCc4ccc(NC(=O)[C@H](CCCNC(=O)O)NC(=O)[C@@H](NC(=O)CCCCCN5C(=O)C=CC5=O)C(C)C)cc4)c3)cc2N=C(N)C1]

From mc-VC-PABA-PNP to afford a white solid. ¹H NMR (CD₃OD) δ 8.78 (s, 1H), 8.21 (s, 1H), 8.11 (s, 1H), 7.89 (m, 2H), 7.64 (dd, J=1.9, 8.1 Hz, 1H), 7.49 (d, J=8.0 Hz, 2H), 7.25-7.15 (m, 7H), 7.06 (s, 1H), 6.77 (s, 2H), 4.96 (s, 2H), 4.48 (m, 1H), 4.49-4.34 (m, 3H), 4.14 (d, J=7.5 Hz, 1H), 3.46-3.44 (m, 6H), 3.22 (m, 1H), 3.11 (m, 1H), 2.90 (m, 1H), 2.33-2.25 (m, 2H), 2.08 (m, 1H), 1.91 (m, 1H), 1.75-1.50 (m, 13H), 1.30 (m, 2H), 1.00-0.85 (m, 12H). LCMS (M+H)=1181.4.

Compound-Linker 2.8

### 4-((R)-2-((R)-2-(5-(2,5-dioxo-2,5-dihydro-1H-pyrrol-1-yl)pentanamido)-3-methylbutanamido)-5-ureidopentanamido)benzyl (2-(1-(5-(2-amino-4-(dipropylcarbamoyl)-3H-benzo[b]azepine-8-carboxamido)pyridin-2-yl)piperidine-4-carboxamido)ethyl)carbamate

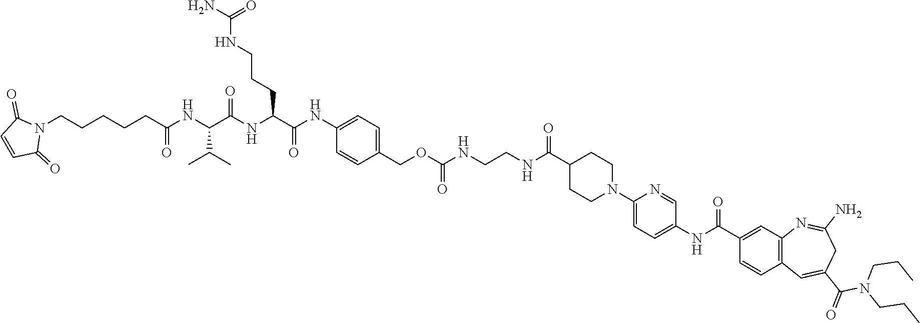 [SMILES: CCCN(CCC)C(=O)C1=Cc2ccc(C(=O)Nc3ccc(N4CCC(C(=O)NCCNC(=O)OCc5ccc(NC(=O)[C@H](CCCNC(N)=O)NC(=O)[C@@H](NC(=O)CCCCCN6C(=O)C=CC6=O)C(C)C)cc5)CC4)nc3)cc2N=C(N)C1]

From Compound 1.61 and mc-VC-PABA-PNP to afford a white solid. ¹H NMR (CD₃OD) δ 10.1 (s, 1H), 9.49 (s, 1H), 9.33 (bs, 2H), 7.88 (d, J=8.0 Hz, 1H), 7.80 (s, 1H), 7.64 (s, 1H), 7.61 (s, 1H), 7.45 (d, J=8.0 Hz, 1H), 7.35 (d, J=8.0 Hz, 1H), 7.02 (s, 1H), 6.85-6.80 (m, 2H), 6.75 (s, 1H), 4.25 m, 2H), 3.54-3.34 (m, 10H), 3.05 (s, 4H), 2.85-2.75 (m, 4H), 2.44 (m, 1H), 1.99 (m, 1H), 1.70-1.60 (m, 12H), 0.95 (bs, 6H).

Compound-Linker 2.9

### 4-((R)-2-((R)-2-(5-(2,5-dioxo-2,5-dihydro-1H-pyrrol-1-yl)pentanamido)-3-methylbutanamido)-5-ureidopentanamido)benzyl (1-(5-(2-amino-4-(dipropylcarbamoyl)-3H-benzo[b]azepine-8-carboxamido)pyridin-2-yl)piperidin-4-yl)carbamate

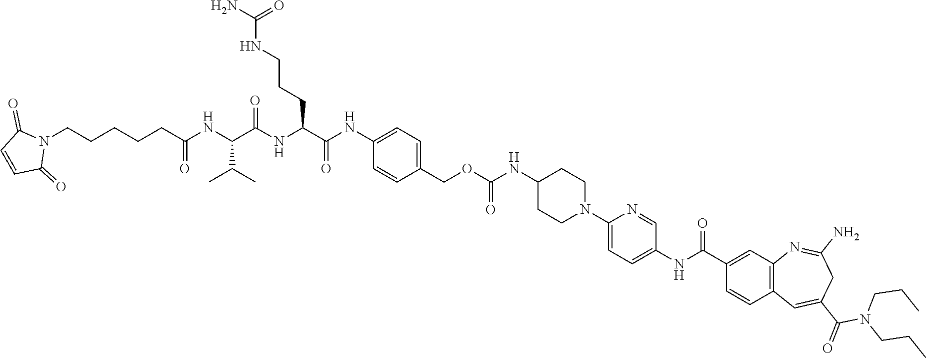 [SMILES: CCCN(CCC)C(=O)C1=Cc2ccc(C(=O)Nc3ccc(N4CCC(NC(=O)OCc5ccc(NC(=O)[C@H](CCCNC(N)=O)NC(=O)[C@@H](NC(=O)CCCCCN6C(=O)C=CC6=O)C(C)C)cc5)CC4)nc3)cc2N=C(N)C1]

From Compound 1.57 and mc-VC-PABA-PNP to afford a white solid. ¹H NMR (CD₃OD) δ 8.37 (d, J=2.5 Hz, 1H), 7.88 (dd, J=8.0, 2.5 Hz, 1H), 7.57-7.54 (m, 3H), 7.43 (d, J=8.0 Hz, 1H), 7.31 (d, J=8.0 Hz, 2H), 6.89 (s, 1H), 6.85-6.80 (m, 1H), 6.78 (s, 2H), 5.03 (s, 2H), 4.45 (m, 2H), 4.12 (m, 3H), 3.65 (m, 1H), 3.54 (t, J=7.5 Hz, 2H), 3.44 (m, 4H), 3.20-2.96 (m, 4H), 2.26 (t, J=7.5 Hz, 2H), 2.05 (m, 1H), 1.99-1.50 (m, 18H), 1.30 (m, 2H), 0.97 (t, J=7.5 Hz, 6H), 0.89 (bs, 6H).

Compound-Linker 2.20

### 4-((S)-2-((S)-2-(6-(2,5-dioxo-2,5-dihydro-1H-pyrrol-1-yl)hexanamido)-3-methylbutanamido)-5-ureidopentanamido)benzyl (2-(((5-(2-amino-4-(dipropylcarbamoyl)-3H-benzo[b]azepine-8-carboxamido)pyridin-3-yl)methyl)amino)-2-oxoethyl)carbamate

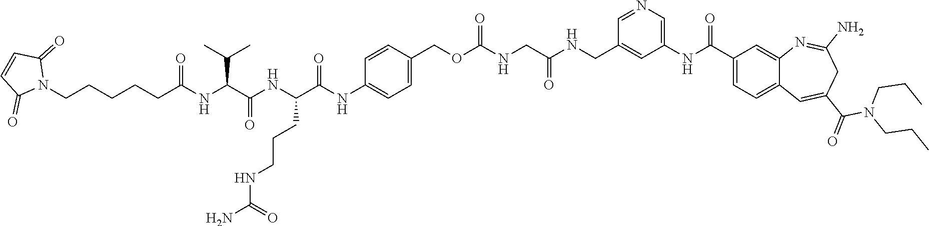 [SMILES: CCCN(CCC)C(=O)C1=Cc2ccc(C(=O)Nc3cncc(CNC(=O)CNC(=O)OCc4ccc(NC(=O)[C@H](CCCNC(N)=O)NC(=O)[C@@H](NC(=O)CCCCCN5C(=O)C=CC5=O)C(C)C)cc4)c3)cc2N=C(N)C1]

From Compound 1.64 and mc-VC-PABA-PNP to afford a white solid. ¹H NMR (CD₃OD) δ 8.81 (s, 1H), 8.25 (s, 1H), 8.21 (s, 1H), 7.72 (s, 1H), 7.58 (m, 2H), 7.45 (d, J=8.2 Hz, 2H), 7.33 (d, J=8.4 Hz, 2H), 6.91 (s, 1H), 6.75 (s, 2H), 4.96 (s, 2H), 4.48 (m, 1H), 4.49-4.34 (m, 3H), 4.14 (d, J=7.5 Hz, 1H), 3.46-3.44 (m, 6H), 3.22 (m, 1H), 3.11 (m, 1H), 2.90 (m, 1H), 2.33-2.25 (m, 2H), 2.08 (m, 1H), 1.91 (m, 1H), 1.75-1.50 (m, 13H), 1.30 (m, 2H), 1.00-0.85 (m, 12H). LCMS (M+H)=1090.2.

Compound-Linker 2.21

### 2-amino-N⁸-(6-(4-((2-(4-((2,5-dioxo-2,5-dihydro-1H-pyrrol-1-yl)methyl)cyclohexane-1-carboxamido)ethyl)carbamoyl)piperidin-1-yl)pyridin-3-yl)-N⁴,N⁴-dipropyl-3H-benzo[b]azepine-4,8-dicarboxamide

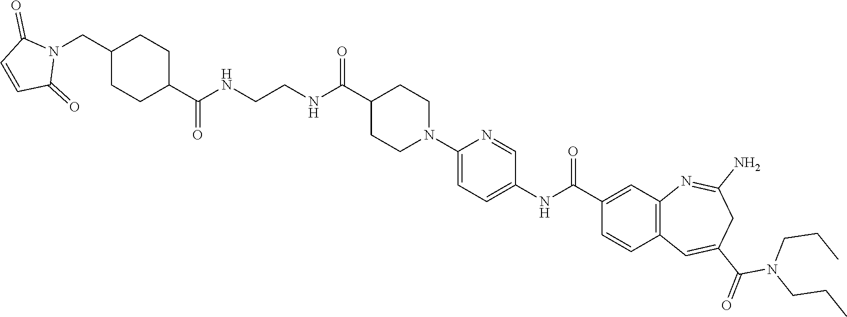 [SMILES: CCCN(CCC)C(=O)C1=Cc2ccc(C(=O)Nc3ccc(N4CCC(C(=O)NCCNC(=O)C5CCC(CN6C(=O)C=CC6=O)CC5)CC4)nc3)cc2N=C(N)C1]

From Compound 1.61 and succinimidyl 4-(N-maleimidomethyl)cyclohexane-1-carboxylate to provide a white solid. ¹H NMR (DMSO-d₆) δ 10.1 (s, 1H), 8.46 (s, 1H), 8.61 (bs, 2H), 7.92 (dd, J=8.0, 2.5 Hz, 1H), 7.81 (m, 1H), 7.72 (m, 1H), 7.61 (s, 1H), 7.53 (d, J=8.0 Hz, 1H), 7.41 (d, J=8.0 Hz, 2H), 7.03 (s, 2H), 6.85-6.80 (m, 2H), 6.78 (s, 1H), 4.25 (m, 2H), 3.65 (m, 1H), 3.54 (t, J=7.5 Hz, 2H), 3.44 (m, 4H), 3.20-2.96 (m, 4H), 2.26 (t, J=7.5 Hz, 2H), 2.05 (m, 1H), 1.99-1.50 (m, 18H), 1.30 (m, 2H), 0.97 (t, J=7.5 Hz, 6H), 0.89 (bs, 6H). LCMS (M+H)=794.5.

### Example 7B: Synthesis of 4-((S)-2-((S)-2-(6-(2,5-dioxo-2,5-dihydro-1H-pyrrol-1-yl)hexanamido)-3-methylbutanamido)-5-ureidopentanamido)benzyl (2-(4-((3-(2-amino-4-(dipropylcarbamoyl)-3H-benzo[b]azepine-8-carboxamido)-7,8-dihydro-1,6-naphthyridin-6(5H)-yl)methyl)benzamido)ethyl)carbamate (Compound-Linker 2.10)

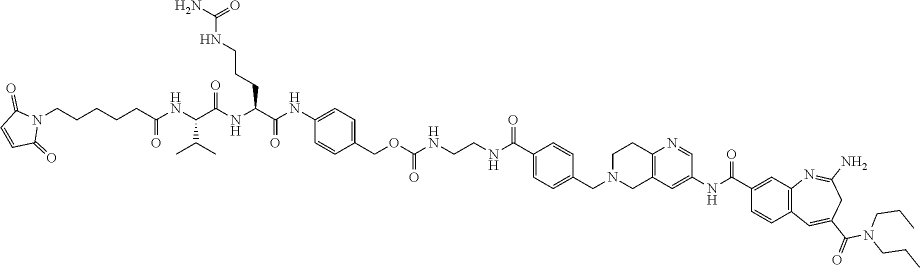 [SMILES: CCCN(CCC)C(=O)C1=Cc2ccc(C(=O)Nc3cnc4c(c3)CN(Cc3ccc(C(=O)NCCNC(=O)OCc5ccc(NC(=O)[C@H](CCCNC(N)=O)NC(=O)[C@@H](NC(=O)CCCCCN6C(=O)C=CC6=O)C(C)C)cc5)cc3)CC4)cc2N=C(N)C1]

Step A: Preparation of Int 7B-1

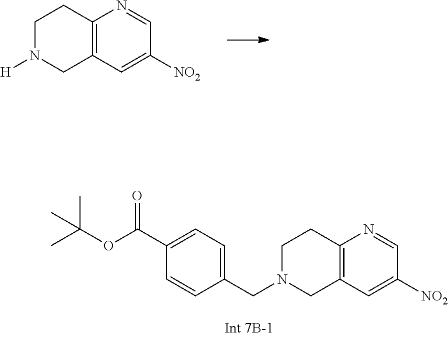 [SMILES: O=[N+]([O-])c1cnc2c(c1)CNCC2 ; CC(C)(C)OC(=O)c1ccc(CN2CCc3ncc([N+](=O)[O-])cc3C2)cc1]

To a stirred solution of 2-nitro-5,6,7,8-tetrahydro-1,6-naphthyridine dihydrochloride (1.0 g, 3.97 mmol) and tert-butyl 4-(bromomethyl)benzoate (1.18 g, 4.36 mmol) in DMF (40 mL) cooled in an ice-water bath was added dropwise TEA (2.76 mL, 19.8 mmol). The resulting clear solution was stirred overnight while cooling bath expired. LC-MS showed mostly desired product with small amount of SM remaining. The reaction mixture was concentrated in vacuo and the residue was diluted with water (45 mL) and saturated NaHCO₃ solution (5 mL) then extracted with EtOAc (3×). The combined extracts were dried (Na₂SO₄), filtered, and concentrated. The residue was absorbed on silica gel and purified by flash column chromatography (ISCO Gold 40 g; dry load, 0-20% CH₂Cl₂/MeOH) to afford 1.32 g of tert-butyl 4-((3-nitro-7,8-dihydro-1,6-naphthyridin-6(5H)-yl)methyl)benzoate as an orange colored syrup. ¹H NMR (DMSO-d₆) δ 9.15 (d, J=2.5 Hz, 1H), 8.36 (d, J=2.5 Hz, 1H), 7.88 (d, J=8.0 Hz, 2H), 7.49 (d, J=8.0 Hz, 2H), 4.00 (s, 3H), 3.79 (s, 2H), 3.71 (s, 2H), 3.04 (m, 2H), 2.85 (m, 2H), 1.55 (s, 9H).

Step B: Preparation of Int 7B-2

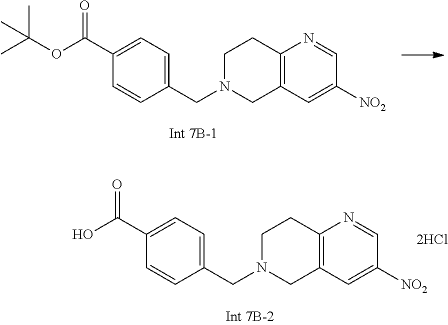 [SMILES: CC(C)(C)OC(=O)c1ccc(CN2CCc3ncc([N+](=O)[O-])cc3C2)cc1 ; O=C(O)c1ccc(CN2CCc3ncc([N+](=O)[O-])cc3C2)cc1]

To a stirred solution of tert-butyl 4-((3-nitro-7,8-dihydro-1,6-naphthyridin-6(5H)-yl)methyl)benzoate (1.32 g, 3.57 mmol) in 27 mL of DCM was added 4M HCl (9 mL, 36.0 mmol) in dioxane at room temperature. The reaction mixture was stirred for 3 h then concentrated under reduced pressure. The residue dried in vacuo to afford a light yellow solid which was used directly without further purification. ¹H NMR (CD₃OD) δ 9.33 (d, J=2.5 Hz, 1H), 8.53 (d, J=2.5 Hz, 1H), 8.19 (d, J=8.0 Hz, 2H), 7.72 (d, J=8.0 Hz, 2H), 4.82 (m, 2H), 4.66 (m, 2H), 4.61 (s, 2H), 3.44 (m, 2H).

Step C: Preparation of Int 7B-3

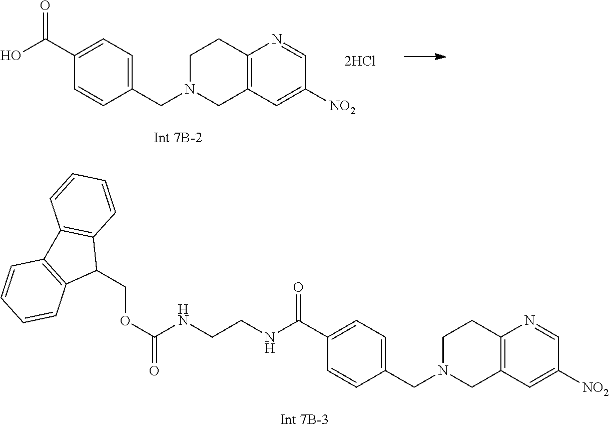 [SMILES: O=C(O)c1ccc(CN2CCc3ncc([N+](=O)[O-])cc3C2)cc1 ; O=C(NCCNC(=O)c1ccc(CN2CCc3ncc([N+](=O)[O-])cc3C2)cc1)OCC1c2ccccc2-c2ccccc21]

To a stirred solution of 4-((3-nitro-7,8-dihydro-1,6-naphthyridin-6(5H)-yl)methyl)benzoic acid dihydrochloride (1.28 g, 3.32 mmol), (9H-fluoren-9-yl)methyl (2-aminoethyl)carbamate hydrochloride (1.060 g, 3.32 mmol), and diisopropylethylamine (4.65 ml, 26.6 mmol) in 30 mL of DCM cooled in an ice-water bath was added dropwise 2,4,6-tripropyl-1,3,5,2,4,6-trioxatriphosphinane 2,4,6-trioxide (T3P®; 3.0 ml, 5.0 mmol). The mixture was stirred overnight while the cooling bath expired. The reaction mixture was partitioned between saturated NaHCO₃ and EtOAc. The aqueous layer was extracted with EtOAc (2×) and the combined organic extracts were washed with brine, dried over Na₂SO₄, filtered and concentrated to give 2.2 g of the desired product as an orange-red solid.

Step D: Preparation of Int 7B-4

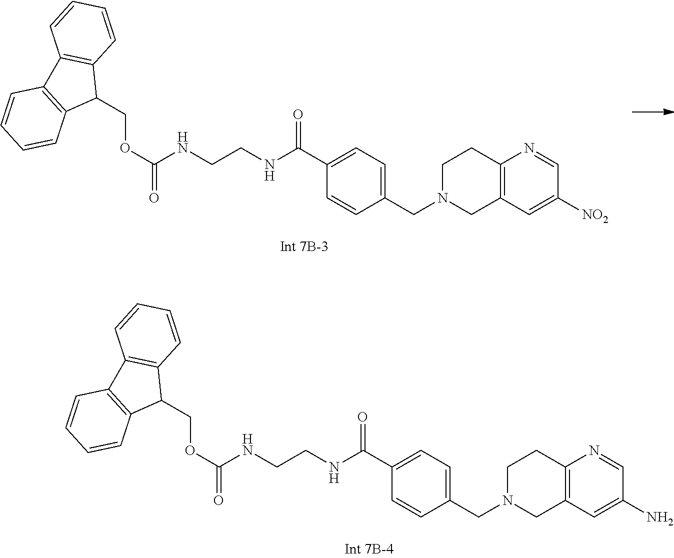 [SMILES: O=C(NCCNC(=O)c1ccc(CN2CCc3ncc([N+](=O)[O-])cc3C2)cc1)OCC1c2ccccc2-c2ccccc21 ; Nc1cnc2c(c1)CN(Cc1ccc(C(=O)NCCNC(=O)OCC3c4ccccc4-c4ccccc43)cc1)CC2]

A mixture of (9H-fluoren-9-yl)methyl (2-(4-((3-nitro-7,8-dihydro-1,6-naphthyridin-6(5H)-yl)methyl)benzamido)ethyl)carbamate (2.0 g, 3.5 mmol) and iron (1.930 g, 34.6 mmol) in acetic acid (30 mL)/water (3 mL) was stirred at 50° C. for 45 min. The reaction mixture was cooled to room temperature, filtered and concentrated. The residue was diluted with saturated NaHCO₃ (90 mL) and EtOAc (90 mL). The precipitate was collected, washed with water and EtOAc, and dried in vacuo to afford 1.9 g of a yellow-brown solid which was suspended in 1:1 CH₂Cl₂/MeOH and absorbed on silica gel. Purification by flash column chromatography (ISCO Gold 80 g; dry load, 0-50% B in CH₂Cl₂ gradient, B: 80:18:2 CH₂Cl₂/MeOH/conc. NH₄OH) gave 1.12 g of the desired product as an off-white solid.

Step E: Preparation of Int 7B-5

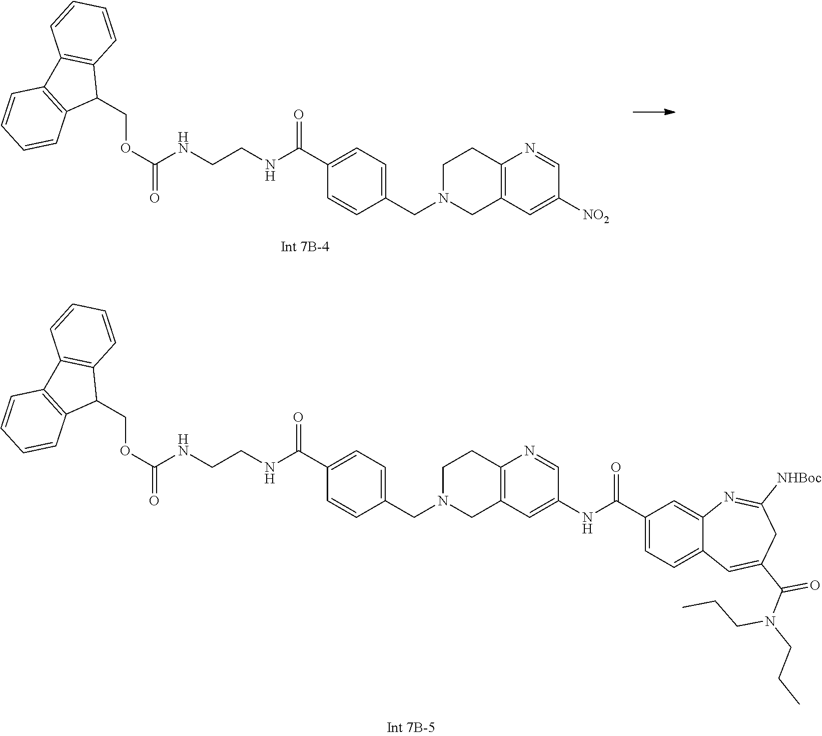 [SMILES: O=C(NCCNC(=O)c1ccc(CN2CCc3ncc([N+](=O)[O-])cc3C2)cc1)OCC1c2ccccc2-c2ccccc21 ; CCCN(CCC)C(=O)C1=Cc2ccc(C(=O)Nc3cnc4c(c3)CN(Cc3ccc(C(=O)NCCNC(=O)OCC5c6ccccc6-c6ccccc65)cc3)CC4)cc2N=C(NC(=O)OC(C)(C)C)C1]

To a stirred solution of 2-((tert-butoxycarbonyl)amino)-4-(dipropylcarbamoyl)-3H-benzo[b]azepine-8-carboxylic acid (350 mg, 0.815 mmol) in DMF (5 mL) at rt was added HATU (341 mg, 0.896 mmol). The reaction was stirred for 15 min before the addition of 669 mg (1.22 mmol) of (9H-fluoren-9-yl)methyl (2-(4-((3-amino-7,8-dihydro-1,6-naphthyridin-6(5H)-yl)methyl)benzamido)ethyl)carbamate in DMF (11 mL) was added. The reaction was stirred for 35 min before the addition of 0.427 mL (2.44 mmol) of Hunig's base. The resulting yellow solution was stirred for 18 h then concentrated in vacuo. The residue was purified by flash column chromatography (ISCO Gold 40 g; dry load, 0-50% B in CH₂Cl₂ gradient, B: 80:18:2 CH₂Cl₂/MeOH/conc. NH₄OH) to afford 435 mg of the desired product as a light yellow solid.

Step F. Preparation of Int 7B-6

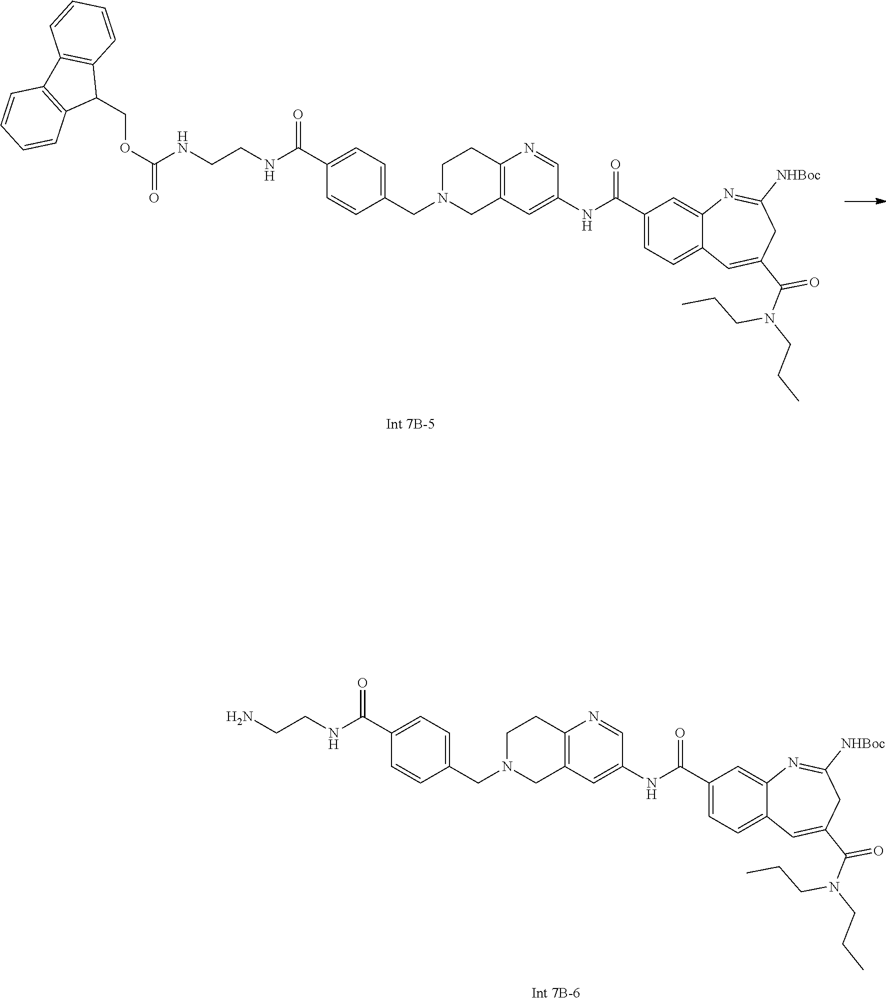 [SMILES: CCCN(CCC)C(=O)C1=Cc2ccc(C(=O)Nc3cnc4c(c3)CN(Cc3ccc(C(=O)NCCNC(=O)OCC5c6ccccc6-c6ccccc65)cc3)CC4)cc2N=C(NC(=O)OC(C)(C)C)C1 ; CCCN(CCC)C(=O)C1=Cc2ccc(C(=O)Nc3cnc4c(c3)CN(Cc3ccc(C(=O)NCCN)cc3)CC4)cc2N=C(NC(=O)OC(C)(C)C)C1]

To a stirred solution of tert-butyl (8-((6-(4-((2-((((9H-fluoren-9-yl)methoxy)carbonyl)amino)ethyl)carbamoyl)benzyl)-5,6,7,8-tetrahydro-1,6-naphthyridin-3-yl)carbamoyl)-4-(dipropylcarbamoyl)-3H-benzo[b]azepin-2-yl)carbamate (435 mg, 0.454 mmol) in 3.6 mL of DMF was added 0.90 mL (9.1 mmol) of piperidine at room temperature. The reaction was stirred for 1 h then concentrated. The residue was purified by flash column chromatography (ISCO Gold 24 g, 0-50% B in CH₂Cl₂ gradient, B: 80:18:2 CH₂Cl₂/MeOH/conc. NH₄OH) to afford 241 mg of the desired product as a light yellow solid.

Step G: Preparation of Compound-Linker 2.10

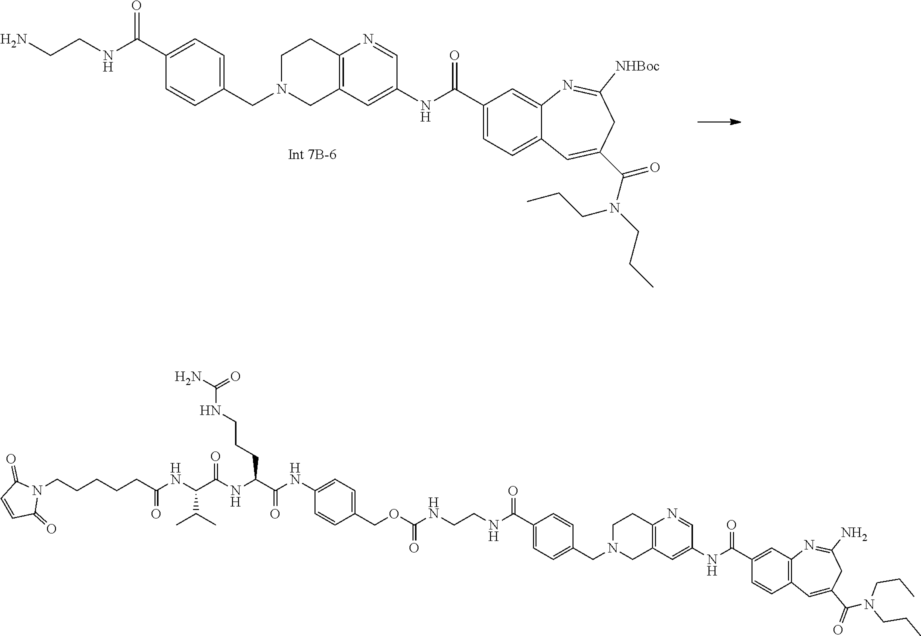 [SMILES: CCCN(CCC)C(=O)C1=Cc2ccc(C(=O)Nc3cnc4c(c3)CN(Cc3ccc(C(=O)NCCN)cc3)CC4)cc2N=C(NC(=O)OC(C)(C)C)C1 ; CCCN(CCC)C(=O)C1=Cc2ccc(C(=O)Nc3cnc4c(c3)CN(Cc3ccc(C(=O)NCCNC(=O)OCc5ccc(NC(=O)[C@H](CCCNC(N)=O)NC(=O)[C@@H](NC(=O)CCCCCN6C(=O)C=CC6=O)C(C)C)cc5)cc3)CC4)cc2N=C(N)C1]

To a stirred solution of tert-butyl (8-((6-(4-((2-aminoethyl)carbamoyl)benzyl)-5,6,7,8-tetrahydro-1,6-naphthyridin-3-yl)carbamoyl)-4-(dipropylcarbamoyl)-3H-benzo[b]azepin-2-yl)carbamate (80 mg, 0.109 mmol) and Hunig's base (0.057 mL, 0.326 mmol) in DMF (3.4 mL) under nitrogen cooled in an ice-water bath was added dropwise a solution of 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (80 mg, 0.109 mmol) in DMF (2 mL). The reaction was stirred overnight while cooling bath expired. The reaction mixture was then concentrated and the residue neutralized with saturated NaHCO₃ and purified by reverse phase column (Gold C18 30 g; 5-60% CH₃CN in water, no TFA). Fractions pooled, concentrated to afford 100 mg of an off-yellow solid which was directly dissolved in 50 mL of DCM and treated with 10 mL of TFA. The resulting solution was stirred for 1 h then concentrated under reduced pressure. The residue was dried in vacuo, neutralized with saturated NaHCO₃, and purified by reverse phase column chromatography (ISCO Gold C18 30 g; 5-70% MeCN in water gradient, no TFA). Major fractions were combined and lyophilized to provide 22 mg of an off-white solid. ¹H NMR (CD₃OD) δ 8.67 (d, J=2.5 Hz, 1H), 7.91 (d, J=2.5 Hz, 1H), 7.80 (d, J=8.0 Hz, 1H), 7.69 (d, J=2.5 Hz, 1H), 7.58-7.50 (m, 5H), 7.45 (d, J=8.0 Hz, 1H), 7.26 (d, J=8.5 Hz, 2H), 6.89 (s, 1H), 6.77 (s, 2H), 5.04 (s, 2H), 4.90 (m, 1H), 4.14 (d, J=7.5 Hz, 1H), 3.81 (s, 2H), 3.69 (s, 2H), 3.51-3.40 (m, 8H), 3.34 (m, 2H), 3.22 (m, 1H), 3.11 (m, 2H), 2.97 (m, 2H), 2.90 (m, 3H), 2.25 (t, J=7.5 Hz, 2H), 2.06 (m, 1H), 1.88 (m, 1H), 1.75-1.52 (m, 12H), 1.28 (m, 2H), 0.95 (t, J=7.5 Hz, 6H), 0.89 (bs, 6H). LCMS (M+H)=1235.9.

### Example 7C: Synthesis of 4-((S)-2-((S)-2-(6-(2,5-dioxo-2,5-dihydro-1H-pyrrol-1-yl)hexanamido)-3-methylbutanamido)-5-ureidopentanamido)benzyl (2-(4-((3-(2-amino-4-(dipropylcarbamoyl)-3H-benzo[b]azepine-8-carboxamido)-7,8-dihydro-1,6-naphthyridin-6(5H)-yl)methyl)benzamido)ethyl)carbamate (Compound-Linker 2.11)

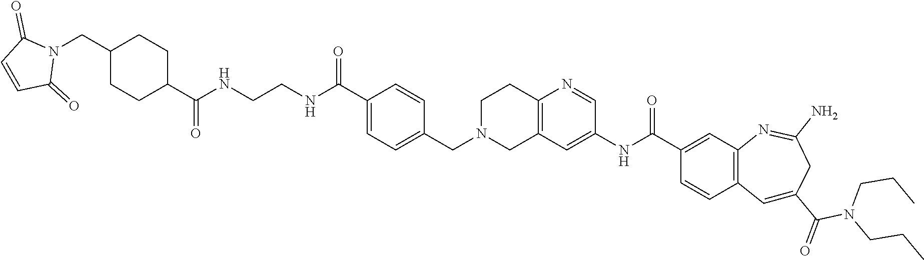 [SMILES: CCCN(CCC)C(=O)C1=Cc2ccc(C(=O)Nc3cnc4c(c3)CN(Cc3ccc(C(=O)NCCNC(=O)C5CCC(CN6C(=O)C=CC6=O)CC5)cc3)CC4)cc2N=C(N)C1]

Step A: Preparation of Compound-Linker 2.11

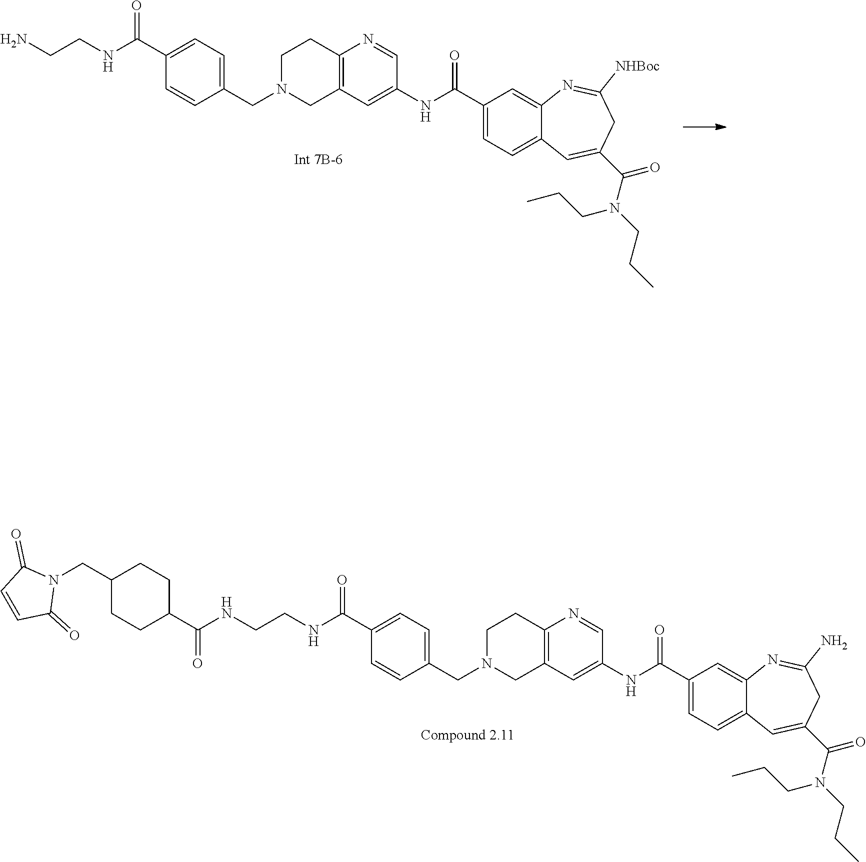 [SMILES: CCCN(CCC)C(=O)C1=Cc2ccc(C(=O)Nc3cnc4c(c3)CN(Cc3ccc(C(=O)NCCN)cc3)CC4)cc2N=C(NC(=O)OC(C)(C)C)C1 ; CCCN(CCC)C(=O)C1=Cc2ccc(C(=O)Nc3cnc4c(c3)CN(Cc3ccc(C(=O)NCCNC(=O)C5CCC(CN6C(=O)C=CC6=O)CC5)cc3)CC4)cc2N=C(N)C1]

A solution of 84.5 mg (0.115 mmol) of tert-butyl (8-((6-(4-((2-aminoethyl)carbamoyl)benzyl)-5,6,7,8-tetrahydro-1,6-naphthyridin-3-yl)carbamoyl)-4-(dipropylcarbamoyl)-3H-benzo[b]azepin-2-yl)carbamate from step F above, 2,5-dioxopyrrolidin-1-yl 4-((2,5-dioxo-2,5-dihydro-1H-pyrrol-1-yl)methyl)cyclohexane-1-carboxylate (38.3 mg, 0.115 mmol), and Hunig's base (0.040 mL, 0.229 mmol) in DCM (2.5 mL) was stirred at rt for 16 h. The reaction mixture was concentrated to dryness and the residue was purified by reverse phase column chromatography (ISCO Gold C18 100 g, 5-70% MeCN in water gradient, no TFA). The desired fractions were pooled and concentrated to provide 79 mg of the desired product as a yellow solid which was subsequently dissolved in 2.5 mL of DCM at rt then treated with TFA (500 μL, 6.49 mmol). After 1 h, the reaction mixture was concentrated, the residue dried in vacuo, neutralized with saturated NaHCO₃, and purified by reverse phase column chromatography (ISCO Gold C18 100 g; 5-60% MeCN in water gradient, no TFA). The main fractions were pooled and concentrated. The residue was lyophilized from MeCN/water to afford 25 mg of the desired product as an off-white solid. ¹H NMR (CD₃OD) δ 8.67 (d, J=2.5 Hz, 1H), 7.91 (d, J=2.5 Hz, 1H), 7.80 (d, J=8.0 Hz, 1H), 7.68 (d, J=2.5 Hz, 1H), 7.55 (dd, J=2.0, 8.0 Hz, 1H), 7.53 (d, J=8.0 Hz, 2H), 7.45 (d, J=8.0 Hz, 1H), 6.89 (s, 1H), 6.77 (s, 2H), 4.57 (s, 1H), 3.81 (s, 2H), 3.49-3.38 (m, 8H), 3.00 (m, 2H), 2.90 (m, 2H), 2.84 (m, 1H), 2.11 (m, 1H), 1.88 (m, 1H), 1.70-1.58 (m, 8H), 1.39 (m, 2H), 1.0-0.89 (m, 10H). LCMS (M+H)=856.8.

### Example 7D: Synthesis of Perfluorophenyl 4-((3-((2-(4-((3-(2-amino-4-(dipropylcarbamoyl)-3H-benzo[b]azepine-8-carboxamido)-7,8-dihydro-1,6-naphthyridin-6(5H)-yl)methyl)benzamido)ethyl)thio)-2,5-dioxopyrrolidin-1-yl)methyl)cyclohexane-1-carboxylate Tris TFA Salt (Compound-Linker 2.12)

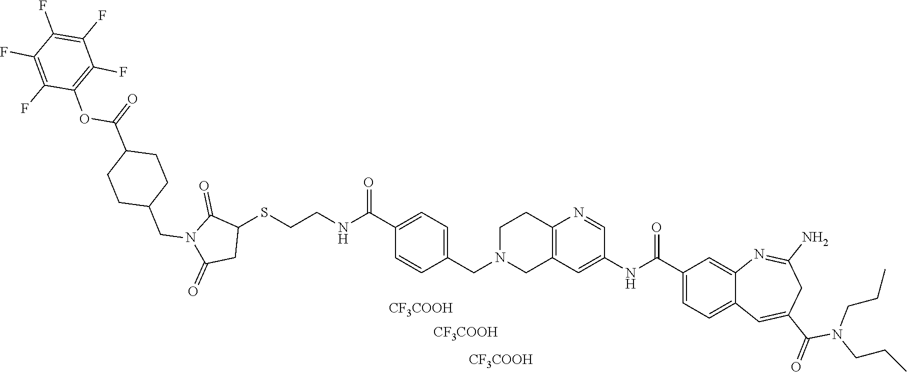 [SMILES: CCCN(CCC)C(=O)C1=Cc2ccc(C(=O)Nc3cnc4c(c3)CN(Cc3ccc(C(=O)NCCSC5CC(=O)N(CC6CCC(C(=O)Oc7c(F)c(F)c(F)c(F)c7F)CC6)C5=O)cc3)CC4)cc2N=C(N)C1 ; "CF3COOH" ; "CF3COOH" ; "CF3COOH"]

Preparation of Compound-Linker 2.12

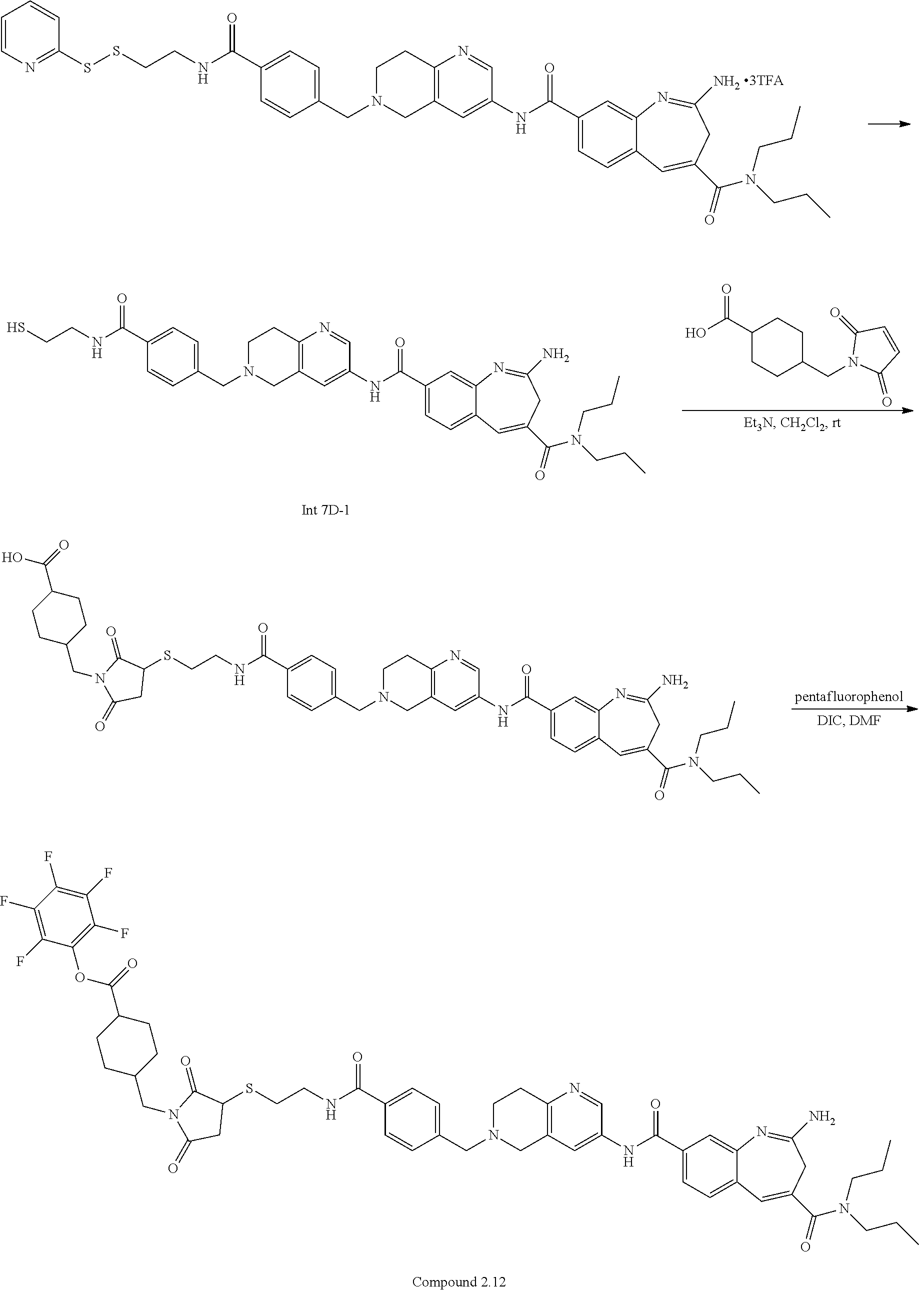 [SMILES: CCCN(CCC)C(=O)C1=Cc2ccc(C(=O)Nc3cnc4c(c3)CN(Cc3ccc(C(=O)NCCSSc5ccccn5)cc3)CC4)cc2N=C(N)C1 ; CCCN(CCC)C(=O)C1=Cc2ccc(C(=O)Nc3cnc4c(c3)CN(Cc3ccc(C(=O)NCCS)cc3)CC4)cc2N=C(N)C1 ; O=C(O)C1CCC(CN2C(=O)C=CC2=O)CC1 ; CCCN(CCC)C(=O)C1=Cc2ccc(C(=O)Nc3cnc4c(c3)CN(Cc3ccc(C(=O)NCCSC5CC(=O)N(CC6CCC(C(=O)O)CC6)C5=O)cc3)CC4)cc2N=C(N)C1 ; CCCN(CCC)C(=O)C1=Cc2ccc(C(=O)Nc3cnc4c(c3)CN(Cc3ccc(C(=O)NCCSC5CC(=O)N(CC6CCC(C(=O)Oc7c(F)c(F)c(F)c(F)c7F)CC6)C5=O)cc3)CC4)cc2N=C(N)C1]

A solution of 2-amino-N⁴,N⁴-dipropyl-N⁸-(6-(4-((2-(pyridin-2-yldisulfanyl)ethyl)carbamoyl)benzyl)-5,6,7,8-tetrahydro-1,6-naphthyridin-3-yl)-3H-benzo[b]azepine-4,8-dicarboxamide (100 mg, 0.090 mmol) (tri-TFA salt) and 3,3′,3″-phosphanetriyltripropionic acid hydrochloride (38.9 mg, 0.136 mmol) in 3 mL of 1:1 acetonitrile/water was stirred at room temperature for 0.5 h. The reaction mixture was concentrated in vacuo to dryness to provide Int 7D-1 a yellow foamy solid which was used directly without further any purification. This intermediate was converted to the final Compound-Linker 2.12 according to the scheme above. ¹H NMR (CD₃OD) δ 8.77 (d, J=2.0 Hz, 1H), 8.22 (d, J=2.5 Hz, 1H), 8.00-7.95 (m, 3H), 7.10 (s, 1H), 4.57 (bs, 2H), 4.47 (bs, 2H), 4.11 (dd, J=9.0, 3.5 Hz, 1H), 3.76-3.62 (m, 3H), 3.45-3.35 (m, 4H), 3.40-3.35 (m, 4H), 3.24-3.18 (m, 4H), 2.98 (m, 1H), 2.71 (m, 1H), 2.54 (d, J=3.5 Hz, 0.5H), 2.50 (d, J=3.5 Hz, 0.5H), 2.15 (m, 2H), 1.83-1.79 (m, 2H), 1.74-1.64 (m, 6H), 1.55-1.45 (m, 2H), 1.17-1.10 (m, 2H), 0.96 (bs, 3H), 0.91 (bs, 3H). LCMS (M+H)=1057.7.

### Example 7E: Preparation of 4-((S)-2-((S)-2-(6-(2,5-dioxo-2,5-dihydro-1H-pyrrol-1-yl)hexanamido)-3-methylbutanamido)-5-ureidopentanamido)benzyl 3-(2-amino-4-(dipropylcarbamoyl)-7-methoxy-3H-benzo[b]azepine-8-carboxamido)-7,8-dihydro-1,6-naphthyridine-6(5H)-carboxylate (Compound-Linker 2.14)

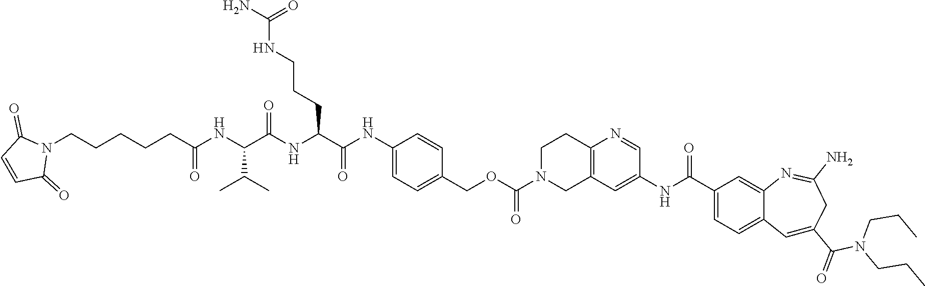 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1]

Prepared in a manner similar to Compound 2.1 (example 5) using 2-amino-4-(dipropylcarbamoyl)-3H-benzo[b]azepine-8-carboxylic acid and commercially available tert-Butyl 3-amino-7,8-dihydro-1,6-naphthyridine-6(5H)-carboxylate (CAS No. 355819-02-2). ¹H NMR (DMSO-d₆) δ 12.1 (s, 1H), 10.4 (s, 1H), 10.0 (s, 1H), 9.14 (s, 1H), 8.73 (d, J=2.4 Hz, 1H), 8.08 (d, J=7.6 Hz, 2H), 7.80 (d, J=8.8 Hz, 1H), 7.70 (s, 1H), 7.60 (d, J=8.4 Hz, 2H), 7.41 (s, 1H), 7.34 (d, J=8.8 Hz, 2H), 7.03 (s, 1H), 7.00 (s, 1H), 5.99 (bs, 1H), 5.07 (s, 2H), 4.65 (m, 4H), 4.40 (m, 2H), 4.21 (m, 2H), 3.97 (s, 3H), 3.74 (bt, 2H), 3.37 (t, J=6.8 Hz, 5H), 3.29 (s, 2H), 3.11-2.95 (m, 4H), 2.22-1.95 (m, 4H), 1.60-1.15 (m, 12H), 0.88 (d, J=7.0 Hz, 6H), 0.82 (d, J=7.0 Hz, 6H). LCMS [M+H]=1090.5.

### Example 7F: Preparation of 4-((S)-2-((S)-2-(6-(2,5-dioxo-2,5-dihydro-1H-pyrrol-1-yl)hexanamido)-3-methylbutanamido)-5-ureidopentanamido)benzyl 3-(2-amino-4-(dipropylcarbamoyl)-7-methoxy-3H-benzo[b]azepine-8-carboxamido)-7,8-dihydro-1,6-naphthyridine-6(5H)-carboxylate (Compound-Linker 2.15)

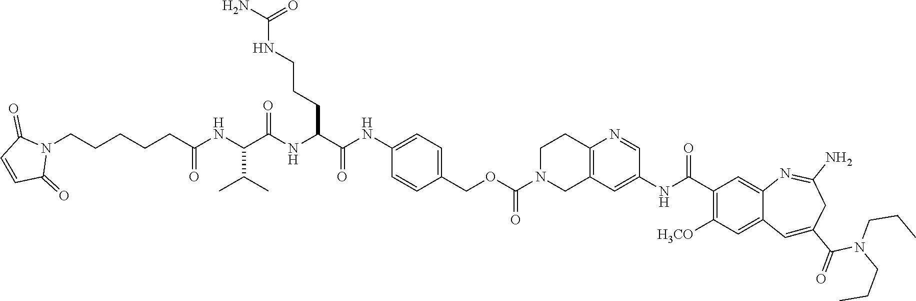 [SMILES: CCCN(CCC)C(=O)C1=Cc2cc(OC)c(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1]

Prepared in a manner similar to Compound 2.1 (example 5) starting from 2-amino-4-(dipropylcarbamoyl)-7-methoxy-3H-benzo[b]azepine-8-carboxylic acid.

### Example 7G: Preparation of 4-((S)-2-((S)-2-(6-(2,5-dioxo-2,5-dihydro-1H-pyrrol-1-yl)hexanamido)-3-methylbutanamido)-5-ureidopentanamido)benzyl 3-(2-amino-4-(dipropylcarbamoyl)-7-fluoro-3H-benzo[b]azepine-8-carboxamido)-7,8-dihydro-1,6-naphthyridine-6(5H)-carboxylate (Compound-Linker 2.16)

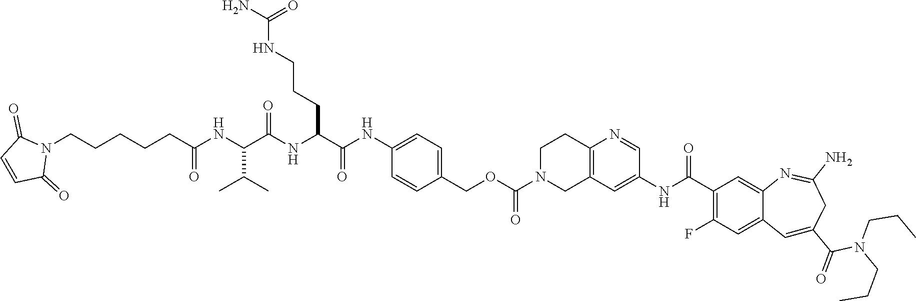 [SMILES: CCCN(CCC)C(=O)C1=Cc2cc(F)c(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1]

Prepared in a manner similar to Compound 2.1 (example 5) starting from 2-amino-4-(dipropylcarbamoyl)-7-fluoro-3H-benzo[b]azepine-8-carboxylic acid.

¹H NMR (DMSO-d₆) δ 12.2 (s, 1H), 10.8 (s, 1H), 10.0 (s, 1H), 9.89 (s, 1H), 9.27 (s, 1H), 8.66 (s, 1H), 8.08 (d, J=2.4 Hz, 1H), 8.03 (s, 1H), 7.80 (d, J=8.8 Hz, 2H), 7.70-7.64 (m, 2H), 7.60 (d, J=8.4 Hz, 1H), 7.34 (d, J=8.8 Hz, 2H), 7.02 (s, 1H), 7.00 (s, 2H), 5.99 (bs, 1H), 5.07 (s, 2H), 4.65 (m, 4H), 4.40 (m, 2H), 4.21 (m, 2H), 3.73 (bt, 2H), 3.36 (m, 5H), 3.29 (s, 2H), 3.11-2.95 (m, 4H), 2.22-1.95 (m, 4H), 1.60-1.15 (m, 12H), 0.88 (d, J=7.0 Hz, 6H), 0.82 (d, J=7.0 Hz, 6H). LCMS [M+H]=1079.5.

### Example 7H: Preparation of 4-((S)-2-((S)-2-(6-(2,5-dioxo-2,5-dihydro-1H-pyrrol-1-yl)hexanamido)-3-methylbutanamido)-5-ureidopentanamido)benzyl (3-(4-((3-(2-amino-4-(dipropylcarbamoyl)-3H-benzo[b]azepine-8-carboxamido)-7,8-dihydro-1,6-naphthyridin-6(5H)-yl)methyl)benzamido)-2,2-difluoropropyl)carbamate (Compound-Linker 2.17)

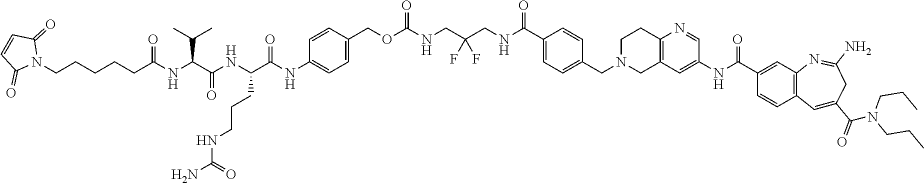 [SMILES: CCCN(CCC)C(=O)C1=Cc2ccc(C(=O)Nc3cnc4c(c3)CN(Cc3ccc(C(=O)NCC(F)(F)CNC(=O)OCc5ccc(NC(=O)[C@H](CCCNC(N)=O)NC(=O)[C@@H](NC(=O)CCCCCN6C(=O)C=CC6=O)C(C)C)cc5)cc3)CC4)cc2N=C(N)C1]

Prepared in a manner similar to Compound 2.1 (example 5)

Table 2 shows Compound-Linkers 2.1-2.21.

TABLE 2
Compound-Linkers 2.1-2.21

| Com- | |
|---|---|
| pound- | |
| Linkers | Structure |
| 2.1 | 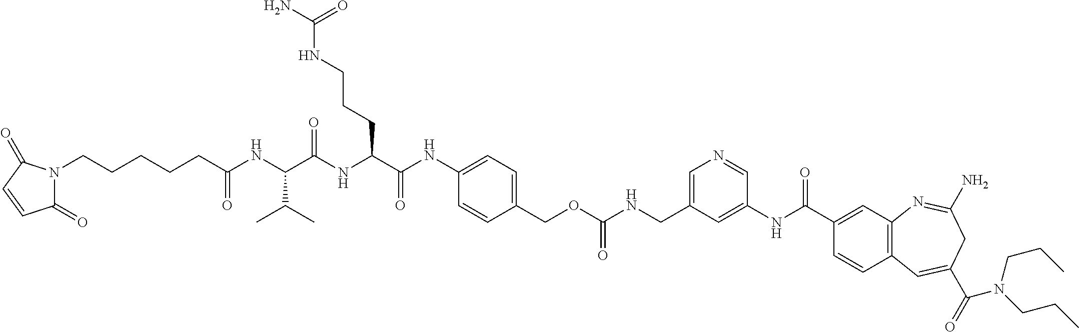 [SMILES: CCCN(CCC)C(=O)C1=Cc2ccc(C(=O)Nc3cncc(CNC(=O)OCc4ccc(NC(=O)[C@H](CCCNC(N)=O)NC(=O)[C@@H](NC(=O)CCCCCN5C(=O)C=CC5=O)C(C)C)cc4)c3)cc2N=C(N)C1] |
| 2.2 | 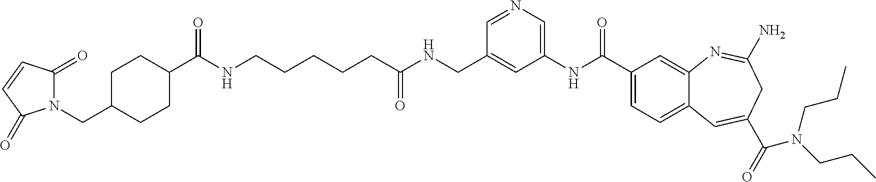 [SMILES: CCCN(CCC)C(=O)C1=Cc2ccc(C(=O)Nc3cncc(CNC(=O)CCCCCNC(=O)C4CCC(CN5C(=O)C=CC5=O)CC4)c3)cc2N=C(N)C1] |
| 2.3 | 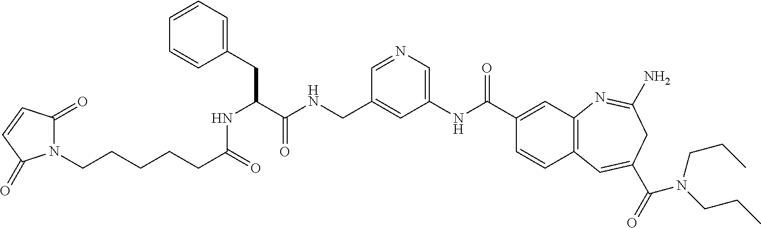 [SMILES: CCCN(CCC)C(=O)C1=Cc2ccc(C(=O)Nc3cncc(CNC(=O)[C@H](Cc4ccccc4)NC(=O)CCCCCN4C(=O)C=CC4=O)c3)cc2N=C(N)C1] |
| 2.4 | 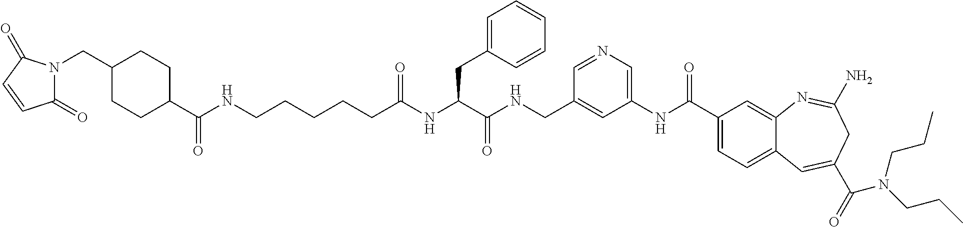 [SMILES: CCCN(CCC)C(=O)C1=Cc2ccc(C(=O)Nc3cncc(CNC(=O)[C@H](Cc4ccccc4)NC(=O)CCCCCNC(=O)C4CCC(CN5C(=O)C=CC5=O)CC4)c3)cc2N=C(N)C1] |
| 2.5 | 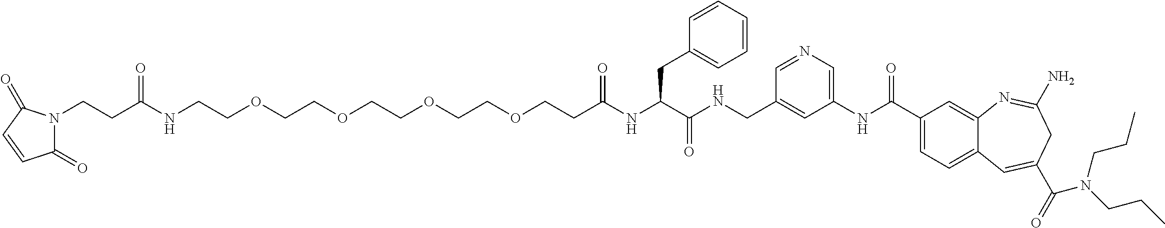 [SMILES: CCCN(CCC)C(=O)C1=Cc2ccc(C(=O)Nc3cncc(CNC(=O)[C@H](Cc4ccccc4)NC(=O)CCOCCOCCOCCOCCNC(=O)CCN4C(=O)C=CC4=O)c3)cc2N=C(N)C1] |
| 2.6 | 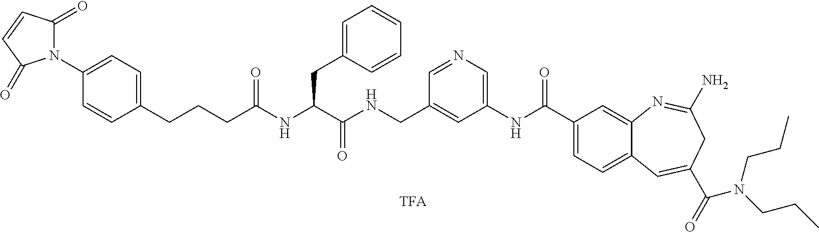 [SMILES: CCCN(CCC)C(=O)C1=Cc2ccc(C(=O)Nc3cncc(CNC(=O)[C@H](Cc4ccccc4)NC(=O)CCCc4ccc(N5C(=O)C=CC5=O)cc4)c3)cc2N=C(N)C1] |
| 2.7 | 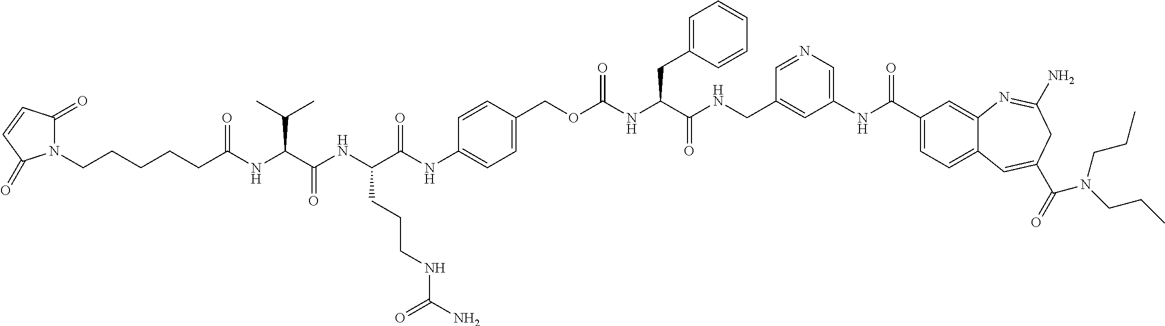 [SMILES: CCCN(CCC)C(=O)C1=Cc2ccc(C(=O)Nc3cncc(CNC(=O)[C@H](Cc4ccccc4)NC(=O)OCc4ccc(NC(=O)[C@H](CCCNC(N)=O)NC(=O)[C@@H](NC(=O)CCCCCN5C(=O)C=CC5=O)C(C)C)cc4)c3)cc2N=C(N)C1] |
| 2.8 | 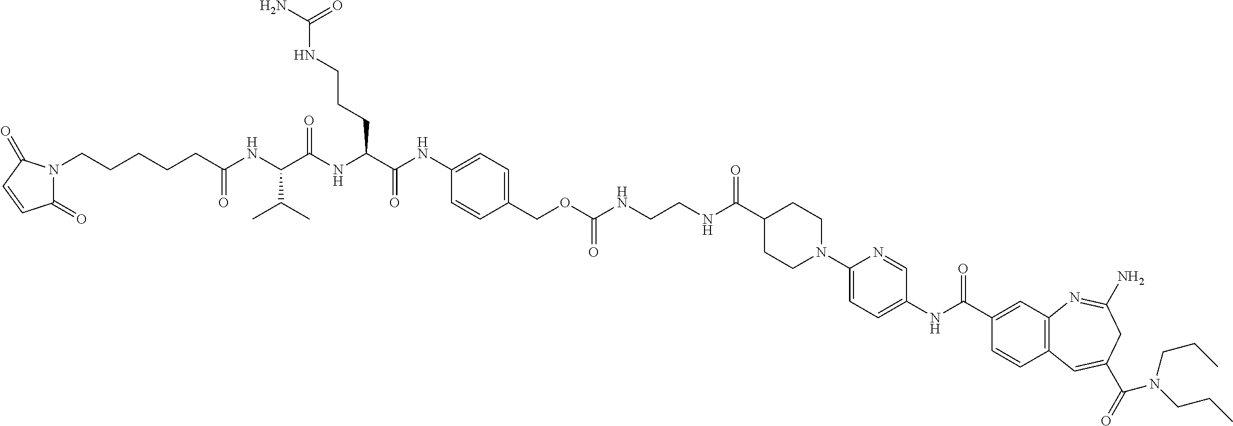 [SMILES: CCCN(CCC)C(=O)C1=Cc2ccc(C(=O)Nc3ccc(N4CCC(C(=O)NCCNC(=O)OCc5ccc(NC(=O)[C@H](CCCNC(N)=O)NC(=O)[C@@H](NC(=O)CCCCCN6C(=O)C=CC6=O)C(C)C)cc5)CC4)nc3)cc2N=C(N)C1] |
| 2.9 | 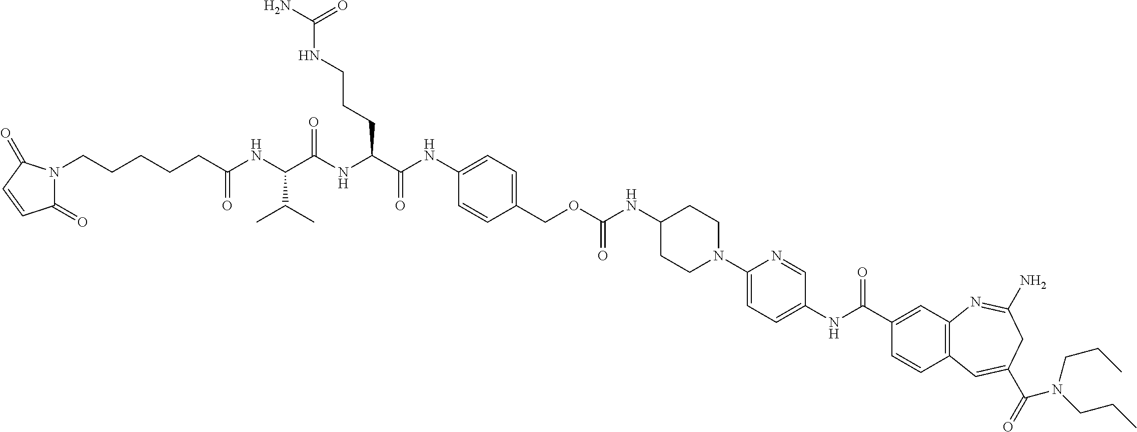 [SMILES: CCCN(CCC)C(=O)C1=Cc2ccc(C(=O)Nc3ccc(N4CCC(NC(=O)OCc5ccc(NC(=O)[C@H](CCCNC(N)=O)NC(=O)[C@@H](NC(=O)CCCCCN6C(=O)C=CC6=O)C(C)C)cc5)CC4)nc3)cc2N=C(N)C1] |
| 2.10 | 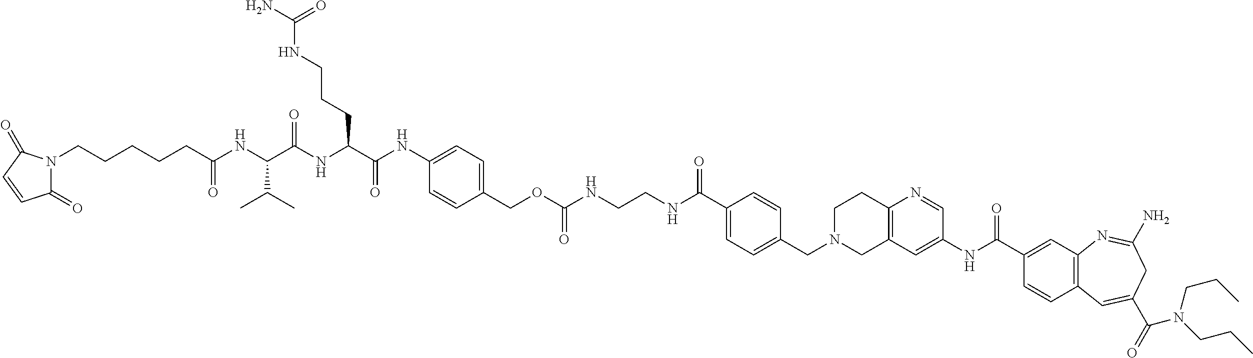 [SMILES: CCCN(CCC)C(=O)C1=Cc2ccc(C(=O)Nc3cnc4c(c3)CN(Cc3ccc(C(=O)NCCNC(=O)OCc5ccc(NC(=O)[C@H](CCCNC(N)=O)NC(=O)[C@@H](NC(=O)CCCCCN6C(=O)C=CC6=O)C(C)C)cc5)cc3)CC4)cc2N=C(N)C1] |
| 2.11 | 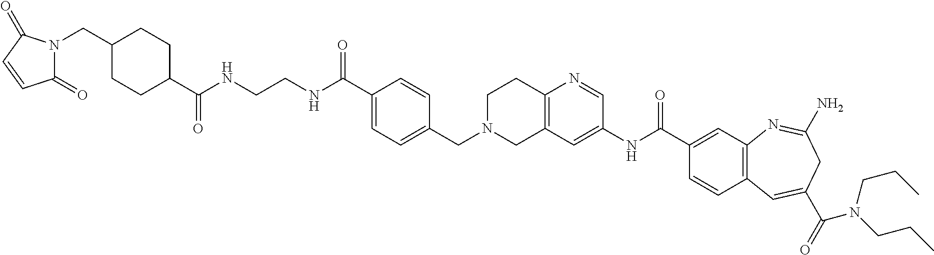 [SMILES: CCCN(CCC)C(=O)C1=Cc2ccc(C(=O)Nc3cnc4c(c3)CN(Cc3ccc(C(=O)NCCNC(=O)C5CCC(CN6C(=O)C=CC6=O)CC5)cc3)CC4)cc2N=C(N)C1] |
| 2.12 | 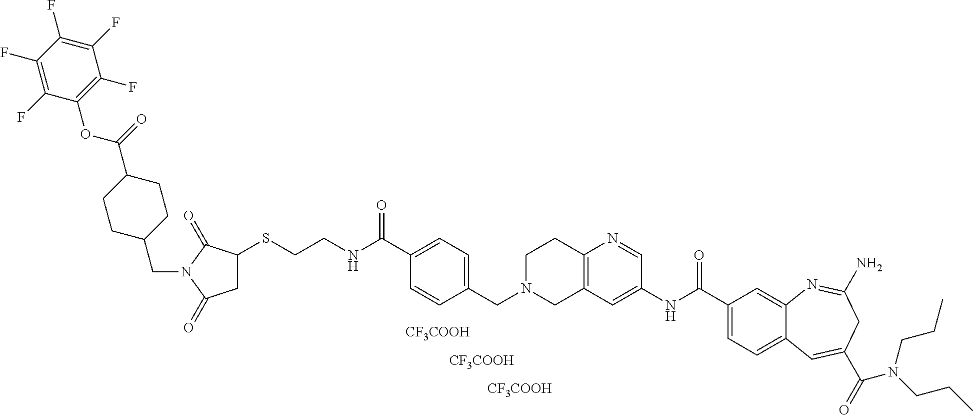 [SMILES: CCCN(CCC)C(=O)C1=Cc2ccc(C(=O)Nc3cnc4c(c3)CN(Cc3ccc(C(=O)NCCSC5CC(=O)N(CC6CCC(C(=O)Oc7c(F)c(F)c(F)c(F)c7F)CC6)C5=O)cc3)CC4)cc2N=C(N)C1 ; "CF3COOH" ; "CF3COOH" ; "CF3COOH"] |
| 2.14 | 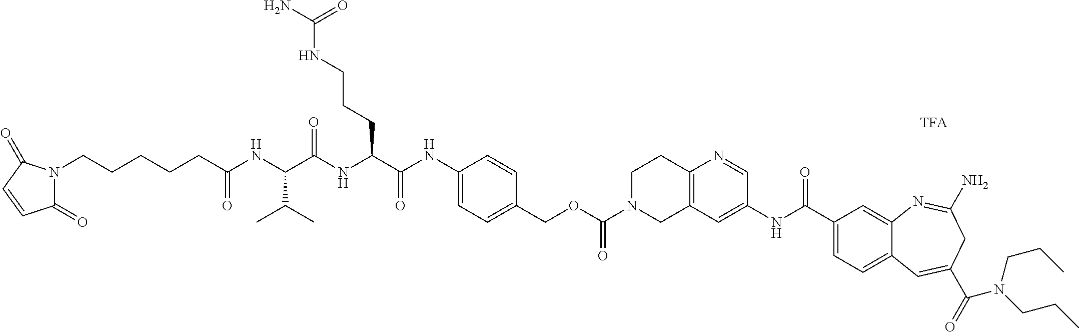 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1] |
| 2.15 | 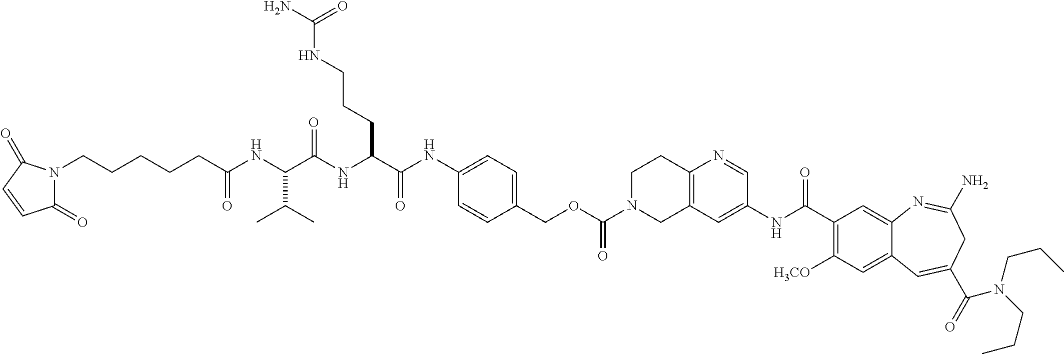 [SMILES: CCCN(CCC)C(=O)C1=Cc2cc(OC)c(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1] |
| 2.16 | 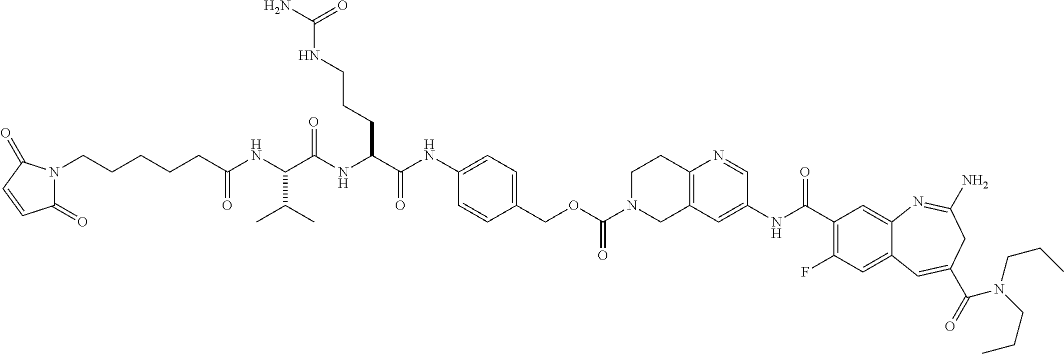 [SMILES: CCCN(CCC)C(=O)C1=Cc2cc(F)c(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1] |
| 2.17 | 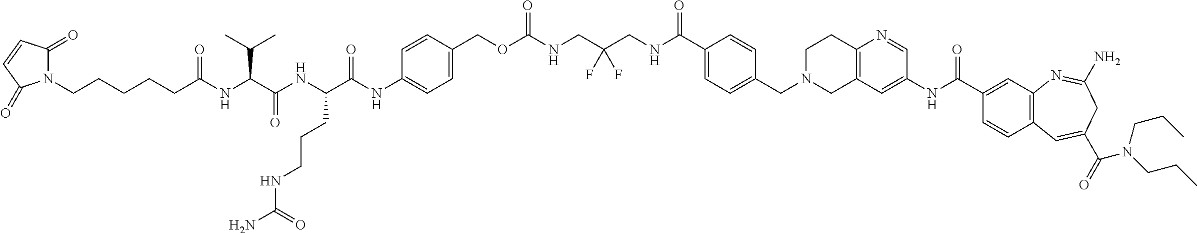 [SMILES: CCCN(CCC)C(=O)C1=Cc2ccc(C(=O)Nc3cnc4c(c3)CN(Cc3ccc(C(=O)NCC(F)(F)CNC(=O)OCc5ccc(NC(=O)[C@H](CCCNC(N)=O)NC(=O)[C@@H](NC(=O)CCCCCN6C(=O)C=CC6=O)C(C)C)cc5)cc3)CC4)cc2N=C(N)C1] |
| 2.20 | 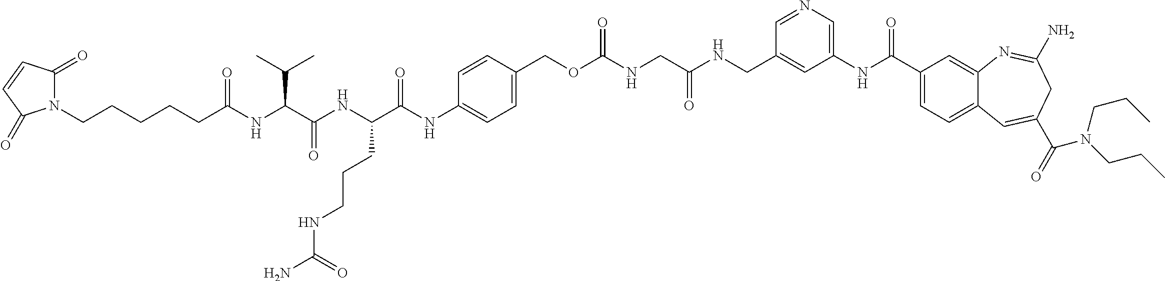 [SMILES: CCCN(CCC)C(=O)C1=Cc2ccc(C(=O)Nc3cncc(CNC(=O)CNC(=O)OCc4ccc(NC(=O)[C@H](CCCNC(N)=O)NC(=O)[C@@H](NC(=O)CCCCCN5C(=O)C=CC5=O)C(C)C)cc4)c3)cc2N=C(N)C1] |
| 2.21 | 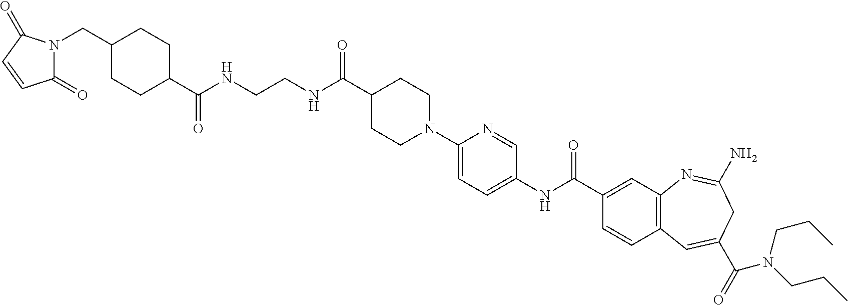 [SMILES: CCCN(CCC)C(=O)C1=Cc2ccc(C(=O)Nc3ccc(N4CCC(C(=O)NCCNC(=O)C5CCC(CN6C(=O)C=CC6=O)CC5)CC4)nc3)cc2N=C(N)C1] |
| 2.22 | 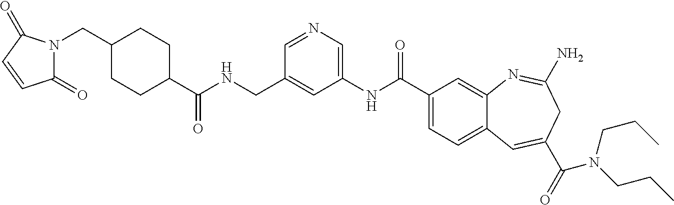 [SMILES: CCCN(CCC)C(=O)C1=Cc2ccc(C(=O)Nc3cncc(CNC(=O)C4CCC(CN5C(=O)C=CC5=O)CC4)c3)cc2N=C(N)C1] |

### Example 8: Linking Antibody Constructs to Myeloid Cell Agonists Via a Linker

This example shows different methods of linking an antibody construct to a myeloid cell agonist via a linker to form a conjugate.

A linker, such as a maleimidocaproyl)-(valine-citrulline)-(para-aminobenzyloxycarbonyl) linker or disulfide linker (e.g., as disclosed in formulas Ig to Il) can be first attached to a myeloid cell agonist to form a myeloid cell agonist-linker compound. Subsequently, a myeloid cell agonist-linker is conjugated to an antibody construct.

A linker is attached to an antibody construct, in which the linker is a disulfide linker (e.g., in formula Ig-Il) or a hydrazone linker to form a linker-antibody construct. Subsequently, a myeloid cell agonist is conjugated to the linker linked with the antibody construct.

### Example 9: Lysine-Based Bioconjugation

The antibody construct is exchanged into an appropriate buffer, for example, phosphate, borate, PBS, or Tris-Acetate, at a concentration of about 2 mg/mL to about 10 mg/mL. An appropriate number of equivalents of the myeloid cell agonist-linker are added as a solution with stirring. Dependent on the physical properties of the myeloid cell agonist-linker construct, a co-solvent can be introduced prior to the addition of the myeloid cell agonist-linker construct to facilitate solubility. The reaction is stirred at room temperature for 2 hours to about 12 hours depending on the observed reactivity. The progression of the reaction is monitored by LC-MS. Once the reaction is deemed complete, the remaining myeloid cell agonist-linker constructs are removed by applicable methods and the lysine-linked myeloid cell agonist conjugate is exchanged into the desired formulation buffer.

Lysine-linked conjugates are synthesized starting with 10 mg of antibody construct (mAb) and 10 equivalents of myeloid cell agonist-linker using the conditions described in Scheme 34 below (ADC=conjugate; ATAC=myeloid cell agonist-linker). Monomer content and drug-antibody ratios can be determined by methods described below.

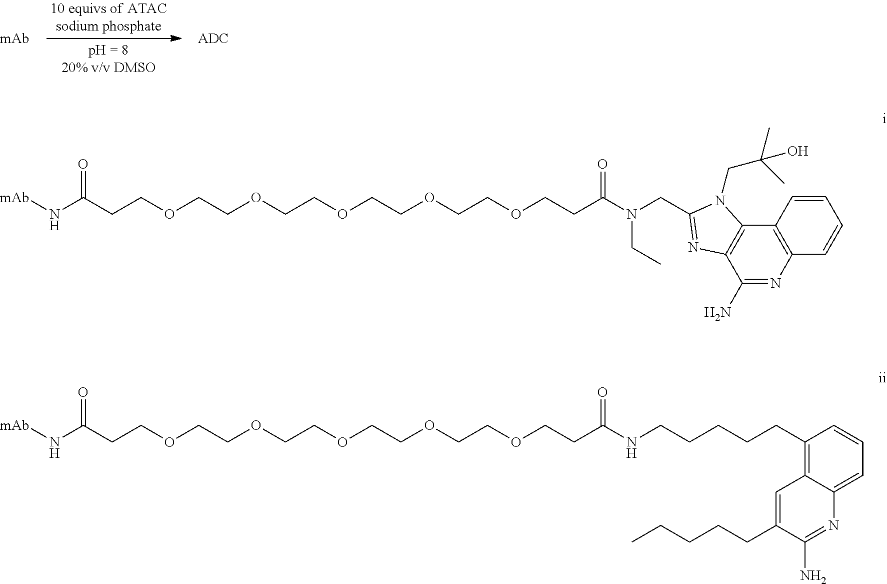

### EXAMPLE 10: Cysteine-Based Bioconjugation

The antibody is exchanged into an appropriate buffer, for example, phosphate, borate, PBS, or Tris-Acetate, at a concentration of about 2 mg/mL to about 10 mg/mL with an appropriate number of equivalents of a reducing agent, for example, dithiothreitol or tris(2-carboxyethyl)phosphine. The resultant solution is stirred for an appropriate amount of time and temperature to effect the desired reduction. The myeloid cell agonist-linker construct is added as a solution with stirring. Dependent on the physical properties of the myeloid cell agonist-linker construct, a co-solvent is introduced prior to the addition of the myeloid cell agonist-linker construct to facilitate solubility. The reaction is stirred at room temperature for about 1 hour to about 12 hours depending on the observed reactivity. The progression of the reaction is monitored by liquid chromatography-mass spectrometry (LC-MS). Once the reaction is deemed complete, the remaining free immune stimulatory compound-linker construct is removed by applicable methods and the conjugate is exchanged into the desired formulation buffer. Such cysteine-based conjugates are synthesized starting with 10 mg of antibody construct (mAb) and 7 equivalents of myeloid cell agonist-linker using the conditions described in Scheme 35 below (ADC=conjugates; ATAC=myeloid cell agonist-linker). Monomer content and drug-antibody ratios can be determined herein.

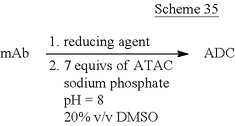

### Example 11: Determination of Molar Ratio

This example illustrates one method by which the molar ratio is determined. One microgram of conjugate is injected into an LC/MS such as an Agilent 6550 iFunnel Q-TOF equipped with an Agilent Dual Jet Stream ESI source coupled with Agilent 1290 Infinity UHPLC system. Raw data is obtained and is deconvoluted with software such as Agilent MassHunter Qualitative Analysis Software with BioConfirm using the Maximum Entropy deconvolution algorithm. The average mass of intact conjugates is calculated by the software, which can use top peak height at 25% for the calculation. This data is then imported into another program to calculate the molar ratio of the myeloid cell agonist:conjugate, such as Agilent molar ratio calculator.

### Example 12: Additional Method for Determination of Molar Ratio

Another method for determination of molar ratio is as follows. First, 10 μL of a 5 mg/mL solution of a conjugate is injected into an HPLC system set-up with a TOSOH TSKgel Butyl-NPR TM hydrophobic interaction chromatography (HIC) column (2.5 μM particle size, 4.6 mm×35 mm) attached. Then, over the course of 18 minutes, a method is run in which the mobile phase gradient is run from 100% mobile phase A to 100% mobile phase B over the course of 12 minutes, followed by a six minute re-equilibration at 100% mobile phase A. The flow rate is 0.8 mL/min and the detector is set at 280 nM. Mobile phase A is 1.5 M ammonium sulfate, 25 mM sodium phosphate (pH 7). Mobile phase B is 25% isopropanol in 25 mM sodium phosphate (pH 7). Post-run, the chromatogram is integrated and the molar ratio is determined by summing the weighted peak area.

### Example 13: TNFα Expression by PBMCs is Induced by TLR8 Myeloid Cell Agonist Conjugates

This example shows that myeloid cell agonist conjugates can increase production of a pro-inflammatory cytokine, TNFα, by PBMCs in the presence of cells expressing an antigen recognized by the conjugate.

PBMCs are isolated from humans by standard methods. Briefly, PBMCs are isolated by Ficoll gradient centrifugation, resuspended in RPMI, and plated in 96-well flat bottom microtiter plates (˜125,000/well). Recombinant cells expressing an antigen (e.g., HER2) are then added (˜25,000/well) along with titrating concentrations of conjugates or unconjugated parental antibodies as controls. The conjugates contain an antibody against the antigen; the antibody is attached to a TLR8 benzazepine agonist. After overnight culture, supernatants are harvested, and TNFα levels are determined by AlphaLISA. Expression of TNFα is increased in the presence of the conjugates.

### Example 14: Murine TNFα Production by Murine Macrophages is Induced by Immune Stimulatory Conjugates

General procedure for immune stimulatory conjugate screening. This example shows that immune-stimulatory conjugates can increase production of a pro-inflammatory cytokine, murine TNFα, from bone marrow-derived murine macrophages in the presence of antigen expressing tumor cells.

Murine bone marrow cells are differentiated into macrophages. After differentiation, bone marrow-derived murine macrophages are plated in 96-well flat bottom microtiter plates (80,000/well) in cRPMI assay media. Antigen-expressing or antigen non-expressing tumor cells are then added (40,000/well) along with titrating concentrations of conjugates or control antibodies ranging from 100 to 0.006 nM in cRPMI media. After overnight culture, supernatants are harvested and murine TNFα levels are determined by ELISA (BioLegend). Data is analyzed using GraphPad Prism 7.01 software (GraphPad Software) and EC50 values calculated using non-linear regression. The data will show conjugates are active, stimulating production of murine TNFα in a dose-dependent manner from the murine macrophages in the presence of antigen expression. In contrast, the conjugates do not stimulate production of murine TNFα from the murine macrophages in the absence of antigen on non-expressing cells.

### Example 15: HER2-TLR7 and HER2-TLR8 Immune Agonist Conjugates

The myeloid cell agonist-linker construct for the anti-HER2 humanized antibody-TLR7 conjugate (“HER2-TLR7”) referred to in the following examples is as shown below. Conjugation is via cysteine-based bioconjugation as described herein.

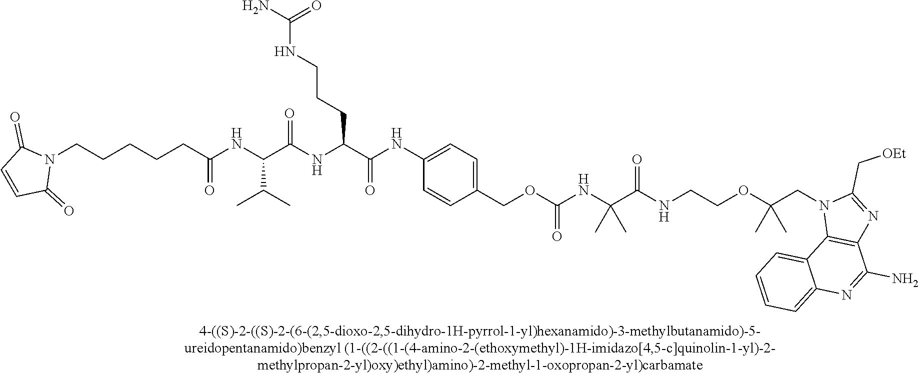 [SMILES: CCOCc1nc2c(N)nc3ccccc3c2n1CC(C)(C)OCCNC(=O)C(C)(C)NC(=O)OCc1ccc(NC(=O)[C@H](CCCNC(N)=O)NC(=O)[C@@H](NC(=O)CCCCCN2C(=O)C=CC2=O)C(C)C)cc1]

The myeloid cell agonist-linker construct for the anti-HER2 humanized antibody-TLR8 conjugate (“HER2-TLR8”) referred to in the following examples is as shown below. Conjugation is via cysteine-based bioconjugation as described herein.

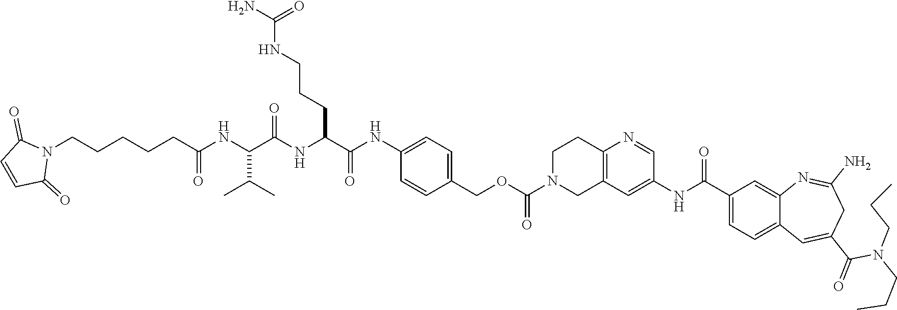 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1]

### Example 16—Subcutaneous Administration of Immune Agonist Conjugate Avoids the Anaphylaxis-Like Response Observed with Intravenous Administration in Mouse Models. Anaphylaxis-Like Response in Mice Requires B Cells

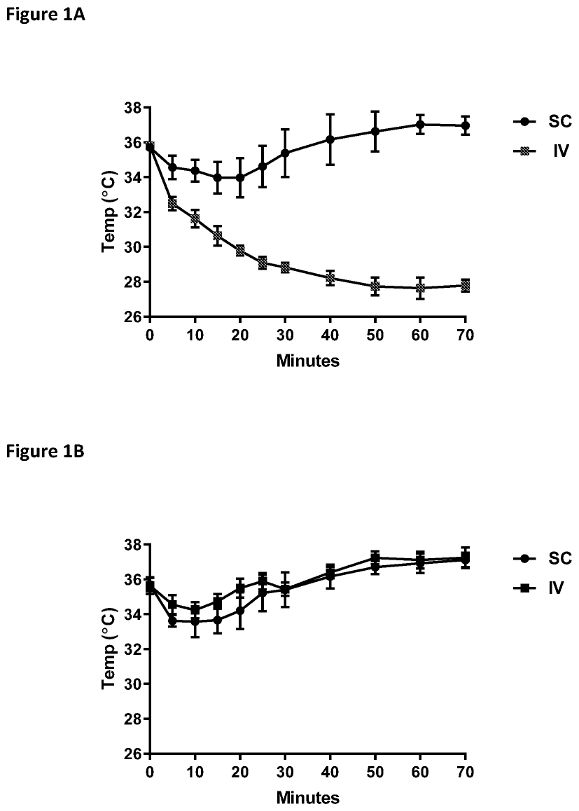

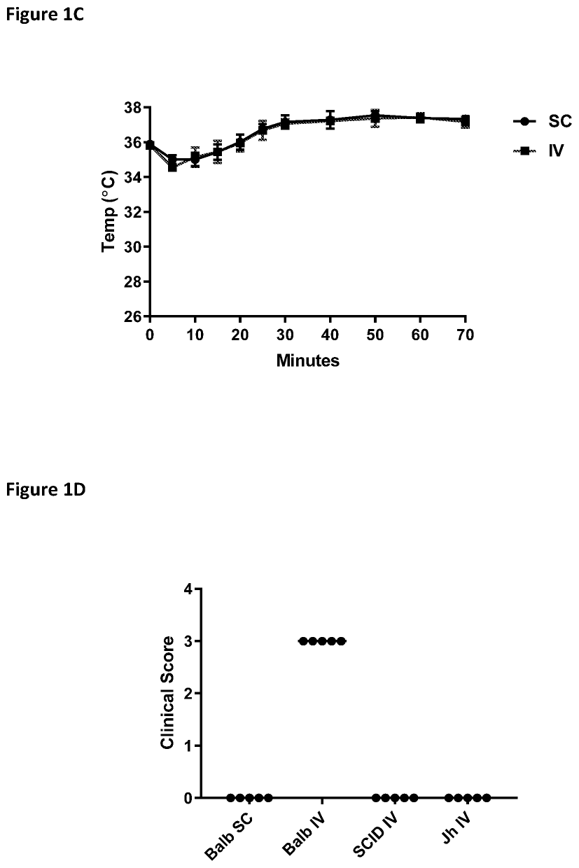

This example shows that mice given HER2-TLR7 by intravenous (IV), but not subcutaneous (SC), administration, experience symptoms of an anaphylaxis-like toxicity upon repeat dosing, as evidenced by hypothermia. Tumor-free Balb/c females (Jackson Laboratory) were given 2 doses of HER2-TLR7 bolus IV or SC at 5 mg/kg on day 0 and day 7. Immediately following the second dose (Day 7), rectal temperatures were recorded every 5-10 minutes for one hour. Mice administered drug IV, but not SC, exhibited a steady drop in temperature indicative of an anaphylaxis-like response (FIG. 1A). Note, no clinical symptoms or change in body temperature was observed after the first dose, regardless of route (data not shown).

Tumor-free female T- and B-cell deficient SCID mice (Jackson Laboratory; FIG. 1B), and B-cell deficient J_{H}−/− mice (Taconic Laboratory; FIG. 1C) were administered HER2-TLR7 IV or SC at 5 mg/kg on days 0 and 7. Rectal temperatures were assessed every five-ten minutes following the second dose. Unlike Balb/c mice, SCID and J_{H}−/− mice did not exhibit a steep, sustained drop in body temperature (FIG. 1B, 1C, respectively), following administration by either route indicating that B cells are required for this response. These results suggest an antibody-mediated anaphylactic response with IV HER2-TLR7. In addition to body temperature, clinical signs of anaphylaxis were scored according to Table 3 below. Importantly, only IV-dosed, B cell competent Balb/c mice showed outward signs of anaphylaxis and a sustained drop in body temperature that required euthanasia (FIG. 1D).

TABLE 3

| Clinical | |
|---|---|
| Score | Description |
| 0 | Normal |
| 1 | Hunched, lethargic |
| 2 | Fully immobilized |
| 3 | Severe, sustained temp drop requiring |
| | euthanasia |
| 4 | Spontaneous Death |

### Example 17: Pre-Treatment with B Cell Depleting Antibody Protects Mice from Anaphylactic Response

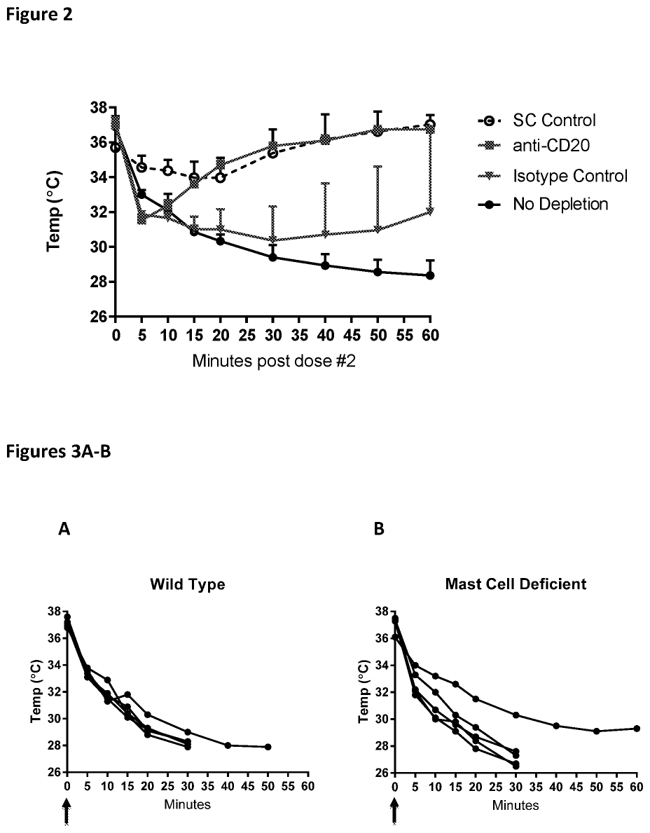

To evaluate the effect of prophylactic B cell depletion on the anaphylactic response, tumor-free female Balb/c mice (Jackson Laboratory) were treated with 250 μg of B cell depleting anti-CD20. 48 hours later they were given the first IV dose of HER2-TLR7 at 5 mg/kg. Seven days later a second IV dose of HER2-TLR7 was given, and rectal temperatures were assessed every five-ten minutes. As is shown in FIG. 2, B cell depleted mice were protected from the anaphylactic response.

### Example 18: Anaphylaxis-Like Response in Mice does not Require Mast Cells

Tumor-free female mast cell deficient mice (WBB6F, 1/J-KitW/KitW-v/J) and their wild-type litermates (Jackson Laboratory) were administered HER2-TLR7 IV at 5 mg/kg on days 0 and 7. Rectal temperatures were assessed every five-ten minutes following the second dose. A significant, sustained temperature drop is a surrogate indicator of anaphylaxis in mice.

As shown in FIG. 3, both wild-type (3A) and mast cell deficient (3B) mice showed clinical symptoms of anaphylaxis following IV administration of HER2-TLR7. These results suggest that mast cells are not required for the anaphylaxis response observed in mice following IV administration of HER2-TLR7.

### Example 19: Macrophages/Monocytes are Required for Anaphylactic Response to Repeat IV Dosing of HER2-TLR7

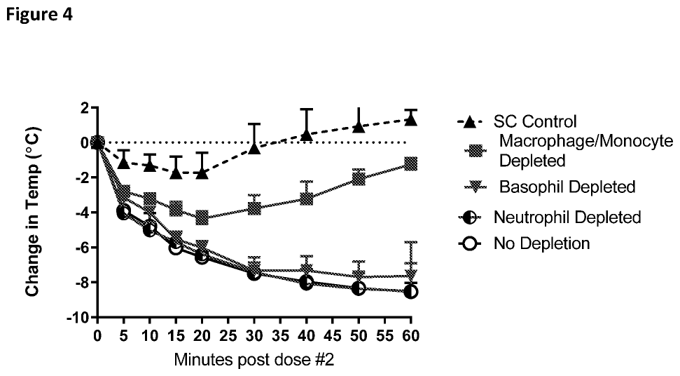

Because mast cells were not required for the anaphylactic response, we next asked which other effector cells could be responsible. Tumor-free female Balb/c mice (Jackson Laboratory) were administered two IV doses of HER2-TLR7 at 5 mg/kg, seven days apart. 24-48 hours prior to the second dose, effector cells were depleted by IV administration of 150 μL clodronate liposomes (macrophages/monocytes), 25 μg anti-CD200R3 clone Ba103 (basophils) or 500 μg anti-Ly6G clone 1A8 (neutrophils). Rectal temps were monitored every 5-10 minutes following the second dose. As shown in FIG. 4, only macrophage/monocyte depletion protected mice from the anaphylactic response.

### Example 20—ADAs Against HER2-TLR7 are Detected in Mouse Plasma, Regardless of the Route of Administration (Old FIGS. 8A-8B)

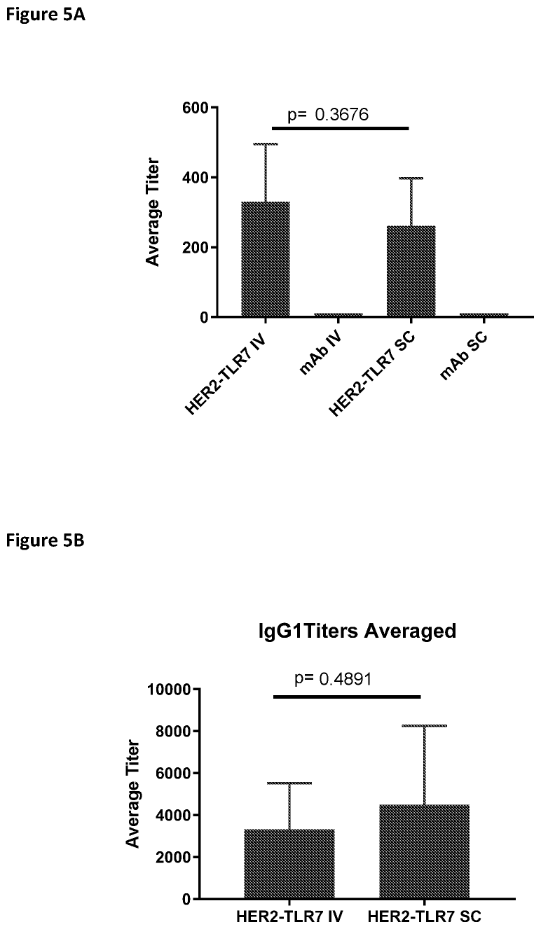

To determine the relevance of anti-drug antibody (ADA) levels to the anaphylactic response, we performed ELISAs to compare ADA levels generated following IV or SC dosing. Female Balb/c mice (Jackson Laboratories) were administered two doses of HER2-TLR7 or HER2 naked antibody at 5 mg/kg IV or SC, one week apart. Seven days after the second dose, blood was drawn and plasma was analyzed for ADAs using a bridging ELISA. In this example, the HER2 naked antibody was used to capture and detect plasma ADAs. In mice given HER2-TLR7, ADAs were generated against the antibody backbone at equal levels, regardless of the route of administration (FIG. 5A). Importantly, no ADAs were formed when mice were administered naked antibody, underscoring the importance of the adjuvancy of the TLR7 agonist. To further demonstrate this, a second ELISA was performed on these samples to assess IgG1 antibody levels against HER2-TLR7. In this assay, HER2-TLR7 was used to capture plasma ADAs and anti-mouse IgG1 was used for detection. ADAs were generated to approximately the same titer following IV and SC administration of HER2-TLR7, suggesting that the observed responses were not explained by ADA level alone (FIG. 5B).

### Example 21: Anaphylactic Response is not Due to Cmax, but is Associated with a Fast Tmax

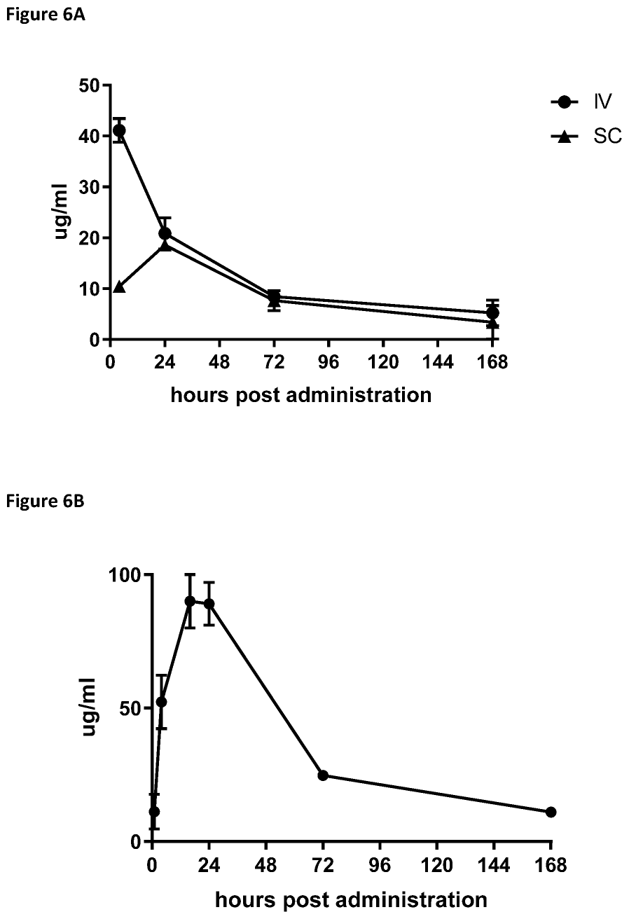

Pharmacokinetic parameters, such as the time to peak plasma level (Tmax) and peak plasma concentration (Cmax), differ between the SC and IV routes of administration. To test the relevance of these parameters to the lack of anaphylactic response observed with SC administration, we performed PK analysis on CT26-Her2 tumor-bearing Balb/c mice (Jackson Laboratory) administered HER2-TLR7 at 5 mg/kg either IV or SC (FIG. 6A) and at 50 mg/kg SC (FIG. 6B). Blood was drawn at 4, 24, 72 hours, and 7 days post-injection. Plasma levels of HER2-TLR7 were assayed by ELISA. As is shown in Table 4, peak plasma level of HER2-TLR7 is reached at 4 hours after IV injection and at 24 hours after SC injection. Cmax at 5 mg/kg in the IV-dosed animals is approximately twice that of SC-dosed mice. Importantly, the Cmax in mice given a 50 mg/kg SC dose, a level that did not result in anaphylaxis when administered repeatedly to tumor-bearing mice (data not shown), is approximately 2.2 times that of the Cmax in the animals administered 5 mg/kg IV (90 vs 41 ug/mL). Taken together, this suggests that the anaphylactic response is not due to Cmax, but is associated with a rapid Tmax.

TABLE 4

| Dose Level | | | | AUC |
|---|---|---|---|---|
| (mg/kg) | Route | Cₘₐₓ (μg/mL) | Tₘₐₓ (h) | (h*μg/mL) |
| 5 | IV | 41 | 4 | 2563 |
| 5 | SC | 19 | 24 | 1698 |
| 50 | SC | 90 | 24 | 6593 |

Example 22: Neutralization of PAF or Histamine Reduces Anaphylaxis-Like Response in Mice

Upon binding antigen-antibody complexes, effector cells such as mast cells, basophils, neutrophils, monocytes, and macrophages are triggered to release PAF and/or histamine, chemical mediators that increase vascular permeability and vasodilation associated with anaphylaxis. To determine which of these mediators is involved in the anaphylactic response, tumor-free female Balb/c mice (Jackson Laboratory) were administered two doses of IV HER2-TLR7 at 5 mg/kg, 7 days apart. A PAF inhibitor, CV6209, at 200 μg per mouse (IP), or an anti-histamine, triprolidine-HCl, at 125 μg per mouse (IP), were given 30 minutes prior to the second dose. Rectal temperatures were taken every five-ten minutes for one hour. Additionally, clinical scores were assessed using the following criteria in Table 5.

TABLE 5

| Clinical Score | Description |
|---|---|
| 0 | Normal |
| 1 | Hunched, lethargic |
| 2 | Fully immobilized |
| 3 | Severe, sustained temp drop |
| | requiring euthanasia |
| 4 | Spontaneous Death |

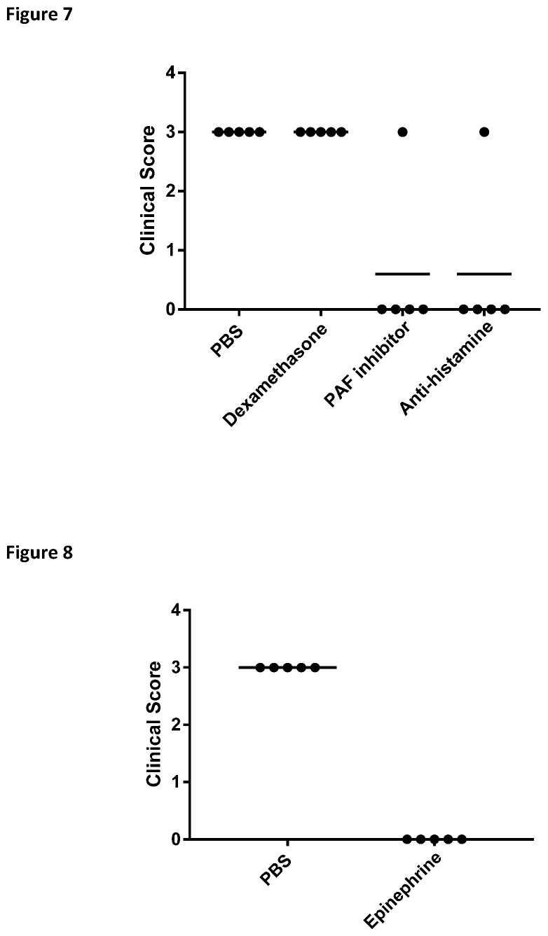

As shown in FIG. 7, neutralization of PAF and histamine prior to IV administration of HER2-TLR7 mitigates the toxicity.

### Example 23: Epinephrine, but not Dexamethasone, Reduces Anaphylactic Response in Mice

Epinephrine is commonly used to treat anaphylactic shock. To determine if the anaphylactic response in mice is mitigated by epinephrine, tumor-free female Balb/c mice (Jackson Laboratory) were administered two doses of IV HER2-TLR7 at 5 mg/kg, 7 days apart. IV epinephrine was administered 5 minutes following the second dose at 10 μg per mouse. Rectal temperatures and clinical scores were assessed as previously shown.

As shown in FIG. 8, administration of epinephrine 5 minutes after IV administration of HER2-TLR7 mitigates the toxicity, whereas a prophylactic dose of the anti-inflammatory agent, dexamethasone, at 60 μg per mouse SC had no effect (FIG. 7).

### Example 24: Repeat-Dose Anaphylaxis is Driven by Non-Self Reactivity in Mice

Tumor-free female Balb/c mice (Jackson Laboratory) were administered a SC dose of HER2-TLR7 at 5 mg/kg, and seven days later were administered an IV dose of HER2-TLR7 at 5 mg/kg, an IV dose of naked anti-HER2 antibody at 5 mg/kg, an IV dose of a mouse naked antibody directed to a non-HER2 cancer antigen (Mouse Antibody 1) at 5 mg/kg, or an IV dose of a TLR7 agonist conjugated to Mouse Antibody 1 at 5 mg/kg. As a negative control, some mice were given a second SC dose of HER2-TLR7 at 5 mg/kg. Rectal temperatures were assessed every five-ten minutes post-second dose. A significant, sustained temperature drop is a surrogate indicator of anaphylaxis in mice. Mice administered IV HER2-TLR7, naked anti-HER2 antibody, or the TLR7 agonist conjugated to Mouse Antibody 1 exhibited an anaphylaxis-like response, while the mice administered SC HER2-TLR7 or IV Mouse Antibody 1 did not. These results suggest that anaphylaxis is driven by non-self reactivity to the humanized components of HER2-TLR7 or to the linker payload itself, but not to the mouse components of either antibody. (Data not shown).

### Example 25: Improved Survival in Her2-CT26 Tumor Bearing Mice Treated with HER2-TLR7

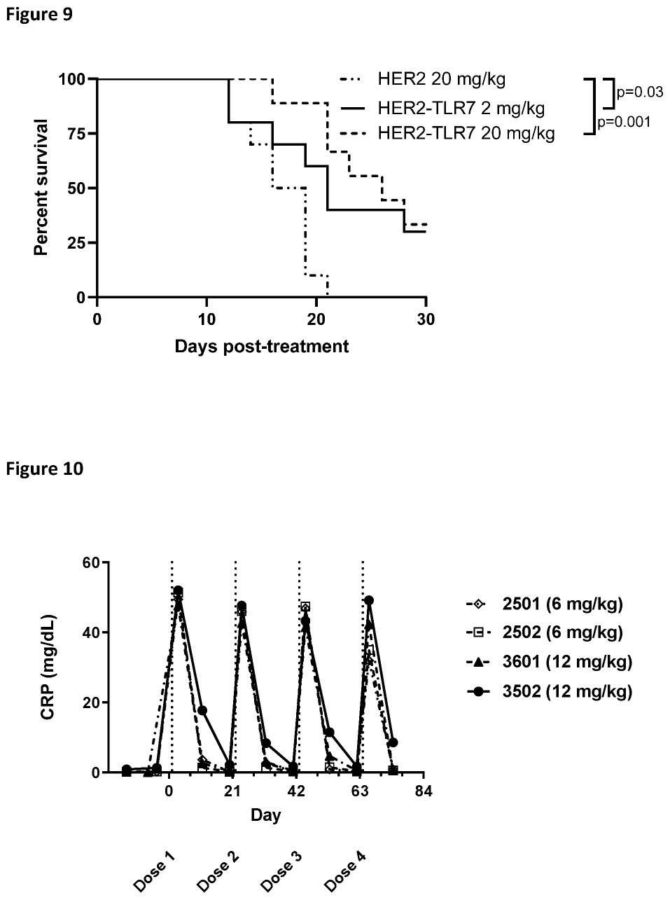

To avoid potential interference by anti-drug antibodies against the test article, we performed an efficacy experiment in Jh mice (Taconic), a B cell deficient strain with a Balb/c background. After tumor formation, mice were administered anti-HER2 antibody at 20 mg/kg or HER2-TLR7 agonist at 20 and 2 mg/kg, QW for 4 weeks, SC. Kaplan-Meier survival curves demonstrate improved survival after subcutaneous administration of 20 mg/kg or 2 mg/kg HER2-TLR7 when compared to HER2 antibody alone (FIG. 9)

### Example 26: Repeat-Dose Intravenous Administration of an Immune-Stimulatory Conjugate Containing a Benzazepine TLR8 Agonist to Non-Human Primates Results in an Acute Anaphylaxis-Like Reaction

This example shows that repeat intravenous administration of a bolus of HER2-TLR8 results in an acute anaphylaxis-like response in cynomolgus monkey. The monkey used in this study was purpose-bred, treatment-naïve, and housed and treated at Charles River Laboratories in Reno, Nev., in accordance with US FDA GLP regulations. The target age and weight of the animal at time of dosing was 2 to 4 years and 2.5 to 3.5 kg, respectively, and the animal used on study was female. HER2-TLR8 was evaluated on a repeat dose schedule as follows: 1 mg/kg was dosed on Day 1, followed by a 7.5 mg/kg dose administered one week later on Day 8 and a third 7.5 mg/kg dose administered three weeks later on Day 29. After each dose, the animal was observed cage-side for clinical signs and symptoms associated with dosing. The first two doses, occurring one week apart, did not produce an anaphylaxis-like response. The administration of the third intravenous dose of HER2-TLR8 on Day 29 resulted in mortality. This animal was euthanized due to the rapid onset of clinical signs of pale mucus membranes and face, hunched posture, decreased activity, hypothermia (as assessed by being cool to the touch), and labored, shallow breathing. These clinical signs presented within 2.5 hour following dosing on Day 29. The nature of the clinical observations and timing in relation to dosing were suggestive of a potential anaphylaxis-like response.

### Example 27: Repeat-Dose Subcutaneous Administration of an Immune-Stimulatory Conjugate Containing a Benzazepine Agonist to Non-Human Primates does not Produce an Acute Anaphylaxis-Like Reaction

This example shows that repeat subcutaneous administration of HER2-TLR8 does not result in an acute anaphylaxis-like response in cynomolgus monkey. The monkey used in this study was purpose-bred, treatment-naïve, and housed and treated at Charles River Laboratories in Reno, Nev., in accordance with US FDA GLP regulations. The target age and weight of the animal at time of dosing was 2 to 4 years and 2.5 to 3.5 kg, respectively, and the animal used on study was female. HER2-TLR8 was evaluated on a repeat dose schedule as follows: 2 mg/kg dose was subcutaneously administered on a Q2W dosing schedule over 4 dosing cycles, 6 mg/kg was subcutaneously administered on a Q3W dosing schedule over 4 dosing cycles and 12 mg/kg was subcutaneously administered on a Q3W dosing schedule over 4 dosing cycles. After each dose, the animal was observed cage-side for clinical signs and symptoms associated with dosing. HER2-TLR8 was well-tolerated at all dose levels and through all dosing cycles. No clinical, anaphylaxis-like signs or symptoms were observed after subcutaneous dosing of HER2-TLR8 through the hours and days following either the first or repeat dose at any dose level.

### Example 28: Repeat-Dose Subcutaneous Administration of an Immune-Stimulatory Conjugate Containing a Benzazepine Agonist to Non-Human Primates does not Produce an Acute Anaphylaxis-Like Reaction at Pharmacologically Active Drug Levels as Measured by CRP

Repeat-dose subcutaneous administration of HER2-TLR8 to non-human primates at 2 mg/kg, 6 mg/kg, and 12 mg/kg result in a consistent pharmacodynamic response demonstrating active drug exposure with each dosing cycle. Blood samples were collected by venous puncture at various timepoints and analyzed for blood chemistries, including C-reactive protein (CRP), using a standard blood analyzer. As shown in FIG. 10, HER2-TLR8 results in a consistent, modest elevation of C-reactive protein (CRP) with each dosing cycle.

### Example 29: Repeat-Dose Subcutaneous Administration of an Immune-Stimulatory Conjugate Containing a Benzazepine Agonist to Non-Human Primates does not Produce an Acute Anaphylaxis-Like Reaction at Drug Exposure Levels Equivalent to Those Associated with Toxicity Upon Repeat-Dose IV Administration

The pharmacokinetic profile of a single dose of HER2-TLR8 administered subcutaneously at 2, 6, or 12 mg/kg or intravenously at 7.5 mg/kg in cynomolgus monkey was studied. Blood was collected by venous puncture at a series of timepoints pre and post-dosing. Serum was prepared and HER2-TLR8 was measured by an ELISA assay in which serum samples were added to an assay plate coated with recombinant HER2 and HER2-TLR8 was detected using a labelled antibody directed against the TLR8 payload. As shown in Table 6, similar levels of HER2-TLR8 were reached in serum with either a 12 mg/kg dose delivered subcutaneously or a 7.5 mg/kg dose delivered intravenously.

TABLE 6

| RoA | Dose Level | AUC (h*ug/mL) |
|---|---|---|
| IV | 7.5 mpk | 3774.91 |
| SubC | 2 mpk | 714.18 |
| | 6 mpk | 2399.8 |
| | 12 mpk | 3654.3 |

### Example 30: Repeat-Dose Subcutaneous Administration of an Immune-Stimulatory Conjugate Containing a Benzazepine Agonist to Non-Human Primates does not Produce an Acute Anaphylaxis-Like Reaction at Drug Exposure Levels Equivalent to Those Associated with Toxicity Upon Repeat-Dose IV Administration

The ability of a TLR7 antibody conjugate to alter tumor cell growth in mouse syngeneic tumor was assessed as follows. Six to seven-week-old Balb/cJ mice were inoculated subcutaneously (SC) in the mammary fat pad with 1×10⁵ HER2+EMT6 cells. Six days later, tumors were measured with calipers and volume was calculated using the formula: Volume=((Minimum Length)²×(Maximum Length))/2. Mice with tumor volumes ranging from 44.25 to 175.71 mm³ were organized into three groups of 10 with average tumor size 97.14 mm³. Mice were administered anti-HER2 mAb (mIgG2a) at 10 mg/kg, anti-HER2 conjugated to cleavable linker-TLR7 agonist (HER2-TLR7, structure as shown in Example 15 above) at 10 mg/kg, or PBS, SC, once weekly for four weeks. Tumor volumes were measured three times per week. Mice were euthanized when tumor volumes reached 1500 mm³ or if the tumors metastasized. The study was terminated approximately five weeks after the first dose (day 34). Volumes and survival were plotted using GraphPad Prism. Survival curves were analyzed using the Log rank (Mantel-Cox) test. p<0.05 was considered statistically significant.

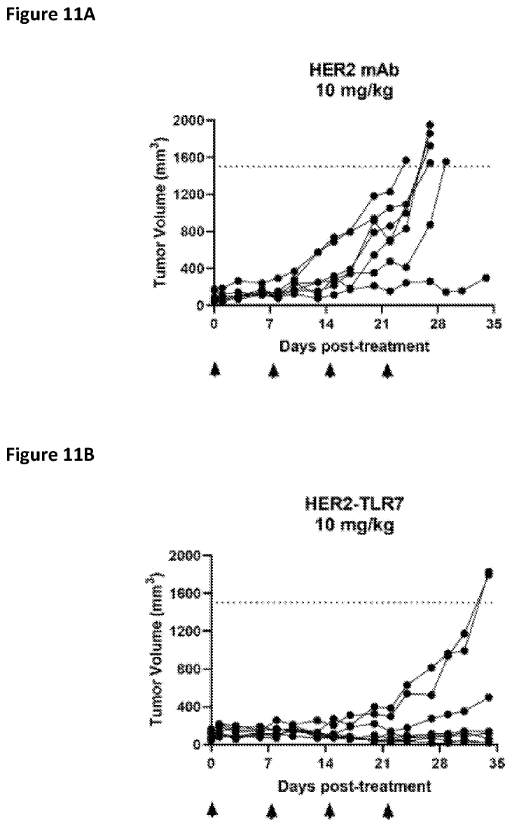

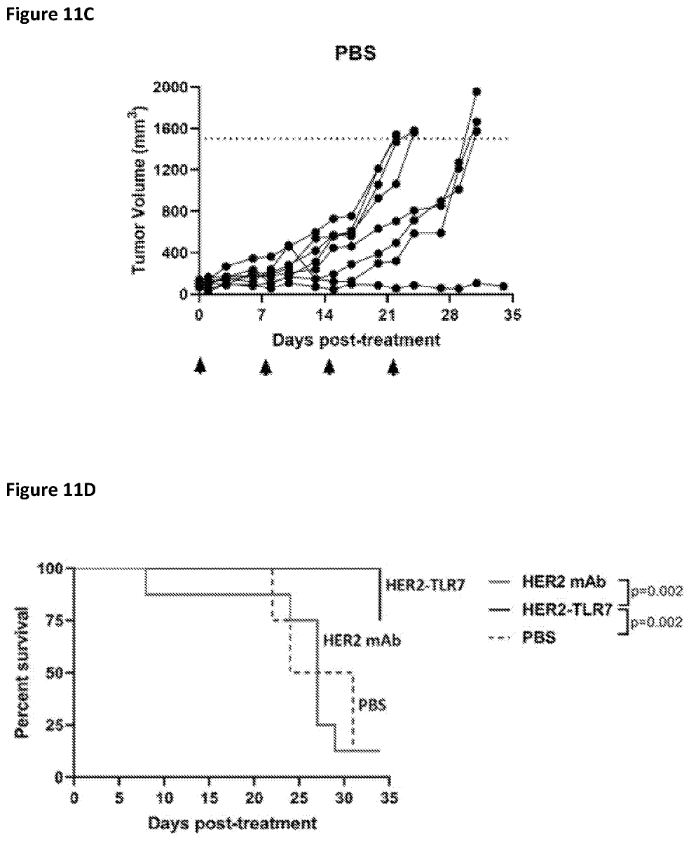

The cohort treated with the HER2-TLR7 agonist conjugate showed slowed tumor growth (compare FIG. 11B to FIGS. 11A and 11C) and a significant survival advantage (FIG. 11D) compared to HER2 and PBS controls.

### Example 31: Mice that have Cleared HER2pos CT26 Tumors in Response to HER2-TLR7 Reject HER2pos CT26 Tumors Upon Re-Challenge

These studies were designed to test the durability of the anti-tumor responses in mice treated with HER2-TLR7 (structure as shown in Example 15 above). Mice inoculated with HER2 positive CT26 colon carcinoma cells were treated with HER2-TLR7 or unconjugated HER2 mAb SC at 5 mg/kg and 20 mg/kg. Mice that had completely cleared tumors with HER2-TLR7 treatment were re-challenged with the same HER2 positive CT26 cell line approximately 60 days after primary tumor clearance. The half-life of the surrogate is approximately 48 hours and is no longer present at the time of re-challenge. HER2-TLR7 conjugate treated mice for re-challenge were obtained as follows. BALB/cJ mice were inoculated subcutaneously in the right flank with 5×105 HER2+CT26 cells in PBS. Fourteen days later, tumors were measured with calipers and volume was calculated using the formula: Volume=((Minimum Length)²×(Maximum Length))/2. Mice were sorted into control and treatment cohorts with size-matched tumors. The mice were treated with PBS, 5 mg/kg anti-HER2 antibody, 5 mg/kg anti-HER2-TLR7 conjugate, 20 mg/kg anti-HER2 antibody, or 20 mg/kg anti-HER2-TLR7 conjugate.

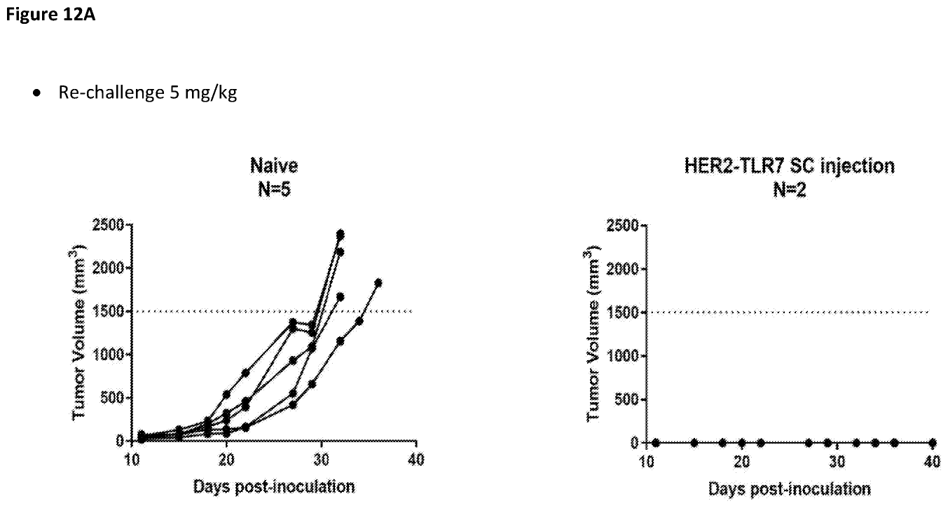

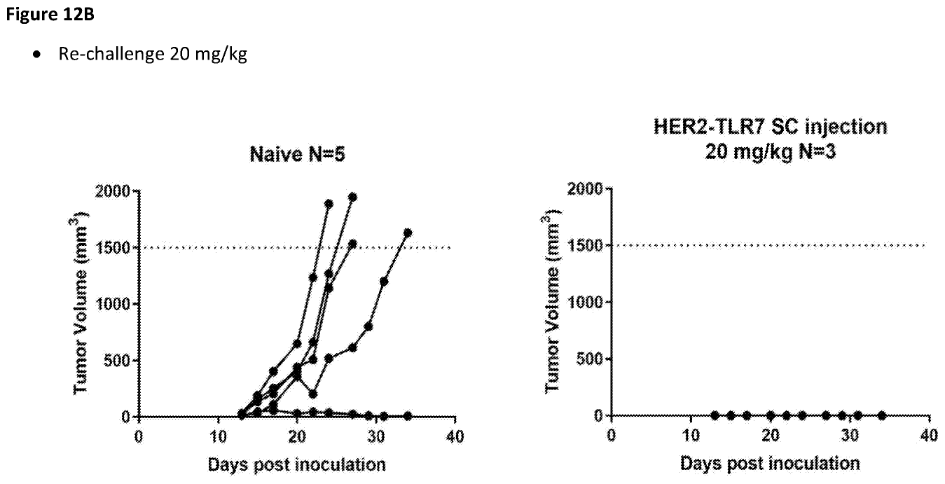

Some 5 mg/kg and 20 mg/kg conjugate-treated animals cleared their tumors, 25% and 30% respectively, while there were no clearances in the unconjugated HER2 antibody or PBS groups. Those exhibiting complete clearance were re-challenged with 5×105 HER2+CT26 cells injected into the left flank along with cohorts of naïve animals that were similarly challenged. Results are shown in FIG. 12A (re-challenge of 5 mg/kg treated mice vs. naïve) and FIG. 12B (re-challenge of 20 mg/kg treated mice vs. naïve). Mice re-challenged with HER2 positive CT26 tumors were 100% protected, indicating that the HER2-TLR7 surrogate can induce a durable anti-tumor memory response at a dose as low as 5 mg/kg.

### Example 32: Mice that have Cleared HER2+CT26 Tumors in Response to HER2-TLR7 Reject HER2neg CT26 Tumors Upon Re-Challenge

To test the durability and epitope spreading of the anti-tumor responses in mice treated with HER2-TLR7 conjugate, mice that had completely cleared tumors with HER2-TLR7 treatment (HER2-TLR7, structure as shown in Example 15 above) were re-challenged with wild-type (HER2 negative) CT26 cells in the left flank 60 days after primary tumor clearance. The mice for re-challenge were obtained as follows. Female BALB/cJ mice were inoculated SC in the right flank with 5×10⁵ HER2⁺ CT26 cells in PBS. Fourteen days later, tumors were measured with calipers and volume was calculated using the formula: Volume=((Minimum Length)²×(Maximum Length))/2. Mice with tumor volumes ranging from 96.5 to 146.3 mm³ were organized into two groups of 10 with average tumor size 126.8 mm³. Cohorts of 10 mice were treated SC with PBS or 50 mg/kg anti-HER2-TLR7 conjugate qW×4. The HER2-TLR7 conjugate-treated mice that were tumor-free (30%) were then inoculated SC after approximately 60 days on the left flank with 5×10⁶ HER2-negative CT26 cells. As a control, a cohort of naïve BALB/cJ mice were similarly inoculated with Her2-negative CT26 cells.

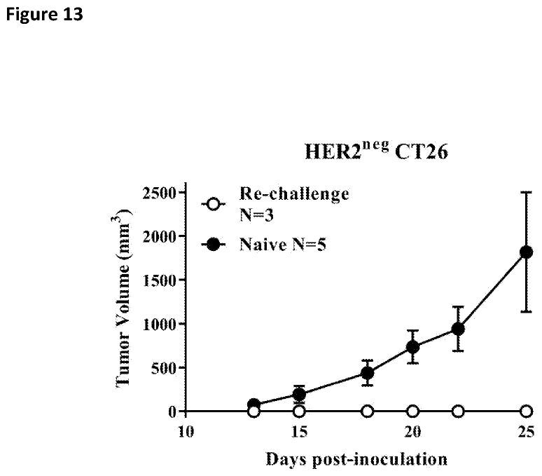

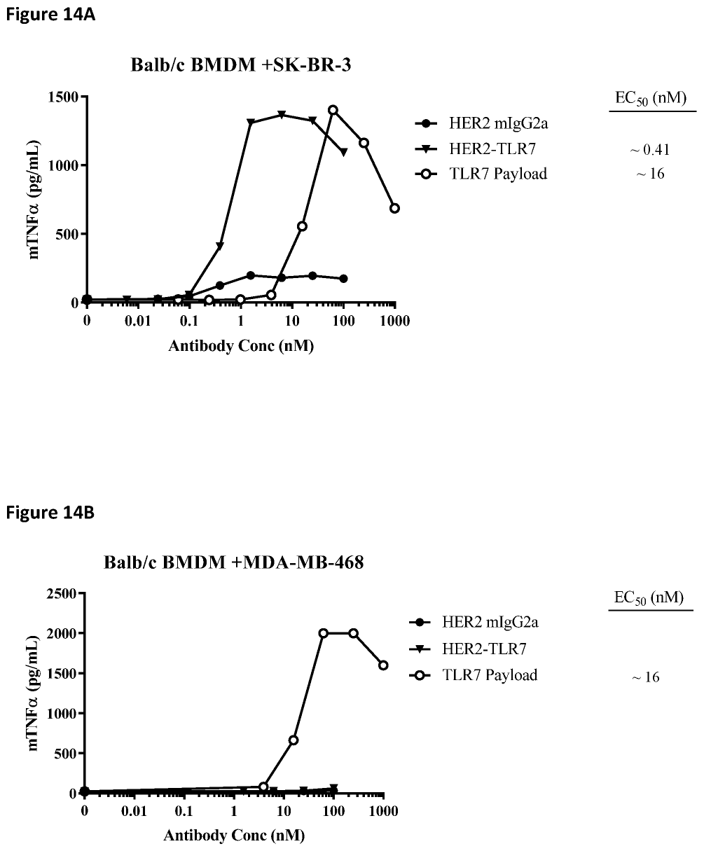

Unlike the naïve controls, all the re-challenged mice were protected from growth of wild-type CT26 tumor cells, indicating a durable and broad neo-antigen T cells response that is independent of HER2. Results are shown in FIG. 13.

### Example 33: HER2-TLR7 Induces TNF-α from Mouse Bone Marrow-Derived Macrophages in the Presence of HER2pos Cells

The ability of an anti-HER2-TLR7 conjugate to specifically activate mouse macrophages when bound to tumor cells by HER2 was assessed in vitro as follows. Bone marrow cells were harvested from BALB/cJ mouse femurs and tibias using a 27-gauge needle attached to a 3 mL syringe filled with growth media (DMEM supplemented with 10% Fetal Bovine Serum, 1 mM Sodium Pyruvate, 1× GlutaMAX-1, 1× Non-Essential Amino Acids, 10 mM HEPES and 0.5% Penicillin/Streptomycin). Bone marrow cells were centrifuged, and RBC were lysed before being counted and resuspended at a concentration 5×10⁵/mL in growth media. Ten mL of cell suspension was placed in 10 cm dishes and 20 ng/mL murine macrophage-colony-stimulating factor (mM-CSF) was added. Cells were incubated for two days, media was replaced with fresh growth media containing 20 ng/mL mM-CSF, and then cells were cultured for a further four days. Bone marrow derived macrophage cells lines (BMDM) and tumor cell lines SK-BR-3 (HER2pos) or MDA-MB-468 (HER2neg) were removed from plates with Accutase cell detachment solution and counted. BMDM were plated in 96-well flat bottom microtiter plates at 80,000 cells/well in assay media (RPMI-1640 Medium supplemented with 10% Fetal Bovine Serum, 1 mM Sodium Pyruvate, 1× GlutaMAX-1, 1× non-essential Amino Acids, 10 mM HEPES and 0.5% Penicillin/Streptomycin). Tumor cell lines were plated at 40,000 cells/well in assay media along with 100-0.001 nM HER2-TLR7 conjugate (HER2-TLR7, structure as shown in Example 15 above), or anti-HER2 m1gG2a, or 1000-0.001 nM TLR7 payload compound, and incubated together for 24 hours at 37° C., 5% CO₂. The TLR7 payload compound was 2-amino-N-((1-(4-amino)-2-(ethoxymethyl)-1H-imidazo[4,5-c]quinolin-1-yl)-2-methylpropan-2-yl)oxy)ethyl)-2-methylpropanamide (structure shown below).

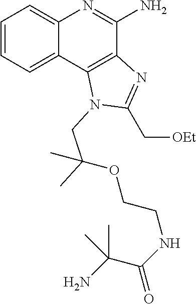 [SMILES: CCOCc1nc2c(N)nc3ccccc3c2n1CC(C)(C)OCCNC(=O)C(C)(C)N]

¹H NMR (DMSO, 400 MHz) δ 14.08 (bs, 1H), 9.14 (bs, 2H), 8.53 (d, 1H, J=8.0 Hz), 8.08 (bs, 3H), 8.03 (t, 1H, J=5.6 Hz), 7.78 (dd, 1H, J=8.4, 1.2 Hz), 7.70 (td, 1H, J=7.2, 1.2 Hz), 7.58 (td, 1H, J=7.2, 1.2 Hz), 4.84 (bs, 4H), 3.54 (q, 3H, J=6.8 Hz), 3.23 (t, 2H, J=6.4 Hz), 2.96 (m, 2H), 1.35 (s, 3H), 1.19 (bs, 3H), 1.13 (t, 3H, J=6.8 Hz). LCMS (M+H)=443.6.

After culture, supernatants were collected and frozen at −80° C. until cytokine analysis was performed. Murine TNFα (mTNFα) levels in the supernatant were determined by mTNFα ELISA Kit (BioLegend) and read on an Envision Plate Reader (Perkin Elmer, Waltham, Mass.) according to manufacturer's instructions. mTNFα levels were then graphed using GraphPad Prism 7.01 software (GraphPad Software, San Diego, Calif.) and EC₅₀ values were generated using non-linear regression curve fit.

The anti-HER2-TLR7 conjugate potently activated the mouse bone marrow cells when bound to the HER2pos cell line but not when unbound in the presence of the HER2neg cell line. The TLR7 payload compound was capable of potently activating the macrophages in the presence of both cell lines. Results are shown in FIGS. 14A (BMDM+SK-BR-3) and 14B (BMDM+MDA-MB-468).

### Example 34: Elevated Intratumoral Cytokines, Chemokines, and Infiltration/Activation of Immune Cells in HER2+CT26 Tumor Bearing Mice after Treatment with HER2-TLR7

To demonstrate the ability of tumor targeted TLR7 immune activation, mice bearing HER2+ tumors were treated with an anti-HER2-TLR7 conjugate (HER2-TLR7, structure as shown in Example 15 above) or anti-HER2 antibody control, and tumors were excised and analyzed for immune activation by measuring immune cells, cytokines, and chemokines. Six to eight-week-old BALB/cJ mice were inoculated SC in the right flank with 5×10⁵ HER2+CT26 cells. Seventeen days later, tumors were measured with calipers and volume was calculated using the formula: Volume=((Minimum Length)²×(Maximum Length))/2. Mice with tumor volumes ranging from 120.4 to 314.9 mm³ were organized into 4 groups of 6 to 7 with average tumor size of 213.2 mm³. Mice were administered HER2 mAb or HER2-TLR7 IV at 5 mg/kg and tumors were harvested on the schedule outlined in Table 7. Intratumoral cytokines and chemokines were assayed by Luminex and infiltrating immune cells were assessed by flow cytometry as follows. For Luminex analysis, tumors were weighed, placed into 500 μL RPMI and mechanically dissociated on ice. The resulting supernatants were stored at −80 OC for future analysis. Data was expressed as picogram of analyte per gram of starting tissue. A subset of tumors were also enzymatically digested using the Miltneyi mouse digest kit and filtered through a 70 m filter. Single cell suspensions were divided across three flow cytometry panels. For intracellular T cell analysis, cells were stimulated with 2 μM AH-1 peptide (AnaSpec (AS-64798)) in the presence of 1× brefeldin A for 4 hours at 37° C., stained for surface markers, permeabilized with FoxP3 Staining Buffer (eBioscience), and stained with antibodies against IFNγ, IL-1α, MCP-1, MIP1α, IL-6, IP-10, CXCL1, and CXCL2 at various timepoints. All data was analyzed in GraphPad Prism. In some cases, HER2-TLR7-treated tumor material was limiting and was not available for all analyses.

TABLE 7

| | Test | | |
|---|---|---|---|
| Group | Materials | Dose, Route and Schedule | N |
| A | HER2 mAb | 5 mg/kg, IV, 1x, harvest at 48 h | 7 |
| B | HER2-TLR7 | 5 mg/kg, IV, 1x, harvest at 48 h | 6-7* |
| C | HER2 mAb | 5 mg/kg, IV, 3x, days 0, 2, 4. Harvest day 6 | 6 |
| | | (48 hours post-dose #3) | |
| D | HER2-TLR7 | 5 mg/kg, IV, 3x, days 0, 2, 4. Harvest day 6 | 5-6* |
| | | (48 hours post-dose #3) | |

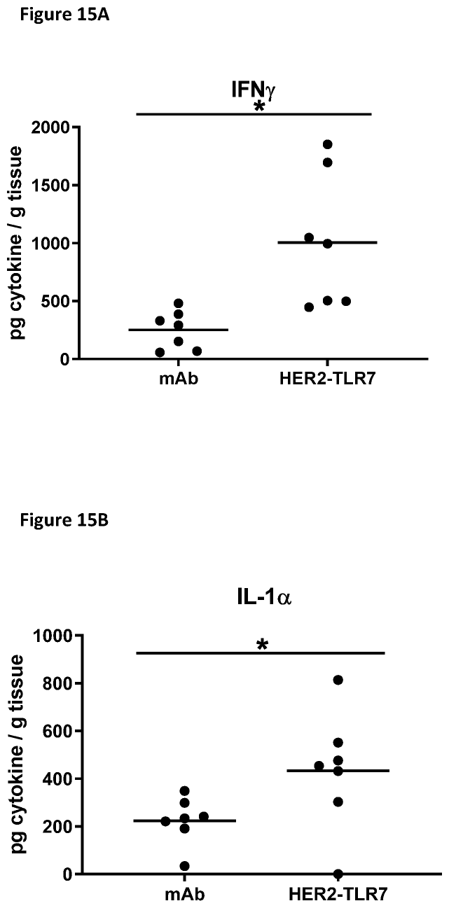

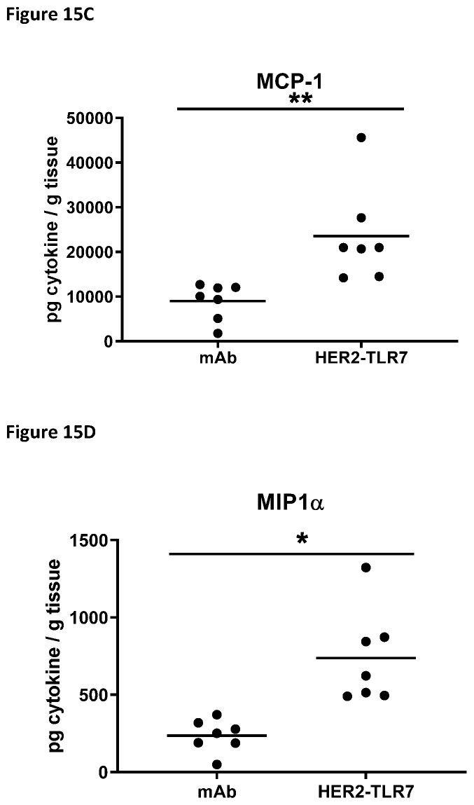

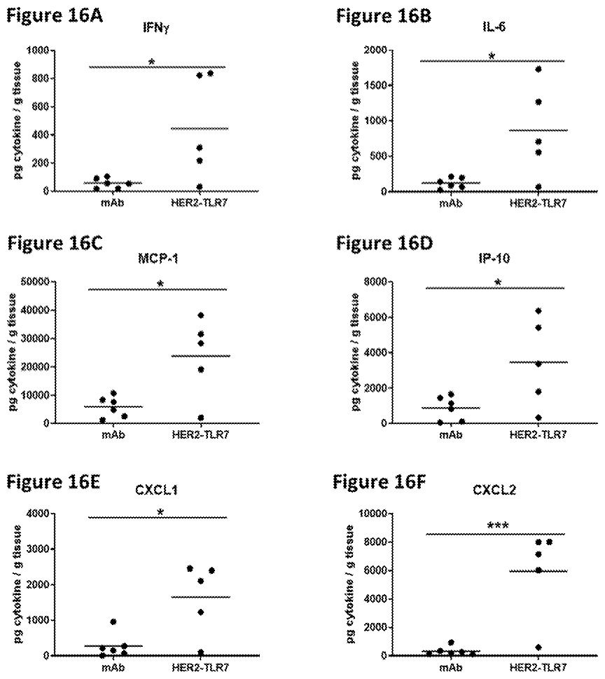

Compared to controls, intratumoral levels of the indicated chemokines and cytokines were found to be elevated 48 hours post a single dose (FIG. 15A, IFNγ; 15B, IL-1α; 15C, MCP-1; 15D, MIP1α) or three doses (FIG. 16A, IFNγ; 16B, IL-6; 16C, MCP-1; 16D, IP-10; 16E, CXCL1; and 16F, CXCL2) of the anti-HER2-TLR7 conjugate, indicating increased immune activation. Statistical significance was determined by unpaired T-test (*p<0.05, **p<0.01, p<0.001).

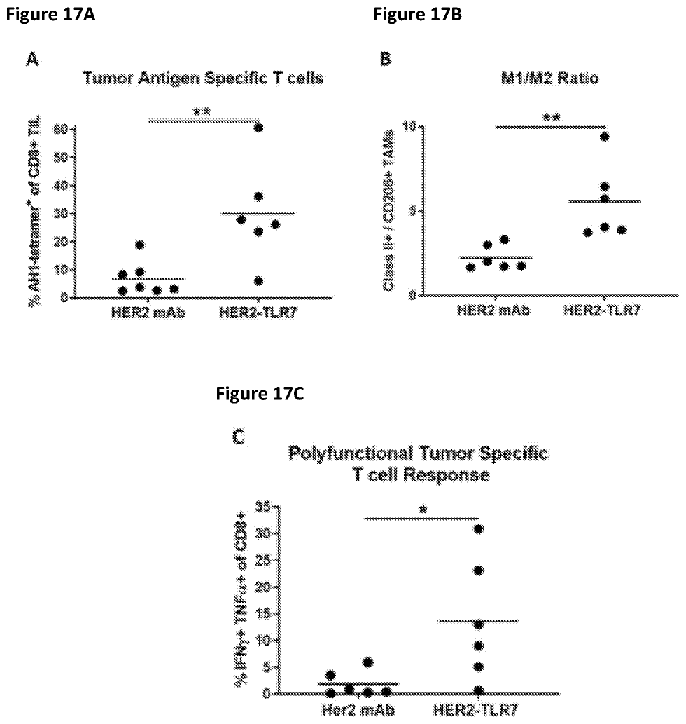

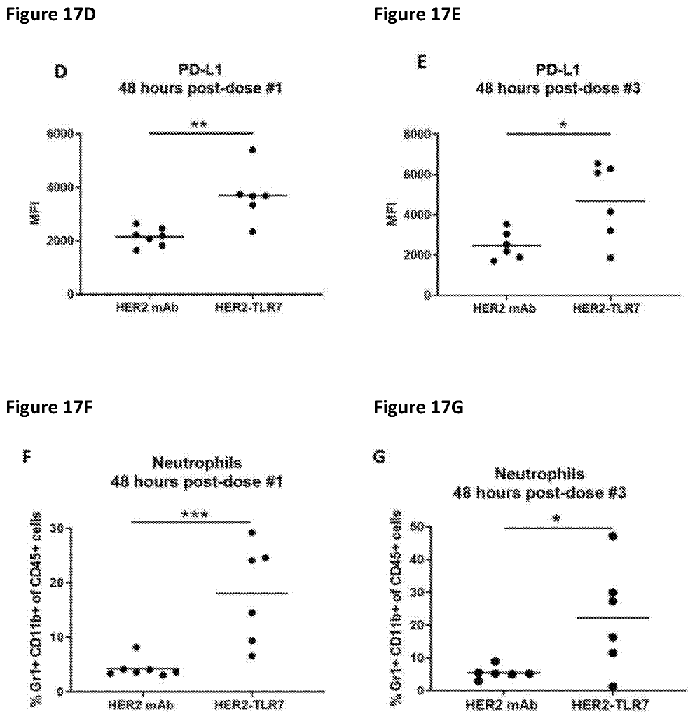

Compared to controls, FACS analysis indicated intratumoral innate and adaptive immune cell activation was increased 48 hours post a single dose or three doses (day 6). By 48 hours, an expanded AH-1+ tumor antigen T cell population was identified by tetramer staining (FIG. 17A). At day 6 there was an increase in the macrophage M1 to M2 ratio (MHC Class II+: CD206+) (FIG. 17B) and an expansion of AH-1 responsive CD8 T cells (FIG. 17C). Elevated tumor cell surface PD-L1 expression (FIGS. 17D-E) and neutrophil infiltrate (FIGS. 17F-G) were observed at both timepoints. Statistical significance was determined by unpaired T-test (*p<0.05, **p<0.01, p<0.001.)

Together these data indicate that treatment with the TLR7 conjugate increased broad intratumoral immune activation.

While aspects of the present disclosure have been shown and described herein, it will be apparent to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

1. (canceled)

2. (canceled)

3. (canceled)

4. A method for treating cancer, comprising administering to a human subject with cancer an effective regimen of an immune-stimulatory conjugate comprising (a) an antibody that specifically binds to a tumor antigen or a tumor associated antigen and (b) an immune-stimulatory compound that is a myeloid cell agonist selected from a TLR agonist, a STING agonist, a RIG-I-Like receptor (RLR) agonist, a c-type lectin receptor agonist, and a cytosolic DNA Sensor agonist, wherein the effective regimen comprises at least two cycles of administration of the conjugate to the subject, and wherein the effective regimen results in a Tmax of the immune-stimulatory conjugate in the subject of greater than 4 hours following each administration of the immune-stimulatory conjugate.

5. (canceled)

6. (canceled)

7. A method for treating a viral infection, comprising administering to a human subject with a viral infection an effective regimen of an immune-stimulatory conjugate comprising (a) an antibody that specifically binds to (i) an antigen present on an cell infected with the virus or (ii) a viral antigen from a virus infecting a cell and (b) an immune-stimulatory compound that is a TLR agonist, wherein the effective regimen comprises at least two cycles of administration of the conjugate to the subject, and wherein the effective regimen results in a Tmax of the immune-stimulatory conjugate in the subject of greater than about 4 hours following each administration of the immune-stimulatory conjugate.

8. (canceled)

9. (canceled)

10. (canceled)

11. The method of claim 4, further comprising administering a B-cell depleting agent.

12. (canceled)

13. The method of claim 11, wherein the B-cell depleting agent is an anti-CD19 or anti-CD20 antibody.

14. The method of claim 11, wherein the B-cell depleting agent is administered to the subject:
(a) at the same time as the first administration of the immune-stimulatory conjugate;
(b) within about 14 days, within about 7 days, or within about 1 day of the first administration of the immune-stimulatory conjugate;
(c) within about 24 hours, about 12 hours, about 6 hours, about 4 hours, about 3 hours, about 2 hours, or about 1 hour of the first administration of the immune-stimulatory conjugate; or
(d) prior to administration of the immune-stimulatory conjugate.

15. (canceled)

16. The method of claim 1, wherein B cells are depleted prior to administration of the immune-stimulatory conjugate.

17. The method of claim 4, wherein the effective regimen comprises a total dose of greater than about 0.4 mg/kg of the immune-stimulatory conjugate per cycle.

18. The method of claim 1, wherein the effective regimen comprises three or more administrations of the immune-stimulatory conjugate, and wherein the Tmax of the immune-stimulatory conjugate is greater than about 4 hours following each administration.

19. (canceled)

20. The method of claim 1, wherein the immune-stimulatory conjugate is administered:
(a) subcutaneously at each administration; or
(b) intravenously by a slow infusion, and wherein the effective regimen results in a Tmax of the immune-stimulatory conjugate greater than about 4 hours following each administration.

21. (canceled)

22. The method of claim 20, wherein the effective regimen results in a Tmax greater than 6 hours, greater than about 8 hours, greater than about 10 hours, greater than about 12 hours, or greater than about 15 hours following each dose.

23. The method of claim 1, wherein Tmax is reached at or prior to about 72 hours following each administration.

24.-38. (canceled)

39. The method of claim 1, wherein the myeloid cell agonist is a TLR agonist selected from a TLR7 agonist, a TLR8 agonist, and an agonist of TLR7 and TLR8.

40. (canceled)

41. (canceled)

42. The method of claim 39, wherein the TLR agonist is selected from a benzazepine, an imidazoquinoline, a thiazoloquinoline, an aminoquinoline, an aminoquinazoline, a pyrido[3,2-d]pyrimidine-2,4-diamine, a pyrimidine-2,4-diamine, a 2-aminoimidazole, an 1-alkyl-1H-benzimidazol-2-amine, a tetrahydropyridopyrimidine, a pyrido[3,2-d]pyrimidine, a dihydropyrimidinyl benzazepine carboxamide, a benzo[b]azepine, benzazepine dicarboxamide derivatives with a tertiary amide, benzazepine dicarboxamide derivatives with a secondary amide, a quinazoline, a pyrido[3,2-d]pyrimidine, a diamino-pyrimidine, an amino-quinazoline, a heterocyclic-substituted 2-amino-quinazoline, a diamino-pyrimidine, a piperidino-pyrimidine, an alkylamino-pyrimidine, an 8-substituted benzoazepine, an amino-diazepine, an amino-benzo-diazepine, an amido-indole, an amido-benzimidazole, a phenyl sulfonamide, a dihydropteridinone, a fused amino-pyrimidine, a quinazoline, a pyrido-pyrimidine, an amino-substituted benzazepine, a pyrrolo-pyridine, an imidazo-pyridine derivatives, and an amino-benzazepine, and pharmaceutically acceptable salts thereof.

43.-54. (canceled)

55. The method of claim 1, wherein the immune-stimulatory conjugate is represented by Formula (I): 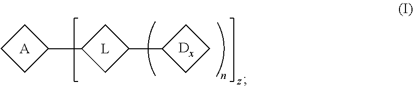
wherein:
A is the antibody,
L is a linker;
Dₓ is the immune-stimulatory compound;
n is selected from 1 to 20; and
z is selected from 1 to 20.

56. The method of claim 55, wherein n is 1 and z is from 1 to 8.

57. (canceled)

58. The method of claim 55, wherein L and Dx together are a compound of Formula (IVC): 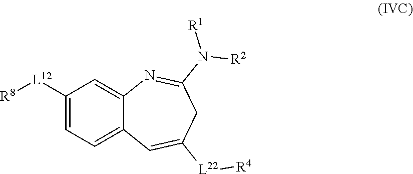
or a pharmaceutically acceptable salt thereof,
wherein:
R¹ and R² are hydrogen;
L²² is —C(O)—
R⁴ is —N(R¹⁰)₂;
R¹⁰ is independently selected at each occurrence from hydrogen, —NH₂, —C(O)OCH₂C₆H₅; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
L¹² is —C(O)N(R¹⁰)—*, wherein * represents where L¹² is bound to R⁸;
R⁸ is an optionally substituted fused 5-5, fused 5-6, or fused 6-6 bicyclic heterocycle bound to linker moiety L³,
and wherein optional substituents are independently selected at each occurrence from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle, and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl.

59. (canceled)

60. (canceled)

61. The method of claim 55, wherein L and Dx together have a structure selected from: 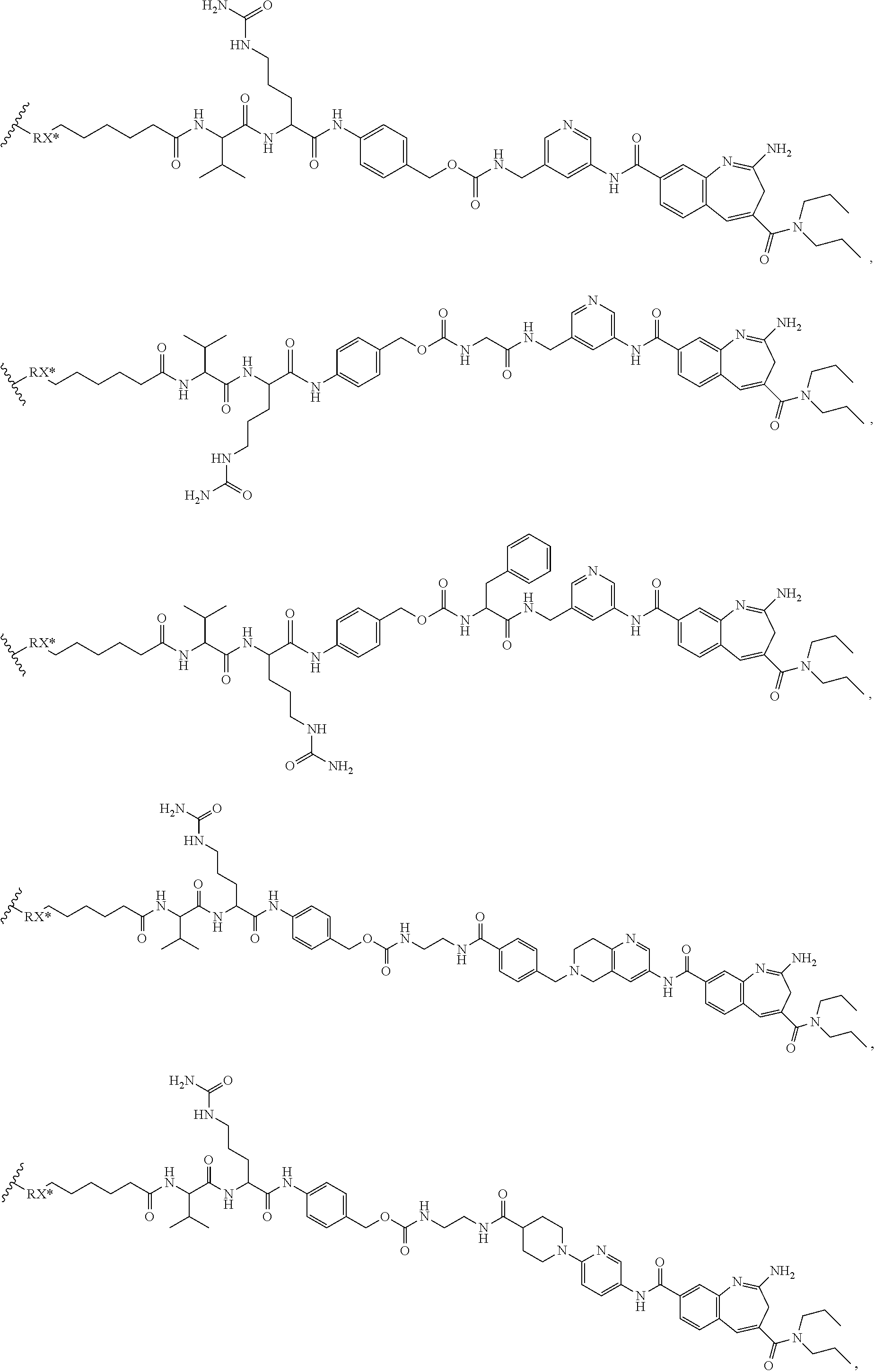 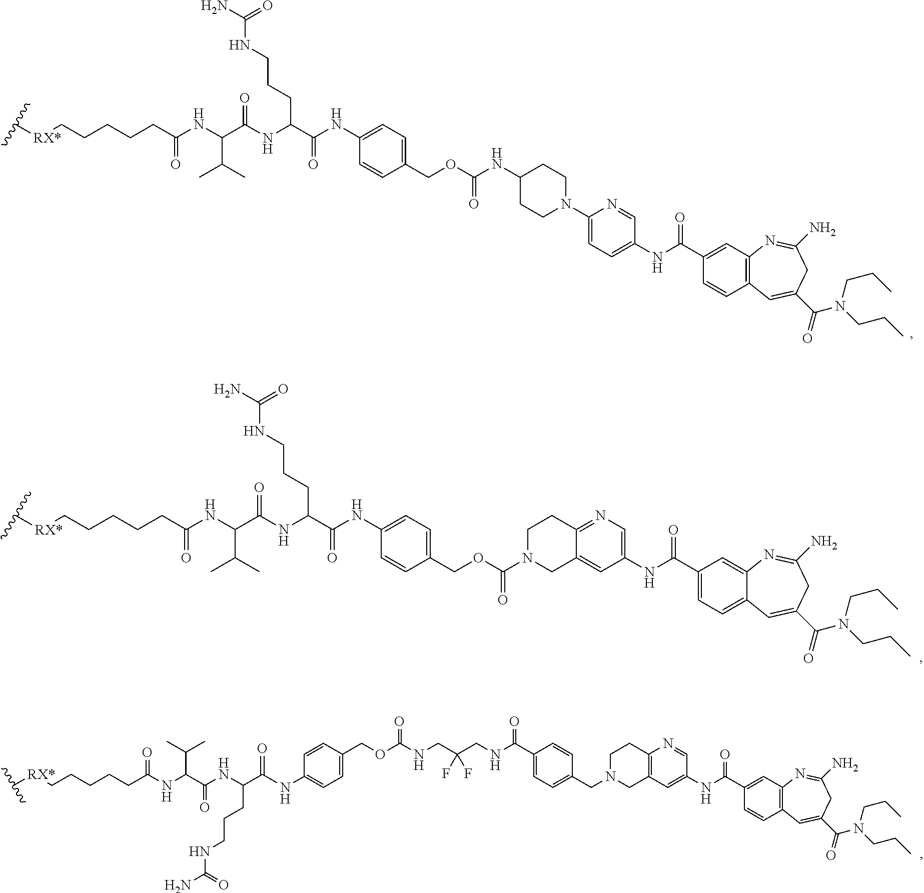
and salts thereof,
wherein the RX* is a bond, a succinimide moiety, or a hydrolyzed succinimide moiety bound to a residue of an antibody construct,
wherein 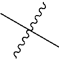
on RX* represents the point of attachment to the residue of the antibody construct.

62. The method of claim 61, wherein L and Dx together have a structure selected from: 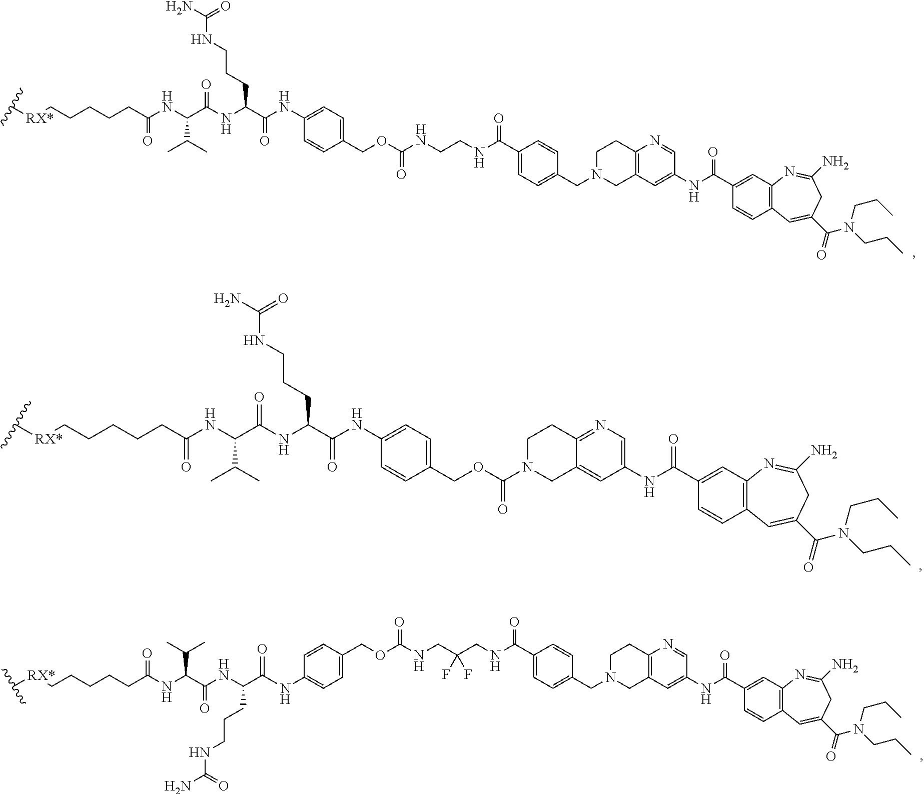
and salts thereof,
wherein the RX* is a bond, a succinimide moiety, or a hydrolyzed succinimide moiety bound to a residue of an antibody construct,
wherein 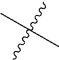
on RX* represents the point of attachment to the residue of the antibody construct.

63.-68. (canceled)

69. The method of claim 1, wherein the tumor antigen or the tumor associated antigen is selected from:
(i) an antigen present on lung cancer, wherein the antigen is optionally selected from mesothelin, HER2, EGFR, PD-L1, MSLN, LY6K, CD56, PTK7, FOLR1, DLL3, SLC34A2, CECAM5, MUC16, LRRC15, ADAM12, EGFRvIII, LYPD3, EFNA4, and MUC1;
(ii) an antigen present on liver cancer, wherein the antigen is optionally selected from GPC3, EPCAM, and CECAM5;
(iii) an antigen present on kidney cancer, wherein the antigen is optionally selected from HAVCR1, ENPP3, CDH6, CD70, and cMET;
(iv) an antigen present on pancreatic cancer, wherein the antigen is optionally selected from PTK7, MUC16, MSLN, LRRC15, ADAM12, EFNA4, MUC5A, and MUC1;
(v) an antigen present on colorectal cancer, wherein the antigen is optionally selected from EPHB2, TMEM238, CECAM5, LRRC15, ADAM12, EFNA4, and GPA33;
(vi) an antigen present on ovarian cancer, wherein the antigen is optionally selected from MUC16, MUC1, MSLN, FOLR1, sTN, VTCN1, HER2, PTK7, FAP, TMEM238, LRRC15, CLDN6, SLC34A2, and EFNA4;
(vii) an antigen present on head and neck cancer, wherein the antigen is optionally selected from LY6K, PTK7, LRRC15, ADAM12, LYPD3, EFNA4 and TNC;
(viii) an antigen present on bone cancer, wherein the antigen is optionally selected from EPHA2, LRRC15, ADAM12, GPNMB, TP-3, and CD248;
(ix) an antigen present on mesothelioma, wherein the antigen is optionally MSLN;
(x) an antigen present on bladder cancer, wherein the antigen is optionally selected from LY6K, PTK7, UPK1B, UPK2, TNC, Nectin4, SLITRK6, LYPD3, EFNA4, and HER2;
(xi) an antigen present on stomach cancer, wherein the antigen is optionally selected from HER2, EPHB2, TMEM238, CECAM5, and EFNA4;
(xii) an antigen present on prostate cancer, wherein the antigen is optionally selected from PSMA, FOLH1, PTK7, STEAP, TMEFF2 (TENB2), OR51E2, SLC30A4, and EFNA4;
(xiii) an antigen present on thyroid cancer, wherein the antigen is optionally PTK7;
(xiv) an antigen present on uterine cancer, wherein the antigen is optionally selected from LY6K, PTK7, EPHB2, FOLR1, ALPPL2, MUC16, and EFNA4;
(xv) an antigen present on cervical/endometrial cancer, wherein the antigen is optionally selected from LY6K, PTK7, MUC16, LYPD3, EFNA4, and MUC1; and
(xvi) an antigen present on breast cancer, wherein the antigen is optionally selected from HER2, TROP2, LIV-1, CDH3 (p-cadherin), MUC1, Sialo-epitope CA6, PTK7, GPNMB, LAMP-1, LRRC15, ADAM12, EPHA2, TNC, LYPD3, EFNA4, and CLDN6;
(xvii) an antigen present on breast cancer, wherein the antigen is optionally selected from HER2, LIV-1, CDH3 (p-cadherin), MUC1, Sialo-epitope CA6, PTK7, GPNMB, LAMP-1, LRRC15, ADAM12, EPHA2, TNC, LYPD3, EFNA4, and CLDN6, or
(xviii) HER2, Nectin4, LRRC15, mesothelin, or PSMA.

70. (canceled)

71. (canceled)

72. The method of claim 1, wherein the method is for treating a HER2 expressing cancer, and the antibody is an anti-HER2 antibody.

73. (canceled)

74. (canceled)

75. The method of claim 1, wherein the antibody is pertuzumab, trastuzumab, sacituzumab, or ladiratuzumab or comprises an antigen binding fragment of pertuzumab, trastuzumab, sacituzumab, or ladiratuzumab.

76. (canceled)

77. (canceled)

78. The method of claim 7, wherein the method is for treating a viral infection and the antigen is ASGR1 or ASGR2.

79. The method of claim 78, wherein the viral infection is HBV or HCV.

80.-88. (canceled)

89. The method of claim 1, wherein the total dose of the immune-stimulatory conjugate administered per cycle of the regimen is from about 0.5 mg/kg to about 7.5 mg/kg.

90.-111. (canceled)
